(19) United States
(12) Patent Application Publication
(10) Pub. No.: US 2020/0390889 A1
(43) Pub. Date: Dec. 17, 2020

(54) REMOTELY TRIGGERED THERAPY

(71) Applicant: Bambu Vault LLC, Lowell, MA (US)

(72) Inventors: Glenn HORNER, West Roxbury, MA (US); Prakash RAI, Lowell, MA (US); Satish AGRAWAL, Sudbury, MA (US); Bethany PARKER, New Ipswich, NH (US)

(73) Assignee: Bambu Vault LLC, Lowell, MA (US)

(21) Appl. No.: 17/000,205

(22) Filed: Aug. 21, 2020

Related U.S. Application Data
(63) Continuation-in-part of application No. PCT/US2020/019348, filed on Feb. 21, 2020.
(60) Provisional application No. 62/852,664, filed on May 24, 2019.
(60) Provisional application No. 62/852,674, filed on May 24, 2019.
(60) Provisional application No. 62/852,659, filed on May 24, 2019.
(60) Provisional application No. 62/852,690, filed on May 24, 2019.
(60) Provisional application No. 62/852,670, filed on May 24, 2019.
(60) Provisional application No. 62/808,753, filed on Feb. 21, 2019.
(60) Provisional application No. 62/808,737, filed on Feb. 21, 2019.

(51) Int. Cl. A61K 41/00; A61P 35/00; A61K 31/337; A61K 47/42; A61K 47/32; A61K 47/34

(52) U.S. Cl. CPC A61K 41/0042; A61P 35/00; A61K 47/34; A61K 47/42; A61K 47/32; A61K 31/337

(57) ABSTRACT

This disclosure provides particles that are suitable for remotely-triggered therapy for cancer and microbial infection.

### CROSS-REFERENCE TO RELATED APPLICATIONS

This application claims priority to International Application No. PCT/2020/019348, filed on Feb. 21, 2020, U.S. Provisional Patent Application No. 62/808,737, filed on Feb. 21, 2019, U.S. Provisional Patent Application No. 62/808,753, filed on Feb. 21, 2019, U.S. Provisional Patent Application No. 62/852,664, filed on May 24, 2019, U.S. Provisional Patent Application No. 62/852,670, filed on May 24, 2019, U.S. Provisional Patent Application No. 62/852,674, filed on May 24, 2019, U.S. Provisional Patent Application No. 62/852,690, filed on May 24, 2019, U.S. Provisional Patent Application No. 62/852,659, filed on May 24, 2019, each of which is hereby incorporated by reference in its entirety.

### FIELD OF INVENTION

This disclosure provides particle heaters and methods of use thereof for the remotely-triggered therapies for treating cancer and microbial infections and synergistic combination therapies thereof.

### BACKGROUND OF THE INVENTION

Conventional chemotherapies for cancer treatment have their inherent drawbacks due to dose-limiting toxicities and poor targeting. A formidable challenge in curing cancer is the difficulty in administering a sufficiently high dose of anticancer agents while minimizing the adverse effects on normal tissues. Tumor-targeted delivery can increase the efficacy of cytotoxic agent and thereby improve patient survival. Further, all anticancer agents have a specific minimum dose or concentration to impart functional activity at the tumor tissue. Following administration, the body's natural defense mechanisms clear a large percent of the anticancer agents. Therefore, the dose or amount of the anticancer agent administered is often an excess amount to achieve the desired functional effect at the targeted tumor tissue. Anticancer agents generally have various degrees of toxicity to the body. Sometimes such anticancer agents are encapsulated to minimize toxicity to the body, like Abraxane®. Even with such encapsulation, in general, there can be some leakage of the anticancer agent out of the particle. This leakage reduces efficacy and increases side-effects impacting patient survival and quality of life.

Many infectious human diseases are caused by human pathogens such as bacteria, fungi and viruses. Bacteria are tiny single-celled organisms, some of which can be useful to humans while some can be pathogenic. Serious infections can be treated with antibiotics, which work by disrupting the bacterium's metabolic processes. Microbial infections pose serious risks to patients and are one of the leading causes of morbidity and mortality worldwide. In particular, microbial infections occurring after surgery can cause serious complications, including septicemia. Such post-operative microbial infections usually occur at incision sites or at sites where medical devices have been implanted and can spread to other sites through the vascular system. The appearance of antibiotic-resistant bacterial strains is a serious problem in medical treatment that adds to the burgeoning healthcare burden on our society. Existing treatments of these infections require integrated, interdisciplinary clinical approaches that include long-term systemic antimicrobial therapy and surgical intervention to debride infected tissues and/or remove the infected implants. The local antibiotic delivery allows for high concentrations of antibiotic accumulation at the site of infection without causing systemic toxicity. These existing treatments come at a great emotional and economic cost to the patient and even after such intensive intervention, the failure rate in bone and implant-associated infections remains relatively high.

A virus is an even smaller microorganism that can only reproduce inside a host's living cell. It is very difficult to kill a virus. Antibiotics are useless against viral infections. This is because viruses hijack the host cells to perform their activities for them. So antiviral drugs work differently to antibiotics, by interfering with the viral enzymes instead. Antiviral drugs are currently only effective against a few viral diseases, such as influenza, herpes, hepatitis B and C and HIV

Using relatively non-toxic agents that can be triggered exogenously to cause the death of unwanted cells including cancer cells and microbes is a very attractive way to treat diseases caused by the unwanted cells with reduced collateral damage to the body. Remotely-triggered therapies like photodynamic therapy (PDT) and photothermal therapy (PTT) have been explored for cancer treatment, killing microbes, as well as wound treatment. PDT involves the generation of reactive molecular species like reactive oxygen species (ROS) to localize the destruction of cells. PDT is a clinically approved modality for treating several cancers.

Many inorganic photothermal agents, e.g., gold, silver, platinum and transitional metal sulfide or oxide nanoparticles, have been used for PTT. These inorganic photothermal agents achieve high therapeutic efficacy in many preclinical animal models, however, the clinical application is significantly limited due to their non-biodegradability and potential long-term toxicities. Organic molecules can also be used as PTT agents but usually suffer from poor bioavailability and non-specific toxicity. Encapsulation of organic PTT agents into particles has been explored and these particles can overcome some of these shortcomings of the small organic molecules. Indocyanine green (ICG) is a clinically used diagnostic contrast agent that can also produce heat following laser irradiation. The use of particles encapsulating ICG for PTT has been explored for cancer, but these particles tend to be leaky, thus reducing the PTT efficacy, and causing unwanted cytotoxicity. Moreover, a large amount of ICG is needed for the desired efficacy because of body chemicals breaking down the ICG in the leaky particles. Further, the clinical application of the ICG based photothermal particles is also limited due to their lack of targeting abilities.

Moreover, due to the heterogeneous distribution of particle heaters in the diseased tissues and the limited penetration depth of near infrared (NIR) light in deep tissues, it remains a great challenge to use PTT or PDT alone to achieve complete eradication of tumor cells, or microbes.

Therefore, there exists a need for a clinically effective therapy with low toxicity and low collateral damage to unwanted cells like cancer cells and pathogenic microbes. The present invention provides remotely-triggered synergistic combination therapy meeting such need with synergistic therapeutic effects and reduced drug-related toxicity, that can overcome multidrug resistance through the use of multiple, different mechanisms of inducing death of unwanted cells than either PTT, PDT, or chemotherapy alone.

### SUMMARY OF THE INVENTION

In an embodiment, this disclosure provides a particle heater comprising a carrier admixed with a material that interacts with an exogenous source; wherein the material absorbs and converts the energy from the exogenous source into heat, then the heat travels outside the particle heater to induce localized hyperthermia at a temperature sufficient to selectively kill unwanted cells, and further wherein the particle heater structure is constructed such that it passes the Extractable Cytotoxicity Test.

In some embodiments, the particle heater further passes the Efficacy Determination Protocol.

In some embodiments, the particle heater further passes the Thermal Cytotoxicity

Test.

In some embodiments, the material exhibits at least 20% efficiency of conversion of the energy from the exogenous source to heat. In some embodiments, the material exhibits at least 20% photothermal conversion efficiency.

In some embodiments, the exogenous source is selected from the group of an electromagnetic radiation, an electrical field, a microwave, a radio wave, an ultrasound, a magnetic field, and combinations thereof.

In some embodiments, the particle heater has a median particle size ranging from about 1 nm to about 250 nm. In some embodiments, the particle heater has a median particle size ranging from about 1 nm to about 50 nm.

In some embodiments, the particle heater maintains integrity or its structure is altered after interacting with the exogenous source.

In some embodiments, the particle heater has a core-shell structure. In some embodiments, the core comprises a plasmonic absorber or iron oxide nanoparticles. In some embodiments, the shell comprises a plasmonic absorber or iron oxide. In some embodiments, the plasmonic absorber comprises plasmonic nanomaterials of noble metal including gold (Au) nanostructure, silver (Ag) nanoparticle, and copper (Cu) nanoparticle having a plasmonic resonance at a NIR wavelength. In some embodiments, the shell comprises an agent selected from the group of gold nanostructures, silver nanoparticles, iron oxide film, iron oxide nanoparticle, and combinations thereof. In some embodiments, the shell comprises an agent selected from the group of inorganic polymers, silicates, mesoporous silica, organosilicate, organo-modified silicone polymers derived from condensation of organotrisilanol or halotrisilanol, cross-linked organic polymers, and combinations thereof.

In some embodiments, the material has significant absorption of photonic energy in the near infrared spectrum region having a wavelength from 750 nm to 1100 nm. In some embodiments, the material interacting with the exogenous source has significant absorption of photonic energy in the visible range. In some embodiments, the material absorbs light at a wavelength ranging from 400 nm to 750 nm. In some embodiments, the material absorbs light at a wavelength selected from the group of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm. In some embodiments, the material is selected from the group of a tetrakis aminium dye, a cyanine dye, a squaraine dye, a squarylium dye, iron oxide, a plasmonic absorber, a zinc iron phosphate pigment, and combinations thereof.

In some embodiments, the carrier comprises a biocompatible material selected from the group of inorganic polymers and organic polymers. In some embodiments, the carrier comprises an organic polymer. In some embodiments, the organic polymer comprises a methyl methacrylate/butyl methacrylate copolymer comprising 96% methyl methacrylate repeating units and 4% butyl methacrylate repeating units. In some embodiments, the carrier comprises a crosslinked biocompatible and biodegradable polymer. In some embodiments, the crosslinked biocompatible polymer comprises a crosslinked polysaccharide. In some embodiments, the polysaccharide is selected from the group of hyaluronic acid, alginate, alginic acid, starch, and carrageenan.

In some embodiments, the carrier comprises an inorganic polymer. In some embodiments, the inorganic material is selected from the group of mesoporous silica, organo-modified silicate polymer derived from condensation of organotrisilanol or halotrisilanol, and combinations thereof.

In some embodiments, the particle heater further comprises an active agent. In some embodiments, the active agent is selected from the group of agents capable of generating reactive oxygen species, therapeutic drugs, antimicrobial agent, anti-cancer agent, anti-scarring agent, anti-inflammatory agent, metalloprotease inhibitors, treatment sensitizing the unwanted cells to remotely triggered thermal therapy, and combinations thereof.

In an embodiment, this disclosure provides a method for inducing localized hyperthermia at a tissue site in a subject comprising: administering an effective amount of the particle heater described herein to the tissue site in the subject; exposing the material to an exogenous source to absorb energy and covert it to heat which diffuses out of the particle heater to induce localized hyperthermia at a temperature ranging from about 38.0° C. to about 52.0° C. for a sufficient period of time to kill unwanted cells.

In some embodiments, the exogenous source comprises a LED light or a laser light. In some embodiments, the laser light is a pulsed laser light. In some embodiments, the exogenous source comprises a LED light. In some embodiments, the laser pulse duration is in a range from milliseconds to femtoseconds, and the laser has an oscillation wavelength at 805 nm, 808 nm, or 1064 nm. In some embodiments, the particle heater absorbs the visible light having a wavelength ranging from 400 nm to 750 nm. In some embodiments, the particle heater absorbs the laser light having a wavelength ranging from 750 nm to 1400 nm.

In some embodiments, the material is a tetrakis aminium dye. In some embodiments, the material is indocyanine green. In some embodiments, the material is a squaraine dye. In some embodiments, the material is a squarylium dye. In some embodiments, the material is iron oxide. In some embodiments, the material is a plasmonic absorber. In some embodiments, the plasmonic absorber is selected from the group of gold nanostructures including gold nanorod, gold nanocage, gold nanosphere, gold thin film, silver nanoparticle, and combinations thereof.

In some embodiments, the method further comprises heating a surrounding area in proximity to the particle heater by transferring heat from the particle heater to the surrounding area. In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C.

In an embodiment, this disclosure provides a particle for use in treating a cancer comprising: (a) an anticancer agent, (b) a carrier, (c) a material that interacts with an exogenous source, wherein the anticancer agent is encapsulated by the carrier, wherein the anticancer agent and the material in the particle exhibit stability such that the particle passes the Efficacy Determination Protocol; wherein the particle structure is constructed such that it passes the Extractable Cytotoxicity Test; wherein the material absorbs the energy from the exogenous source and converts the energy into heat; and then the anticancer agent is released outside the particle.

In an embodiment, the carrier comprises a polymer having labile bonds susceptible to hydrolysis. In an embodiment the hydrolytic degradation of the carrier is accelerated by the heat.

In some embodiments, the carrier comprises a polymer that undergoes end-chain depolymerization (unzipping or scission). In some embodiments, the end-chain depolymerization is caused by or accelerated by heat.

In some embodiments, the carrier comprises a polymer that undergoes random-chain depolymerization (unzipping or scission). In some embodiments, the random-chain depolymerization is caused by or accelerated by heat.

In some embodiments, the carrier comprises a polymer that undergoes both end-chain and random-chain depolymerization. In some embodiments, the depolymerization is caused by or accelerated by heat.

In some embodiments, the anticancer agent has a plasma half-life of less than 30 minutes. In some embodiments, the anticancer agent is a Class II, Class III, or Class IV compound according to the Biopharmaceutics Classification System. In some embodiments, the anticancer agent is selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelalisib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

In some embodiments, the carrier comprises polymer with heat-labile moieties, or polymer having labile bonds susceptible to hydrolysis. In some embodiments, the heat-liable moiety comprises substituted and unsubstituted carbonates, carbamates, esters, lactams, lactones, amides, imides, oximes, sulfonates, phosphates, or phosphonates. In some embodiments, the labile bonds susceptible to hydrolysis are selected from the group of an ester bond, an amide bond, an anhydride bond, an acetal bond, a ketal bond, and combinations thereof.

In some embodiments, the carrier is selected from the group of a polyester, a polyanhydride, a polysaccharide, a polyphosphoester, a poly(ortho ester), a poly(amino acid), a protein, polyurea, and combinations thereof. In some embodiments, the polymer is selected from the group of poly(lactic acid) (PLA), poly(glycolic acid) (PGA), poly(lactide-co-glycolide) (PLGA), poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (ε-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

In some embodiments, the polymer comprises a mixture of (i) a first PLGA having a number average molecular weight ranging from 2000 Da to 3000 Da, and (ii) a second PLGA having a number average molecular weight ranging from 570 Da to 1667 Da. In some embodiments, the first and second PLGA have a lactide:glycolide molar ratio ranging from 5:95 to 95:5, 10:90 to 90:10, 15:85 to 85:15, 25:75 to 75:25, 40:60 to 60:40, or 45:55 to 55:45. In some embodiments, the mixture comprises the first PLGA and the second PLGA in a weight ratio of first PLGA to second PLGA ranging from 10:1 to 1:10. In some embodiments, the polymer comprises a PLGA having a lactide:glycolide molar ratio ranging from 5:95 to 95:5, 10:90 to 90:10, 15:85 to 85:15, 25:75 to 75:25, 40:60 to 60:40, or 45:55 to 55:45 and has a number average molecular weight ranging from 570 Da to 3000 Da.

In some embodiments, the anticancer agent is present in an amount ranging from about 1 wt. % to about 99 wt. % by the total weight of the particle. In some embodiments, the anticancer agent has a weight ratio to the polymer ranging from about 1:99 to about 99:1, or from about 5:95 to about 95:5.

In some embodiments, the material does not have significant optical absorption in the visible spectrum region. In some embodiments, the material has significant optical absorption in the near infrared spectrum region. In some embodiments, the material has optical absorption in the range of 700-1500 nm. In some embodiments, the material is a tris-aminium dye, a di-imonium dye, or a tetrakis aminium dye. In some embodiments, the material is a zinc iron phosphate pigment.

In some embodiments, the particle further comprises a targeting group on the particle surface selected from the group of tumor targeting folate, antibodies, proteins, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, peptidomimetics, glycopeptides, peptoids, aptamer, and combinations thereof. In some embodiments, the targeting group is selected from the group of EGFR binding peptides, claudin, HYNIC-(Ser)₃-J18, FROP-1, and combinations thereof.

In some embodiments, the particle further comprises a shell to enclose the particle.

In some embodiments, the particle further comprises a hydrophilic polymer on the particle surface selected from the group of polyethylene glycols, hyperbranched polyglycerol, hyaluronic acid, and combinations thereof.

In some embodiments, the exogenous source is a microwave. In some embodiments, the exogenous source is an electrical field. In some embodiments, the exogenous source is a magnetic field. In some embodiments, the exogenous source is a sound wave (ultrasonic).

In an embodiment, this disclosure provides a particle for use in treating a cancer comprising: (a) an anticancer agent selected from the group of cisplatin, docetaxel, carboplatin, doxorubicin, etoposide, paclitaxel, and combinations thereof; (b) a carrier comprising a polymer selected from the group of poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA), and combinations thereof; (c) an IR absorbing agent selected from the group of a tris-aminium dye, a di-imonium dye, a tetrakis aminium dye, a zinc iron phosphate pigment, and combinations thereof, wherein the particle has a median particle size less than 5 μm, wherein the anticancer agent is encapsulated by the carrier, wherein the anticancer agent and the material in the particle exhibit stability such that the particle is considered passing the Efficacy Determination Protocol; wherein the particle structure is constructed such that it passes the Extractable Cytotoxicity Test; wherein the anticancer agent is released outside the particle when the exogenous source is applied. In some embodiments, the polymer comprises PLGA having a lactide:glycolide molar ratio from 5:95 to 95:5, 10:90 to 90:10, 15:85 to 85:15, 25:75 to 75:25, 40:60 to 60:40, or 45:55 to 55:45 and has a number average molecular weight ranging from 570 Da to 3000 Da. In some embodiments, the particle has a targeting group selected from the group of EGFR binding peptides, claudin, HYNIC-(Ser)₃-J18, FROP-1, and combinations thereof. In some embodiments, the surface of the particle is modified with a hydrophilic polymer selected from the group of polyethylene glycols, hyperbranched polyglycerol, hyaluronic acid, and combinations thereof.

In some embodiments, the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma, glioblastoma multiforme, non-small-cell lung cancer, hepatocellular carcinoma, metastatic colon cancer, multiple myeloma, small-cell lung cancer, and combinations thereof.

In an embodiment, this disclosure provides a method for treating a cancer in a patient in need thereof comprising: (1) administering to the patient according to the present invention, and (2) activating the particle with the exogenous source, wherein the material absorbs the energy from the exogenous source and converts the energy into heat; and wherein the heat causes degradation of the carrier, and then the anticancer agent is released outside the particle. In some embodiments, the carrier is degraded via hydrolysis. In some embodiments, the carrier is degraded by random-chain/end-chain depolymerization.

In an embodiment, this disclosure provides a particle heater for use in the remotely-triggered thermal treatment of a cancer comprising: a material interacting with an exogenous source admixed with a carrier, wherein the material in the particle exhibits stability such that the particle passes the Efficacy Determination Protocol; wherein the particle is constructed such that it passes the Extractable Cytotoxicity Test; wherein the material absorbs the energy from the exogenous source and converts the energy into heat; and then the heat travels outside the particle to induce sufficient localized hyperthermia to selectively kill cancer cells.

In some embodiments, the particle heater further passes the Thermal Cytotoxicity Test.

In some embodiments, the particle maintains its integrity after its exposure to the exogenous source. In some embodiments, the particle is a nanoparticle. In some embodiments, the particle is a microparticle.

In some embodiments, the particle further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof. In some embodiments, the shell comprises a material selected from the group of Au, Ag, Cu, iron oxide, and combinations thereof.

In some embodiments, the carrier comprises biocompatible and biodegradable polymer.

In some embodiments, the carrier comprises a biodegradable polymer having labile bonds that are selected from the group of an ester bond, an amide bond, an anhydride bond, an acetal bond, a ketal bond, and combinations thereof. In some embodiments, the carrier is selected from the group of a polyester, a polyanhydride, a polysaccharide, a polyphosphoester, a poly(ortho ester), a poly(amino acid), a protein, polyurea, and combinations thereof. In some embodiments, the polymer selected from the group of poly(lactic acid) (PLA), poly(glycolic acid) (PGA), poly-lactic acid-co-glycolic acid (PLGA), poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolactone, poly(hydroxyvalerate), polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(butylene terephthalate), poly(β-hydroxyalkanoate)s, poly(hydroxybutyrate), poly(hydroxybuthyrate-co-hydroxyvalerate), poly (ε-lysine), diblock copolymer of poly(sebacic acid) and polyethylene glycol (PSA-PEG), trimethylene carbonate, poly(β-hydroxybutyrate), poly(g-ethyl-L-glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

In some embodiments, the carrier is selected from the group of lipid, polymer-lipid conjugate, carbohydrate-lipid conjugate, peptide-lipid conjugate, protein-lipid conjugate, and combinations thereof.

In some embodiments, the carrier comprises a lipid selected from the group of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group of DPPC, MPPC, PEG, DMPC, DMPG, DSPE, DOPC, DOPE, DSPC, DSPE-PEG, MSPC, cholesterol, PS, PC, PE, PG, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the material does not have significant optical absorption in the visible spectrum region. In some embodiments, the material has significant optical absorption in the near infrared spectrum region. In some embodiments, the material has optical absorption in the range of 750-1100 nm. In some embodiments, the material is a tris-aminium dye, a di-imonium dye, a cyanine dye, a squaraine dye, a squarylium dye, gold nanoparticle, iron oxide, or a tetrakis aminium dye. In some embodiments, the material is a zinc iron phosphate pigment.

In some embodiments, the particle further comprises a tumor cell targeting group on the particle surface selected from the group of folate, antibodies, proteins, EGFR binding antibodies, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, peptidomimetics, glycopeptides, peptoids, aptamer, and combinations thereof. In some embodiments, the targeting group is selected from the group of an EGFR binding antibody, an EGFR binding peptide, and combinations thereof. In some embodiments, the targeting group is an EGFR binding antibody selected from the group of cetuximab, panitumumab, and combinations thereof. In some embodiments, the targeting group is an EGFR binding peptides selected from the group of YHWYGYTPQNVI, YRWYGYTPQNVI, L-AE (L amino acids in the sequence-FALGEA), D-AE (D-amino acids in the sequence-FALGEA), and combinations thereof. In some embodiments, the targeting group is covalently conjugated to the surface of the particle via a disulfide bond.

In some embodiments, the particle further comprises a hydrophilic polymer on the particle surface selected from the group of polyethylene glycols, hyperbranched polyglycerol, hyaluronic acid, and combinations thereof.

In some embodiments, the exogenous source is selected from the group of a microwave, an electrical field, a magnetic field, sound wave (ultrasonic), and combinations thereof.

In an embodiment, this disclosure provides a particle heater for use in the remotely-triggered thermal treatment of a cancer comprising:

(a) a material that interacts with an exogenous source, wherein the material is an IR absorbing agent selected from the group of a tris-aminium dye, a di-imonium dye, a tetrakis aminium dye, a cyanine dye, a squaraine dye, a zinc iron phosphate pigment, and combinations thereof,

(b) a carrier comprising a polymer selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof,

wherein the particle has a median particle size less than 5 μm,

wherein the material interacting with an exogenous source is encapsulated by the carrier to form a particle,

wherein the material in the particle heater exhibits stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells.

In some embodiments, the particle surface further comprises a targeting group selected from the group of an EGFR binding antibodies (cetuximab, and panitumumab); EGFR binding peptides (YHWYGYTPQNVI or YRWYGYTPQNVI or the L-AE (L amino acids in the sequence-FALGEA), D-AE (D-amino acids in the sequence-FALGEA)), and combinations thereof.

In some embodiments, the particle surface is further modified with a hydrophilic polymer selected from the group of polyethylene glycols, hyperbranched polyglycerol, hyaluronic acid, and combinations thereof.

In an embodiment, this disclosure provides a method for causing remotely-triggered thermal killing of tumor cells at a tumor site in a subject in need thereof comprising: (1) administering an effective amount of the particle heater comprising a carrier admixed with a material to the subject and waiting for a period of time to allow the particle heater to reach the tumor site, and (2) exposing the particle heater to an exogenous source that heats the particle heater for a sufficient period of time, wherein the material in the particle exhibits stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to cause a temperature increase in a tissue area surrounding the particle thereby to induce localized hyperthermia at a temperature ranging from about 38.0° C. to about 52.0° C. that is sufficient to selectively kill cancer cells, and wherein the collateral damage to the non-cancer cells is minimized. In some embodiments, the subject is a warm-blooded animal. In some embodiments, the subject is a human.

In some embodiments, the induced hyperthermia is a mild hyperthermia at a temperature ranging from about 38.0° C. to about 41.0° C. In some embodiments, the induced hyperthermia is a moderate hyperthermia at a temperature ranging from about 41.1° C. to about 45.0° C., wherein the hyperthermia does not cause collateral damage to healthy cells. In some embodiments, the induced hyperthermia is a profound hyperthermia at a temperature ranging from about 45.1° C. to about 52.0° C.

In some embodiments, the cancer is selected from the group of bladder cancer, head and neck cancer, pancreatic ductal adenocarcinoma (PDA), pancreatic cancer, colon cancer, breast cancer, fibrosarcoma, mesothelioma, lung cancer, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, cervical cancer, kidney cancer, liver cancer, esophageal cancer, testicular cancer, gynecological cancer, thyroid cancer, Kaposi's sarcoma, viral-induced cancer, glioblastoma multiforme, non-small-cell lung cancer, metastatic colon cancer, small-cell lung cancer, and combinations thereof. In some embodiments, the cancer is selected from the group of breast cancer, lung cancer, and glioblastoma multiforme.

In an embodiment, this disclosure provides a synergistic combination therapy for the treatment of cancer comprising: (a) an anticancer agent, and (b) a particle heater having a material interacting with an exogenous source admixed with a carrier, wherein the material absorbs the energy from the exogenous source and converts the energy into heat; and then the heat travels outside the particle to induce localized hyperthermia, wherein the heat causes the release of the anticancer agent outside of the particle, wherein the localized hyperthermia and the anticancer agent exhibit synergy in killing cancer cells, and wherein the particle is constructed such that it passes the Extractable Cytotoxicity Test.

In some embodiments, the localized hyperthermia and the anticancer agent exhibit coefficient of drug interaction (CDI)<1.0. In some embodiments, the CDI of the localized hyperthermia and the anticancer agent is about 0.1, about 0.2, about 0.3, about 0.4, about 0.5, about 0.6, about 0.7, about 0.8, about 0.9, or about 1.0.

In some embodiments, the anticancer agent is further encapsulated by the particle heater having the material, and wherein the heat causes the particle heater to alter its structure to release the anticancer agent outside of the particle. In some embodiments, the anticancer agent is in a conventional pharmaceutical dosage.

In some embodiments, the particle heater further passes the Thermal Cytotoxicity Test. In some embodiments, the particle heater further passes the Efficacy Determination Protocol.

In some embodiments, the exogenous source is selected from the group of an electromagnetic radiation, an electrical field, a microwave, a radio wave, an ultrasound, a magnetic field, and combinations thereof.

In some embodiments, the particle maintains its integrity and/or alters its structure after its exposure to the exogenous source.

In some embodiments, the particles are nanoparticles or microparticles. In some embodiments, the nanoparticle has a median particle size ranging from about 1 nm to about 250 nm. In some embodiments, the nanoparticle has a median particle size ranging from about 10 nm to about 50 nm.

In some embodiments, the particle further comprises a shell to enclose the particle to form a core-shell particle. In some embodiments, the shell comprises a cross-linked inorganic polymer selected from the group of mesoporous silica, organo-modified silicate polymer derived from condensation of organotrisilanol or halotrisilanol, and combinations thereof. In some embodiments, the shell comprises a plasmonic absorber selected from the group of a thin film of noble metals including gold (Au), silver (Ag), copper (Cu), nanoporous gold thin film, and combinations thereof. In some embodiments, the particle further comprises a coating formed of polydopamine that is capable of converting exogenous energy to heat.

In some embodiments, the unencapsulated anticancer agent has a plasma half-life of less than 30 minutes. In some embodiments, the anticancer agent is a Class II, Class III or Class IV compound according to the Biopharmaceutics Classification System. In some embodiments, the anticancer agent is selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelali sib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

In some embodiments, the anticancer agent is a PI3K inhibitor selected from the group of wortmannin, temsirolimus, everolimus, buparlisib (BMK-120), 5-(2,6-dimorpholinopyrimidin-4-yl)-4-(trifluoromethyl)pyridin-2-amine), pictilisib, gedatolisib, apitolisib, pilaralisib, copanli sib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), and combinations thereof.

In some embodiments, the anticancer agent is a proteasome inhibitor selected from the group of bortezomib, ixazomib, marizomib, oprozomib, delanzomib, epoxomicin, disulfiram, lactacystin, beta-hydroxy beta-methylbutyrate, and combinations thereof. In some embodiments, the anticancer agent is an EGFR inhibitor selected from the group of erlotinib, gefitinib, neratinib, osimertinib, vandetanib, dacomitinib, lapatinib, and combinations thereof.

In some embodiments, the material has significant absorption of photonic energy in the visible spectrum region having a wavelength range from 400 nm to 750 nm. In some embodiments, the material has significant absorption of photonic energy in the near infrared spectrum region having a wavelength range from 750 nm to 1100 nm. In some embodiments, the material is selected from the group of a tetrakis aminium dye, a cyanine dye, a squarylium dye, indocyanine green (ICG), new ICG (IR 820), squaraine dye, IR 780 dye, IR 193 dye, Epolight™ IR 1117, zinc iron phosphate pigment, iron oxide, and combinations thereof.

In some embodiments, the carrier comprises a biocompatible and/or a biodegradable sub stance.

In some embodiments, the biocompatible substance and/or biodegradable substance is selected from the group of a lipid, an inorganic polymer, an organic polymer, and combinations thereof. In some embodiments, the carrier comprises a polymer having labile bonds susceptible to hydrolysis. In some embodiments, the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polylysine; and combinations thereof.

In some embodiments, the carrier comprises a cross-linked biocompatible and biodegradable polymer. In some embodiments, the cross-linked biocompatible polymer comprises a cross-linked polysaccharide. In some embodiments, the polysaccharide is selected from chitosan, hyaluronic acid, alginate, alginic acid, starch, carrageenan, and combinations thereof.

In some embodiments, the carrier comprises an inorganic polymer. In some embodiments, the inorganic material is selected from the group of mesoporous silica, organo-modified silicate polymer derived from the condensation of organotrisilanol or halotrisilanol, and combinations thereof.

In some embodiments, the carrier is a lipid. In some embodiments, the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the lipid comprises thermoresponsive lipid/polymer hybrid. In some embodiments, the thermoresponsive lipid/polymer hybrid is selected from the group of triblock copolymer of [poly(2-isopropyl-2-oxazoline)-b-poly(dimethylsiloxane)-b-poly(2-isopropyl-2-oxazoline]) and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

In some embodiments, the particle heater further has a thin film of noble metal on the particle surface, wherein the noble metal is selected from the group of gold, silver, copper, and combinations thereof. In some embodiments, the particle heater further comprises iron oxide.

In some embodiments, comprising a cancer targeting group on the particle surface selected from the group of folate, antibodies, proteins, EGFR binding antibodies, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, p32-protein binding peptide, peptidomimetics, glycopeptides, peptoids, aptamer, and combinations thereof. In some embodiments, the targeting group is selected from the group of an EGFR antibody, an EGFR binding peptide, p32-protein binding peptide, and combinations thereof. In some embodiments, the cancer-targeting group is an EGFR binding antibody selected from the group of cetuximab, panitumumab, and combinations thereof. In some embodiments, the cancer targeting group is an EGFR binding peptide selected from the group of YHWYGYTPQNVI, YRWYGYTPQNVI, L-AE (L amino acids in the sequence-FALGEA), D-AE (D-amino acids in the sequence-FALGEA), and combinations thereof.

In some embodiments, the cancer-targeting group is covalently conjugated to the surface of the particle heater via a disulfide bond, or NHS-EDC chemistry.

In some embodiments, comprising a hydrophilic polymer on the particle heater surface selected from the group of polyethylene glycols, hyperbranched polyglycerol, hyaluronic acid, and combinations thereof.

In some embodiments, the particle heater comprises the anticancer agent selected from the group of gefitinib, gefitinib, erlotinib, lapatinib, neratinib, osimertinib, vandetanib, dacomitinib, abemaciclib, trastuzumab, cetuximab, panitumumab, and combinations thereof; and the material is an IR absorbing agent selected from the group of a indocyanine green (ICG), new ICG (IR 820), IR 193 dye, Epolight™ 1117, Epolight™ 1175, and combinations thereof, (c) a carrier comprising a polymer selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof; and wherein the particle heater has a median particle size less than 200 nm.

In an embodiment, this disclosure provides a composition for use in a remotely-triggered synergistic combination therapy for treatment of a cancer comprising: (a) a particle heater having a material interacting with an exogenous source and a carrier; and (b) a pharmaceutical dosage having an anticancer agent.

In some embodiments, the particle heater and the pharmaceutical dosage forms a unitary dosage. In some embodiments, the particle heater and the pharmaceutical dosage are two discrete preparations.

In some embodiments, the pharmaceutical dosage is selected from the group of a capsule, a tablet, a buccal tablet, an oral disintegrating tablet, a liquid formulation, a dispersion, an injection preparation, powder for injection, and suppository.

In some embodiments, the particle heaters are nanoparticles or microparticles.

In some embodiments, the particle heater further combined with a pharmaceutically acceptable excipient to form a particle heater preparation. In some embodiments, the particle heater preparation is selected from the group of a capsule, a tablet, a buccal tablet, an oral disintegrating tablet, a liquid formulation, a dispersion, an injection preparation, powder for injection, and suppository.

In an embodiment, this disclosure provides a method for causing remotely-triggered synergistic combination therapy for the treatment of cancer in a subject comprising: (1) administering a therapeutically effective amount of any one of the herein described particle heaters to the tumor site in the subject in need thereof and allowing the synergistic combination therapy to associate with cancer cells, and (2) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the anticancer agent exhibit synergy in killing cancer cells, and wherein the particle is constructed such that it passes the Extractable Cytotoxicity Test.

In some embodiments, the anticancer agent is further encapsulated by the particle heater having the material, and wherein the heat causes the particle heater to alter its structure to release the anticancer agent outside of the particle. In some embodiments, the anticancer agent further comprises the carrier to form a chemotherapy particle free of the material, and wherein the heat causes the chemotherapy particle to alter its structure to release the anticancer agent outside of the particle.

In some embodiments, the particle heater and the anticancer agent are administered to the patient simultaneously. In some embodiments, the particle heater and the anticancer agent are administered to the patient sequentially. In some embodiments, the anticancer agent is administered before the administering of the particle heater. In some embodiments, the particle heater is administered before the administering the anticancer agent.

In some embodiments, the method further comprises performing radiation therapy or surgery.

In some embodiments, the method further comprises performing surgery. Particle heater is used for the imaging guided surgery of the tumor followed by the remotely-triggered destruction of cancer cells along the surgical margins.

In some embodiments, the induced hyperthermia is a mild hyperthermia at a temperature ranging from about 38.0° C. to about 41.0° C. In some embodiments, the induced hyperthermia is a moderate hyperthermia at a temperature ranging from about 41.1° C. to about 45.0° C., wherein the hyperthermia does not cause collateral damage to healthy cells. In some embodiments, the induced hyperthermia is a profound hyperthermia at a temperature ranging from about 45.1° C. to about 52.0° C.

In some embodiments, the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, intraocular melanoma, oral cavity and oropharyngeal cancers, stomach cancer, cervical cancer, kidney cancer, liver cancer, esophageal cancer, testicular cancer, gynecological cancer, thyroid cancer, Kaposi's sarcoma, glioblastoma multiforme, non-small-cell lung cancer, hepatocellular carcinoma, multiple myeloma, small-cell lung cancer, melanoma, and combinations thereof. In some embodiments, the cancer is breast cancer, lung cancer or glioblastoma multiforme.

In an embodiment, this disclosure provides a method of treating a cancer with synergistic combination therapy in a subject comprising the steps of sensitizing the cancer by administering to the subject in need thereof a treatment that will (i) induce apoptosis or autophagy in tumor cells, (ii) induce ferroptosis in tumor cells, (iii) induce necrotic cell death in tumor, (iv) modify the tumor environment, (v) stimulate tumor-infiltrating immune cells, or (vi) a combination of two or more thereof.

In some embodiments, the treatment is a hyperthermia or an anticancer agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat travels outside the particle to induce localized hyperthermia sufficient to selectively kill cancer cells.

In some embodiments, the anticancer agent is encapsulated in the particle heater and the heat causes the particle to alter its structure to release of the anticancer agent. In some embodiments, the anticancer agent is not encapsulated in the particle heater. In some embodiments, the anticancer agent is present in a separate pharmaceutical composition from the particle heater. In some embodiments, the particle heater is administered before the administration of the anticancer agent. In some embodiments, the particle heater is administered after the administration of the anticancer agent. In some embodiments, the particle heater is administered concurrently with the administration of the anticancer agent.

In some embodiments, the method further comprises the step of activating the particle heater remotely with an exogenous source, wherein the exogenous source is selected from the group of an electromagnetic radiation, an electrical field, a microwave, a radio wave, an ultrasound, a magnetic field, and combinations thereof.

In some embodiments, the particle heater is used to guide the imaging-based surgical debulking of the tumor followed by remotely triggering the particles for the destruction of cancer cells along the surgical margins.

In some embodiments, the activation of the particle heater occurs before the administration of the anticancer agent. In some embodiments, the activation of the particle heater occurs after the administration of the anticancer agent. In some embodiments, sensitizing the tumor comprises administering to the subject a treatment that will induce apoptosis, autophagy, ferroptosis, or necrotic cell death in tumor cells. In some embodiments, the tumor sensitizing treatment is selected from the group of thermotherapy, radiation therapy, surgery, chemotherapy, immunotherapy, photodynamic therapy, or a combination thereof. In some embodiments, the tumor sensitizing treatment is thermotherapy. In some embodiments, tumor sensitizing treatment is thermotherapy and chemotherapy. In some embodiments, the tumor sensitizing treatment is photodynamic therapy.

In some embodiments, the present disclosure provides compositions for treating localized microbial infections in a patient. The compositions comprise: a particle comprising: (a) an antimicrobial agent, (b) a carrier, (c) a material that interacts with an exogenous source, wherein the antimicrobial agent and the material in the particle exhibit stability such that the particle is considered passing the Efficacy Determination Protocol; wherein the particle structure is constructed such that it passes the Extractable Cytotoxicity Test; and wherein the antimicrobial agent is released outside the particle when the exogenous source is applied.

In some embodiments, the particle is amorphous or partially amorphous or partially crystalline.

In some embodiments, the particle further comprises a shell enclosing the particle to form a core-shell particle.

In some embodiments, the particle further comprises a microbial targeting group on the particle surface. In some embodiments, the microbial targeting group is selected from the group of an antibody targeting the surface antigen of the bacteria, a cationic antimicrobial peptide, cell penetrating peptides including apidaecin, tat, buforin, magainin, and combinations thereof. In some embodiments, the microbial targeting group is targeting the host (human) macrophages that harbor the microbes.

In some embodiments, the antimicrobial agent is an inorganic compound or an organic compound. In some embodiments, the antimicrobial agent is an inorganic compound selected from the group of silver particles, gold particles, gallium particles, zinc oxide particles, copper oxide particles, and combinations thereof. In some embodiments, the antimicrobial agent is an organic compound selected from the group of an organic acid, a phenolic compound, a phyto-antibiotic, an amino acid, a quaternary ammonium compound, a surfactant, an antibiotic, and combinations thereof. In some embodiments, the antimicrobial agent is an antibiotic selected from the group of ampicillin, sulbactam, cefotaxime, telithromycin, temafloxacin, trovafloxacin, praziquantel, amikacin, ciprofloxacin, vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

In some embodiments, the antimicrobial agent is present in an amount ranging from about 1 wt. % to about 99 wt. % by the total weight of the particle.

In some embodiments, the antimicrobial agent has a weight ratio to the polymer ranging from about 1:99 to about 99:1, or from about 5:95 to about 95:5.

In some embodiments, the antimicrobial agent is chemically conjugated to the carrier via a heat-labile linker. In some embodiments, the heat-labile linker is selected from the group of substituted and unsubstituted carbonates, substituted and unsubstituted carbamates, substituted and unsubstituted esters, substituted and unsubstituted lactams, substituted and unsubstituted lactones, substituted and unsubstituted amides, substituted and unsubstituted imides, substituted and unsubstituted oximes, substituted and unsubstituted sulfonates, substituted and unsubstituted phosphonates, and combinations thereof.

In some embodiments, the carrier comprises a polymer with heat-labile moieties, or a polymer having labile bonds susceptible to hydrolysis. In some embodiments, the labile bonds are selected from the group of an ester bond, an amide bond, an anhydride bond, an acetal bond, a ketal bond, and combinations thereof. In some embodiments, the polymer is selected from the group of a polyester, a polyanhydride, a polysaccharide, a polyphosphoester, a poly(ortho ester), a poly(amino acid), a protein, and combinations thereof. In some embodiments, the polymer is selected from the group of a polyester including poly(lactic acid) (PLA), poly(glycolic acid) (PGA), poly(lactide-co-glycolide) (PLGA), poly(lactic acid)-polyethylene glycol-poly(lactic acid) (PLA-PEG-PLA), poly (L-co-D,L lactic acid) 70:30 (PLDLA), poly-L-lactic acid-co-glycolic acid, poly-D,L-lactic acid-co-glycol acid, poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (ε-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof. In some embodiments, the polyester comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45 and has a number average molecular weight ranging from 2000 Da to 10500 Da.

In some embodiments, the material does not have significant optical absorption in the visible spectrum region. In some embodiments, the material has significant optical absorption in the range of 700-1500 nm. In some embodiments, the material has significant optical absorption in the range of 750-1400 nm. In some embodiments, the material is a tri-aminium dye, a di-imonium dye, or a tetrakis aminium dye. In some embodiments, the material is a zinc iron phosphate pigment.

In some embodiments, the exogenous source is selected from the group of electromagnetic radiation, microwaves, an electric field, a magnetic field, radiowaves, and ultrasound. In some embodiments, the exogenous source is electromagnetic radiation (EMR). In some embodiments, the exogenous source is laser pulse radiation at a determined thermal relaxation time (TRT). In some embodiments, the TRT is selected from the group of picoseconds and nanoseconds. In some embodiments, the TRT is selected from the group of microseconds and milliseconds.

The present disclosure also provides methods and materials for treating localized bacterial infections. The methods comprise administering to a patient infected with bacteria one or more particles comprising an antimicrobial agent, a carrier, and a material interacting with an exogenous source; and activating the particles with the exogenous source, wherein the material absorbs the energy from the exogenous source and converts the energy into heat; and wherein the heat causes degradation of the carrier, and then the antimicrobial agent is released outside the particle.

In some embodiments, the particle further comprises a shell enclosing the particle to form a core-shell particle.

In some embodiments, the particle comprises a zinc iron phosphate pigment.

In some embodiments, the particle further comprises a microbial targeting group on the particle surface. In some embodiments, the microbial targeting group is selected from the group of an antibody targeting the surface antigen of the bacteria, a cationic antimicrobial peptide, cell penetrating peptides including apidaecin, tat, buforin, magainin, and combinations thereof.

In some embodiments, the antimicrobial agent is an inorganic compound or an organic compound. In some embodiments, the antimicrobial agent is an inorganic compound selected from the group of silver particles, gold particles, gallium particles, zinc oxide particles, copper oxide particles, and combinations thereof. In some embodiments, the antimicrobial agent is an organic compound selected from the group of an organic acid, a phenolic compound, a phyto-antibiotic, amino acids, quaternary ammonium compounds, a detergent, antibiotics, and combinations thereof. In some embodiments, the antimicrobial agent is an antibiotic selected from the group of ampicillin, sulbactam, cefotaxime, telithromycin, temafloxacin, trovafloxacin, praziquantel, amikacin, ciprofloxacin, or vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

In some embodiments, the antimicrobial agent is present in an amount ranging from about 1 wt. % to about 95 wt. % by the total weight of the particle. In some embodiments, the antimicrobial agent has a weight ratio to the polymer ranging from about 1:99 to about 99:1, or from about 5:95 to about 95:5.

In some embodiments, the carrier comprises a polymer with heat-labile moieties, or a polymer having labile bonds susceptible to hydrolysis. In some embodiments, the labile bonds are selected from the group of an ester bond, an amide bond, an anhydride bond, an acetal bond, a ketal bond, and combinations thereof. In some embodiments, the polymer is selected from the group of a polyester, a polyanhydride, a polyphosphoester, a poly(ortho ester), a poly(amino acid), a polysaccharide, a protein, and combinations thereof. In some embodiments, the polymer is selected from the group of a polyester including poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA), poly-L-lactic acid-co-glycolic acid, poly-D,L-lactic acid-co-glycol acid, poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (ε-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof. In some embodiments, the polyester comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45 and has a number average molecular weight ranging from 2000 Da to 10500 Da.

In some embodiments, the carrier comprises a polymer that undergoes end-chain depolymerization (unzipping or scission). In some embodiments, the end-chain depolymerization is caused by or accelerated by heat.

In some embodiments, the material absorbs light having a wavelength ranging from 700 nm to 1500 nm. In some embodiments, the material is a tri-aminium dye, a di-imonium dye, or a tetrakis aminium dye.

In some embodiments, the exogenous source is a laser light. In some embodiments, the laser light is a pulsed laser light. In some embodiments, the laser has a pulse duration less than the TRT of the particle. In some embodiments, the laser pulse duration is selected from the group of picoseconds, nanoseconds, microseconds, and milliseconds, and the laser has an oscillation wavelength at 1064 nm. In some embodiments, the exogenous source is laser pulse radiation at a determined thermal relaxation time (TRT). In some embodiments, the TRT is selected from the group of picoseconds and nanoseconds. In some embodiments, the TRT is selected from the group of microseconds and milliseconds

In some embodiments, the bacteria are multidrug resistant bacteria. In some embodiments, the multidrug resistant bacteria comprise Gram positive bacteria. In some embodiments, the multidrug resistant bacteria comprise Gram negative bacteria. In some embodiments, the multidrug resistant bacteria comprise both Gram positive and Gram negative bacteria. In some embodiments, the multidrug resistant bacteria comprise one or more species selected from the group of E. coli, K. pneumonia, M. tuberculosis, Streptococcus aureus, P. aeruginosa, Streptococcus epidermidis, and Streptococcus haemolyticus.

In some embodiments, this disclosure provides compositions for the synergistic combination therapy for treating microbial infection comprising: (a) an antimicrobial agent, and (b) a particle heater having a material interacting with an exogenous source admixed with a carrier, wherein the material absorbs the energy from the exogenous source and converts the energy into heat; and then the heat travels outside the particle to induce localized hyperthermia, wherein the hyperthermia and the antimicrobial agent exhibit synergy in killing microbes, and wherein the particle is constructed such that it passes the Extractable Cytotoxicity Test.

In some embodiments, the synergistic combination therapy for treating microbial infection, wherein the localized hyperthermia and the antimicrobial agent exhibit coefficient of drug interaction (CDI)<1.0.

In some embodiments, the synergistic combination therapy for treating microbial infection, wherein the CDI of the localized hyperthermia and the antimicrobial agent is about 0.1, about 0.2, about 0.3, about 0.4, about 0.5, about 0.6, about 0.7, about 0.8, about 0.9, or about 1.0.

In some embodiments, the carrier encapsulates the material and the antimicrobial agent to form a single particle heater. In some embodiments, the antimicrobial agent is a conventional pharmaceutical dosage. In some embodiments, the heat causes the release of the antimicrobial agent.

In some embodiments, the particle heater further passes the Efficacy Determination Protocol. In some embodiments, the particle heater further passes the Thermal Cytotoxicity Test.

In some embodiments, the exogenous source is selected from the group of an electromagnetic radiation, an electrical field, a microwave, a radio wave, an ultrasound, a magnetic field, and combinations thereof.

In some embodiments, the particle heater maintains integrity after its exposure to the exogenous source. In some embodiments, the particle alters its structure to release the antimicrobial agent after exposure to the exogenous source.

In some embodiments, the particle further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof. In some embodiments, the shell comprises a plasmonic absorber selected from the group of a thin film of noble metals including gold (Au), silver (Ag), copper (Cu), nanoporous gold thin film, and combinations thereof. In some embodiments, the shell comprises iron oxide. In some embodiments, the particle further comprises a coating formed of polydopamine that can convert exogenous energy into heat.

In some embodiments, the antimicrobial agent is an inorganic compound or an organic compound. In some embodiments, the antimicrobial agent is an inorganic compound selected from the group of silver particles, gold particles, gallium particles, zinc oxide particles, copper oxide particles, and combinations thereof. In some embodiments, the antimicrobial agent is an organic compound selected from the group of an organic acid, a phenolic compound, a phyto-antibiotic, an amino acid, a quaternary ammonium compound, a surfactant, an antibiotic, and combinations thereof. In some embodiments, the antimicrobial agent is an antibiotic selected from the group of ampicillin, sulbactam, cefotaxime, telithromycin, temafloxacin, trovafloxacin, praziquantel, amikacin, ciprofloxacin, vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

In some embodiments, the antimicrobial agent is chemically conjugated to the particle surface via a heat-labile linker. In some embodiments, the heat-labile linker is selected from the group of substituted and unsubstituted carbonates, substituted and unsubstituted carbamates, substituted and unsubstituted esters, substituted and unsubstituted lactams, substituted and unsubstituted lactones, substituted and unsubstituted amides, substituted and unsubstituted imides, substituted and unsubstituted oximes, substituted and unsubstituted sulfonates, substituted and unsubstituted phosphonates, and combinations thereof. In some embodiments, the antimicrobial agent is encapsulated within the particle.

In some embodiments, the material has significant absorption of photonic energy in the near infrared spectral region having a wavelength range from 750 nm to 1100 nm. In some embodiments, the material is selected from the group of a tetrakis aminium dye, a cyanine dye, a squarylium dye, indocyanine green (ICG), new ICG (IR 820), a squaraine dye, IR 780 dye, IR 193 dye, Epolight™ IR1117, iron oxide, zinc iron phosphate pigment, and combinations thereof.

In some embodiments, the carrier comprises a biocompatible substance selected from the group of a lipid, an inorganic polymer, an organic polymer, and combinations thereof.

In some embodiments, the carrier comprises an organic polymer. In some embodiments, the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polylysine; and combinations thereof. In some embodiments, the carrier comprises a crosslinked biocompatible and biodegradable polymer. In some embodiments, the crosslinked biocompatible polymer comprises a crosslinked polysaccharide. In some embodiments, the polysaccharide is selected from chitosan, hyaluronic acid, alginate, alginic acid, starch, carrageenan, and combinations thereof.

In some embodiments, the carrier comprises an inorganic polymer. In some embodiments, the inorganic material is selected from the group of mesoporous silica, organo-modified silicate polymer derived from condensation of organotrisilanol or halotrisilanol, and combinations thereof.

In some embodiments, the particle heater further has a thin film of noble metal on the particle surface, wherein the noble metal is selected from the group of gold, silver, copper, and combinations thereof. In some embodiments, the particle heater has a layer of iron oxide on the particle surface.

In some embodiments, the carrier is a lipid. In some embodiments, the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the lipid comprises a thermoresponsive lipid/polymer hybrid. In some embodiments, the thermoresponsive lipid/polymer hybrid is selected from the group of a triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

In some embodiments, the particle heater further comprises a microbe-targeting group on the particle surface. In some embodiments, the microbe-targeting group is selected from the group of antibody targeting the surface antigen of microbe, group of antibody targeting microbial Toll Like Receptor (TLR), cationic antimicrobial peptide, cell penetrating peptides including apidaecin, TAT ((GRKKRRQRRRPQ), buforin, magainin, RGD peptide, and combinations thereof.

In some embodiments, the particle heater comprises the antimicrobial agent is selected from the group of antibiotics, antiseptic agents, cationic surfactants, biocides, and combinations thereof, (b) the material is an IR absorbing agent selected from the group of a indocyanine green (ICG), new ICG (IR 820), IR 780 dye, IR 193 dye, a squaraine dye, Epolight™ IR 1117, Epolight™ IR 1175, iron oxide, and combinations thereof, (c) the carrier is selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof; wherein the particle heater has a median particle size ranging from about 1 nm to 6 μm.

In an embodiment, this disclosure provides a composition for use in a remotely-triggered antimicrobial combination therapy comprising (a) a particle heater having a material interacting with an exogenous source and a carrier; and (b) a pharmaceutical dosage of an antimicrobial agent.

In some embodiments, the particle heater and the pharmaceutical dosage forms a unitary dosage. In some embodiments, the particle heater and the pharmaceutical dosage are two discrete preparations. In some embodiments, the pharmaceutical dosage is selected from the group of a capsule, a tablet, a buccal tablet, a sublingual tablet, an orally disintegrating tablet, a liquid formulation, a dispersion, an injection preparation, powder for injection, and suppository.

In some embodiments, the particle heaters are nanoparticles or microparticles. In some embodiments, the particle heater is further combined with a pharmaceutically acceptable excipient to form a particle heater formulation. In some embodiments, the particle heater formulation is selected from the group of a capsule, a tablet, a buccal tablet, a sublingual tablet, an orally disintegrating tablet, a liquid formulation, a dispersion, an injectable formulation, powder for injection, and suppository.

In an embodiment, this disclosure provides a method for treating microbial infection with a synergistic combination therapy in a subject comprising: (1) administering a therapeutically effective amount of the synergistic combination therapy as disclosed herein to the subject in need thereof and allowing the synergistic combination therapy to associate with the microbes at the infection site, and (2) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the antimicrobial agent exhibit synergy in killing microbes, and wherein the particle is constructed such that it passes the Extractable Cytotoxicity Test. In some embodiments, the antimicrobial agent is further encapsulated by the particle heater, and the heat causes the release of the antimicrobial agent outside of the particle.

In some embodiments, the particle heater and the antimicrobial agent are administered to the patient simultaneously. In some embodiments, the particle heater and the antimicrobial agent are administered to the patient sequentially. In some embodiments, the antimicrobial agent is administered before administering of the particle heater. In some embodiments, the particle heater is administered before administering the antimicrobial agent.

In some embodiments, the exogenous source is selected from an electromagnetic radiation, an electrical field, a microwave, a radio wave, an ultrasound, a magnetic field, or combinations thereof.

In some embodiments, the exogenous source comprises a LED light or a laser light. In some embodiments, the exogenous source comprises a LED light. In some embodiments, the first material absorbs optical energy at a wavelength from 400 nm to 750 nm. In some embodiments, the material is a squaraine dye, or a squarylium dye.

In some embodiments, the laser light is a pulsed laser light. In some embodiments, the laser pulse duration is in a range from milliseconds to microseconds, and the laser has an oscillation wavelength at 805 nm, 808 nm, or 1064 nm. In some embodiments, the particle heater absorbs the laser light having a wavelength from 750 nm to 1100 nm. In some embodiments, the particle heater comprises indocyanine green (ICG), new ICG (IR 820), IR 780 dye, IR 193 dye, squaraine dye, Epolight™ IR 1117, Epolight™ IR 1175, iron oxide, and combinations thereof. In some embodiments, the particle heater comprises a zinc iron phosphate pigment.

In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C., wherein the hyperthermia does not cause collateral damage.

In some embodiments, the pathogenic microbes are bacteria. In some embodiments, the bacteria are multidrug resistant bacteria. In some embodiments, the multidrug resistant bacteria are selected from the group of Gram-positive bacteria, Gram-negative bacteria, and combinations thereof. In some embodiments, the multidrug resistant bacteria are selected from the group of E. coli, K. pneumonia, M. tuberculosis, Streptococcus aureus, P. aeruginosa, Streptococcus epidermidis, Streptococcus haemolyticus, Bacillus anthracis, Clostridium difficile, Streptococcus pyogenes, Streptococcus pneumonia, Enterococcus faecalis, and combinations thereof.

In an embodiment, this disclosure provides a method of treating a microbial infection in a subject in need thereof comprising the steps of sensitizing the microbes by administering to the subject a treatment that will (i) induce apoptosis in pathogenic microbial cells at an infection site, (ii) induce autolysis in pathogenic microbial cells at an infection site (iii) induce the generation of reactive oxygen species, (iv) stimulate infection-infiltrating immune cells, or (v) a combination of two or more thereof.

In some embodiments, the treatment is a particle heater and an antimicrobial agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat travels outside the particle to induce localized hyperthermia, wherein the hyperthermia and the antimicrobial agent exhibit synergy in killing microbes.

In some embodiments, the antimicrobial agent is encapsulated in the particle heater and the heat causes the release of the antimicrobial agent. In some embodiments, the antimicrobial agent is not encapsulated in the particle heater. In some embodiments, the antimicrobial agent is present in a separate pharmaceutical composition from the particle heater. In some embodiments, the particle heater is administered before the administration of the antimicrobial agent. In some embodiments, the particle heater is administered after the administration of the antimicrobial agent. In some embodiments, the particle heater is administered concurrently with the administration of the antimicrobial agent.

In some embodiments, the method further comprises the step of exposing the particle heater remotely to an exogenous source, wherein the exogenous source is selected from the group of an electromagnetic radiation, an electrical field, a microwave, a radio wave, an ultrasound, a magnetic field, and combinations thereof.

In some embodiments, sensitizing the microbe comprises administering to the subject a treatment that will induce autolysis or apoptosis in microbes. In some embodiments, the treatment that will induce autolysis or apoptosis in microbes is selected from the group of thermal therapy, antibiotic therapy, immunotherapy, phototherapy, or a combination thereof. In some embodiments, the treatment that will induce autolysis or apoptosis in microbes is thermal therapy. In some embodiments, the treatment that will induce autolysis or apoptosis in cells is thermal therapy and antibiotic.

### BRIEF DESCRIPTION OF THE DRAWINGS

FIG. 1A is a flowchart of the feedback loop (Feedback Loop 1A) for identifying optimal particle structure guided by ECT/EDP.

FIG. 1B is a flowchart of the feedback loop (Feedback Loop 1B) for identifying optimal particle structure guided by ECT/EDP/TCT.

FIG. 2 illustrates the particle size distribution measured by Horiba LA-950 particle size analyzer in de-ionized water with pH 7.4.

FIG. 3 illustrates the Infrared absorbance spectra for the degradation of IR absorbing agent in a neutrophil medium.

FIG. 4 illustrates the Infrared absorbance spectra for the degradation of IR absorbing agent in a macrophage medium.

FIG. 5 illustrates the degradation of Epolight™ 1117 measured at 1064 nm wavelength after exposure to 80° C.

FIG. 6 is a schematic of the transwell plate for TCT with a cross-section showing the two cell types.

FIG. 7 illustrates the controlled heat generation from laser excited Epolight™ IR 1117 loaded particles dispersed in gelatin. A red 50° C. thermochromic dye was suspended in gelatin as an indicator of heat generation by the color change from red color to colorless. Spots 1, 4, 5, 6, 7 of FIG. 5 were exposed to laser irradiation from a Lutronic laser with a pulse width of 10 ns operated under Q-switched mode. Spots 2 and 3 were exposed with the Lutronic laser with a pulse width of 350 μs. Spots 8-16 were exposed with a semiconductor laser using various pulse widths from 10-250 ms.

FIG. 8 illustrates the suspension of red thermochromic dye prior to laser exposure.

FIG. 9 illustrates the color change at spot 9 after two exposures with a semiconductor laser operated at a wavelength of 980 nm with a pulse width of 250 ms to produce a total fluence of 70.7 J/cm2.

FIG. 10A illustrates the melting of gelatin and decolorization of red dye without any clearing of the IR absorbing agent at the spots 15 and 16 after laser irradiation at 980 nm and a total fluence of 14.7 J/cm² (FIG. 7, Spot 15) and 14.1 J/cm² (FIG. 7, Spot 16).

FIG. 10B illustrates the color state at spot 15 after irradiating Spot 15 with seven exposures of 30 ms at 980 nm and a total fluence of 14.7 J/cm2.

FIG. 10C illustrates the color state at spot 16 after irradiating Spot 16 with a single exposure of 200 ms at 980 nm and a total fluence of 14.1 J/cm2.

FIG. 11 illustrates the Biopharmaceutics Classification System for poorly watersoluble drugs.

FIG. 12 illustrates the particle size distribution of the resulting VTMS encased curcumin/Epolight™ IR 1117/MMA/BMA copolymer particles of Example 13(b) were measured with Horiba LA-950 Particle Size Analyzer in distilled water with pH 7.4.

FIG. 13. Illustrates the leaching testing results demonstrated that the VTMS shell reduced the leaching of the curcumin by 70% and reduced the leaching of Epolight™ 1117 by 96%.

FIGS. 14A-B illustrate the TEM images for curcumin-Epolight™ IR 1117 loaded PMMA-BMA B-805 particle without VTMS shell.

FIGS. 15A-B illustrate the TEM images for curcumin-Epolight™ IR 1117 loaded PMMA-BMA B-805 particle having VTMS shell.

FIGS. 16A-B: illustrate the Efficacy Determination Protocol testing results for stability of Epolight™ IR 1117 and curcumin inside the B-805 particles with or without the VTMS shell. The testing results demonstrated that the degradation of Epolight™ IR 1117 after incubation in DMEM media in particles without shell than that in particles with the VTMS shell. Little degradation was observed for curcumin after incubation in DMEM media in particles with or without the VTMS shell.

FIGS. 17A-B: illustrate the laser triggered release of Epolight™ IR 1117 and curcumin inside the B-805 particles with the VTMS shell.

FIG. 18 illustrated the Extractable Cytotoxicity Test results at neat and 1× dilution for B-805 particles having VTMS shell as compared with the control particles without VTMS shell.

FIG. 19 illustrated the Extractable Cytotoxicity Test results for released curcumin at neat for laser treated B-805 particles having VTMS shell as compared with the control particles without laser treatment.

FIG. 20 illustrated the cytotoxicity test results for supernantant at neat for laser treated B-805 particles having VTMS shell as compared with the control particles without laser treatment.

FIGS. 21A and 21B illustrate the results of the efficacy determination protocol (EDP) for core particles with 8% IR dye.

FIGS. 22A and 22B illustrate the results of the EDP for core-shell particles with 8% IR dye.

FIGS. 23A and 23B illustrate the results of the EDP for core particles with 2% curcumin and 8% IR dye.

FIGS. 24A and 24B illustrate the results of the EDP for core-shell particles with 2% curcumin and 8% IR dye.

FIGS. 25A and 25B illustrate the results of the EDP for core particles with 5% curcumin and 5% IR dye.

FIGS. 26A and 26B illustrate the results of the EDP for core-shell particles with 5% curcumin and 5% IR dye.

FIG. 27 illustrates the ECT results for core and core-shell particles containing either IR dye or IR dye and curcumin.

FIG. 28 schematically illustrates the irradiation pattern in which cells in a given well are irradiated.

FIGS. 29A-29C show the results of thermal cytotoxicity cells for irradiation by light of 805 nm wavelength.

FIGS. 30A-30C show the results of thermal cytotoxicity cells for irradiation by light of 1064 nm wavelength.

FIGS. 31A and 31B show the results for synergestic combination treatment in which particles with only free curcumin and IR dye were respectively compared to particles containing curcumin and IR dye.

### DETAILED DESCRIPTION OF THE INVENTION

This disclosure provides particles, methods and compositions for the remotely triggered treatment of cancer and microbial infections. In some embodiments, the disclosure achieves this using particles with structures modified to retain efficacy and reduce collateral toxicity. Feedback loop protocols are used to modify the particles to improve efficacy and reduce toxicity.

In some embodiments, particles are loaded with an active agent and a material that responds to an exogenous source by producing heat inside the particle that can trigger release of the active agent from the particles to kill unwanted cells like cancer cells and pathogenic microbes. Killing of unwanted cells is primarily mediated by the triggered release of the active agent.

In some embodiments, particles are loaded with a material that responds to an exogenous source to produce heat that travels outside the particle to kill unwanted cells like cancer cells and pathogenic microbes, also called particle heater. Killing of unwanted cells is primarily mediated by hyperthermia.

In some embodiments, particles are loaded with an active agent and a material that responds to an exogenous source by producing heat—the heat can travel outside the particle and trigger release of the active agent from the particles to synergistically kill unwanted cells like cancer cells and pathogenic microbes using combination therapies. Killing of unwanted cells is mediated by the combination of hyperthermia and triggered release of the active agent.

In some embodiments, structure of particles described above is modified iteratively using feedback loop A which involves use of two tests—efficacy determination protocol (EDP) and extractable cytotoxicity test (ECT). EDP evaluate the ability of the particle structure to retain efficacy of the payload (by reducing intrusion of body chemicals into the particles) while ECT evaluates the ability of the particle structure to limit unwanted toxicity of the payload to the body.

In some embodiments, particles described above are each modified iteratively using feedback loop B which involves use of a third test in addition to the EDP and ECT—the thermal cytotoxicity test (TCT) which is designed to enhance killing of the unwanted cells and reducing killing of healthy cells using hyperthermia.

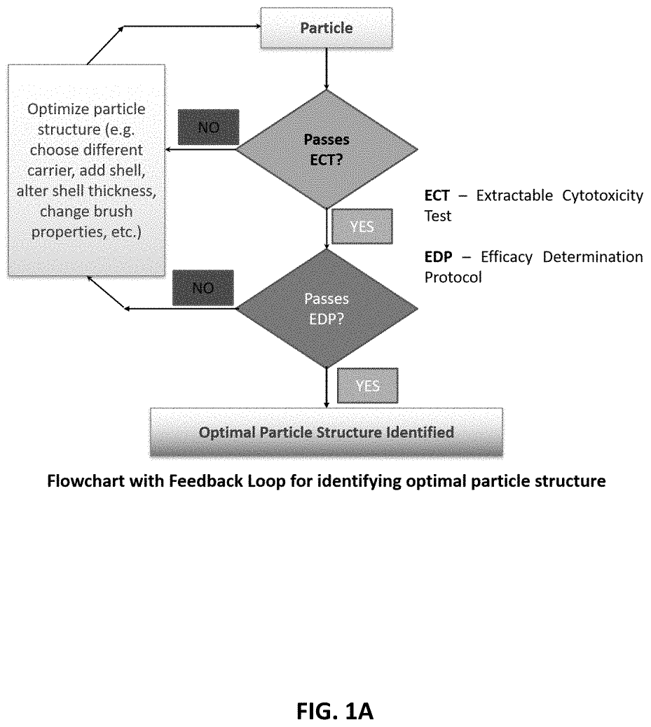

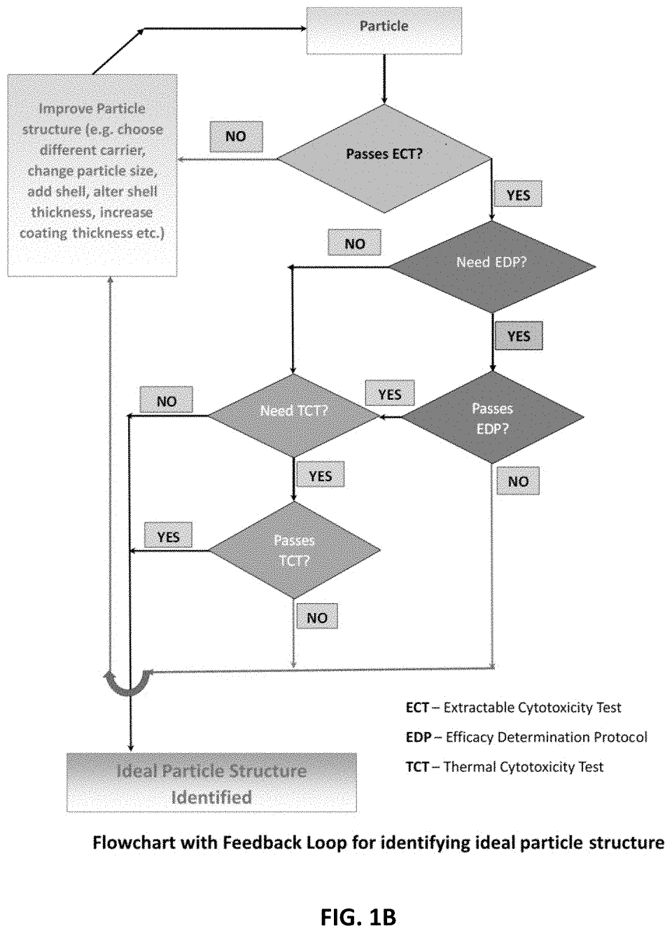

In some embodiments, disclosed herein are particles with desired properties guided by the feedback loop protocols (Feedback Loop 1A described in FIG. 1A and Feedback Loop 1B described in FIG. 1B) that are virtually impenetrable until activation by the application of an exogenous source. The exogenous source causes the particle to release the chemoactive agentactive agent outside of the particle.

The conventional particles formed by encapsulation of the material and/or active agent (anticancer agent or antimicrobial agent as described herein) with a carrier have some limitations: such as the degradation caused by the body fluids' incursion into the particles, and the cytotoxicity caused by leakage of the material and/or active agent before the particles reach the infection site, given the inherent porosity of the particles.

The encapsulation of the material and the active agent with the carrier may reduce the degradation and the leakage mentioned above. The porosity of a particle depends on various factors, including the molecular weight of the polymer, the structure of the polymer, the crosslinker and the amount thereof, the polymerization temperature, and solvent, etc. Therefore, it is desirable to have an efficient method of controlling the particle porosity. To this end, the present invention provides a method of controlling the porosity of the particles via feedback loop protocols depicted in FIGS. 1A-B, resulting in much safer particles for human use. As shown in FIGS. 1A-B, the particle structure is sequentially designed to reduce: (1) the toxicity of the materials and active agent that leak out of the particle to healthy cells, and (2) the loss of energy conversion efficiency of the materials and the loss of efficacy of the active agent due to their breakdown from the entry of body chemicals into the particle. (3) the thermal toxicity to healthy cells while maximizing toxicity to unwanted cells.

The encapsulated material and active agent within a particle may be protected from degradation by limiting their exposure to the chemicals from the surrounding environment. However, due to the inherent porosity of the carrier of the particle, degrading body chemicals can still to some extent diffuse into the particle, causing the degradation of the encapsulated material and active agent. Further, the encapsulated material and active agent can also leak outside the particle, causing toxicity to the surrounding environment.

Judicious choice of the carrier can provide some control over such incursion or leakage, but may not be enough to assure passing the Efficacy Determination Protocol or the Extractable Cytotoxicity Test. The disclosed inventions provide embodiments of methods for designing particle structure to achieve the desired level of cytotoxicity and active agent efficacy guided by the feedback loop protocol as illustrated in FIGS. 1A-B. In some embodiments, an additional shell may be needed to enclose the particle if the carrier does not provide sufficient protection as determined by the feedback loop protocols.

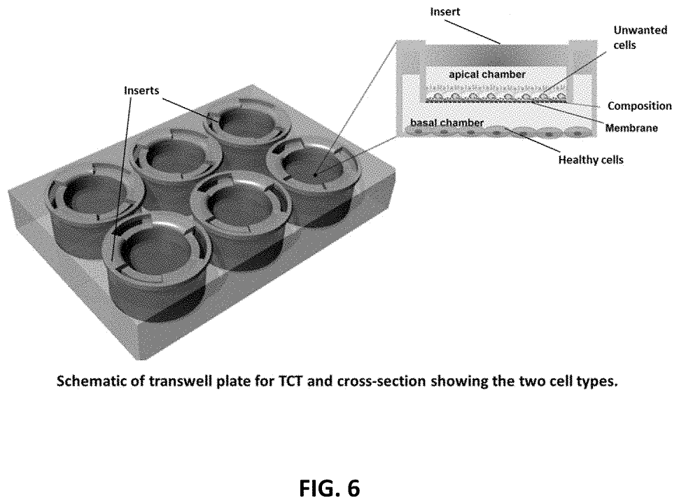

The final particle structure is designed using three tests or assays: 1. Extractable Cytotoxicity Test which evaluates the ability of body chemicals (like serum) to extract the material that interacts with the exogenous source and tests the ability of these extracts to kill cells. Particle structure that limits leakage of the material such that no more than 30% of the cells are killed are considered safe for further use. 2. Efficacy Determination Protocol—In this assay particles are incubated with physiologically relevant media (e.g. cell culture media containing serum proteins) such that chemicals present in these media may enter the particle and breakdown or reduce the efficacy of the material to absorb exogenous energy and convert it to heat. The particle structure is iteratively modified such that the chemicals break down no more than 25% of the material in the physiologically relevant media. 3. Thermal Cytotoxicity Test—This is an in vitro test specifically designed to test the particles and the specific exogenous source(s) for their ability to kill the unwanted cells while sparing the healthy cells. The Thermal Cytotoxicity Test is a transwell assay wherein two different cells types, e.g., one being the unwanted cells with the other type being the healthy cells, are grown in the same well and exposed to different doses of the particles and the exogenous source (see FIG. 6). Viabilities of the two cells types are assessed a day after exposure of the cells to the particles and the exogenous source using standard colorimetric assays. Different types of unwanted cells and/or normal cells can be selected for this test for different therapy applications. The particle and the exogenous source that do not kill any more than 30% of the healthy cells but kill at least 70% of the unwanted cells are considered passing the Thermal Cytotoxicity Test. Use of these rigid tests to improve particle structural design has not been explored in the prior art.

In some embodiments, this disclosure provides a composition comprising a particle heater having a carrier admixed with a material that interacts with an exogenous source; wherein the material absorbs and converts the energy from the exogenous source to heat, and the heat then causes cell death, and further wherein the particle structure is constructed such that it passes the Extractable Cytotoxicity Test.

In some embodiments, the material exhibits at least 20% efficiency of conversion of the energy from the exogenous source to heat. In some embodiments, the material exhibits at least 20% photothermal conversion efficiency.

In some embodiments, the material interacts with the exogenous source to produce heat for selective killing unwanted or diseased cells and tissues.

In some embodiments, the particle heater further comprises an active agent. In some embodiments, the active agent is selected from the group of agents capable of generating reactive oxygen species, therapeutic drugs, antimicrobial agent, anti-cancer agent, anti-scarring agent, anti-inflammatory agent, metalloprotease inhibitors, treatment sensitizing the unwanted cells to remotely triggered thermal therapy, and combinations thereof.

In an embodiment, this disclosure provides a method for inducing localized hyperthermia at a tissue site in a subject comprising: administering an effective amount of the particle heater described herein to the tissue site in the subject; exposing the material to an exogenous source to absorb energy and covert it to heat which diffuses out of the particle heater to induce localized hyperthermia at a temperature ranging from about 38.0° C. to about 52.0° C. for a sufficient period of time to kill unwanted cells.

In some embodiments, the exogenous source is electromagnetic radiation, microwaves, radio waves, sound waves, electrical or magnetic field. In some embodiments, the exogenous source comprises a LED light or a laser light. In some embodiments, the laser light is a pulsed laser light. In some embodiments, the exogenous source comprises a LED light. In some embodiments, the laser pulse duration is in a range from milliseconds to nanoseconds, and the laser has an oscillation wavelength at 805 nm, 808 nm, or 1064 nm. In some embodiments, the laser pulse duration is in a range from milliseconds to femtoseconds, and the laser has an oscillation wavelength at 805 nm, 808 nm, or 1064 nm. In some embodiments, the particle heater absorbs the visible light having a wavelength ranging from 400 nm to 750 nm. In some embodiments, the particle heater absorbs the laser light having a wavelength ranging from 750 nm to 1400 nm.

In some embodiments, the material is a tetrakis aminium dye. In some embodiments, the material is indocyanine green. In some embodiments, the material is a squaraine dye. In some embodiments, the material is a squarylium dye. In some embodiments, the material is iron oxide. In some embodiments, the material is a plasmonic absorber. In some embodiments, the plasmonic absorber is selected from the group of gold nanostructures including gold nanorod, gold nanocage, gold nanosphere, gold thin film, silver nanoparticle, and combinations thereof.

In some embodiments, the method further comprises heating a surrounding area in proximity to the particle heater by transferring heat from the particle heater to the surrounding area. In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C.

In some embodiments, at least a portion of the exterior surface of the particle heater has a modification that is polar, non-polar, charged, ionic, basic, acidic, reactive, hydrophobic, or hydrophilic.

In some embodiments, the particle heater maintains integrity after interacting with the exogenous source. In some embodiments, the particle structure is altered after interacting with the exogenous source.

In some embodiments, the present disclosure provides particle heaters having a core-shell structure to reduce particle porosity and to protect the material from the degradation by the body chemicals. Therefore, the stability of the material inside the particles are improved due to the reduced incursion of the body chemicals. In some embodiments, the shell comprises a crosslinked inorganic polymer. In some embodiments, the crosslinked inorganic polymer comprises organo-modified polysilicates. The shell may comprise inorganic polymers such as silicates, organosilicate, and organo-modified silicone polymer derived from condensation of organotrisilanol or halotrisilanol. The process to apply the crosslinked shell must be designed so as to maximize the stability of the particle heater components to the chemistry required in shell construction, at least until the growing shell protects the components encapsulated in the particle heater. For example, to protect the IR absorbing agent Epolight™ 1117 encapsulated in a NeoCryl® 805 particle when introduced into human skin, a sol-gel organo-modified silicate polymer shell derived from alkyltrimethoxysilane is formed on the surface of the polymeric particle to block the free exchange of nucleophiles and free radical species between the particles and the surrounding environment.

In some embodiments, the trialkoxysilane used for making the shell is selected from the group of C2-C7 alkyl-trialkoxysilane, C2-C7 alkenyl-trialkoxysilane, C2-C7 alkynyl-trialkoxysilane, aryl-trialkoxysilane, and combinations thereof. In some embodiments, the trihalosilane used for making the shell is selected from the group of trichlorosilane, tribromosilane, triiodosilane, and combinations thereof. In some embodiments, the crosslinked organo-silicate polymer is derived from vinyl-trimethoxysilane.

In some embodiments, the shell comprises an agent selected from the group of inorganic polymers, organic polymers including polyureas or polyurethanes, silicates, mesoporous silica, organosilicate, organo-modified silicone polymers, cross-linked organic polymers, and combinations thereof. In some embodiments, the shell is formed of an agent selected from the group of protein, polysaccharide, lipid, and combinations thereof.

In some embodiments, the particle heater core comprises a plasmonic absorber or iron oxide nanoparticles. In some embodiments, the shell comprises a plasmonic absorber or iron oxide. In some embodiments, the plasmonic absorber comprises plasmonic nanomaterials selected from the group of noble metal including gold (Au) nanostructure, silver (Ag) nanoparticle, copper (Cu) nanoparticle having a plasmonic resonance at a NIR wavelength, and combinations thereof. In some embodiments, the shell comprises an agent selected from the group of gold nanostructures, silver nanoparticles, iron oxide film, iron oxide nanoparticle, and combinations thereof.

In some embodiments, this disclosure provides a method of remotely triggered thermal killing of unwanted cells comprising the steps of: (1) administering an therapeutically effective does of heat delivery particles and waiting for a period of time to allow distribution of the particles to the unwanted cells, (2) exposing the tissue site having unwanted cells to an exogenous source for sufficient period of time, wherein the material absorbs the energy from the exogenous source and converts the energy to heat, wherein the heat induces localized hyperthermia at the tissue site, wherein the localized hyperthermia causes the death of the unwanted cells.

In some embodiments, this disclosure provides a method for effecting remotely triggered thermal killing of unwanted cells at a tissue site comprising: (1) administering a therapeutically effective amount of the particle heaters as described herein to the tissue site having the unwanted cells and allowing the cells to associate with the particle heaters, and (2) exposing the particle heaters at the tissue site to an exogenous source for a sufficient period of time, wherein the particle is constructed such that it passes the Extractable Cytotoxicity Test, and the material absorbs the energy from the exogenous source and converts the energy into heat; then the heat travels outside the particle to cause a temperature increase in a tissue area surrounding the particle heaters thereby to induce localized hyperthermia at a temperature ranging from about 38.0° C. to about 52.0° C. that is sufficient to selectively kill the unwanted cells. In some embodiments, the material in the particle exhibits stability such that the particle is considered passing the Efficacy Determination Protocol.

In some embodiments, the particle exhibits energy-to-heat conversion stability such that the loss in absorbance of the material is less than 50% as measured by the Material Process Stability Test after exposure to a pulsed laser light.

In some embodiments, for any herein described methods, the “unwanted cells” comprise cancer cells. In some embodiments, for any herein described methods, the “unwanted cells” comprise pathogenic microbial cells.

In an embodiment, this disclosure provides a particle heater for use in the remotely-triggered thermotherapy for killing unwanted cells comprising: the herein described material admixed with the carrier described herein, wherein the material in the particle heater exhibits stability such that the particle is considered passing the Efficacy Determination Protocol; wherein the particle is constructed such that it passes the Extractable Cytotoxicity Test; wherein the particle and specific dose(s) of the exogenous source pass the Thermal Cytotoxicity Test; wherein the material absorbs the energy from the exogenous source and converts the energy into heat; and then the heat travels outside the particle to induce localized hyperthermia sufficient to selectively kill the unwanted cells.

In some embodiments, the material exhibits at least 20% efficiency of conversion of the energy from the exogenous source to heat. In some embodiments, the material exhibits at least 20% photothermal conversion efficiency.

In some embodiments, this disclosure provides synergistic combination therapies combining remotely-triggered particle heaters with conventional chemotherapy. The synergistic combination therapy as described herein overcomes the limitations of conventional chemotherapies or targeted chemotherapy because it produces synergistic therapeutic effects, reduces drug-related toxicity and inhibits multidrug resistance through different mechanisms: e.g., via new pathway to kill unwanted cells by localized hyperthermia. The synergistic thermo-chemo therapeutic effects can reduce the required does of the chemoactive agent and has the potential to overcome drug resistance. The remotely-triggered synergistic combination therapy as disclosed herein show the potentials in applications for treating drug resistant disease conditions.

In an embodiment, this disclosure provides a method for causing remotely-triggered combination therapy in a subject in need thereof comprising: (1) administering a therapeutically effective amount of a particle heaters comprising a carrier admixed with a material and a active agent to the diseased tissue site in the subject, and (2) activating the particles with an exogenous source for a sufficient period of time to produce heat, wherein the particle is constructed such that it passes the Extractable Cytotoxicity Test and/or the Thermal Cytotoxicity Test; wherein the material absorbs the energy from the exogenous source and converts the energy into heat; and then the heat travels outside the particle to induce localized hyperthermia at a temperature ranging from about 38.0° C. to about 52.0° C. that is sufficient to selectively kill unwanted cells, wherein the heat causes the particle to become permeable to liquid whereby the release of the active agent occurs outside the particle, and wherein the collateral damage to the healthy cells is minimized.

In some embodiments, the active agent and the hyperthermia may be administered concurrently or sequentially. In some embodiments, the active agent and the hyperthermia may be administered concurrently. the active agent and the hyperthermia may be administered sequentially. In some embodiments, the hyperthermia may be administered before the administering of the active agent. In some embodiments, the hyperthermia may be administered post the administration of the active agent.

### Definitions

As used in the preceding sections and throughout the rest of this specification, unless defined otherwise, all the technical and scientific terms used herein have the same meaning as is commonly understood by one of skill in the art to which this invention belongs. All patents and publications referred to herein are incorporated by reference in their entireties.

Amino acids are represented in three letter code or one letter code, as illustrated in the Table below.

| Amino acid | Three letter code | One letter code |
|---|---|---|
| alanine | Ala | A |
| arginine | Arg | R |
| asparagine | Asn | N |
| aspartic acid | Asp | D |
| asparagine or aspartic acid | Asx | B |
| cysteine | Cys | C |
| glutamic acid | Glu | E |
| glutamine | Gln | Q |
| glutamine or glutamic acid | Glx | Z |
| glycine | Gly | G |
| histidine | His | H |
| isoleucine | Ile | I |
| leucine | Leu | L |
| lysine | Lys | K |
| methionine | Met | M |
| phenylalanine | Phe | F |
| proline | Pro | P |
| serine | Ser | S |
| threonine | Thr | T |
| tryptophan | Trp | W |
| tyrosine | Tyr | Y |
| valine | Val | V |

The terms “a,” “an,” and “the” as used herein, generally are construed to cover both the singular and the plural forms.

The term “about” as used herein, generally refers to a particular numeric value that includes variation and an acceptable error range as determined by one of ordinary skill in the art, which will depend in part on how the numeric value is measured or determined, i.e., the limitations of the measurement system. For example, “about” can mean zero variation, and a range of ±20%, ±10%, or ±5% of a given numeric value.

The term “absorption” as used herein, generally refers to the process of matter taking up exogenous energy and transforming the state of that matter to a higher electronic state when interacting with an exogenous source described herein. The process of absorption leads to an attenuation in the intensity of the exogenous energy.

In some embodiments, the term “active agent” as used herein refers to therapeutic agent including anticancer agent and antimicrobial agent.

As used herein, the term “antibody” encompasses antibody fragments and derivatives such as polyclonal, monoclonal, chimeric, single chain, Fab fragments and fragments produced by a Fab expression library. Such fragments include fragments of whole antibodies that retain their binding activity for an antigen. Such fragments include Fv, F(ab′) and F(ab′)2 fragments, as well as single chain antibodies (scFv), fusion proteins and other synthetic proteins that comprise the antigen-binding site of the antibody. While the antibodies are principally being used herein as targeting agents, such antibodies and fragments thereof may also be neutralizing antibodies, i.e., those that inhibit biological activity of the polypeptides that they recognize, and therefore may serve the additional purpose of rendering the particle heaters as being useful as diagnostics and therapeutics.

As used herein, the term “aptamers” are DNA or RNA molecules that have been selected from random pools based on their ability to bind other molecules. Aptamers have been selected which bind nucleic acid, proteins, small organic compounds, and even entire organisms.

The term “biocompatibility” as used herein, refers to the capability of a material implanted in the body to exist in harmony with the tissue without causing deleterious changes.

The term “biocompatible polymer” as used herein, generally refers to polymers that are intended to interface with biological systems to evaluate, treat, augment or replace any tissue, organ or function of the body. Some of the characteristic properties of the biocompatible polymers include “not having toxic or injurious effects on biological systems,” “the ability of a polymer to perform with an appropriate host response in a specific application,” and “ability of a polymer to perform its desired function with respect to a medical therapy, without eliciting any undesirable local or systemic effects in the recipient or beneficiary of that therapy, but generating the most appropriate beneficial cellular or tissue response in that specific situation, and modifying the clinically relevant performance of that therapy.”

The term “chromophore” as used herein refers to a chemical group (such as a xanthene group, or an acridine group) that absorbs light at a specific frequency and so imparts color to a molecule.

The term “dye” as used herein include the IR absorbing agent.

The term “IR absorbing material” as used herein is used interchangeably with the term “IR absorbing agent”.

The term “IR dye” is used interchangeably with the term “infrared radiation absorbing agent” (IR absorbing agent).

The term “biodegradable” as used herein, refers to polymers that degrade fully (i.e., down to monomeric species) under physiological or endosomal conditions. Biodegradable polymers are not necessarily hydrolytically degradable and may require enzymatic action to be fully degradable.

The term “body chemicals” as used herein, generally refers to chemicals existing in any one of the bodily fluids, neutrophil media, macrophage media, or any complete cell growth media.

The term “bodily fluid” as used herein, generally refers to a natural fluid found in one of the fluid compartments of the human body. The principal fluid compartments are intracellular and extracellular. A much smaller segment, the transcellular compartment, includes fluid in the tracheobronchial tree, the gastrointestinal tract, and the bladder; cerebrospinal fluid; and the aqueous humor of the eye. Bodily fluid includes blood plasma, serum, cerebrospinal fluid, or saliva. In an embodiment, bodily fluid contains neutrophils and macrophages.

The term “EDC-NHS chemistry” refers to specific chemical reactions that form amide bonds. First, 1-ethyl-3-(3-dimethylaminopropyl)carbodiimide hydrochloride (EDC) reacts with a molecule containing a carboxylic-acid group, forming an amine-reactive O-acyl isourea intermediate. This intermediate may further react with N-hydroxysuccinimide (NHS) or N-hydroxysulfosuccinimide (Sulfo-NHS) to form a semi-stable amine-reactive NHS ester, which further reacts with a compound containing an amine, yielding a conjugate of the two molecules (the carboxylic acid and the amine) joined by a stable amide bond.

The term “Efficacy Determination Protocol” as used herein, generally refers to the method used for determining the degree of the degradation of the material or the active agent inside a particle, wherein the material and/or the active agent interacts with body chemicals, after being treated with body chemicals for a period of time. Various analytical tools, like UV-VIS-NIR, NMR, HPLC, LCMS etc., would be used to quantify the concentration of the IR absorbing agent in the extracts and control. Tools like UV absorbance spectrophotometry and circular dichroism can be used to monitor peptide degradation by body chemicals. The details of Efficacy Determination Protocol are described in the Examples section of the disclosure. In some instances, if the degradation of the material and the active agent each independently is less than 90%, then the particle is considered passing the Efficacy Determination Protocol. In some instances, depending on the potency of the active agent, the thermal conversion efficiency and the physicochemical property of the material, if the degradation of the active agent is less than 85%, 80%, 75%, 70%, 65%, 60%, 55%, 50%, 45%, 40%, 35%, 30%, 25%, 20%, 15%, 10%, or 5%, and the degradation of the material is less than 85%, 80%, 75%, 70%, 65%, 60%, 55%, 50%, 45%, 40%, 35%, 30%, 25%, 20%, 15%, 10%, or 5%, then the particle is considered passing the Efficacy Determination Protocol.

The term “Extractable Cytotoxicity Test” as used herein, generally refers to an in vitro leaching protocol (using physiologically relevant media that contains serum proteins at physiological temperature) that can be used to extract the material from the particles. The extract can then be used in a cytotoxicity test against healthy cells (different cells will be chosen depending upon the application) as is (“neat” or 1×) or in serial dilutions (up to 0.0001× dilutions) with the media. The neat or dilution of the extract that kills 30% of the cells can be measured and is referred to as an IC₃₀. Likewise, the neat or dilution of the extract that kills 10% of the cells can be measured and is referred to as an IC₁₀. The neat or dilution of the extract that kills 20% of the cells can be measured and is referred to as an IC₂₀. The neat or dilution of the extract that kills 40% of the cells can be measured and is referred to as an IC₄₀. The neat or dilution of the extract that kills 50% of the cells can be measured and is referred to as an IC₅₀. The neat or dilution of the extract that kills 60% of the cells can be measured and is referred to as an IC₆₀. The neat or dilution of the extract that kills 70% of the cells can be measured and is referred to as an IC₇₀. The neat or dilution of the extract that kills 80% of the cells can be measured and is referred to as an IC₈₀. The neat or dilution of the extract that kills 90% of the cells can be measured and is referred to as an IC₉₀. Details of the Extractable Cytotoxicity Test are described in the Examples section of this disclosure. The extractable cytotoxicity test is compliant with the international standards: ISO-10993-5 “Tests for cytotoxicity—in vitro methods.” In some instances, if the neat or dilution concentration of the material in the leachate is less than IC₁₀, IC₃₀, IC₄₀, IC₅₀, IC₆₀, IC₇₀, IC₈₀, or IC₉₀, the particle passes the Extractable Cytotoxicity Test.

The term “electromagnetic radiation” (EMR) as used herein, generally refers to a complex system of radiant energy composed of waves and energy bundles that are organized according to the length of the propagating wave. It includes radio waves, microwaves, infrared (IR), visible light, ultraviolet, X-rays, and gamma rays.

The term “energy fluence” as used herein, generally refers to the areal density of the energy contained within the light and expressed in joules/area, for example, joules/m² or joules/cm².

The term “feedback loop” as used herein, generally refers to a feedback loop based on the Extractable Cytotoxicity Test (ECT) and/or Efficacy Determination Protocol (EDP) and/or the Thermal Cytotoxicity Test (TCT), which have been utilized to evaluate if a particle needs to be rendered less porous by altering the chemistry of the particle fabrication. Feedback Loop 1A, describe in FIG. 1A, is a flowchart of the feedback loop for identifying optimal particle structure guided by ECT/EDP. Feedback Loop 1B, described in FIG. 1B, is a flowchart of the feedback loop for identifying optimal particle structure guided by ECT/EDP/TCT. The feedback Loop 1A is used to optimize particle used for application in remotely triggered drug delivery particles for anticancer agent and antimicrobial agents. The feedback Loop 1B is used to optimize particle used for application in remotely triggered thermal therapy, anticancer therapy, antimicrobial therapy and the synergistic combination therapy thereof.

In the Extractable Cytotoxicity Test, when cell death is less than 30% then the particles are considered to have passed the Extractable Cytotoxicity Test. The Extractable Cytotoxicity Test is compliant with the international standards: ISO-10993-5 “Tests for cytotoxicity—in vitro methods.” In Efficacy Determination Protocol, when the degradation of the material and the active agent each independently is less than 20%, then the particle is considered passing the Efficacy Determination Protocol. In Thermal Cytotoxicity Test, when the composition and light dose(s) that do not kill any more than 30% of the healthy cells but kill at least 70% of the unwanted cells are considered passing the Thermal Cytotoxicity Test.

The term “energy-to-heat conversion efficiency” describes the percentage of absorbed exogenous energy that is converted into heat, as determined by a rise in temperature.

As used herein, the term “Ferroptosis” refers to a form of regulated cell death (RCD) initiated by oxidative perturbations of the intracellular microenvironment that is under constitutive control by Glutathione Peroxidase 4 (GPX4) and can be inhibited by iron chelators and lipophilic antioxidant.

The term “hydrophilic,” as used herein, refers to the property of having affinity for water. For example, hydrophilic polymers (or hydrophilic polymer segments) are polymers (or polymer segments) which are primarily soluble in aqueous solutions and/or have a tendency to absorb water. In general, the more hydrophilic a polymer is, the more that polymer tends to dissolve in, mix with, or be wetted by water.

The term “hydrophobic,” as used herein, refers to the property of lacking affinity for, or even repelling water. For example, the more hydrophobic a polymer (or polymer segment), the lower its tendency to dissolve in, to mix with, or be wetted by water.

The term “IR dye” as used herein refers to infrared radiation absorbing dye. It is well known in the art that some IR dyes respond to other exogenous triggers like sound to kill unwanted cells e.g. ICG when triggered using ultrasound produces reactive oxygen species through a process referred to as sonodynamic therapy.

The term “localized surface plasmon resonance” (LSPRs, localized SPRs) as used herein refers to collective electron charge oscillations in metallic nanoparticles that are excited by light. In contrast with the case of bulk metal, when light having various wavelengths is emitted onto an agent existing on a local surface such as metal nanoparticles, polarization occurs on the surface of metal nanoparticles and exhibits a unique characteristic of increasing the intensity of the electric field. Electrons formed by polarization form a group (plasmon) and locally vibrate on the surface of the metal nanoparticles. This phenomenon is called localized surface plasmon resonance (LSPR). They exhibit enhanced near-field amplitude at the resonance wavelength.

The term “macrophage medium” as used herein, generally refers to a complete medium designed for the culture of macrophages. The medium consists of basal medium (containing essential and non-essential amino acids, vitamins, organic and inorganic compounds, hormones, growth factors, trace minerals), supplemented with macrophage growth supplement, antibiotics, and fetal bovine serum.

The term “the material” as used herein, refers to the material that interacts with an exogenous source described in the disclosure.

The term “Material Process Stability” as used herein refers to the preservation of the optical and physical characteristics of the material under conditions of use such that it can deliver heat as intended upon stimulation by the exogenous source.

The term “microbial targeting group” (microbe localizing component) as used herein, refers to a moiety that localizes the particle to a specific microbe. The moiety may be, for example, a protein, peptide, aptamer, nucleic acid, nucleic acid analog, carbohydrate, or small molecule. The targeting group directs the localization of the particle heaters.

The term polymer “molecular weight” as used herein might mean any one of three different things. The term might refer (1) to “average molecular weight” (Mi) that is the molecular weight as calculated by the weight of the molecule that is most prevalent in the mix that makes up copolymer. The term might refer (2) to “number average molecular weight” (Mn) that is the molecular weight as calculated by taking all the different-sized molecules in the mix that makes up polymer and calculating the average weight, i.e., adding up the weight of each molecule and dividing by the number of molecules. Or, the term might refer (3) to “weight average molecular weight” (Mw) that is the molecular weight as calculated by taking all the different-sized molecules in the mix that makes up copolymer and calculating their average weight while giving heavier molecules a weight-related bonus when doing so. The unit for the molecular weight is Dalton (Da), kilodalton (KDa, plural kilodaltons).

The term “neutrophil medium” as used herein, generally refers to a complete medium designed for the culture of neutrophils. The medium contains a basal medium (containing essential and non-essential amino acids, vitamins, organic and inorganic compounds, hormones, growth factors, trace minerals), supplemented with neutrophil culture supplement, antibiotics (i.e. penicillin, streptomycin), L-glutamine, and fetal bovine serum (FBS).

The term “near infrared radiation” (NIR) as used herein, generally refers to commonly used subdivision scheme for Infrared EMR with wavelengths extending from 750 nm (400 THz) to 1400 nm (214 THz).

As used herein, the term “necrotic cell death” refers to a ROS-dependent modality of RCD restricted to cells of hematopoietic derivation and associated with NET extrusion.

The term “Nd:YAG” as used herein, generally refers to Neodymium-doped Yttrium Aluminum Garnet (YAG) a widely used solid-state crystal composed of yttrium and aluminum oxides and a small amount of the rare earth neodymium.

The terms “peptide” and “protein” as used herein, generally refer to a chain of amino acids that are held together by peptide bonds (also called amide bonds). Proteins and peptides are fundamental components of cells that carry out important biological functions. Proteins give cells their shape, for example, and they respond to signals transmitted from the extracellular environment. Certain types of peptides play key roles in regulating the activities of other molecules. The basic distinguishing factors for proteins and peptides are size and structure. Peptides are smaller than proteins. Traditionally, peptides are defined as molecules that consist of between 2 and 50 amino acids, whereas proteins are made up of 50 or more amino acids. In addition, peptides tend to be less well defined in structure than proteins, which can adopt complex conformations known as secondary, tertiary, and quaternary structures. Functional distinctions may also be made between peptides and proteins.

The term “pharmaceutically or pharmacologically acceptable” as used herein refers to molecular entities and compositions that do not produce an adverse, allergic or other untoward reaction when administered to a subject, or a human, as appropriate.

“Pharmaceutically acceptable carrier” or “pharmaceutically acceptable excipient” is intended to include all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in the therapeutic compositions of the invention is contemplated. Additional active pharmaceutical ingredients, such as other drugs, can also be incorporated into the described compositions and methods.

The term “photothermal conversion efficiency” describes the percentage of absorbed radiant energy that is converted into heat, as determined by a rise in temperature.

The term polymer “polydispersity (PD)” as used herein, generally is used as a measure of the broadness of a molecular weight distribution of a polymer and is defined by the formula polydispersity

$$PD = \frac{Mw}{Mn} .$$

The larger the polydispersity, the broader the molecular weight. A monodisperse polymer where all the chain lengths are equal (such as endogenous protein) has an Mw/Mn=1. The best-controlled synthetic polymers have Mw/Mn of 1.02 to 1.10.

The term “Polydispersity Index (PdI)” is defined as the square of the ratio of standard deviation (σ) of the particle diameter distribution divided by the mean particle diameter (2a), as illustrated by the formula: PdI=(σ/2a)². PdI is used to estimate the degree of non-uniformity of a size distribution of particles, and larger PdI values correspond to a larger size distribution in the particle sample. PdI can also indicate particle aggregation along with the consistency and efficiency of particle surface modifications. A sample is considered monodisperse when the PdI value is less than 0.1.

The term “power” as used herein, generally refers to the rate at which energy is emitted from a laser.

The term “power density (irradiance)” as used herein, generally refers to the quotient of incident laser power on a unit surface area, expressed as watts/cm² (W/cm²).

The term “pulse” as used herein, generally refers to the brief span of time for which, the focused and scanned laser beam interacts with a given point on the skin (usually ranging from femtoseconds to milliseconds).

The term “synergistic,” or “synergistic effect” or “synergism” as used herein, generally refers to an effect such that the one or more effects of the combination of compositions is greater than the one or more effects of each component al one, or they can be greater than the sum of the one or more effects of each component alone. The synergistic effect can be greater than a percent value selected from the group of about 10%, 20%, 30%, 40%, 50%, 60%, 75%, 100%, 110%, 120%, 150%, 200%, 250%, 350%, and 500% more than the effect on a subject with one of the components alone, or the additive effects of each of the components when administered individually. The effect can be any of the measurable effects described herein. Advantageously, such synergy between the agents when combined, may allow for the use of smaller doses of one or both agents, may provide greater efficacy at the same doses, and may prevent or delay the build-up of multi-drug resistance. The combination index (CI) method of Chou and Talalay may be used to determine the synergy, additive or antagonism effect of the agents used in combination. When the CI value is less than 1, there is synergy between the compounds used in the combination; when the CI value is equal to 1, there is an additive effect between the compounds used in the combination and when CI value is more than 1, there is an antagonistic effect. The synergistic effect may be attained by co-formulating the agents of the pharmaceutical combination. The synergistic effect may be attained by administering two or more agents as separate formulations or in one particle, administered simultaneously or sequentially.

The term “Q-Switch” as used herein, generally refers to an optical device (Pockels cell) that controls the storage or release of laser energy from a laser optical cavity. Q-switching is a means of creating very short pulses (5-100 ns) with extremely high peak powers. Q stands for quality.

The term “Thermal Cytotoxicity Test” as used herein refers to an in vitro test specifically designed to test the compositions and the specific exogenous source(s) for their ability to spare healthy cells during use while killing the cancer cells. The thermal cytotoxicity test is a trans-well assay wherein healthy cells are grown, with cancer cells grown on an insert, and exposed to different doses of the composition and the exogenous source. Viability of the cancer and healthy cells are assessed using standard colorimetric assays 24 hours after exposure of the cells to the compositions and exogenous source. Different types of healthy and cancer cells can be selected for this test for different cancer applications. The composition and light dose(s) that do not kill any more than 30% of the healthy cells but kill at least 70% of the unwanted cells are considered passing the Thermal Cytotoxicity Test.

The term “thermal relaxation time (TRT)” as used herein, generally refers to a simplified mathematical model to estimate the time taken for the target to dissipate about 50% of the incident thermal energy. It is related to the size of the targeted particle, e.g., 10 picoseconds (4 nm particle), 400 picoseconds (50 nm particle), a few nanoseconds (particles ranging in size from 40-300 nm), 200-1000 nanoseconds (melanosomes, 0.5 μm), to hundreds of milliseconds (leg venules, 10 μm to 100 μm diameters). Longer TRT means the target takes longer time to cool to 50% of the temperature achieved. For spherical targets with radius R, the TRT may be determined using Eqn. (I). TRT=R²/6.75 k, Eqn. (I) where k is thermal diffusivity. For R=10 nanometers, 50 nanometers, and 5 nanometers, TRT is about 160 picoseconds, 4 nanoseconds, and 40 picoseconds, respectively. Even if the epidermis is a strong competing absorber, it can be spared as long as the TRT of the target is longer than that of epidermis (thickness is about 100 μm, pulse duration is about 3-5 milliseconds).

The term “therapeutic index” (TI) as used herein refers to a quantitative measurement of the relative safety of a drug. It is a comparison of the amount of a active agent that causes the therapeutic effect to the amount that causes toxicity

$$TI = \frac{TD 50}{ED 50} ,$$

where ED₅₀ is median effective dose and TD₅₀ is the median toxic dose. The median effective dose (ED₅₀) is the dose at which 50% of the subjects exhibit the required effect of the drug. The median toxic dose (TD₅₀) is the dose required to produce a defined toxic effect in 50% of subjects. For many drugs, there are severe toxicities that occur at sublethal doses in humans, and these toxicities often limit the maximum dose of a drug. A high therapeutic index (TI) is preferable for a drug to have a favorable safety and efficacy profile.

The term “tumor microenvironmental factor” as used herein, generally refers to the unique physiological features found in all tumors, such as abnormal acidic pH, hypoxia, elevated level of enzymes, including over expressed kinase receptors, proteases, elevated level of reducing agents like glutathione, and elevated level of ROS (tumor microenvironment stimuli).

The term “tumor targeting group” (cancer cell localizing component) as used herein, refers to a moiety that localizes the particle to a specific tumor site. The moiety may be, for example, a protein, peptide, aptamer, nucleic acid, nucleic acid analog, carbohydrate, or small molecule. The targeting group directs the localization of the particle heaters.

The term “unwanted cells” as used herein refers to host or foreign cells that are not required for the normal functioning of the body and can be removed for improving health or cosmetic outcomes. Unwanted cells include diseased host cells like cancer cells or macrophage cells, or foreign cells such as bacterial cells, pathogens, viruses, fungal cells, protozoan cells etc.

### 1. Particle Ingredients

Material Interacting with Exogenous Sources

In some embodiments, the material interacting with the exogenous source produces heat that performs a function, like inducing cytotoxicity by raising the temperature to above normal body temperature. In some embodiments, the exogenous source is electromagnetic radiation, microwaves, radio waves, sound waves, electrical, or magnetic field. Currently, several energy sources (e.g. laser light, focused ultrasound and microwave) have been employed in thermal cancer therapy.

In some embodiments, the exogenous source may be electromagnetic radiation (EMR). In some embodiments, the material interacting with the exogenous source does not have significant optical absorption in the visible region of EMR. In some embodiments, the material interacting with the exogenous source comprises a IR absorbing agent capable of absorbing EMR and converting the energy to heat (photothermal conversion). In some embodiments, the exogenous source comprises a laser light. In some embodiments, the exogenous source comprises a LED light. In some embodiments, the laser light is a pulsed laser light. In some embodiments, the laser pulse duration is in a range from milliseconds to femtoseconds, and the laser has an oscillation wavelength at 1064 nm. In some embodiments, the laser emits light at 808 nm. In some embodiments, the laser emits light at 805 nm.

To treat deep-tissue buried tumors by photoactive agents, it is necessary to develop molecules/nanomaterials that are able to absorb NIR light in the biological windows I (650 nm-950 nm) and II (1000 nm to 1350 nm), where the biological components (i.e., melanin, hemoglobin, blood, water etc.) have minimal absorbance.

In some embodiments, the spectroscopic probe has absorption in the visible range (400 nm to 750 nm) and the material interacting with the exogenous source has significant absorption in the near infrared spectrum region (NIR) (750 nm to 1500 nm). In some embodiments, the spectroscopic probe has absorption in the visible range (400 nm to 750 nm) and the material has significant absorption in the near infrared spectrum region (NIR) (400 nm to 750 nm). In some embodiments, the material has significant absorption of LED light having a wavelength of 750 nm to 1050 nm. In some embodiments, the material interacting with the exogenous source has significant absorption of LED light having a wavelength of 750 nm to 940 nm (infrared LEDs or IR LEDs). In some embodiments, the LED light source is a LE7-IR™ instrument by Image Engineer having 480 LED channels including 11 IR channels that create different spectra not only in the visible but also in the near infrared spectrum up to 1050 nm.

In some embodiments, the material interacting with the exogenous source does not have significant optical absorption in the visible region of EMR. In some embodiments, the material interacting with the exogenous source comprises a IR absorbing agent capable of absorbing EMR and converting the energy to heat (photothermal conversion). In some embodiments, the material interacting with the exogenous source has significant absorption in the near infrared spectrum region (NIR). In some embodiments, the material interacting with the exogenous source has significant absorption at a NIR wavelengths in the range from 700 nm to 1500 nm. In some embodiments, the material interacting with the exogenous source has significant absorption at a NIR wavelength in the range from 700 nm to 1400 nm. In some embodiments, the material interacting with the exogenous source has significant absorption at a NIR wavelength in the range from 700 nm to 1300 nm. In some embodiments, the material interacting with the exogenous source has significant absorption at a NIR wavelength in the range from 750 nm to 850 nm. In some embodiments, the material interacting with the exogenous source has significant absorption at a NIR wavelength in the range from 750 nm to 900 nm. In some embodiments, the material interacting with the exogenous source has significant absorption at a NIR wavelength in the range from 750 nm to 950 nm. In some embodiments, irradiating the particle comprises an irradiation wavelength of 780 nm to 810 nm. In some embodiments, the material interacting with the exogenous source has significant absorption at a NIR wavelength in the range from 800 nm to 1100 nm. In some embodiments, the material interacting with the exogenous source has significant absorption at a NIR wavelength in the range from 750 nm to 850 nm. In some embodiments, the material interacting with the exogenous source has significant absorption at a NIR wavelength in the range from 1000 nm to 1400 nm. In some embodiments, the material interacting with the exogenous source has significant absorption at a NIR wavelength in the range from 1000 nm to 1300 nm. In some embodiments, the material interacting with the exogenous source has significant absorption at a NIR wavelength in the range from 1000 nm to 1100 nm. In some embodiments, the material interacting with the exogenous source has significant absorption at a wavelength selected from the group of 750 nm, 751 nm, 752 nm, 753 nm, 754 nm, 755 nm, 756 nm, 757 nm, 756 nm, 756 nm, 758 nm, 759 nm, 760 nm, 761 nm, 762 nm, 763 nm, 764 nm, 765 nm, 766 nm, 767 nm, 768 nm, 769 nm, 770 nm, 771 nm, 772 nm, 773 nm, 774 nm, 775 nm, 776 nm, 777 nm, 778 nm, 779 nm, 780 nm, 781 nm, 782 nm, 783 nm, 784 nm, 785 nm, 786 nm, 787 nm, 789 nm, 790 nm, 791 nm, 792 nm, 793 nm, 794 nm, 795 nm, 796 nm, 797 nm, 798 nm, 799 nm, 800 nm, 801 nm, 802 nm, 803 nm, 804 nm, 805 nm, 806 nm, 807 nm, 808 nm, 809 nm, 810 nm, 811 nm, 812 nm, 813 nm, 814 nm, 815 nm, 816 nm, 817 nm, 818 nm, 819 nm, 820 nm, 821 nm, 822 nm, 823 nm, 824 nm, 825 nm, 826 nm, 827 nm, 828 nm, 829 nm, 830 nm, 831 nm, 832 nm, 833 nm, 834 nm, 835 nm, 836 nm, 837 nm, 838 nm, 839 nm, 840 nm, 841 nm, 842 nm, 843 nm, 844 nm, 845 nm, 846 nm, 847 nm, 848 nm, 849 nm, 850 nm, 851 nm, 852 nm, 853 nm, 854 nm, 855 nm, 856 nm, 857 nm, 858 nm, 859 nm, 860 nm, 861 nm, 862 nm, 863 nm, 864 nm, 865 nm, 866 nm, 867 nm, 868 nm, 869 nm, 870 nm, 871 nm, 872 nm, 873 nm, 874 nm, 875 nm, 876 nm, 877 nm, 878 nm, 879 nm, 880 nm, 881 nm, 882 nm, 883 nm, 884 nm, 885 nm, 886 nm, 887 nm, 888 nm, 889 nm, 890 nm, 891 nm, 892 nm, 893 nm, 894 nm, 895 nm, 896 nm, 897 nm, 898 nm, 899 nm, 900 nm, 901 nm, 902 nm, 903 nm, 904 nm, 905 nm, 906 nm, 907 nm, 908 nm, 909 nm, 910 nm, 911 nm, 912 nm, 913 nm, 914 nm, 915 nm, 916 nm, 917 nm, 918 nm, 919 nm, 920 nm, 921 nm, 922 nm, 923 nm, 924 nm, 925 nm, 926 nm, 927 nm, 928 nm, 929 nm, 930 nm, 931 nm, 932 nm, 933 nm, 934 nm, 935 nm, 936 nm, 937 nm, 938 nm, 939 nm, 940 nm, 941 nm, 942 nm, 943 nm, 944 nm, 945 nm, 946 nm, 947 nm, 948 nm, 949 nm, 950 nm, 951 nm, 952 nm, 953 nm, 954 nm, 955 nm, 956 nm, 957 nm, 958 nm, 959 nm, 960 nm, 961 nm, 962 nm, 963 nm, 964 nm, 965 nm, 966 nm, 967 nm, 968 nm, 969 nm, 970 nm, 971 nm, 972 nm, 973 nm, 974 nm, 975 nm, 976 nm, 977 nm, 978 nm, 979 nm, 980 nm, 981 nm, 982 n, 983 nm, 984 nm, 985 nm, 986 nm, 987 nm, 988 nm, 989 nm, 990 nm, 991 nm, 992 nm, 993 nm, 994 nm, 995 nm, 996 nm, 997 nm, 998 nm, 999 nm, 1000 nm, 1001 nm, 1002 nm, 1003 nm, 1004 nm, 1005 nm, 1006 nm, 1007 nm, 1008 nm, 1009 nm, 1010 nm, 1011 nm, 1012 nm, 1013 nm, 1014 nm, 1015 nm, 1016 nm, 1017 nm, 1018 nm, 1019 nm, 1020 nm, 1021 nm, 1022 nm, 1023 nm, 1024 nm, 1025 nm, 1026 nm, 1027 nm, 1028 nm, 1029 nm, 1030 nm, 1031 nm, 1032 nm, 1033 nm, 1034 nm, 1035 nm, 1036 nm, 1037 nm, 1038 nm, 1039 nm, 1040 nm, 1041 nm, 1042 nm, 1043 nm, 1044 nm, 1045 nm, 1046 nm, 1047 nm, 1048 nm, 1049 nm, 1050 nm, 1051 nm, 1052 nm, 1053 nm, 1054 nm, 1055 nm, 1056 nm, 1057 nm, 1058 nm, 1059 nm, 1060 nm, 1061 nm, 1062 nm, 1063 nm, 1064 nm, 1065 nm, 1066 nm, 1067 nm, 1068 nm, 1069 nm, 1070 nm, 1071 nm, 1072 nm, 1073 nm, 1074 nm, 1075 nm, 1076 nm, 1077 nm, 1078 nm, 1079 nm, 1080 nm, 1081 nm, 1082 nm, 1083 nm, 1084 nm, 1085 nm, 1086 nm, 1087 nm, 1088 nm, 1089 nm, 1090 nm, 1091 nm, 1092 nm, 1093 nm, 1094 nm, 1095 nm, 1096 nm, 1097 nm, 1098 nm, 1099 nm, and 1100 nm. In some embodiments, the material interacting with the exogenous source has significant absorption at a wavelength selected from the group of 700 nm, 766 nm, 777 nm, 780 nm, 783 nm, 785 nm, 800 nm, 805 nm, 808 nm, 810 nm, 820 nm, 825 nm, 900 nm, 948 nm, 950 nm, 960 nm, 980 nm, 1000 nm, 1064 nm, 1065 nm, 1070 nm, 1071 nm, 1073 nm, 1098 nm, and 1100 nm.

In some embodiments, the material interacting with the exogenous source has significant absorption of photonic energy in the visible range. In some embodiments, the material absorbs light at a wavelength ranging from 400 nm to 750 nm. In some embodiments, the material absorbs light at a wavelength selected from the group of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

In some embodiments, the material interacting with the exogenous source has significant absorption at 805 nm wavelength. In some embodiments, the material interacting with the exogenous source has significant absorption at 808 nm wavelength. In some embodiments, the material interacting with the exogenous source has significant absorption at 1064 nm wavelength.

In some embodiments, the material is an infrared radiation absorbing agent such as those Epolight™ aminium dyes made by Epolin Inc. of Newark, N.J. In some embodiments, the IR absorbing agent is an di-imonium dye (also aminium dye) having formula (I)

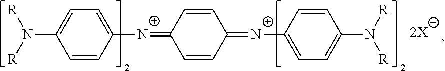

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from the group of hexafluoroarsenate (AsF₆⁻), hexafluoroantimonate (SbF₆⁻), hexafluorophosphate (PF₆⁻), tetrakis(perfluorophenyl)borate (C₆F₅)₄B⁻, and tetrafluoroborate (BF₄⁻). In some embodiments, the di-imonium dye of formula (I) has hexafluorophosphate as counterion. In some embodiments, the di-imonium dye of formula (I) has hexafluoroantimonate as counterion. In some embodiments, the di-imonium dye of formula (I) has tetrakis(perfluorophenyl)borate as counterion. In some embodiments, the IR absorbing agent is a tetrakis aminium dye, with a counterion containing metal element such as boron or antimony. In some embodiments, the tetrakis aminium dye compounds have formula (II)

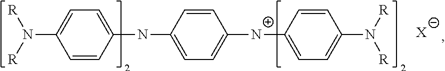

wherein R is a substituted or unsubstituted aryl, heteroaryl, C1-C8 alkyl, C1-C8 alkenyl, or C1-C8 alkynyl group, wherein the C1-C8 alkyl, C1-C8 alkenyl, or C1-C8 alkynyl group may be linear or branched, wherein X⁻ is a counterion selected from hexafluoroarsenate (AsF₆⁻), hexafluoroantimonate (SbF₆⁻), hexafluorophosphate (PF₆⁻), (C₆F₅)₄B⁻, or tetrafluoroborate (BF₄⁻). In some embodiments, the tetrakis aminium dyes are narrow band absorbers including commercially available dyes sold under the trademark names Epolight™ 1117 (tetrakis aminium dye having hexafluorophosphate counterion, peak absorption, 1071 nm), Epolight™ 1151 (tetrakis aminium dye, peak absorption, 1070 nm), or Epolight™ 1178 (tetrakis aminium dye, peak absorption, 1073 nm). Epolight™ 1151 (tetrakis aminium dye, peak absorption, 1070 nm), or Epolight™ 1178 (tetrakis aminium dye, peak absorption, 1073 nm). In some embodiments, the tetrakis aminium dyes are broad band absorbers including commercially available dyes sold under the trademark names Epolight™ 1175 (tetrakis aminium dye, peak absorption, 948 nm), Epolight™ 1125 (tetrakis aminium dye, peak absorption, 950 nm), and Epolight™ 1130 (tetrakis aminium dye, peak absorption, 960 nm).

In some embodiments, the tetrakis aminium dye is Epolight™ 1178 made by Epolin. In some embodiments, the IR absorbing agent is a tetrakis aminium dye, which has minimal visible color. In some embodiments, the tetrakis aminium dye is Epolight™ 1117 (molecular weight, 1211 Da, peak absorption 1098 nm).

Other suitable aminium and/or di-imonium dyes suitable for the invention in this disclosure may be found in U.S. Pat. Nos. 3,440,257, 3,484,467, 3,400,156, 5,686,639, all of which are hereby fully incorporated by reference herein in their entirety. Additional counterions for the aminium and/or di-imonium dyes may be found in U.S. Pat. No. 7,498,123, which is hereby fully incorporated by reference herein in its entirety.

In some embodiments, the material is an IR dye selected from the group of 1-butyl-2-(2-[3-[2-(1-butyl-1H-benzo[cd]indol-2-ylidene)-ethylidene]-2-chloro-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclopent-1-enyl]-vinyl)-benzo[cd]indolium tetrafluoroborate, 1-butyl-2-(2-[3-[2-(1-butyl-1H-benzo[cd]indol-2-ylidene)-ethylidene]-2-phenyl-cyclohex-1-enyl]-vinyl)-benzo[cd]indolium tetrafluoroborate, 1-butyl-2-(2-[3-[2-(1-butyl-1H-benzo[cd]indol-2-ylidene)-ethylidene]-2-diphenylamino-cyclopent-1-enyl]vinyl)-benzo[cd]indolium tetrafluoroborate, 1-Butyl-2-[2-[3-[(1-butyl-6-chlorobenz[cd]indol-2(1H)-ylidene)ethylidene]-2-chloro-1-cyclohexen-1-yl]ethenyl]-6-chlorobenz[cd]indolium tetrafluoroborate (IR 1048), 1-butyl-2-[2-[3-[(1-butyl-6-chlorobenz[cd]indol-2(1H)-ylidene)ethylidene]-2-chloro-5-methyl-1-cyclohexen-1-yl]ethenyl]-6-chlorobenz[cd]indolium tetrafluoroborate (Lumogen™ IR 1050 by BASF), 4-[2-[2-chloro-3-[(2,6-diphenyl-4H-thiopyran-4-ylidene)ethylidene]-1-cyclohexen-1-yl]ethenyl]-2,6-diphenylthiopyrylium tetrafluoroborate (IR 1061), dimethyl {4-[1,7,7-tris(4-dimethylaminophenyl)-2,4,6-heptatrienylidene]-2,5-cyclohexadien-1-ylidene}ammonium perchlorate (IR 895), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), 4-hydroxybenzoic acid appended heptamethine cyanine, amine functionalized heptamethine cyanine, hemicyanine rhodamine, cryptocyanine, diketopyrrolopyrole, diketopyrrolopyrole-croconaine, 1,3-bis(5-(ethyl(2-(prop-2-yn-1-yloxy)ethyl)amino)thiophen-2-yl)-4,5-dioxocyclopent-2-en-1-ylium-2-olate (diaminothiophene-croconaine dye), potassium 1,1′-((2-oxido-4,5-dioxocyclopent-2-en-1-ylium-1,3-diyl)bis(thiophene-5,2-diyl))bis(piperidine-4-carboxylate) (dipiperidylthiophene-croconaine dye), indocyanine green (ICG), Cyanine 7 (Cy7®), and combinations thereof.

In some embodiments, the squarylium dye is a benzopyrylium squarylium dyes having formula (III)

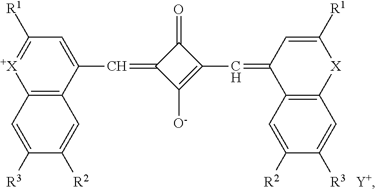

wherein each X is independently O, S, Se; Y⁺ is a counterion selected from the group of hexafluoroarsenate (AsF₆⁻), hexafluoroantimonate (SbF₆⁻), hexafluorophosphate (PF₆⁻), (C₆F₅)₄B⁻, and tetrafluoroborate (BF₄⁻); each R¹ is a non-aromatic organic substituent, each R²═H or OR³, R³=cycloalkyl, alkenyl, acyl, silyl; each R³═—NR⁴R⁵, each R⁴, R⁵ is independently H, C1-8 alkyl. In some embodiments, the squarylium dye of formula (III) is a compound when R¹═—CMe₃, R²═OCHMeEt, X═O with a strong absorption at 788 nm. In some embodiments, the squarylium dye of formula (III) is a compound when R¹═—CMe₃, R²═H, R³═—NEt₂, X═O with a strong absorption at 808 nm (IR 193 dye).

In some embodiments, the IR absorbing agent comprises cyanine dyes selected from the group indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof. In some embodiments, the IR absorbing agent may include indocyanine green (ICG).

In some embodiments, the IR absorbing agent may include a squarylium dye. In some embodiments, the IR absorbing agent may include squaraine dye. In some embodiments, the IR absorbing agent may include a squarylium dye selected from the group of IR 193 dye, 1,3-bis[[2-(1,1-dimethylethyl)-4H-1-benzopyran-4-ylidene]methyl]-2,4-dihydroxy-cyclobutenediylium salt, 1,3-dihydroxy-2,4-bis[(2-phenyl-4H-1-benzopyran-4-ylidene)methyl]-cyclobutenediylium salt, 1,3-bis[[2-(1,1-dimethylethyl)-6-methyl-4H-1-benzopyran-4-ylidene]methyl]-2,4-dihydroxy-cyclobutenediylium salt, 1,3-bis[[2-(1,1-dimethylethyl)-7-hydroxy-4H-1-benzopyran-4-ylidene]methyl]-2,4-dihydroxy-cyclobutenediylium salt, 1,3-bis[[2-(1,1-dimethylethyl)-6-(1-methylethyl)-4H-1-benzopyran-4-ylidene]methyl]-2,4-dihydroxy-cyclobutenediylium salt, 1,3-dihydroxy-2,4-bis[1-(2-phenyl-4H-1-benzopyran-4-ylidene)ethyl]-cyclobutenediylium salt, 1,3-dihydroxy-2,4-bis[(2-phenyl-4H-naphtho[1,2-b]pyran-4-ylidene)methyl]-cyclobutenediylium salt, 1,3-dihydroxy-2,4-bis[[6-(1-methylethyl)-2-phenyl-4H-1-benzopyran-4-ylidene]methyl]-cyclobutenediylium salt, 1,3-bis[[6-(1,1-dimethylethyl)-2-phenyl-4H-1-benzopyran-4-ylidene]methyl]-2,4-dihydroxy-cyclobutenediylium salt, 1,3-bis[(2-cyclohexyl-7-methoxy-4H-1-benzopyran-4-ylidene)methyl]-2,4-dihydroxy-cyclobutenediylium salt, 1,3-bis[[2-(1,1-dimethylethyl)-6-(1-methylpropoxy)-4H-1-benzopyran-4-ylidene]methyl]-2,4-dihydroxy-cyclobutenediylium salt, 1,3-bis[[8-chloro-2-(1,1-dimethylethyl)-6-(1-methylethyl)-4H-1-benzopyran-4-ylidene]methyl]-2,4-dihydroxy-cyclobutenediylium salt, 1,3-bis[[7-(dimethylamino)-2-(1,1-dimethylethyl)-4H-1-benzopyran-4-ylidene]methyl]-2,4-dihydroxy-cyclobutenediylium salt, 1-[[7-(diethylamino)-2-(1,1-dimethylethyl)-4H-1-benzopyran-4-ylidene]methyl]-3-[[7-(dimethylamino)-2-(1,1-dimethylethyl)-4H-1-benzopyran-4-ylidene]methyl]-2,4-dihydroxy-cyclobutenediylium salt, 1,3-bis[[7-(diethylamino)-2-(1,1-dimethylethyl)-4H-1-benzopyran-4-ylidene]methyl]-2,4-dihydroxy-cyclobutenediylium salt, 1,3-bis[1-[7-(diethylamino)-2-(1,1-dimethylethyl)-4H-1-benzopyran-4-ylidene]ethyl]-2,4-dihydroxy-cyclobutenediylium salt, 1-[[7-(diethylamino)-2-(1,1-dimethylethyl)-4H-1-benzopyran-4-ylidene]methyl]-3-[[2-(1,1-dimethylethyl)-7-(2-ethylbutoxy)-4H-1-benzopyran-4-ylidene]methyl]-2,4-dihydroxy-cyclobutenediylium salt, 1,3-bis[[2-cyclohexyl-7-(diethylamino)-4H-1-benzopyran-4-ylidene]methyl]-2,4-dihydroxy-cyclobutenediylium salt, 1,3-bis[[2-(1,1-dimethylethyl)-7-(1-piperidinyl)-4H-1-benzopyran-4-ylidene]methyl]-2,4-dihydroxy-cyclobutenediylium salt, 1,3-bis[[2-(1,1-dimethylethyl)-7-(hexahydro-1H-azepin-1-yl)-4H-1-benzopyran-4-ylidene]methyl]-2,4-dihydroxy-cyclobutenediylium salt, 1,3-bis[[2-(1,1-dimethylethyl)-7-(4-morpholinyl)-4H-1-benzopyran-4-ylidene]methyl]-2,4-dihydroxy-cyclobutenediylium salt, 1,3-bis[[11-(1,1-dimethylethyl)-2,3,6,7-tetrahydro-1H,5H,9H-[1]benzopyrano[6,7,8-ij]quinolizin-9-ylidene]methyl]-2,4-dihydroxy-cyclobutenediylium salt, 1,3-bis[[2-(1,1-dimethylethyl)-6-(4-morpholinyl)-4H-1-benzopyran-4-ylidene]methyl]-2,4-dihydroxy-cyclobutenediylium salt, 1,3-bis[[2-bicyclo[2.2.1]hept-5-en-2-yl-7-(diethylamino)-4H-1-benzopyran-4-ylidene]methyl]-2,4-dihydroxy-cyclobutenediylium salt, 1,3-bis[[7-(2,3-dihydro-1Hindol-1-yl)-2-(1,1-dimethyl ethyl)-4H-1-benzopyran-4-ylidene]methyl]-2,4-dihydroxy-cyclobutenediylium salt, 1,3-bis[[7-(diethylamino)-2-[(1R,5S)-6,6-dimethylbicyclo[3.1.1]hept-2-en-2-yl]-4H-1-benzopyran-4-ylidene]methyl]-2,4-dihydroxy-cyclobutenediylium salt, 1,3-bis[[7-(diethylamino)-2-(6,6-dimethylbicyclo[3.1.1]hept-2-en-3-yl)-4H-1-benzopyran-4-ylidene]methyl]-2,4-dihydroxy-cyclobutenediylium salt, 1,3-dihydroxy-2,4-bis[[7-(4-morpholinyl)-2-tricyclo[3.3.1.13,7]dec-1-yl-4H-1-benzopyran-4-ylidene]methyl]-cyclobutenediylium salt, 2,4-bis[[7-(diethylamino)-2-(1,1-dimethylethyl)-4H-1-benzopyran-4-ylidene]methyl]-1,3-cyclobutanedione, and combinations thereof.

In some embodiments, the material is an IR-dye selected from the group of phthalocyanines, naphthalocyanines, and combinations thereof. In some embodiments, the material is selected from the group of a tri-aminium dye, a tetrakis aminium dye, a cyanine dye, a squarylium dye, an inorganic IR absorbing agent, and combinations thereof. In some embodiments, the material is a squaraine dye. In some embodiments, the material is a tetrakis aminium dye. In some embodiments, the material is a squarylium dye. In some embodiments, the material is an inorganic IR absorbing agent. In some embodiments, the IR absorbing agent is an organic IR absorbing agent. In some embodiments, the IR absorbing agent is an aminium and/or di-imonium dye having hexafluoroantimonate, tetrafluoroborate, or hexafluorophosphate as counterion. In some embodiments, an IR absorbing agent, N,N,N,N-tetrakis(4-dibutylaminophenyl)-p-benzoquinone bis(iminium hexafluoroantimonate), commercially available as ADS1065 from American Dye Source, Inc., may be utilized. The absorption spectrum of ADS1065 dye has a maximum absorption at about 1065 nm, with low absorption in the visible region of the spectrum. In some embodiments, the IR absorbing agent is indocyanine green (ICG) or new ICG dye IR820. After the ICG particles are irradiated with pulsed laser light, the excited ICG dye can produce singlet oxygen species in the presence of cellular water. ROS is lethal for unwanted cells like tumor cells or microbes.

In some embodiments, the infrared radiation absorbing materials are inorganic substances that contain specific chemical elements having an incomplete electronic d-shell (i.e. atoms or ions of transition elements), and whose infrared absorption is a consequence of electronic transitions within the d-shell of the atom or ion. In some embodiments, the inorganic IR absorbing agents comprise one or more transition metal elements in the form of an ion such as a palladium(II), a platinum(II), a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the materials are inorganic IR absorbing agents with near-infrared absorbing properties selected from the group of iron oxide nanoparticle, zinc copper phosphate pigment ((Zn,Cu)₂P₂O₇), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing agent is a zinc iron phosphate pigment. In some embodiments, the inorganic IR absorbing agent may include palladate (e.g. barium tetrakis(cyano-C)palladate tetrahydrate, BaPd(CN)₄.4H₂O, [Pd(dimit)₂]²⁻, bis(1,3-dithiole-2-thione-4,5-dithiolate)palladate(II). In some embodiments, the inorganic IR absorbing agent may include platinate, e.g. platinum-based polypyridyl complexes with dithiolate ligands, Pt(II)(diamine)(dithiolate) with 3,3′-, 4,4′-, 5,5′-bipyridyl substituents.

In some embodiments, the inorganic infrared radiation absorbing material comprises iron oxide nanoparticle (also known to function as MRI contrast agent, magnetic energy absorbing agent).

In some embodiments, the infrared radiation absorbing material is admixed within the carrier to form a homogeneous dispersion or a solid solution. In some embodiments, the infrared radiation absorbing material and the carrier may have oppositely charged functional group(s) (e.g. infrared radiation absorbing material is positively charged tetrakis aminium dye, and the carrier has negatively charged functional group such as carboxylate anion of polymethacrylate polymers) such that the infrared radiation absorbing material attaches to the carrier via hydrogen bond or via ionic electrostatic interactions.

In some embodiments, the material is selected from the group of a tetrakis aminium dye, a cyanine dye, a squarylium dye, indocyanine green (ICG), new ICG (IR 820), squaraine dye, IR 780 dye, IR 193 dye, Epolight™ IR 1117, Epolight™ 1175, iron oxide, zinc iron phosphate pigment, and combinations thereof.

In some embodiments, the infrared radiation absorbing material is a tetrakis aminium dye. In some embodiments, the tetrakis aminium dye is a narrow band absorber including commercially available dyes sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broadband absorber including commercially available dyes sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the tetrakis aminium dye is Epolight™ 1178. In some embodiments, the IR absorbing agent is a tetrakis aminium dye has minimal visible color. In some embodiments, the tetrakis aminium dye is Epolight™ 1117 ((hexafluorophosphate as counterion, molecular weight, 1211 Da, peak absorption 1098 nm).

In some embodiments, the material interacting with exogenous comprises a plasmonic absorber. In some embodiments, the plasmonic absorbers comprise plasmonic nanomaterials of noble metal gold (Au), silver (Ag) and copper (Cu) nanoparticles doped with sulfur (S), selenium (Se) or tellurium (Te) having a plasmonic resonance at a NIR wavelength. In some embodiments, the plasmonic absorbers comprise gold nanostructures such as nanoporous gold thin films, or gold nanospheres, gold nanorods, gold nanoshells, gold nanocages, silver nanoparticles, Cu₉S₅ nanoparticle, and iron oxide. In some embodiments, the plasmonic absorbers comprise gold nanostructures. Compared to non-metallic nanoparticles, plasmonic nanomaterials exhibit a unique photophysical phenomenon, called localized surface plasmon resonance (LSPR) because of the absorption of light at a resonant frequency. Upon exposure with electromagnetic radiation, strong surface fields are induced due to the coherent excitation of the electrons in the metallic nanoparticles. The rapid relaxation of these excited electrons produces strong localized heat capable of destroying the surrounding unwanted cells via hyperthermia or other thermal-based effects. By changing the structure (e.g. size) and shape, the LSPR frequency of the noble metal nanostructures can be tuned to shift the resulting plasmonic resonance wavelength in the NIR therapeutic window (750-1300 nm), where light penetration in the tissue is optimal. The endogenous absorption coefficient of the tissue in the NIR band is nearly two orders of magnitude lower than that in the visible band of EM spectrum. In some embodiments, the plasmonic absorbers may have LSPR ranging from about 700 nm to about 900 nm. In some embodiments, the plasmonic absorbers may have LSPR raging from about 900 nm to about 1064 nm.

In some embodiments, the particle heaters comprise core particles of 100-200 nm in size formed from the carrier and the material as described above, and a thin layer of noble metal film (5-20 nm) as particle surface coatings, wherein the noble metal is selected from the group of gold, silver, copper doped with S, Se and Te, and combinations thereof, wherein the heat delivery composition exhibits additive or synergistic thermotherapy resulting from LSPR of film coated particle and the conventional thermotherapy from organic dye in the core. The LSPR wavelength is tunable by decreasing the shell thickness-to-core radius ratio, wherein LSPR wavelength shift is independent of shell size, core material, shell metal or surrounding medium.

In some embodiments, the particle heaters further comprise a shell to form core-shell particles, wherein the material interacting with the exogenous source is plasmonic absorber disposed in the shell, wherein the plasmonic absorbers are embedded within, either ionically associated with, or covalently bound to the shell. In some embodiments, the plasmonic absorbers are particles having a thin and porous gold wall with hollow interior, wherein the LSPR wavelength can be tuned by changing the wall thickness, pore size and porosity. In some embodiments, the plasmonic absorbers are core-shell particles having a gold nanoparticle core having the shape of sphere, shell, or rod, and a shell of hydrophilic polymer (e.g. chitosan, PEG) to enclose the gold nanoparticle core. In some embodiments, the particles may have a shell made out of iron oxide.

In some embodiments, the particle exhibits energy-to-heat conversion stability such that the loss in absorbance of the IR absorbing agent is less than 50% as measured by the Material Process Stability Test after exposure to a pulsed laser light, and the particle is considered as passing the Material Process Stability Test.

The preferred concentration of the material responsive to the exogenous source is dependent on the amount required to obtain the desired response to the source. For example, in the case of an IR absorbing agent needed to absorb incident IR radiation, then too little dye can limit the temperature rise that would be desired. Likewise, too high a concentration can lead to dye aggregation, which can shift the absorption, such that the dye no longer absorbs the wavelength provided by the laser. In some embodiments, the material responsive to the exogenous source is present in an amount ranging from about 0.01 wt. % to about 25.0 wt. % by the total weight of the particle. In some embodiments, the material responsive to the exogenous source is present in an amount ranging from about 1.0 wt. % to about 20.0 wt. % by the total weight of the particle. In some embodiments, the material responsive to the exogenous source is present in an amount ranging from about 5.0 wt. % to about 20.0 wt. % by the total weight of the particle. In some embodiments, the material responsive to the exogenous source is present in an amount ranging from about 5.0 wt. % to about 15.0 wt. % by the total weight of the particle. In some embodiments, the material responsive to the exogenous source is present in an amount ranging from about 5.5 wt. % to about 15.0 wt. % by the total weight of the particle. In some embodiments, the material responsive to the exogenous source is present in an amount ranging from about 6.0 wt. % to about 15.0 wt. % by the total weight of the particle. In some embodiments, the material responsive to the exogenous source is present in an amount ranging from about 6.5 wt. % to about 15.0 wt. % by the total weight of the particle. In some embodiments, the material responsive to the exogenous source is present in an amount ranging from about 7.0 wt. % to about 15.0 wt. % by the total weight of the particle. In some embodiments, the material responsive to the exogenous source is present in an amount ranging from about 7.5 wt. % to about 15.0 wt. % by the total weight of the particle. In some embodiments, the material responsive to the exogenous source is present in an amount ranging from about 8.0 wt. % to about 15.0 wt. % by the total weight of the particle. In some embodiments, the material responsive to the exogenous source is present in an amount ranging from about 8.5 wt. % to about 15.0 wt. % by the total weight of the particle. In some embodiments, the material responsive to the exogenous source is present in an amount ranging from about 9.0 wt. % to about 15.0 wt. % by the total weight of the particle. In some embodiments, the material responsive to the exogenous source is present in an amount ranging from about 9.5 wt. % to about 15.0 wt. % by the total weight of the particle. In some embodiments, the material responsive to the exogenous source is present in an amount ranging from about 10.0 wt. % to about 15.0 wt. % by the total weight of the particle. In some embodiments, the material responsive to the exogenous source is present in an amount ranging from about 10.5 wt. % to about 15.0 wt. % by the total weight of the particle. In some embodiments, the material responsive to the exogenous source is present in an amount ranging from about 11.0 wt. % to about 15.0 wt. % by the total weight of the particle. In some embodiments, the material responsive to the exogenous source is present in an amount ranging from about 11.5 wt. % to about 15.0 wt. % by the total weight of the particle. In some embodiments, the material responsive to the exogenous source is present in an amount ranging from about 12.0 wt. % to about 15.0 wt. % by the total weight of the particle. In some embodiments, the material responsive to the exogenous source is present in an amount ranging from about 12.5 wt. % to about 15.0 wt. % by the total weight of the particle. In some embodiments, the material responsive to the exogenous source is present in an amount ranging from about 13.0 wt. % to about 15.0 wt. % by the total weight of the particle. In some embodiments, the material responsive to the exogenous source is present in an amount ranging from about 13.5 wt. % to about 15.0 wt. % by the total weight of the particle. In some embodiments, the material responsive to the exogenous source is present in an amount ranging from about 14.0 wt. % to about 15.0 wt. % by the total weight of the particle. In some embodiments, the material responsive to the exogenous source is present in an amount ranging from about 5.0 wt. % to about 14.0 wt. % by the total weight of the particle. In some embodiments, the material responsive to the exogenous source is present in an amount ranging from about 5.5 wt. % to about 14.0 wt. % by the total weight of the particle. In some embodiments, the material responsive to the exogenous source is present in an amount ranging from about 6.0 wt. % to about 14.0 wt. % by the total weight of the particle. In some embodiments, the material responsive to the exogenous source is present in an amount ranging from about 6.5 wt. % to about 14.0 wt. % by the total weight of the particle. In some embodiments, the material responsive to the exogenous source is present in an amount ranging from about 7.0 wt. % to about 14.0 wt. % by the total weight of the particle. In some embodiments, the material responsive to the exogenous source is present in an amount ranging from about 7.5 wt. % to about 14.0 wt. % by the total weight of the particle. In some embodiments, the material responsive to the exogenous source is present in an amount ranging from about 8.0 wt. % to about 14.0 wt. % by the total weight of the particle. In some embodiments, the material responsive to the exogenous source is present in an amount ranging from about 8.5 wt. % to about 14.0 wt. % by the total weight of the particle. In some embodiments, the material responsive to the exogenous source is present in an amount ranging from about 9.0 wt. % to about 14.0 wt. % by the total weight of the particle. In some embodiments, the material responsive to the exogenous source is present in an amount ranging from about 9.5 wt. % to about 14.0 wt. % by the total weight of the particle. In some embodiments, the material responsive to the exogenous source is present in an amount ranging from about 10.0 wt. % to about 14.0 wt. % by the total weight of the particle. In some embodiments, the material responsive to the exogenous source is present in an amount ranging from about 10.5 wt. % to about 14.0 wt. % by the total weight of the particle. In some embodiments, the material responsive to the exogenous source is present in an amount ranging from about 11.0 wt. % to about 14.0 wt. % by the total weight of the particle. In some embodiments, the material responsive to the exogenous source is present in an amount ranging from about 11.5 wt. % to about 14.0 wt. % by the total weight of the particle. In some embodiments, the material responsive to the exogenous source is present in an amount ranging from about 12.0 wt. % to about 14.0 wt. % by the total weight of the particle. In some embodiments, the material responsive to the exogenous source is present in an amount ranging from about 12.5 wt. % to about 14.0 wt. % by the total weight of the particle. In some embodiments, the material responsive to the exogenous source is present in an amount ranging from about 13.0 wt. % to about 14.0 wt. % by the total weight of the particle. In some embodiments, the material responsive to the exogenous source is present in an amount ranging from about 13.5 wt. % to about 14.0 wt. % by the total weight of the particle. In some embodiments, the material responsive to the exogenous source is present in an amount ranging from about 5.0 wt. % to about 13.0 wt. % by the total weight of the particle. In some embodiments, the material responsive to the exogenous source is present in an amount ranging from about 5.5 wt. % to about 13.0 wt. % by the total weight of the particle. In some embodiments, the material responsive to the exogenous source is present in an amount ranging from about 6.0 wt. % to about 13.0 wt. % by the total weight of the particle. In some embodiments, the material responsive to the exogenous source is present in an amount ranging from about 6.5 wt. % to about 13.0 wt. % by the total weight of the particle. In some embodiments, the material responsive to the exogenous source is present in an amount ranging from about 7.0 wt. % to about 13.0 wt. % by the total weight of the particle. In some embodiments, the material responsive to the exogenous source is present in an amount ranging from about 7.5 wt. % to about 13.0 wt. % by the total weight of the particle. In some embodiments, the material responsive to the exogenous source is present in an amount ranging from about 8.0 wt. % to about 13.0 wt. % by the total weight of the particle. In some embodiments, the material responsive to the exogenous source is present in an amount ranging from about 8.5 wt. % to about 13.0 wt. % by the total weight of the particle. In some embodiments, the material responsive to the exogenous source is present in an amount ranging from about 9.0 wt. % to about 13.0 wt. % by the total weight of the particle. In some embodiments, the material responsive to the exogenous source is present in an amount ranging from about 9.5 wt. % to about 13.0 wt. % by the total weight of the particle. In some embodiments, the material responsive to the exogenous source is present in an amount ranging from about 10.0 wt. % to about 13.0 wt. % by the total weight of the particle. In some embodiments, the material responsive to the exogenous source is present in an amount ranging from about 10.5 wt. % to about 13.0 wt. % by the total weight of the particle. In some embodiments, the material responsive to the exogenous source is present in an amount ranging from about 11.0 wt. % to about 13.0 wt. % by the total weight of the particle. In some embodiments, the material responsive to the exogenous source is present in an amount ranging from about 11.5 wt. % to about 13.0 wt. % by the total weight of the particle. In some embodiments, the material responsive to the exogenous source is present in an amount ranging from about 12.0 wt. % to about 13.0 wt. % by the total weight of the particle.

In some embodiments, the material responsive to the exogenous source is present in an amount ranging from about 5.0 wt. % to about 12.0 wt. % by the total weight of the particle. In some embodiments, the material responsive to the exogenous source is present in an amount ranging from about 5.5 wt. % to about 12.0 wt. % by the total weight of the particle. In some embodiments, the material responsive to the exogenous source is present in an amount ranging from about 6.0 wt. % to about 12.0 wt. % by the total weight of the particle. In some embodiments, the material responsive to the exogenous source is present in an amount ranging from about 6.5 wt. % to about 12.0 wt. % by the total weight of the particle. In some embodiments, the material responsive to the exogenous source is present in an amount ranging from about 7.0 wt. % to about 12.0 wt. % by the total weight of the particle. In some embodiments, the material responsive to the exogenous source is present in an amount ranging from about 7.5 wt. % to about 12.0 wt. % by the total weight of the particle. In some embodiments, the material responsive to the exogenous source is present in an amount ranging from about 8.0 wt. % to about 12.0 wt. % by the total weight of the particle. In some embodiments, the material responsive to the exogenous source is present in an amount ranging from about 8.5 wt. % to about 12.0 wt. % by the total weight of the particle. In some embodiments, the material responsive to the exogenous source is present in an amount ranging from about 9.0 wt. % to about 12.0 wt. % by the total weight of the particle. In some embodiments, the material responsive to the exogenous source is present in an amount ranging from about 9.5 wt. % to about 12.0 wt. % by the total weight of the particle. In some embodiments, the material responsive to the exogenous source is present in an amount ranging from about 10.0 wt. % to about 12.0 wt. % by the total weight of the particle. In some embodiments, the material responsive to the exogenous source is present in an amount ranging from about 10.5 wt. % to about 12.0 wt. % by the total weight of the particle. In some embodiments, the material responsive to the exogenous source is present in an amount ranging from about 11.0 wt. % to about 12.0 wt. % by the total weight of the particle. In some embodiments, the material responsive to the exogenous source is present in an amount ranging from about 11.5 wt. % to about 12.0 wt. % by the total weight of the particle.

In some embodiments, the material responsive to the exogenous source is present in an amount ranging from about 5.0 wt. % to about 11.0 wt. % by the total weight of the particle. In some embodiments, the material responsive to the exogenous source is present in an amount ranging from about 5.5 wt. % to about 11.0 wt. % by the total weight of the particle. In some embodiments, the material responsive to the exogenous source is present in an amount ranging from about 6.0 wt. % to about 11.0 wt. % by the total weight of the particle. In some embodiments, the material responsive to the exogenous source is present in an amount ranging from about 6.5 wt. % to about 11.0 wt. % by the total weight of the particle. In some embodiments, the material responsive to the exogenous source is present in an amount ranging from about 7.0 wt. % to about 11.0 wt. % by the total weight of the particle. In some embodiments, the material responsive to the exogenous source is present in an amount ranging from about 7.5 wt. % to about 11.0 wt. % by the total weight of the particle. In some embodiments, the material responsive to the exogenous source is present in an amount ranging from about 8.0 wt. % to about 11.0 wt. % by the total weight of the particle. In some embodiments, the material responsive to the exogenous source is present in an amount ranging from about 8.5 wt. % to about 11.0 wt. % by the total weight of the particle. In some embodiments, the material responsive to the exogenous source is present in an amount ranging from about 9.0 wt. % to about 11.0 wt. % by the total weight of the particle. In some embodiments, the material responsive to the exogenous source is present in an amount ranging from about 9.5 wt. % to about 11.0 wt. % by the total weight of the particle. In some embodiments, the material responsive to the exogenous source is present in an amount ranging from about 10.0 wt. % to about 11.0 wt. % by the total weight of the particle. In some embodiments, the material responsive to the exogenous source is present in an amount ranging from about 10.5 wt. % to about 11.0 wt. % by the total weight of the particle.

In some embodiments, the material responsive to the exogenous source is present in an amount ranging from about 5.0 wt. % to about 10.0 wt. % by the total weight of the particle. In some embodiments, the material responsive to the exogenous source is present in an amount ranging from about 5.5 wt. % to about 10.0 wt. % by the total weight of the particle. In some embodiments, the material responsive to the exogenous source is present in an amount ranging from about 6.0 wt. % to about 10.0 wt. % by the total weight of the particle. In some embodiments, the material responsive to the exogenous source is present in an amount ranging from about 6.5 wt. % to about 10.0 wt. % by the total weight of the particle. In some embodiments, the material responsive to the exogenous source is present in an amount ranging from about 7.0 wt. % to about 10.0 wt. % by the total weight of the particle. In some embodiments, the material responsive to the exogenous source is present in an amount ranging from about 7.5 wt. % to about 10.0 wt. % by the total weight of the particle. In some embodiments, the material responsive to the exogenous source is present in an amount ranging from about 8.0 wt. % to about 10.0 wt. % by the total weight of the particle. In some embodiments, the material responsive to the exogenous source is present in an amount ranging from about 8.5 wt. % to about 10.0 wt. % by the total weight of the particle. In some embodiments, the material responsive to the exogenous source is present in an amount ranging from about 9.0 wt. % to about 10.0 wt. % by the total weight of the particle.

In some embodiments, the material responsive to the exogenous source is present in an amount ranging from about 8.0 wt. % to about 10.0 wt. % by the total weight of the particle. In some embodiments, the material responsive to the exogenous source is present in an amount ranging from about 7.0 wt. % to about 10.0 wt. % by the total weight of the particle. In some embodiments, the material responsive to the exogenous source is present in an amount ranging from about 6.0 wt. % to about 10.0 wt. % by the total weight of the particle. In some embodiments, the material responsive to the exogenous source is present in an amount ranging from about 5.0 wt. % to about 10.0 wt. % by the total weight of the particle. In some embodiments, the material responsive to the exogenous source is present in an amount ranging from about 4.0 wt. % to about 10.0 wt. % by the total weight of the particle. In some embodiments, the material responsive to the exogenous source is present in an amount ranging from about 3.0 wt. % to about 10.0 wt. % by the total weight of the particle.

In some embodiments, the material responsive to the exogenous source is present in an amount ranging from about 10.0 wt. % to about 15.0 wt. % by the total weight of the particle. In some embodiments, the material responsive to the exogenous source is present in an amount selected from the group of: about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. %. In some embodiments, the material responsive to the exogenous source is present in an amount selected from the group of: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. %. In some embodiments, the material responsive to the exogenous source is present in an amount selected from the group of: about 1.0 wt. %, about 5.0 wt. %, about 10.0 wt. %, and about 15.0 wt. %.

In some embodiments, the particle having a ratio of the weight amount of the material responsive to the exogenous source to the active agent of 10:1 to 1:10. In some embodiment, the ratio of the weight amount of the material responsive to the exogenous source to the active agent is 10:1, 9:1, 8:1, 7:1, 6:1, 5:1, 4:1, 3:1, 2:1, 1:1, 1;2, 1:3, 1:4, 1:5, 1:6, 1:7, 1:8, 1:9, or 1:10. In some embodiments, the ratio of the weight amount of the material responsive to the exogenous source to the active agent is 1:1.

In some embodiments, the particles comprise IR absorbing agent in an amount ranging from about 5.0 wt. % to about 15.0 wt. % by the total weight of the particles. In some embodiments, the IR absorbing agent is present in an amount ranging from about 5.5 wt. % to about 15.0 wt. % by the total weight of the particle. In some embodiments, the IR absorbing agent is present in an amount ranging from about 6.0 wt. % to about 15.0 wt. % by the total weight of the particle. In some embodiments, the IR absorbing agent is present in an amount ranging from about 6.5 wt. % to about 15.0 wt. % by the total weight of the particle. In some embodiments, the IR absorbing agent is present in an amount ranging from about 7.0 wt. % to about 15.0 wt. % by the total weight of the particle. In some embodiments, the IR absorbing agent is present in an amount ranging from about 7.5 wt. % to about 15.0 wt. % by the total weight of the particle. In some embodiments, the IR absorbing agent is present in an amount ranging from about 8.0 wt. % to about 15.0 wt. % by the total weight of the particle. In some embodiments, the IR absorbing agent is present in an amount ranging from about 8.5 wt. % to about 15.0 wt. % by the total weight of the particle. In some embodiments, the IR absorbing agent is present in an amount ranging from about 9.0 wt. % to about 15.0 wt. % by the total weight of the particle. In some embodiments, the IR absorbing agent is present in an amount ranging from about 9.5 wt. % to about 15.0 wt. % by the total weight of the particle. In some embodiments, the IR absorbing agent is present in an amount ranging from about 10.0 wt. % to about 15.0 wt. % by the total weight of the particle. In some embodiments, the IR absorbing agent is present in an amount ranging from about 10.5 wt. % to about 15.0 wt. % by the total weight of the particle. In some embodiments, the IR absorbing agent is present in an amount ranging from about 11.0 wt. % to about 15.0 wt. % by the total weight of the particle. In some embodiments, the IR absorbing agent is present in an amount ranging from about 11.5 wt. % to about 15.0 wt. % by the total weight of the particle. In some embodiments, the IR absorbing agent is present in an amount ranging from about 12.0 wt. % to about 15.0 wt. % by the total weight of the particle. In some embodiments, the IR absorbing agent is present in an amount ranging from about 12.5 wt. % to about 15.0 wt. % by the total weight of the particle. In some embodiments, the IR absorbing agent is present in an amount ranging from about 13.0 wt. % to about 15.0 wt. % by the total weight of the particle. In some embodiments, the IR absorbing agent is present in an amount ranging from about 13.5 wt. % to about 15.0 wt. % by the total weight of the particle. In some embodiments, the IR absorbing agent is present in an amount ranging from about 14.0 wt. % to about 15.0 wt. % by the total weight of the particle. In some embodiments, the IR absorbing agent is present in an amount ranging from about 5.0 wt. % to about 14.0 wt. % by the total weight of the particle. In some embodiments, the IR absorbing agent is present in an amount ranging from about 5.5 wt. % to about 14.0 wt. % by the total weight of the particle. In some embodiments, the IR absorbing agent is present in an amount ranging from about 6.0 wt. % to about 14.0 wt. % by the total weight of the particle. In some embodiments, the IR absorbing agent is present in an amount ranging from about 6.5 wt. % to about 14.0 wt. % by the total weight of the particle. In some embodiments, the IR absorbing agent is present in an amount ranging from about 7.0 wt. % to about 14.0 wt. % by the total weight of the particle. In some embodiments, the IR absorbing agent is present in an amount ranging from about 7.5 wt. % to about 14.0 wt. % by the total weight of the particle. In some embodiments, the IR absorbing agent is present in an amount ranging from about 8.0 wt. % to about 14.0 wt. % by the total weight of the particle. In some embodiments, the IR absorbing agent is present in an amount ranging from about 8.5 wt. % to about 14.0 wt. % by the total weight of the particle. In some embodiments, the IR absorbing agent is present in an amount ranging from about 9.0 wt. % to about 14.0 wt. % by the total weight of the particle. In some embodiments, the IR absorbing agent is present in an amount ranging from about 9.5 wt. % to about 14.0 wt. % by the total weight of the particle. In some embodiments, the IR absorbing agent is present in an amount ranging from about 10.0 wt. % to about 14.0 wt. % by the total weight of the particle. In some embodiments, the IR absorbing agent is present in an amount ranging from about 10.5 wt. % to about 14.0 wt. % by the total weight of the particle. In some embodiments, the IR absorbing agent is present in an amount ranging from about 11.0 wt. % to about 14.0 wt. % by the total weight of the particle. In some embodiments, the IR absorbing agent is present in an amount ranging from about 11.5 wt. % to about 14.0 wt. % by the total weight of the particle. In some embodiments, the IR absorbing agent is present in an amount ranging from about 12.0 wt. % to about 14.0 wt. % by the total weight of the particle. In some embodiments, the IR absorbing agent is present in an amount ranging from about 12.5 wt. % to about 14.0 wt. % by the total weight of the particle. In some embodiments, the IR absorbing agent is present in an amount ranging from about 13.0 wt. % to about 14.0 wt. % by the total weight of the particle. In some embodiments, the IR absorbing agent is present in an amount ranging from about 13.5 wt. % to about 14.0 wt. % by the total weight of the particle. In some embodiments, the IR absorbing agent is present in an amount ranging from about 5.0 wt. % to about 13.0 wt. % by the total weight of the particle. In some embodiments, the IR absorbing agent is present in an amount ranging from about 5.5 wt. % to about 13.0 wt. % by the total weight of the particle. In some embodiments, the IR absorbing agent is present in an amount ranging from about 6.0 wt. % to about 13.0 wt. % by the total weight of the particle. In some embodiments, the IR absorbing agent is present in an amount ranging from about 6.5 wt. % to about 13.0 wt. % by the total weight of the particle. In some embodiments, the IR absorbing agent is present in an amount ranging from about 7.0 wt. % to about 13.0 wt. % by the total weight of the particle. In some embodiments, the IR absorbing agent is present in an amount ranging from about 7.5 wt. % to about 13.0 wt. % by the total weight of the particle. In some embodiments, the IR absorbing agent is present in an amount ranging from about 8.0 wt. % to about 13.0 wt. % by the total weight of the particle. In some embodiments, the IR absorbing agent is present in an amount ranging from about 8.5 wt. % to about 13.0 wt. % by the total weight of the particle. In some embodiments, the IR absorbing agent is present in an amount ranging from about 9.0 wt. % to about 13.0 wt. % by the total weight of the particle. In some embodiments, the IR absorbing agent is present in an amount ranging from about 9.5 wt. % to about 13.0 wt. % by the total weight of the particle. In some embodiments, the IR absorbing agent is present in an amount ranging from about 10.0 wt. % to about 13.0 wt. % by the total weight of the particle. In some embodiments, the IR absorbing agent is present in an amount ranging from about 10.5 wt. % to about 13.0 wt. % by the total weight of the particle. In some embodiments, the IR absorbing agent is present in an amount ranging from about 11.0 wt. % to about 13.0 wt. % by the total weight of the particle. In some embodiments, the IR absorbing agent is present in an amount ranging from about 11.5 wt. % to about 13.0 wt. % by the total weight of the particle. In some embodiments, the IR absorbing agent is present in an amount ranging from about 12.0 wt. % to about 13.0 wt. % by the total weight of the particle.

In some embodiments, the IR absorbing agent is present in an amount ranging from about 5.0 wt. % to about 12.0 wt. % by the total weight of the particle. In some embodiments, the IR absorbing agent is present in an amount ranging from about 5.5 wt. % to about 12.0 wt. % by the total weight of the particle. In some embodiments, the IR absorbing agent is present in an amount ranging from about 6.0 wt. % to about 12.0 wt. % by the total weight of the particle. In some embodiments, the IR absorbing agent is present in an amount ranging from about 6.5 wt. % to about 12.0 wt. % by the total weight of the particle. In some embodiments, the IR absorbing agent is present in an amount ranging from about 7.0 wt. % to about 12.0 wt. % by the total weight of the particle. In some embodiments, the IR absorbing agent is present in an amount ranging from about 7.5 wt. % to about 12.0 wt. % by the total weight of the particle. In some embodiments, the IR absorbing agent is present in an amount ranging from about 8.0 wt. % to about 12.0 wt. % by the total weight of the particle. In some embodiments, the IR absorbing agent is present in an amount ranging from about 8.5 wt. % to about 12.0 wt. % by the total weight of the particle. In some embodiments, the IR absorbing agent is present in an amount ranging from about 9.0 wt. % to about 12.0 wt. % by the total weight of the particle. In some embodiments, the IR absorbing agent is present in an amount ranging from about 9.5 wt. % to about 12.0 wt. % by the total weight of the particle. In some embodiments, the IR absorbing agent is present in an amount ranging from about 10.0 wt. % to about 12.0 wt. % by the total weight of the particle. In some embodiments, the IR absorbing agent is present in an amount ranging from about 10.5 wt. % to about 12.0 wt. % by the total weight of the particle. In some embodiments, the IR absorbing agent is present in an amount ranging from about 11.0 wt. % to about 12.0 wt. % by the total weight of the particle. In some embodiments, the IR absorbing agent is present in an amount ranging from about 11.5 wt. % to about 12.0 wt. % by the total weight of the particle.

In some embodiments, the IR absorbing agent is present in an amount ranging from about 5.0 wt. % to about 11.0 wt. % by the total weight of the particle. In some embodiments, the IR absorbing agent is present in an amount ranging from about 5.5 wt. % to about 11.0 wt. % by the total weight of the particle. In some embodiments, the IR absorbing agent is present in an amount ranging from about 6.0 wt. % to about 11.0 wt. % by the total weight of the particle. In some embodiments, the IR absorbing agent is present in an amount ranging from about 6.5 wt. % to about 11.0 wt. % by the total weight of the particle. In some embodiments, the IR absorbing agent is present in an amount ranging from about 7.0 wt. % to about 11.0 wt. % by the total weight of the particle. In some embodiments, the IR absorbing agent is present in an amount ranging from about 7.5 wt. % to about 11.0 wt. % by the total weight of the particle. In some embodiments, the IR absorbing agent is present in an amount ranging from about 8.0 wt. % to about 11.0 wt. % by the total weight of the particle. In some embodiments, the IR absorbing agent is present in an amount ranging from about 8.5 wt. % to about 11.0 wt. % by the total weight of the particle. In some embodiments, the IR absorbing agent is present in an amount ranging from about 9.0 wt. % to about 11.0 wt. % by the total weight of the particle. In some embodiments, the IR absorbing agent is present in an amount ranging from about 9.5 wt. % to about 11.0 wt. % by the total weight of the particle. In some embodiments, the IR absorbing agent is present in an amount ranging from about 10.0 wt. % to about 11.0 wt. % by the total weight of the particle. In some embodiments, the IR absorbing agent is present in an amount ranging from about 10.5 wt. % to about 11.0 wt. % by the total weight of the particle.

In some embodiments, the IR absorbing agent is present in an amount ranging from about 5.0 wt. % to about 10.0 wt. % by the total weight of the particle. In some embodiments, the IR absorbing agent is present in an amount ranging from about 5.5 wt. % to about 10.0 wt. % by the total weight of the particle. In some embodiments, the IR absorbing agent is present in an amount ranging from about 6.0 wt. % to about 10.0 wt. % by the total weight of the particle. In some embodiments, the IR absorbing agent is present in an amount ranging from about 6.5 wt. % to about 10.0 wt. % by the total weight of the particle. In some embodiments, the IR absorbing agent is present in an amount ranging from about 7.0 wt. % to about 10.0 wt. % by the total weight of the particle. In some embodiments, the IR absorbing agent is present in an amount ranging from about 7.5 wt. % to about 10.0 wt. % by the total weight of the particle. In some embodiments, the IR absorbing agent is present in an amount ranging from about 8.0 wt. % to about 10.0 wt. % by the total weight of the particle. In some embodiments, the IR absorbing agent is present in an amount ranging from about 8.5 wt. % to about 10.0 wt. % by the total weight of the particle. In some embodiments, the IR absorbing agent is present in an amount ranging from about 9.0 wt. % to about 10.0 wt. % by the total weight of the particle.

In some embodiments, the IR absorbing agent is present in an amount ranging from about 8.0 wt. % to about 10.0 wt. % by the total weight of the particle. In some embodiments, the IR absorbing agent is present in an amount ranging from about 7.0 wt. % to about 10.0 wt. % by the total weight of the particle. In some embodiments, the IR absorbing agent is present in an amount ranging from about 6.0 wt. % to about 10.0 wt. % by the total weight of the particle. In some embodiments, the IR absorbing agent is present in an amount ranging from about 5.0 wt. % to about 10.0 wt. % by the total weight of the particle. In some embodiments, the IR absorbing agent is present in an amount ranging from about 4.0 wt. % to about 10.0 wt. % by the total weight of the particle. In some embodiments, the IR absorbing agent is present in an amount ranging from about 3.0 wt. % to about 10.0 wt. % by the total weight of the particle.

In some embodiments, the particles comprise IR absorbing agent in an amount selected from the group of about 5.0 wt. %, about 5.56 wt. %, about 10.4 wt. %, about 12.0 wt. %, about 12.1 wt. %, about 13.64 wt. %, about 14.0 wt. %, or about 15.0 wt. % by the total weight of the particles. In some embodiments, the particles comprise IR absorbing agent in an amount of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, or about 15.0 wt. %.

In some embodiments, the particle heater exhibits stability such that the degradation of the material by body chemicals is less than 20% as measured by the Efficacy Determination Protocol after incubating the particles in the extraction medium (containing serum) for 24 hours at 37° C. In some embodiments, the particle exhibits stability such that the active agent has a degree of degradation selected from the group of about 5.0%, about 10%, about 15%, about 20% as measured by Efficacy Determination Protocol. In some embodiments, the active agent has a degree of degradation in a range selected from the group of less than about 20.0%, less than about 15.0%, less than about 10.0%, less than about 5.0%, less than about 1.0%, less than about 0.5%, less than about 0.1%, and less than about 0.01% as determined by Efficacy Determination Protocol. In some embodiments, the active agent has a degree of degradation less than about 10.0% as determined by Efficacy Determination Protocol. In some embodiments, the active agent has a degree of degradation less than about 5.0% as measured by Efficacy Determination Protocol. In some embodiments, the active agent has a degree of degradation less than about 1.0% as measured by Efficacy Determination Protocol. In some embodiments, the anticancer agent and/or the material responsive to exogenous source has a degree of degradation less than about 0.1% as measured by Efficacy Determination Protocol.

In some embodiments, the particle exhibits stability and carrier matrix integrity such that the degradation of the active agent and/or the material ranges from about 5.0% to about 95% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is 0% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 90% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 85% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 80% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 75% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 70% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 65% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 60% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 55% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 50% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 45% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 40% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 30% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 20% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 10% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 5% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 1% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material is less than 0.1% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material ranges from about 0.01% to 10.0% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material ranges from about 0.01% to 5.0% as measured by the Efficacy Determination Protocol after incubating the particles in the extraction medium (serum) for 24 hours at 37° C. In some embodiments, the particle exhibits stability and carrier matrix integrity such that the degradation of the active agent and/or the material ranges from about 0.01% to 1.0% as measured by the Efficacy Determination Protocol after incubating the particles in the extraction medium (serum) for 24 hours at 37° C. In some embodiments, the particle exhibits stability such that the active agent and the material respectively has a degree of degradation selected from the group of about 0%, about 0.01%, about 0.1%, about 0.5%, about 1.0%, about 2.0%, about 3.0%, about 5.0%, about 6.0%, about 7.0%, about 8.0%, about 9.0%, about 10.0%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, or about 95%. In some embodiments, the particle exhibits stability such that the active agent and the material respectively has a degree of degradation selected from the group of about 5.0%, about 10%, about 15%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, the particle exhibits stability such that the degree of the degradation of the active agent and the material respectively ranges from about 25% to about 50%. In some embodiments, the particle exhibits stability such that the degradation of the active agent and the material respectively is less than about 25.0% as measured by the Efficacy Determination Protocol. In some embodiments, the active agent and the material respectively has a degree of degradation in a range selected from the group of: less than about 25.0%, less than about 20.0%, less than about 15.0%, less than about 10.0%, less than about 5.0%, less than about 1.0%, less than about 0.5%, less than about 0.1%, less than about 0.01%, 0% as determined by the Efficacy Determination Protocol. In some embodiments, the active agent and the material respectively has a degree of degradation less than about 10.0% as determined by the Efficacy Determination Protocol. In some embodiments, the active agent and the material respectively has a degree of degradation less than about 5.0%. In some embodiments, the active agent and the material respectively has a degree of degradation less than about 1.0%. In some embodiments, the active agent and the material respectively has a degree of degradation less than about 0.1%.

### Carrier

To achieve the stability and the cytotoxicity criteria set forth above, it is desirable to provide particles that exhibit carrier matrix integrity. The particle's integrity is the result of the proper choice of the carrier. It is important to select a carrier that is compatible with the material to be encapsulated.

In some embodiments, the carrier comprises a biocompatible and/or biodegradable polymer. In some embodiments, the carrier comprises a lipid, an organic polymer, an inorganic polymer or combinations thereof. In some embodiments, the biocompatible and/or biodegradable polymer contains labile bonds such as ester-, amide-, acetal-, ketal-, and anhydride-bonds that are prone to degradation by the chemistry inside the body.

In some embodiments, the polymers may include, but are not limited to: polymethyl methacrylate, polyester, poly caprolactone (PCL), poly(trimethylene carbonate) or other poly (alpha-esters), polyurethanes, poly(allylamine hydrochloride), poly(ester amides), poly (ortho esters), polyanyhydrides, poly (anhydride-co-imide), cross linked polyanhydrides, pseudo poly(amino acids), poly (alkylcyanoacrylates), polyphosphoesters, polyphosphazenes, chitosan, collagen, natural or synthetic poly(amino acids), elastin, elastin-like polypeptides, albumin, fibrin, polysiloxanes, polycarbosiloxanes, polysilazanes, polyalkoxysiloxanes, polysaccharides, cross-linkable polymers, thermo-responsive polymers, thermo-thinning polymers, thermo-thickening polymers, or block co-polymers of the above polymers with polyethylene glycol, and combinations thereof.

In some embodiments, the carrier comprises a hydrophobic polymer or copolymer of polymethacrylates, polycarbonate, or combinations thereof. In some embodiments, the carrier comprises polymethylmethacrylate (PMMA, Neocryl® 728 sold by DSM, T_{g}=111° C.).

In some embodiments, the carrier comprises copolymer of two different methacrylate monomers. In some embodiments, the carrier comprises copolymer of methyl methacrylate monomer and C2-C6 alkyl methacrylate monomer. In some embodiments, the carrier comprises copolymer of methyl methacrylate monomer and C2-C4 alkyl methacrylate monomer. In some embodiments, the carrier comprises copolymer of methyl methacrylate monomer and C3-C4 alkyl methacrylate monomer. In some embodiments, the polymethacrylate copolymer is made from methyl methacrylate monomer and C4 alkyl methacrylate monomer. In some embodiments, the polymethacrylate copolymer is made from methyl methacrylate (MMA) monomer in an amount ranging from about 80.0 wt. % to about 99.0 wt. % and butyl methacrylate (BMA) monomer in an amount ranging from about 1.0 wt. % to about 20.0 wt. % by the total weight of the polymethacrylate copolymer. In some embodiments, the polymethacrylate copolymer is made from MMA monomer in an amount ranging from about 85.0 wt. % to about 96.0 wt. % and BMA monomer in an amount ranging from about 4.0 wt. % to about 15.0 wt. % by the total weight of the polymethacrylate copolymer. In some embodiments, the polymethacrylate copolymer is made from MMA monomer in an amount ranging from about 90.0 wt. % to about 96.0 wt. % and BMA monomer in an amount ranging from about 4.0 wt. % to about 10.0 wt. % by the total weight of the polymethacrylate copolymer. In some embodiments, the polymethacrylate copolymer is made from MMA monomer in an amount ranging from about 95.0 wt. % to about 96.0 wt. % and BMA monomer in an amount ranging from about 4.0 wt. % to about 5.0 wt. % by the total weight of the polymethacrylate copolymer. In some embodiments, the polymethacrylate copolymer is made from about 99.0 wt. % MMA monomer and about 1.0 wt. % BMA monomer by the total weight of the polymethacrylate copolymer. In some embodiments, the polymethacrylate copolymer is made from about 98.0 wt. % MMA monomer and about 2.0 wt. % BMA monomer by the total weight of the polymethacrylate copolymer. In some embodiments, the polymethacrylate copolymer is made from about 97.0 wt. % MMA monomer and about 3.0 wt. % BMA monomer by the total weight of the polymethacrylate copolymer. In some embodiments, the polymethacrylate copolymer is made from about 96.0 wt. % MMA monomer and about 4.0 wt. % BMA monomer by the total weight of the polymethacrylate copolymer. In some embodiments, the polymethacrylate copolymer is made from about 95.0 wt. % MMA monomer and about 5.0 wt. % BMA monomer by the total weight of the polymethacrylate copolymer. In some embodiments, the polymethacrylate copolymer is made from about 94.0 wt. % MMA monomer and about 6.0 wt. % BMA monomer by the total weight of the polymethacrylate copolymer.

In some embodiments, the weight ratio of the MMA repeating units to the BMA repeating units in the MMA/BMA copolymer is 80:20 to 99:1. In some embodiments, the weight ratio of the MMA repeating units to the BMA repeating units in the MMA/BMA copolymer is 85:15 to 96:4. In some embodiments, the weight ratio of the MMA repeating units to the BMA repeating units in the MMA/BMA copolymer is 90:10 to 96:4. In some embodiments, the weight ratio of the MMA repeating units to the BMA repeating units in the MMA/BMA copolymer is 95:5 to 96:4. In some embodiments, the weight ratio of the MMA repeating units to the BMA repeating units in the MMA/BMA copolymer is 80:20, 81:19, 82:18, 83:17, 84:16, 85:15, 86:14, 87:13, 88:12, 89:11, 90:10, 91:9, 92:8, 93:7, 94:6, 95:5, 96:4, 97:3, 98:2, or 99:1. In some embodiments, the polymethacrylate copolymer is MMA/BMA copolymer and the weight ratio of MMA to BMA is 96:4 (e.g. Neocryl® 805 by DSM, acid value less than 1).

In some embodiments, the hydrophobic polymethacrylate has an acid value less than 10. In some embodiments, the hydrophobic polymethacrylate has an acid value less than 5. In some embodiments, the hydrophobic polymethacrylate has an acid value less than 2. In some embodiments, the hydrophobic polymethacrylate has an acid value less than 1.

In an embodiment, the carrier may comprise a lipid selected from the group of lipid, polymer-lipid blend, polymer-lipid conjugate, carbohydrate-lipid conjugate, peptide-lipid conjugate, protein-lipid conjugate, and combinations thereof. In some embodiments, the lipid may include one or more of the following: phospholipids such as soy lecithin, egg lecithin, phosphatidylcholine, soy phosphatidylcholine, phosphatidylserine, phosphatidylinositide, phosphatidylethanolamine, phosphatidylglycerol, phosphatidic acid; sphingolipid such as sphingomyelin, ceramide, phytoceramide, cerebroside; and sterol such as cholesterol, desmosterol, lanthosterol, stigmasterol, zymosterol, or diosgenin.

In an embodiment, the carrier may comprise a lipid polymer blend, wherein the polymer may include poly(lactide-co-glycolide) (PLGA), polycaprolactone (PCL), polyethylene glycol, and polyoxyethylene-polyoxypropylene block copolymer. For example, the lipid polymer blend contains a blend of polycaprolactone and polyoxyethylene-polyoxypropylene block copolymer (Pluronic® F-68, Pluronic® F-127) with soy phosphatidylcholine.

In some embodiments, the carrier comprises a polymer-lipid conjugate, wherein the polymers are conjugated to polar head groups of the lipid may include polyethylene glycol, polyoxazolines, polyglutamines, polyasparagines, polyaspartamides, polyacrylamides, polyacrylates, polyvinylpyrrolidone, or polyvinylmethyether.

In some embodiments, the carrier comprises lipids or lipid-based materials selected from the group of phospholipids including phosphatidylcholines, phosphatidylserines, phosphatidylinositides, phosphatidylethanolamines, phosphatidylglycerols, phosphatidic acids, sphingolipids including sphingomyelins, ceramides, phytoceramides, cerebrosides, sterols including cholesterol, desmosterol, lanthosterol, stigmasterol, zymosterol, diosgenin, polymer-lipid conjugate of which the polymer conjugated to the polar head groups of the lipid including polyethylene glycol, polyoxazolines, polyglutamines, polyasparagines, polyaspartamides, polyacrylamides, polyacrylates, polyvinylpyrrolidone, or polyvinylmethyether, carbohydrate-lipid conjugate of which the carbohydrate conjugated to the lipid including monosaccharides (glucose, fructose), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectin, chitin, cellulose, starch, peptide-lipid conjugate, protein-lipid conjugate, and combinations thereof.

In some embodiments, the carrier comprises a carbohydrate-lipid conjugate, wherein the carbohydrate conjugated to the lipid may include monosaccharides (glucose, fructose etc.), disaccharides, oligosaccharides or polysaccharides such as glycosaminoglycan (hyaluronic acid, keratan sulfates, heparin sulfate or chondroitin sulfate), carrageenan, microbial exopolysaccharides, alginate, chitosan, pectins, chitin, cellulose, or starch.

In one embodiment, the phospholipid is selected from the group of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof. In an embodiment, the particle comprise the lipid selected from the group of DPPC, MPPC, PEG, DMPC, DMPG, DSPE, DOPC, DOPE, DPPG, DSPC, DSPE-PEG, MSPC, cholesterol, PS, PC, PE, PG, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

In some embodiments, the carrier comprises 2 parts of 1,2-distearoyl-sn-glycero-3-phosphoglycerol (DSPG), 1 part of cholesterol, and 0.2 part of 1,2-distearoyl-sn-glycero-3-phosphoethanolamine (DSPE-PEG2000). In some embodiments, the carrier comprises 2 parts sphingomyelin (egg), 1 part cholesterol and 0.2 parts of 1,2-distearoyl-sn-glycero-3-phosphoethanolamine (DSPE-PEG2000).

In some embodiments, the carrier comprises a biodegradable and/or biocompatible polymer. In some embodiments, the carrier is selected based on the specific material to be encapsulated, e.g., carrier is chemically compatible with the material.

In some embodiments, the biodegradable and/or biocompatible polymer may include, but is not limited to, a polyester, a polyurea, a polyanhydride, a polysaccharide, a polyphosphoester, a poly(ortho ester), a poly(amino acid), a protein, polyurea, and combinations thereof.

In some embodiments, the biodegradable and/or biocompatible polymer comprises a polymer selected from the group of PLA, PGA, PLGA, PCL, polydioxanone, poly(trimethylene carbonate) or other poly (alpha-esters), polyurethanespoly(ester amides), poly (ortho esters), polyanyhydrides, poly (anhydride-co-imide), cross linked polyanhydrides, pseudo poly(amino acids), polyphosphoesters, polyphosphazenes, chitosan, collagen, natural or synthetic poly(amino acids), elastin, elastin-like polypeptides, albumin, fibrin, silk fibroin, keratin, collagen, gelatin, ovalbumin, serum albumin, corn zein, soy protein, gluten, milk protein, polysaccharides, cross-linkable polymers, thermoresponsive polymers (e.g., methacrylate-co-N-isopropylacylamide) sulfobetaine), thermo-thinning polymers, thermo-thickening polymers, or block co-polymers of these with polyethylene glycol, and combinations thereof.

In one embodiment, the carrier is a polyester. Polyesters are a class of polymers characterized by ester linkages in the backbone, such as poly (lactic acid) (PLA), poly (glycolic acid) (PGA), PLGA, etc. PLGA is one of the commonly used polymers in developing particulate active agent delivery systems. PLGA degrades via hydrolysis of its ester linkages in the presence of water. Due to the hydrophobic nature of PLGA, PLGA particles with core-shell structures are prepared through various emulsification processes and hydrophilic active agents could be encapsulated in the hydrophilic shell of the particles, while hydrophobic active agents tend to distribute in the hydrophobic core.

In one embodiment, the carrier is PMMA. In some embodiments, PMMA is degraded by random-chain/end-chain depolymerization caused by or accelerated by heat.

In some embodiments, the carrier comprises a polyester selected from the group of PLA, PGA, PLGA, and combinations thereof. In some embodiments, the carrier comprises a blend of polyester and hydrophilic polymers selected from polyethylene glycol, polymer or block copolymer of polyalkylene oxide, polysaccharides, proteins, and combinations thereof.

In some embodiments, the carrier is selected from the group of PLA; PGA; PLGA; block copolymer of polyethylene glycol-b-poly lactic acid-co-glycolic acid (PEG-PLGA); PCL; poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine; and combinations thereof.

In some embodiments, the carrier comprises a polyester selected from the group of PLA, PGA, PLGA, and combinations thereof.

In some embodiments, copolymers of PEG or derivatives thereof with any of the polymers described above may be used as carrier to make the polymeric particles. In some embodiments, the carrier comprises a polymer blend containing PLGA 75:25 and PLGA-PEG 75:25, lactide:glycolide (L:G) monomer ratio is 75:25.

Blend with or copolymers of PEG or derivatives thereof with any of the biodegradable polymers described above may be used to make the polymeric particles. In certain embodiments, the PEG or derivatives may be located in the interior positions of the triblock copolymer (e.g, PLA-PEG-PLA). Alternatively, the PEG or derivatives may be located near or at the terminal positions of the block copolymer. In certain embodiments, the particles are formed under conditions that allow regions of PEG to phase separate or otherwise to reside on the surface of the particles.

In some embodiments, the leakage of the material from the carrier or the incursion of the body chemicals may be modulated by varying the molar ratio of the hydrophilic repeating unit, glycolide to the hydrophobic repeating unit, lactide in a PLGA copolymer. In some embodiments, the proportion of lactic acid units and glycolic acids units within the copolymer may be in a range selected from the group of 10:90 to 90:10, from 15:85 to 85:15, from 20:80 to 80:20, from 25:75 to 75:25, from 30:70 to 70:30, from 35:65 to 65:35, from 40:60 to 60:40, and from 45:55 to 55:45 and the PLGA has a number average molecular weight ranging from 450 Da to 15,000 Da. In some embodiments, the polymer comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45, and has a number average molecular weight ranging from 450 Da to 10,000 Da. In some embodiments, the polymer comprises a PLGA having a lactide:glycolide (L:G) molar ratio from 5:95 to 95:5, 10:90 to 90:10, 15:85 to 85:15, 25:75 to 75:25, 40:60 to 60:40, or 45:55 to 55:45, and has a number average molecular weight ranging from 10,000 Da to 15,000 Da.

In some embodiments, the polymer comprises a PLGA having a lactide:glycolide (L:G) molar ratio from 15:85 to 85:15, 25:75 to 75:25, 40:60 to 60:40, or 45:55 to 55:45, and has a number average molecular weight ranging from 450 Da to 15,000 Da. In some embodiments, the polymer comprises a PLGA having a lactide:glycolide (L:G) molar ratio from 15:85 to 85:15, 25:75 to 75:25, 40:60 to 60:40, or 45:55 to 55:45, and has a number average molecular weight ranging from 570 Da to 8000 Da. In some embodiments, the polymer comprises a PLGA having a lactide:glycolide molar ratio from 25:75 to 75:25, 40:60 to 60:40, or 45:55 to 55:45 and has a number average molecular weight ranging from 570 Da to 3000 Da. In some embodiments, the polymer comprises a PLGA having a lactide:glycolide molar ratio from 25:75 to 75:25, 40:60 to 60:40, or 45:55 to 55:45 and has a number average molecular weight ranging from 1000 Da to 10,000 Da. In some embodiments, the polymer comprises PLGA having a lactide:glycolide molar ratio from 25:75 to 75:25, 40:60 to 60:40, or 45:55 to 55:45 and has a number average molecular weight selected from the group of 5000 Da, 6000 Da, 7000 Da, 8000 Da, 9000 Da, 10,000 Da, 11, 000 Da, 12,000 Da, 13,000 Da, 14,000 Da, and 15,000 Da.

In some embodiments, the PLGA has a lactide:glycolide monomer ratio ranging from 70:30 to 30:70 and an average molecular weight of 4,000 Da, or 11,000 Da. In some embodiments, the PLGA has a 70:30 lactide:glycolide monomer ratio and a number average molecular weight of 1500 Da, or 4500 Da (PLG 1600HL™). In some embodiments, the PLGA has a 75:25 lactide:glycolide (L:G) monomer ratio and a weight average molecular weight of 90,000 Da to 126,000 Da (PLGA 75:25). In some embodiments, the PLGA has a 50:50 lactide:glycolide monomer ratio and a number average molecular weight 2515 Da (Resomer RG® 502H).

In some embodiments, copolymer of D, L isomers of lactic acid is applied to modulate the polymer water solubility and the leakage property of the material to outside particle, or the incursion of the body chemicals to the particle interior. In some embodiments, the polymer is a poly(L-co-D,L-lactic acid (PLDLA) in a L-LA to D,L-LA monomer ratio selected from the group of 90:10, 85:15, 80:20, 75:25, 70:30, 65:35, 60:40, and 55:45 to form particles that encapsulate hydrophobic material. In some embodiments, PLDLA has a number average molecular weight ranging from 2000 Da to 50,000 Da (or a weight average molecular weight Mw ranging from 3400 Da to 85,000 Da, polydispersity 1.7 (Mw/Mn)). In some embodiments, the polymer is a poly(L-co-D,L-lactic acid (PLDLA) in the 70:30 L-LA to D,L-LA monomer ratio and has a number average molecular weight ranging from 2000 Da to 50,000 Da (or a weight average molecular weight Mw ranging from 3400 Da to 85,000 Da, polydispersity 1.7 (Mw/Mn)). The PLDLA in 70:30 monomer ratio is an amorphous polymer that facilitates the degradation. The PLDLA polymer has excellent biodegradability, biocompatibility and controlled degradation characteristics.

In some embodiments, the carrier comprises a blend of PLGA and PLGA-PEG (PLGA & PLGA-PEG polymer blend). In some embodiments, the PLGA to PEG in the polymer blend has a weight ratio ranging from 10:1 to 1:10. In some embodiments, the PLGA to PEG in the polymer blend has a weight ratio selected from 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, and 1:10. In some embodiments, the PLGA to PEG in the polymer blend has a weight ratio 1:1.

In some embodiments, the hydrophilic polymer segment is incorporated into the hydrophobic PLGA or PLA polymer backbone (PEG-polyester block copolymer) to modulate the leakage of the material to outside of particle or the incursion of the body chemicals to the particle interior. In some embodiments, the hydrophilic segment comprises polyethylene glycol (PEG), polyalkyleneoxide, block copolymer of polyalkyleneoxide, or dendritic polyglycerol. In some embodiments, the hydrophilic segment is polyethylene glycol having a number average molecular weight ranging from 500 Da to 10,000 Da. In some embodiments, the carrier comprises a block copolymer of PLGA with PEG (PLGA-co-PEG block copolymer).

In some embodiments, the triblock copolymer is PLA-PEG-PLA, wherein the PLA block has a number average molecular weight of 450 Da to 5000 Da, and the PEG block has a number average molecular weight of 200 Da to 7500 Da. In some embodiments, the triblock copolymer is PLA-PEG-PLA, wherein the PLA block has a number average molecular weight of 500 Da to 3000 Da, and the PEG block has a number average molecular weight of 200 Da to 3500 Da. In some embodiments, the triblock copolymer is PLA-PEG-PLA, wherein the PLA block has a number average molecular weight of 2000 Da to 3000 Da, and the PEG block has a number average molecular weight of 3000 Da to 3500 Da. In some embodiments, the triblock copolymer is PLA-PEG-PLA, wherein the PLA block has a number average molecular weight of 2000 Da, and the PEG block has a number average molecular weight of 10,000 Da (PLA(2K)-b-PEG(10K)-b-PLA(2K).

In some embodiments, the PEG modified polyester polymer is di-block copolymer of poly(sebacic acid) and polyethylene glycol (PSA-PEG), wherein the PSA has a number average molecular weight ranging from 500 Da to 15,000 Da and the PEG segment has a number average molecular weight ranging from 450 Da to 15,000 Da. In some embodiments, the carrier is a PSA-PEG diblock copolymer, wherein the PSA segment of the diblock copolymer PSA-PEG has a number average molecular weight ranging from 500 Da to 10,000 Da and the PEG segment of the diblock copolymer PSA-PEG has a number average molecular weight ranging from 450 Da to 10,000 Da. In some embodiments, the carrier is a PSA-PEG diblock copolymer, wherein the PSA segment of the diblock copolymer PSA-PEG has a number average molecular weight ranging from 500 Da to 10,000 Da and the PEG segment of the diblock copolymer PSA-PEG has a number average molecular weight ranging from 450 Da to 5,000 Da.

In some embodiments, the carrier comprises a mixture of poly(aspartic acid-co-L-lactide)(PAL) and polyethylene glycol such that the particle formed thereof comprises PEG in its shell to enclose the hydrophobic core. In some embodiments, the carrier comprises poly(aspartic acid-co-L-lactide) and PEG having a weight ratio of poly(aspartic acid-co-L-lactide) to PEG ranging from 1:10 to 10:1. In some embodiments, the weight ratio of poly(aspartic acid-co-L-lactide) to PEG in the particle ranges from 1:1 to 7:1. In some embodiments, the weight ratio of poly(aspartic acid-co-L-lactide) to PEG in the particle is selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1. In some embodiments, the weight ratio of poly(aspartic acid-co-L-lactide) to PEG in the particle is selected from the group of 1:1, 2:1, 3:1, 5:1, and 7:1. In some embodiments, the weight ratio of poly(aspartic acid-co-L-lactide) to PEG in the particle is 1:1.

In some embodiments, the weight ratio of poly(aspartic acid-co-L-lactide) to PEG in the particle is 3:1. In some embodiments, the weight ratio of poly(aspartic acid-co-L-lactide) to PEG in the particle is 5:1. In some embodiments, the weight ratio of poly(aspartic acid-co-L-lactide) to PEG in the particle is 7:1. In some embodiments, the weight ratio of poly(aspartic acid-co-L-lactide) to PEG in the particle is 1:2. In some embodiments, the weight ratio of poly(aspartic acid-co-L-lactide) to PEG in the particle is 1:3. In some embodiments, the weight ratio of poly(aspartic acid-co-L-lactide) to PEG in the particle is 1:4. In some embodiments, the weight ratio of poly(aspartic acid-co-L-lactide) to PEG in the particle is 1:5. In some embodiments, the weight ratio of poly(aspartic acid-co-L-lactide) to PEG in the particle is 1:7.

In some embodiments, the carrier comprises a mixture of poly(L-lactic acid) (PLLA) and poly(aspartic acid-co-L-lactide) (PAL). The degradation rate becomes higher for the carrier containing PAL with higher molar ratios of lactide to aspartic acid units [LA]/[Asp].

In some embodiments, the biodegradable polymers are monodispersed polymers. In some embodiments, the biodegradable polymers has a polydispersity (PD=Mw/Mn) ranging from 1.0 to 10.0. In some embodiments, the biodegradable polymers has a polydispersity ranging from 1.0 to 3.0. In some embodiments, the biodegradable polymers has a polydispersity ranging from 2.0 to 3.0. In some embodiments, the biodegradable polymers has a polydispersity selected from the group of 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0. In some embodiments, the PLGA has a polydispersity ranging from 1.0 to 2.0. In some embodiments, the biodegradable polymers has a polydispersity of about 1.2.

In some embodiments, the carrier is a protein selected from the group of lipoproteins, albumin, fibrin, silk fibroin, keratin, collagen, gelatin, ovalbumin, serum albumin, corn zein, soy protein, gluten, milk protein, and combinations thereof.

In some embodiments, the carrier comprises one or more polysaccharides selected from the group of carrageenan, microbial exopolysaccharides, alginate, chitosan, pectins, chitin, cellulose, starch, and combinations thereof.

In some embodiments, the carrier for encapsulating the material interacting with the exogenous source comprises gelatin or collagen since gelatin or collagen is natural extra cellular matrix protein having endogenous cell membrane binding RGD motif.

In some embodiments, the carrier comprises acrylate polymers which undergo depolymerization (end-chain or random-chain scission) to release the trapped active agent, with the depolymerization initiated by or activated by the heat generated from the interaction between the exogenous source and the material.

The deterioration of the physical and mechanical properties is often the result of bond breaking in the polymer backbone (chain scission) which may occur at the chain ends or at random positions in the chain. In the case of chain-end scission, monomers are released. This process is known as unzipping, depropagation or end-chain depolymerization. Random main chain scission, on the other hand, leads to the formation of both monomers and oligomers (short chains with ten or fewer monomers). This process can be considered the reverse of a step-growth polymerization. Both reactions compete with cross-linking reactions, chain stripping of side groups as well as with substituent and cyclization reactions. Which of these mechanisms dominates depends on the type of polymer and temperature. Polyacrylates often undergo significant chemical changes over time when exposed to sufficiently high temperatures. Polymers with no or only a single (small) substituent in the repeat unit usually decompose by random-chain scission rather than end-chain scission. This is the case polymethyl acrylate. On the other hand, end-chain scission is usually the predominant decomposition mechanism in polymers with two substituents at the same carbon atom because the (large) side groups interfere with hydrogen abstraction which is known as steric hindrance. Thus disubstituted polymers like poly(methyl methacrylate), poly(α-methylstyrene), and poly(methacrylonitrile) usually undergo end-chain scission with high monomer yield (≥90%) whereas polymers with a single large substituent are susceptible to both random chain scission and end-chain scission.

A well-known example of end-chain depolymerization (unzipping) with high monomer yield is the decomposition of polymethyl methacrylate (PMMA). This polymer starts to decompose at about 350° C. (660° F.). Random-chain fragmentation is the main initiation step in the early stages and in the later stages end-chain scission which is usually of first order. The main propagation step is unzipping to monomer which releases large amounts of methyl methacrylate (>90%). This depolymerization can trigger release of the encapsulated active agent to trigger unwanted cell (cancer or microbial cell) death.

In some embodiments, the particle comprises carrier in an amount ranging from about 60.0 wt. % to about 85 wt. % by the total weight of the particle. In some embodiments, the particle comprises carrier in an amount ranging from about 65.0 wt. % to about 85 wt. % by the total weight of the particle. In some embodiments, the particle comprises carrier in an amount ranging from about 70.0 wt. % to about 85 wt. % by the total weight of the particle. In some embodiments, the particle comprises carrier in an amount ranging from about 71.0 wt. % to about 85 wt. % by the total weight of the particle. In some embodiments, the particle comprises carrier in an amount ranging from about 72.0 wt. % to about 85 wt. % by the total weight of the particle. In some embodiments, the particle comprises carrier in an amount ranging from about 72.5 wt. % to about 85 wt. % by the total weight of the particle. In some embodiments, the particle comprises carrier in an amount ranging from about 73.0 wt. % to about 85 wt. % by the total weight of the particle. In some embodiments, the particle comprises carrier in an amount ranging from about 74.0 wt. % to about 85 wt. % by the total weight of the particle. In some embodiments, the particle comprises carrier in an amount ranging from about 75.0 wt. % to about 85 wt. % by the total weight of the particle. In some embodiments, the particle comprises carrier in an amount ranging from about 76.0 wt. % to about 85 wt. % by the total weight of the particle. In some embodiments, the particle comprises carrier in an amount ranging from about 77.0 wt. % to about 85 wt. % by the total weight of the particle. In some embodiments, the particle comprises carrier in an amount ranging from about 78.0 wt. % to about 85 wt. % by the total weight of the particle. In some embodiments, the particle comprises carrier in an amount ranging from about 79.0 wt. % to about 85 wt. % by the total weight of the particle. In some embodiments, the particle comprises carrier in an amount ranging from about 80.0 wt. % to about 85 wt. % by the total weight of the particle.

In some embodiments, the particle comprises carrier in an amount ranging from about 65.0 wt. % to about 80 wt. % by the total weight of the particle. In some embodiments, the particle comprises carrier in an amount ranging from about 64.0 wt. % to about 80 wt. % by the total weight of the particle. In some embodiments, the particle comprises carrier in an amount ranging from about 63.0 wt. % to about 80 wt. % by the total weight of the particle. In some embodiments, the particle comprises carrier in an amount ranging from about 62.0 wt. % to about 80 wt. % by the total weight of the particle. In some embodiments, the particle comprises carrier in an amount ranging from about 61.0 wt. % to about 80 wt. % by the total weight of the particle. In some embodiments, the particle comprises carrier in an amount ranging from about 60.0 wt. % to about 80 wt. % by the total weight of the particle. In some embodiments, the particle comprises carrier in an amount ranging from about 59.0 wt. % to about 80 wt. % by the total weight of the particle. In some embodiments, the particle comprises carrier in an amount ranging from about 58.0 wt. % to about 80 wt. % by the total weight of the particle. In some embodiments, the particle comprises carrier in an amount ranging from about 57.0 wt. % to about 80 wt. % by the total weight of the particle. In some embodiments, the particle comprises carrier in an amount ranging from about 56.0 wt. % to about 80 wt. % by the total weight of the particle. In some embodiments, the particle comprises carrier in an amount ranging from about 55.0 wt. % to about 80 wt. % by the total weight of the particle. In some embodiments, the particle comprises carrier in an amount ranging from about 55.0 wt. % to about 85 wt. % by the total weight of the particle. In some embodiments, the particle comprises carrier in an amount ranging from about 56.0 wt. % to about 84 wt. % by the total weight of the particle. In some embodiments, the particle comprises carrier in an amount ranging from about 57.0 wt. % to about 83 wt. % by the total weight of the particle. In some embodiments, the particle comprises carrier in an amount ranging from about 58.0 wt. % to about 82 wt. % by the total weight of the particle. In some embodiments, the particle comprises carrier in an amount ranging from about 59.0 wt. % to about 81 wt. % by the total weight of the particle. In some embodiments, the particle comprises carrier in an amount ranging from about 60.0 wt. % to about 80 wt. % by the total weight of the particle. In some embodiments, the particle comprises carrier in an amount ranging from about 61.0 wt. % to about 79 wt. % by the total weight of the particle. In some embodiments, the particle comprises carrier in an amount ranging from about 61.0 wt. % to about 78 wt. % by the total weight of the particle

In some embodiments, the particle comprises carrier in an amount ranging from about 62.0 wt. % to about 64.0 wt. % by the total weight of the particle. In some embodiments, the particle comprises carrier in an amount ranging from about 62.0 wt. % to about 74 wt. % by the total weight of the particle. In some embodiments, the particle comprises carrier in an amount ranging from about 61.0 wt. % to about 77 wt. % by the total weight of the particle. In some embodiments, the particle comprises carrier in an amount ranging from about 61.0 wt. % to about 76 wt. % by the total weight of the particle. In some embodiments, the particle comprises carrier in an amount ranging from about 61.0 wt. % to about 75 wt. % by the total weight of the particle. In some embodiments, the particle comprises carrier in an amount ranging from about 61.0 wt. % to about 74 wt. % by the total weight of the particle.

In some embodiments, the particle comprises carrier in an amount ranging from about 70.0 wt. % to about 80.0 wt. % by the total weight of the particle. In some embodiments, the particle comprises carrier in an amount ranging from about 71.0 wt. % to about 79 wt. % by the total weight of the particle. In some embodiments, the particle comprises carrier in an amount ranging from about 72.0 wt. % to about 78 wt. % by the total weight of the particle. In some embodiments, the particle comprises carrier in an amount ranging from about 72.0 wt. % to about 77 wt. % by the total weight of the particle. In some embodiments, the particle comprises carrier in an amount ranging from about 72.0 wt. % to about 76 wt. % by the total weight of the particle. In some embodiments, the particle comprises carrier in an amount ranging from about 72.0 wt. % to about 75 wt. % by the total weight of the particle.

In some embodiments, the particle comprises carrier in an amount selected from the group of 62.0 wt. %, 70.0 wt. %, 75.0 wt. % or 78.3 wt. % by the total weight of the particle. In some embodiments, the particle comprises carrier in an amount selected from the group of about 55.0 wt. %, about 56.0 wt. %, about 57.0 wt. %, about 58.0 wt. %, about 59.0 wt. %, about 60.0 wt. %, about 61.0 wt. %, about 62.0 wt. %, about 63.0 wt. %, about 64.0 wt. %, about 65.0 wt. %, about 66.0 wt. %, about 67.0 wt. %, about 68.0 wt. %, about 69.0 wt. %, about 70.0 wt. %, about 71.0 wt. %, about 72.0 wt. %, about 73.0 wt. %, about 74.0 wt. %, about 75.0 wt. %, about 76.0 wt. %, about 77.0 wt. %, about 78.0 wt. %, about 79.0 wt. %, or about 80 wt. % by the total weight of the particle.

In some embodiments, the particle comprises the carrier to the active agent in a weight ratio ranging from 1:10 to 10:1. In some embodiments, the weight ratio of the carrier to the active agent ranges from 1:1 to 7:1. In some embodiments, the weight ratio of the carrier to the active agent is selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1. In some embodiments, the weight ratio of the carrier to the active agent is selected from the group of 1:1, 2:1, 3:1, 4:1, 5:1, 6:1, and 7:1.

In some embodiments, the particle containing the carrier and the active agent has a weight ratio of the carrier to the active agent ranging from 1:1 to 7:1. In some embodiments, the particle containing the carrier and the active agent has a weight ratio of the carrier to the active agent ranging from 2:1 to 7:1. In some embodiments, the particle containing the carrier and the active agent has a weight ratio of the carrier to the active agent ranging from 3:1 to 7:1. In some embodiments, the particle containing the carrier and the active agent has a weight ratio of the carrier to the active agent ranging from 5:1 to 7:1. In some embodiments, the particle containing the carrier and the active agent has a weight ratio of the carrier to the active agent selected from the group of 1:1, 2:1, 3:1, 4:1, 5:1, 6:1, or 7:1.

In some embodiments, the particle containing the carrier and the active agent has a weight ratio of the carrier to the active agent is 3:1. In some embodiments, the particle containing the carrier and the active agent has a weight ratio of the carrier to the active agent is 4:1. In some embodiments, the particle containing the carrier and the active agent has a weight ratio of the carrier to the active agent is 5:1. In some embodiments, the particle containing the carrier and the active agent has a weight ratio of the carrier to the active agent is 6:1. In some embodiments, the particle containing the carrier and the active agent has a weight ratio of the carrier to the active agent is 7:1.

### Anticancer Agent

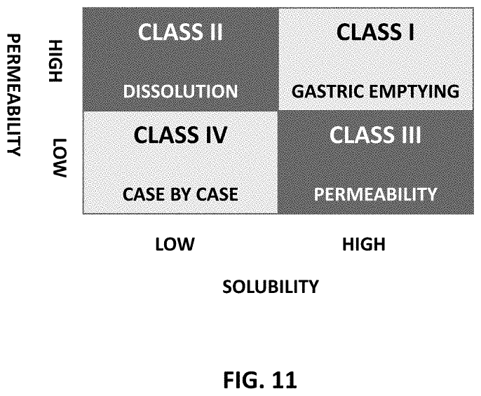

In some embodiments, the anticancer agent has a plasma half-life of less than 30 minutes. In some embodiments, the anticancer agent is a Class II, Class III or Class IV compound according to a Biopharmaceutics Classification System (FIG. 11). In some embodiments, the anticancer agents lack tumor selectivity, thus increase potential toxicity in normal tissues.

In some embodiments, the anticancer agent is a small molecule compound selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelalisib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

In some embodiments, the small molecule anticancer agent is a tyrosine kinase inhibitor (TKI), a targeted therapy for treating lung cancer (e.g., NSCLC). Tyrosine kinases are specific proteins that act as enzymes that may signal cancer cells to grow. The proteins encoded by the ALK, EGFR, ROS1, and BRAF genes are all examples of tyosine kinases. Tyrosine kinase inhibitors are targeted therapies that block these cell signals. By blocking the signals, they keep the cancer from growing and spreading. TKIs are named based on the enzyme, or protein, that they block. The driver mutations for which there are FDA-approved drugs on the market are anaplastic lymphoma kinase (ALK) inhibitors, EGFR inhibitors, ROS1 inhibitor, and BRAF V600E combination inhibitor, and NTRK inhibitor.

In some embodiments, the TKI inhibitor is selected from the group of afatinib, alectinib, brigatinib, ceritinib, crizotinib, dacomitinib, dabrafenib, erlotinib, gefitinib, larotrectinib, lorlatinib, osimertinib, and combinations thereof.

In some embodiments, the small molecule anticancer agent is a PI3K inhibitor selected from the group of wortmannin, temsirolimus, everolimus, buparlisib (BMK-120), 5-(2,6-dimorpholinopyrimidin-4-yl)-4-(trifluoromethyl)pyridin-2-amine), pictilisib, gedatolisib, apitolisib, pilaralisib, copanli sib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), and combinations thereof.

In some embodiments, the small molecule anticancer agent is curcumin. In some embodiments, the small molecule anticancer agent is paclitaxel.

The antitumor activity of curcumin has been extensively investigated, and it has been demonstrated that several proteins involved in cancer signaling pathways were regulated by curcumin, such as tumor suppressors P53, P21 and P27, inflammatory regulator NF-κB, and Akt/mTOR in pancreatic and colon cancer (Hussain et al., Curcumin induces apoptosis via inhibition of PI3′-kinase/AKT pathway in acute T cell leukemias. Apoptosis. 2006; 11:245-254). Several studies reported that curcumin may regulate multiple signaling pathways, including PI3K/AKT, MAPK and nuclear factor (NF)-κB (Nagaraju et al., The impact of curcumin on breast cancer. Integr Biol (Camb) 2012; 4:996-1007). Curcumin exerts synergistic effects when combined with other chemoactive agents. In breast cancer cell lines, curcumin and paclitaxel exert complementary effects on the alteration of proteins involved in apoptotic and inflammatory pathways (Quispe-Soto et al., Effect of curcumin and paclitaxel on breast carcinogenesis. Int J Oncol. 2016; 49:2569-2577). Curcumin was shown to induce endothelial growth factor receptor degradation and potentiate the antitumor activity of gefitinib in non-small-cell lung cancer cell lines and xenograft mouse models; intriguingly, it also attenuated gefitinib-induced gastrointestinal adverse effects via altering p38 activation (Lee et al., Curcumin induces EGFR degradation in lung adenocarcinoma and modulates p38 activation in intestine: The versatile adjuvant for gefitinib therapy. PLoS One. 2011; 6:e23756). Curcumin was also shown to increase the response of pancreatic cancer cells to gemcitabine through attenuating EZH2 and lncRNA PVT1 expression (Yoshida et al., Curcumin sensitizes pancreatic cancer cells to gemcitabine by attenuating PRC2 subunit EZH2, and the lncRNA PVT1 expression. Carcinogenesis. 2017; 38:1036-1046). In addition, curcumin was reported to inhibit epithelial-to-mesenchymal transition (EMT) of breast cancer cells (Gallardo et al., Curcumin inhibits invasive capabilities through epithelial mesenchymal transition in breast cancer cell lines. Int J Oncol. 2016; 49:1019-1027; Gallardo et al., Curcumin and epithelial-mesenchymal transition in breast cancer cells transformed by low doses of radiation and estrogen. Int J Oncol. 2016; 48:2534-2542).

It has been reported that curcumin gives dose-dependently inhibition against a variety of breast cancer cell lines, including T47D, MCF7, MDA-MB-415, SK-BR-3, MDA-MB-231, MDA-MB-468 and BT-20, with different ER, progesterone receptor (PR) and human epidermal growth factor receptor-2 (HER2) statuses. Curcumin was more active on ER+ breast cancer cells, such as T47D, MCF7 and MDA-MB-415, with an IC50 of 2.07±0.08, 1.32±0.06 and 4.69±0.06 μM, respectively (FIG. 1B). With regards to the ER-PR-HER2- cells, such as MDA-MB-231, MDA-MD-468 and BT-20 cells, the IC50 was relatively weaker, namely 11.32±2.13 μM, 18.61±3.12 μM and 16.23±2.16 μM, respectively (Hu et al., Curcumin inhibits proliferation and promotes apoptosis of breast cancer cells, Exp. Ther Med., 2018, vol. 16, pp. 1266-1272).

In some embodiments, the small molecule anticancer agent is a proteasome inhibitor selected from the group of bortezomib, ixazomib, marizomib, oprozomib, delanzomib, epoxomicin, disulfiram, lactacystin, beta-hydroxy beta-methylbutyrate, and combinations thereof.

In some embodiments, the small molecule anticancer drug is an EGFR inhibitor selected from the group of erlotinib, gefitinib, neratinib, osimertinib, vandetanib, dacomitinib, lapatinib, and combinations thereof.

In some embodiments, the small molecule anticancer agent is a PI3K inhibitor selected from the group of alpelisib and buparlisib (BKM-120).

In some embodiments, the anticancer agent is a targeted therapy for breast cancer selected from the group of CDK4 and CDK6 inhibitors, EGFR inhibitor, human epidermal growth factor receptor-2 (HER-2), anti-HER-2 monoclonal antibody, tyrosine kinase inhibitors, and combinations thereof. In some embodiments, the targeted therapy for breast cancer is selected from the group of abemaciclib, trastuzumab, lapatinib, trastuzumab, and combination thereof. In some embodiments, the targeted therapy for breast cancer comprises dual anti-HER2 therapy with lapatinib and trastuzumab.

In some embodiments, the anticancer agent comprises biologic anticancer agent selected from the group of therapeutic peptides, proteins, and combinations thereof. In some embodiments, the biologic anticancer agent is a monoclonal antibody, and fragments, a recombinant or synthetic protein, a peptide, an aptamer, a peptide nucleic acid (PNA), conjugates, variants, and biosimilars thereof.

In some embodiments, the biologic anticancer agent is a protein including cytokines or hematopoietic factors including without limitation IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), interferon-alpha (IFN-alpha), IFN-beta, IFN-gamma, IL-7, IL-8, IL-9, IL-10, IL-12, IL-13, IL-14, IL-15, IL-16, IL-17, IL-18, fibroblast growth factor receptor (FGFR), erythropoietin (EPO), vascular endothelial growth factor (VEGF), CD 19, CD22, CD25, CD30, death protein including bcl2, BH3, TNFα-related apoptosis-inducing ligand (TRAIL), cytokines targeting hematological malignancies including CD2, CD3, CD5, CD7, CD17, CD19, CD20, CD22, CD30, CD38, CD64 (effect for acute myeloid leukemia, AML), CD80 and CD86.

In some embodiments, the biologic anticancer agent is a monoclonal antibody against receptors selected from the group of TNFα, PD-1, CD17, CD20, CD22, CD33, CD34, CD38, CD44, CD47, CD52, CD90, EGFR, PDGFR, VEGF, HER2, and fragments, conjugates, variants, biosimilars thereof, and combinations thereof.

In some embodiments, the biologic anticancer agent is a humanized anti-CD20, humanized anti-CD38, mouse bispecific anti-CD19/anti-CD3, or chimeric anti-GD2 monoclonal antibody. In some embodiments, the biologic anticancer agent is a humanized anti-PD-1 monoclonal antibody. In some embodiments, the biologic anticancer agent is a chimeric anti-IL-6 monoclonal antibody. In some embodiments, the biologic anticancer agent is an anti-epidermal growth factor (EGFR) or an anti-vascular endothelial growth factor (VEGF). In some embodiments, the biologic anticancer agent is a G-CSF growth factor. In some embodiments, the anticancer agent is an EGFR binding antibody selected from the group of cetuximab, panitumumab, and combinations thereof.

Protein can be used to treat different types of cancer (e.g., lung cancer, CRC, pancreatic cancer, gastric cancer, prostate cancer, and breast cancer), from early diagnosis, to treatment, to prognosis. In addition to these types of cancer, peptides can also be used against cancers such as skin cancer, renal cancer, and osteosarcoma.

In some embodiments, the biologic anticancer agent is a peptide. In some embodiments, the biologic anticancer agent is a peptide derived from extracellular matrix proteins, growth factors and growth factor receptors, coagulation cascade proteins, chemokines, Type I Thrombospondin domain-containing proteins, and serpins.

Peptides, derived from natural or synthetic sources, can selectively bind to cell surface receptors because they may share similar structures by containing arginine and lysine. These amino acids can form hydrogen bonds with the negatively charged components on the cell membrane, indicating that amino acids are the main reason why peptides may bind to tumor cell membranes. Peptides are not the only molecule that can bind to tumor cell membranes, but they are the ideal molecules because they have low molecular weights and good cellular uptake. Peptides, which are short chains of amino acid monomers linked by peptide bonds, can specifically bind to tumor cells with low toxicity to normal tissues.

Therapeutic peptides are a promising and novel approach to treat cancer. They are usually less than 50 amino acids in length and are often stabilized by disulfide bonds. Many sequences, structures and pattern interactions of oncogenic proteins are

In some embodiments, the biologic anticancer agent is a peptide cancer vaccine.

Tumor-associated antigens (TAAs) are expressed in tumor cells and can be recognized by T lymphocytes, resulting in activation of the immune system. A TAA peptide vaccine, when injected into cancer patients, binds with the restricted major histocompatibility complex (MHC) molecule expressed in antigen presenting cells (APCs). Then the peptide/MHC complex is transported to the cell surface after intracellular processing and recognized by T cell receptor (TCR) on the surface of T cells, leading to the activation of T lymphocytes. Therefore, a peptide cancer vaccine may elicit a specific immune response against tumors.

In some embodiments, the biologic anticancer agent is selected from the group of Melittin, Buforin IIb, Alloferon-1, Alloferon-2, Magainin 2, Cecropin B, LL-37, Tachyplesin, RGD-Tachyplesin, OLP-1, OLP-4, HPRP-A1-TAT, BR-1, BR-2, ZXR-1, ZXR-2, Mauriporin, Magainin A, DRS B4, YA*GFM, where A*=Cαα-dialkylated glycine (Aaa1,1), Pardaxin, and MB30.

In some embodiments, this disclosure provides particles with two or more anticancer agents, and one or more diagnostic agents enclosed within the particles with each agent providing a distinct function. In some embodiments, the diagnostic agent is an imaging contrast agent selected from fluorescence contrast agent, magnetic responsive contrast agent and combination thereof. In some embodiments, the fluorescence contrast agent is a cyanine dye including ICG and new ICG IR 820 dye. In some embodiments, the imaging contrast agent is iron oxide nanoparticle. In some embodiments, the imaging agent is iodine. In some embodiments the carrier includes iodine e.g. an iodinated polymer

In some embodiments, the particle has a loading amount of the anticancer agent that is measured by spectroscopic absorbance. In some embodiments, the particle has a loading amount of the active agent that is measured by known analytical technology in the art, like UV-VIS-NIR, NMR, HPLC, LCMS, etc. In some embodiments, the anticancer agent is present in an amount ranging from about 0.01 wt. % to about 99 wt. % by the total weight of the particle. In some embodiments, the loading amount for the anticancer agent is in a range from about 0.01 wt. % to about 95.0 wt. % by the total weight of the particle. In some embodiments, the anticancer agent loading amount is in a range from about 0.01 wt. % to about 20.0 wt. % by the total weight of the particle. In some embodiments, the loading amount for the anticancer agent is in a range from about 1.0 wt. % to about 20.0 wt. % by the total weight of the particle. In some embodiments, the loading amount for the anticancer agent is in a range from about 5.0 wt. % to about 20.0 wt. % by the total weight of the particle. In some embodiments, the loading amount for the anticancer agent is in a range from about 10.0 wt. % to about 20.0 wt. % by the total weight of the particle. In some embodiments, the loading amount for the anticancer agent is in a range from about 5.0 wt. % to about 15.0 wt. % by the total weight of the particle. In some embodiments, the loading amount for the anticancer agent is in a range from about 10.0 wt. % to about 15.0 wt. % by the total weight of the particle. In some embodiments, the loading amount for the anticancer agent is in a range from about 5.0 wt. % to about 12.5 wt. % by the total weight of the particle. In some embodiments, the loading amount for the anticancer agent is a value selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, and about 20.0 wt. % by the total weight of the particle. In some embodiments, the loading amount for the anticancer agent is in a range from about 12.5 wt. % by the total weight of the particle. In some embodiments, the loading amount for the anticancer agent is a value selected from the group of about 0.1 wt. %, about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 15.0 wt. %, about 20.0 wt. %, about 25.0 wt. %, about 30.0 wt. %, about 35.0 wt. %, about 40 wt. %, about 45 wt. %, about 50 wt. %, about 55.0 wt. %, about 60 wt. %, about 65.0 wt. %, about 70.0 wt. %, about 75.0 wt. %, about 80.0 wt. %, about 85.0 wt. %, about 90.0 wt. %, and about 95.0 wt. % by the total weight of the particle.

In some embodiments, the particle exhibits stability such that the degradation of the anticancer agent ranges from about 5.0% to about 95% as measured by the Efficacy Determination Protocol after incubating the particles in the extraction medium for 24 hours at 37° C. In some embodiments, the particle exhibits stability such that the anticancer agent has a degree of degradation selected from the group of about 0%, about 0.01%, about 0.1%, about 0.5%, about 1.0%, about 2.0%, about 3.0%, about 5.0%, about 6.0%, about 7.0%, about 8.0%, about 9.0%, about 10.0%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 31%, about 32%, about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, about 80%, about 81%, about 82%, about 83%, about 84%, about 85%, about 86%, about 87%, about 88%, about 89%, about 90%, about 91%, about 92%, about 93%, about 94%, and about 95% as measured by Efficacy Determination Protocol. In some embodiments, the particle exhibits stability such that the anticancer agent has a degree of degradation selected from the group of about 5.0%, about 10%, about 15%, about 20%, about 25%, about 30%, about 35%, about 40%, about 45%, about 50%, about 55%, about 60%, about 65%, about 70%, about 75%, about 80%, about 85%, about 90%, and about 95% as measured by Efficacy Determination Protocol. In some embodiments, the particle exhibits stability such that the degree of the degradation of the anticancer agent ranges from about 25% to about 50% as measured by Efficacy Determination Protocol. In some embodiments, the particle exhibits stability such that the degree of degradation of the anticancer agent is less than about 25.0% as measured by Efficacy Determination Protocol. In some embodiments, the anticancer agent has a degree of degradation in a range selected from the group of less than about 25.0%, less than about 20.0%, less than about 15.0%, less than about 10.0%, less than about 5.0%, less than about 1.0%, less than about 0.5%, less than about 0.1%, and less than about 0.01% as determined by Efficacy Determination Protocol. In some embodiments, the anticancer agent has a degree of degradation less than about 10.0% as determined by Efficacy Determination Protocol. In some embodiments, the anticancer agent has a degree of degradation less than about 5.0% as measured by Efficacy Determination Protocol. In some embodiments, the anticancer agent has a degree of degradation less than about 1.0% as measured by Efficacy Determination Protocol. In some embodiments, the anticancer agent and/or the material responsive to exogenous source has a degree of degradation less than about 0.1% as measured by Efficacy Determination Protocol.

Some additional protein kinase inhibitors are summarized in the Table 1 below.

TABLE 1
Protein Kinase Inhibitors

| | | | | |
|---|---|---|---|---|
| Generic Name Brand | | | Likelihood | |
| Name | Kinase Target | Approval | Score† | Major Uses†† |

CANCER

| | | | | |
|---|---|---|---|---|
| Abemaciclib | Cyclin dependent | 2017 | E* | Breast cancer |
| Verzenio | kinase 4/6 | | | |
| Acalabrutinib | Bruton kinase | 2017 | D | Mantel cell lymphoma |
| Calquence | | | | |
| Afatinib | EGFR, HER2 | 2013 | D | NSCLC |
| Gilotrif | | | | |
| Alectinib | ALK | 2015 | D | NSCLC |
| Alecensa | | | | |
| Axitinib | VEGFR 1-3 | 2012 | E | Renal cell cancer |
| Inlyta | | | | |
| Binimetinib | BRAF | 2018 | E* | Melanoma |
| Mektovi | | | | |
| Bortezomib | Proteasome | 2003 | C | Multiple myeloma, Mantle |
| Velcade | | | | cell lymphoma |
| Bosutinib | BCR-ABL, scr | 2012 | E* | CML, resistant |
| Bosulif | | | | |
| Brigatinib | ALK | 2017 | E* | NSCLC |
| Alunbrig | | | | |
| Cabozantinib | MET, VEGFR-2 | 2012 | E* | Medullary thyroid cancer, |
| Cometriq, Cabometyx | | | | Renal cell cancer |
| Carfilzomib | Proteasome | 2012 | E* | Multiple myeloma, resistant |
| Kyprolis | | | | |
| Ceritinib | ALK | 2014 | D | NSCLC |
| Zykadia | | | | |
| Cobimetinib | MEK | 2015 | D | Melanoma |
| Cotellic | | | | |
| Copanlisib | PI3Kα/δ | 2017 | E* | Follicular lymphoma |
| Aliqopa | | | | |
| Crizotinib | ALK | 2011 | D | NSCLC |
| Xalkori | | | | |
| Dabrafenib | BRAF | 2013 | E* | Melanoma |
| Tafinlar | | | | |
| Dacomitinib | HER1,2,3 | 2018 | E* | NSCLC |
| Vizimpro | | | | |
| Dasatinib | BCR-ABL, src | 2006 | D | CML, resistant |
| Sprycel | | | | |
| Duvelisib | PI3K | 2018 | E* | CLL, Small cell lymphoma |
| Copiktra | | | | |
| Enasidenib | Mutant IDH-2 | 2017 | E* | AML |
| IDHIFA | | | | |
| Encorafenib | BRAF | 2018 | E* | Melanoma |
| Braftovi | | | | |
| Erlotinib | EGFR, HER1 | 2004 | C | NSCLC, Pancreatic cancer |
| Tarceva | | | | |
| Gefitinib | EGFR | 2009 | C | NSCLC |
| Iressa | | | | |
| Gilteritinib | FLT3 | 2018 | E* | AML |
| Xospata | | | | |
| Glasdegib | Hedgehog | 2018 | E* | AML |
| Daurismo | | | | |
| Ibrutinib | Bruton kinase | 2013 | D | Mantle cell lymphoma, |
| Imbruvica | | | | CLL |
| Idelalisib | PI3Kδ | 2014 | D | CLL, Non-Hodgkin |
| Zydelig | | | | lymphoma |
| Imatinib | BCR-ABL, c-Kit | 2001 | B | CML, GIST |
| Gleevec | | | | |
| Ivosidenib | Mutant IHD-1 | 2018 | E* | AML |
| Tibsovo | | | | |
| Ixazomib | 26S Proteasome | 2015 | E* | Multiple myeloma |
| Ninlaro | | | | |
| Lapatinib | EGFR, HER2 | 2007 | D | Breast cancer, HER2 |
| Tykerb | | | | positive |
| Larotrectinib | NTRK | 2018 | E* | Solid tumors |
| Vitrakvi | | | | |
| Lenvatinib | VEGFR 1-3, FGF 1-4, | 2015 | D | Thyroid cancer |
| Lenvima | PDGF, c-Kit, RET | 2016 | | Renal cell cancer |
| | | 2018 | | Hepatocellular cancer |
| Lorlatinib | ALK | 2018 | E* | NSCLC |
| Lorbrena | | | | |
| Midostaurin | FLT3 | 2018 | E* | AML |
| Rydapt | | | | |
| Neratinib | HER2 | 2017 | E* | Breast cancer |
| Nerlynx | | | | |
| Nilotinib | BCR-ABL | 2007 | E* | CML, resistant |
| Tasigna | | | | |
| Niraparib | PARP | 2017 | E* | Ovarian cancer |
| Zejula | | | | |
| Olaoarib | PARP | 2014 | E | Ovarian cancer |
| Lynparza | | 2018 | | Advanced breast cancer |
| Osimertinib | EGFR | 2015 | E* | NSCLC, refractory |
| Tagrisso | | | | |
| Palbociclib | ER+, HER2 | 2015 | E* | Breast cancer, HER2 |
| Ibrance | | | | negative |
| Pazopanib | VEGFR 1-3 | 2009 | C | Renal cell cancer |
| Votrient | | | | |
| Ponatinib | BCR-ABL | 2013 | E* | CML, ALL |
| Iclusig | | | | |
| Regorafenib | VEGFR 1-3, PDGF | 2012 | D | Colorectal cancer, GIST |
| Stivarga | | | | |
| Ribociclib | Cyclin dependent | 2017 | C | Breast cancer |
| Kisqali | kinase 4/6 | | | |
| Rucaparib | PARP | 2016 | D* | Ovarian cancer, advanced |
| Rubraca | | | | |
| Ruxolitinib | Janus kinase | 2011 | E* | Myelofibrosis |
| Jakafi | | | | |
| Sonidegib | Hedgehog | 2015 | E* | Basal cell skin cancer |
| Odomzo | | | | |
| Sorafenib | VEGFR 1-3 | 2005 | C | Renal cell cancer |
| Nexavar | | 2007 | | Hepatocellular cancer |
| | | 2013 | | Thyroid cancer |
| Sunitinib | PDGF, c-Kit | 2006 | D | CML, resistant; GIST, renal |
| Sutent | | | | cell cancer |
| Talazoparib | PARP | 2018 | E* | Breast cancer |
| Talzenna | | | | |
| Trametinib | MEK 1-2 | 2013 | E* | Melanoma |
| Mekinist | | | | |
| Vandetanib | VEGFR 2 | 2011 | E* | Medullary thyroid cancer |
| Caprelsa | | | | |
| Vemurafenib Zelboraf | BRAF | 2011 | E* | Melanoma |
| Vismodegib | Hedgehog | 2012 | D | Basal cell skin cancer |
| Erivedge | | | | |

MISCELLANEOUS

| | | | | |
|---|---|---|---|---|
| Baricitinib | Janus kinase | 2018 | E* | Rheumatoid arthritis |
| Olumiant | | | | |
| Fostamatinib | Spleen tyrosine kinase | 2017 | E* | Immune thrombocytopenia |
| Tavalisse | | | | |
| Nintedanib | VEGFR, FGFR, | 2014 | E* | Pulmonary fibrosis |
| Ofev | PDGFR | | | |
| Pegaptanib | VEGFR 1-3 | 2004 | E | Macular degeneration |
| Macugen | | | | |
| Tofacitinib | Janus kinase | 2012 | E* | Rheumatoid arthritis |
| Xeljanz | | | | |

†Likelihood Score indicates the likelihood of association with drug induced liver injury, based upon the known potential of the drug to cause such injury.
††Abbreviations: ALL, acute lymphocytic leukemia; AML, acute myeloid leukemia; CLL, chronic lymphocytic leukemia; CML, chronic myelogenous leukemia; GIST, gastrointestinal stromal tumor; NSCLC, non-small cell lung cancer.

### Antimicrobial Agent

In some embodiments, the particle heaters comprise an antimicrobial agent that is either encapsulated within the particle heater or covalently bonded to the particle heater surface. In some embodiments, the antimicrobial agent that can be used with the particle heater is selected from the group of small molecule antimicrobial agent, biologic antimicrobial agent, and combinations thereof.

In some embodiments, the antimicrobial agent is a small molecule antimicrobial agent. In some embodiments, the antimicrobial agent comprises H₂O₂.

In some embodiments, the antimicrobial agent is an inorganic compound or an organic compound. In some embodiments, the antimicrobial agent is an inorganic compound selected from the group of silver particles, gold particles, gallium particles, zinc oxide particles, copper oxide particles, and combinations thereof. In some embodiments, the antimicrobial agent is an organic compound selected from the group of an organic acid, a phenolic compound, a phyto-antibiotic, an amino acid, a quaternary ammonium compound, a surfactant, an antibiotic, and combinations thereof.

In some embodiments, the organic acid is selected form the group of acetic acid, ascorbic acid, alpha acids, adipic acid, benzenesulfonic acid, benzoic acid, citric acid, hops, gluconic acid, glutaric acid, hydroxyacetic acid, lactic acid, malic acid, methanesulphonic acid, oxalic acid, propionic acid, salicylic acid, succinic acid, tartaric acid, and combination thereof. In some embodiments, the antimicrobial agent is ascorbic acid.

In some embodiments, the antimicrobial agents may include, but are not limited by, those in the classes of penicillins, including amipicillin, flucloxacillin, dicloxacillin, methicillin, ticarcillin, piperacillin, carbapenems, mecillinams, cephalosporin and cephamycins; sulfonamides; aminoglycosides, including amikacin, gentamicin, kanamycin, neomycin, netilmicin, paromomycin, streptomycin, tobramycin, apramycin; chloramphenicol; erythromycin, azithromycin, clarithromycin, dirithromycin, roxithromycin, carbomycin A, josamycin, iktasamycin, oleandomycin, spiramycin, troleandomycin, tylosin/tylocine, telithromycin, cethromycin, ansamycin, lincomycin, clindamycin, mikamycins, pristinamycins, oestreomycins, virginiamycins, acanthomycin, actaplanin, avoparcin, balhimycin, bleomycin B (copper bleomycin), chloroorienticin, chloropolysporin, demethylvancomycin, enduracidin, galacardin, guanidylfungin, hachimycin, demethylvancomycin, N-nonanoyl-teicoplanin, phleomycin, platomycin, ristocetin, staphylocidin, talisomycin, teicoplanin, vancomycin, victomycin, xylocandin, zorbamycin, rifampicin, rifabutin, rifapentine, metronidazole, nitrothiazoles, nalidixic acid, cinoxacin, flumequine, oxolinic acid, piromidic acid, pipemidic acid, ciprofloxacin, enoxacin, fleroxacin, lomefloxacin, nadifloxacin, norfloxacin, ofloxacin, pefloxacin rufloxacin, balofloxacin, grepafloxacin, levofloxacin, pazufloxacin mesilate, sparfloxacin, temafloxacin, tosufloxacin, clinafloxacin, gemifloxacin, moxifloxacin, gatifloxacin, sitafloxacin, trovafloxacin, trimethoprim, linezolid, eperezolid, gramicidins, polymyxins, surfactin, tetracyclines, including chlortetracycline, oxytetracycline, demeclocycline, doxycycline, lymecycline, meclocycline, methacycline, minocycline, and rolitetracycline.

The antifungal agents include, but are not limited to, polyenes, such as amphotericin, nystatin, pimaricin, and the like; azole drugs, such as fluconazole, Itraconazole, ketoco, and the like; allylamine and morpholine drugs, such as naftifine, terbinafine, amorolfine, and the like; antimetabolite antifungal drugs, such as 5-fluorocytosine, and the like; and analogs, salts and derivatives thereof.

In some embodiments, the antimicrobial agent is a biocide. In some embodiments, the biocide may include silver, such as colloidal silver, silver salts including salts of one or more of the anionic polymers making up the material, silver sulfadiazine, chlorhexidine, hexetidine and cetylpyridinium salts, povidone iodine, triclosan, sucralfate, quaternary ammonium salts, and mixtures thereof. In some embodiments, the silver may include metallic silver as an antimicrobial that may be in the form of thin films, particles or colloidal silver. In some embodiments, the silver antimicrobials may be silver salt selected from silver sulfadiazine, silver norflocoactinate, silver pipemidate, silver thiosalicylate, silver imidazolium chloride, silver oxide, silver carbonate, silver deoxycholate, silver salicylate, silver iodide, silver nitrate, silver para-aminobenzoate, silver para-aminosalicylate, silver acetylsalicylate, silver ethylenediaminetetraacetic acid (“Ag EDTA”), silver picrate, silver protein, silver citrate, silver lactate, silver acetate, silver laurate, and combinations thereof.

In some embodiments, the antimicrobial agent may be a cationic surfactant derived from the condensation of fatty acids and esterified dibasic amino acids. In some embodiments, the cationic surfactant is lauric arginate (LAE). In some embodiments, the antimicrobial agent is curcumin.

In some embodiments, the antimicrobial agent is an antiseptic agent selected from the group of oligomeric or polymeric guanidine, biguanidine salts, and combinations thereof. In some embodiments, polymeric guanidine comprises the polyhexamethylene guanidine hydrochloride.

In some embodiments, the antimicrobial agent comprises a thermal stable antibiotic. In some embodiments, the thermal stable antibiotic comprises vancomycin.

In some embodiments, the particle has a loading amount of the antimicrobial agent that is measured by spectroscopic absorbance. In some embodiments, the particle has a loading amount of the antimicrobial agent that is measured by known analytical technology in the art, like UV-VIS/NIR, NMR, HPLC, LCMS, etc. In some embodiments, the antimicrobial agent is present in an amount ranging from about 0.01 wt. % to about 99 wt. % by the total weight of the particle. In some embodiments, the antimicrobial agent loading amount is in a range from about 0.01 wt. % to about 95.0 wt. % by the total weight of the particle. In some embodiments, the antimicrobial agent loading amount is in a range from about 0.01 wt. % to about 20.0 wt. % by the total weight of the particle. In some embodiments, the antimicrobial agent loading amount in a range from about 1.0 wt. % to about 20.0 wt. % by the total weight of the particle. In some embodiments, the antimicrobial agent loading amount in a range from about 5.0 wt. % to about 20.0 wt. % by the total weight of the particle. In some embodiments, the antimicrobial agent loading amount in a range from about 10.0 wt. % to about 20.0 wt. % by the total weight of the particle. In some embodiments, the antimicrobial agent loading amount in a range from about 5.0 wt. % to about 15.0 wt. % by the total weight of the particle. In some embodiments, the antimicrobial agent loading amount in a range from about 10.0 wt. % to about 15.0 wt. % by the total weight of the particle. In some embodiments, the antimicrobial agent loading amount in a range from about 5.0 wt. % to about 12.5 wt. % by the total weight of the particle. In some embodiments, the antimicrobial agent loading amount is a value selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, and about 20.0 wt. % by the total weight of the particle. In some embodiments, the antimicrobial agent loading amount of about 12.5 wt. % by the total weight of the particle. In some embodiments, the antimicrobial agent loading amount is a value selected from the group of about 0.1 wt. %, about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 15.0 wt. %, about 20.0 wt. %, about 25.0 wt. %, about 30.0 wt. %, about 35.0 wt. %, about 40 wt. %, about 45 wt. %, about 50 wt. %, about 55.0 wt. %, about 60 wt. %, about 65.0 wt. %, about 70.0 wt. %, about 75.0 wt. %, about 80.0 wt. %, about 85.0 wt. %, about 90.0 wt. %, and about 95.0 wt. % by the total weight of the particle.

In some embodiments, the biologic antimicrobial agent is selected from the group of a glycopeptide, a macrocyclic depsipeptide, a monoclonal antibody, a recombinant or synthetic protein, a peptide, an aptamer, peptide nucleic acids (PNA), and antigen-binding fragments, conjugates, variants, and biosimilars thereof.

In some embodiments, the biologic antimicrobial agent is a glycopeptide antibiotic. In some embodiments, the glycopeptide antibiotic includes vancomycin, bleomycin, phleomycin, tallysomycin, pepleomycin, and a mixture containing phleomycin D1, a copper-chelated glycopeptide antibiotic produced by Streptomyces CL990 (Zeoci™).

In some embodiments, the antimicrobial agent is teixobactin, an 11-residue, macrocyclic depsipeptide. This peptide has several unusual features, including four D-amino acids, a methylated phenylalanine, and the non-proteinogenic amino acid enduracididine. The amino acid sequence of teixobactin is MeHN-d-Phe-Ile-Ser-d-Gln-d-Ile-Ile-Ser-d-Thr*-Ala-enduracididine-Ile-COO—*. The carboxyl terminus forms a lactone with the 1-threonine residue (indicated by the asterisk), as is common in microbial non-ribosomal peptides. Teixobactin is effective against the drug-resistant bacterium methicillin-resistant Staphylococcus aureus, as well as Streptococcus pneumoniae, which can cause pneumonia and meningitis.

In some embodiments, the biologic antimicrobial agent is an antibody and variant antibody that targets S. aureus antigen; an antibody that targets the immunodominant ABC transporter in MRSA, which blocks the multi-drug efflux pump; tefibazumab, which targets Clumping Factor A; pagibaximab, which binds lipoteichoic acid present in the membrane of Gram positive bacteria; an antibody; an antibody binding to immunoglobulin binding proteins (IgBPs); a variant anti-microbial IgG antibody; and combinations thereof. In some embodiments, the antibody is a chimeric, or humanized human anti-microbial IgG variant antibody.

Antimicrobial peptides (AMP) have been found in virtually all organisms and display remarkable structural and functional diversity. Besides direct antimicrobial activity, AMPs carry immunomodulatory properties. Antimicrobial peptides are emerging as novel antimicrobial agents because they can combat multidrug resistant (MDR) microbes. Cationic antimicrobial peptides had a checkered history in the clinic and only five have progressed to clinical trials using topical applications, including, for example, protegrin-like peptide, indolicin and indolicin like peptides, gramicidin S and polymyxin B, which have been used in topical creams and solutions. However, these molecules tend to be toxic, which limits their potential for systemic use. Until cationic AMPs can be used systemically, they will not achieve their true therapeutic potential. They face many barriers, including the demonstration of good activity, sufficient stability in vivo, low toxicity, and a cost-effective manufacturing method. The multi-targeting particle of this disclosure provides solutions to at least one of these barriers for achieving the full clinical potential of biologic antimicrobial agents in treating MDR bacteria.

In some embodiments, the biologic antimicrobial agent comprises a cationic AMP. AMPs are not only potent antibiotics, but also effective modulators of inflammation and neutralizers of pathogenic toxins. Cationic AMPs have been recognized as effector molecules of the innate immune system that are integral to the first line of defense to fight microbial infections. Such AMP are widely distributed among species. These peptides are characterized by cationic properties that facilitate interactions with the negatively charged phospholipids of the bacterial membrane. Antimicrobial peptides have been shown to kill by permeabilizing the membrane of microbial organisms. The amphiphilic nature of these molecules facilitates the insertion of the hydrophobic residue into the lipid bilayer by electrostatic attraction, while the polar residues project into and above the membrane.

An advantage of peptide antibiotics as factors of the innate immune system is their ability to function without specificity, and without memory. Their anti-bacterial, anti-viral, and anti-fungal activities allow the host to delay or possibly even avoid microbial growth shortly after infection, before the adaptive immune response can be mobilized.

In some embodiments, the cationic AMP is selected from the group of buforin, magainin, apidaecin, oncocin, bacterial lipopolysaccharide neutralizing peptide Y113WF, a mammalian cathelicidin including fragment LL-37, IGKEFKRIVERIKRFLRELVRPLR (OP-145, derivative of LL-37), LAREYKKIVEKLKRWLRQVLRTLR (Peptide P-10), a cathelicidin indolicidin derivative including ILPWKWPWWPWRR—NH2, a homolog of indolicidin derivative omoganan (MX-226), RGKAKCCK a C-terminal octapeptide fragment of the human beta defensin-1 (HBD-1), human retrocyclin (human θ-defensin) including GICRCICGRGICRCICGR (RC1), GICRCICGRRICRCICGR (RC2), RICRCICGRRICRCICGR (RC3), protegrin, protegrin derivatives including NH₂-RGGRLCYCRRRFCVCVGR—CO—NH2 (protegrin-1, PG1), RGGRLCYCRRRFCICV (PG2), RGGGLCYCRRRFCVCVGRG (PG3), RGGRLCYCRGWICFCVGRG (PG4), RGGRLCYCRPRFCVCVGRG (PG5), RGGLCYCRGRFCVCVGR (iseganan, IB-367), pexiganan GIGKFLKKAKKFGKAFVKILKK-NH₂, gaegurin-5 FLGWLFKVASK, phyloseptin I FLSLIPHAINAVSAIAKHN, demaseptin S4 NH₂-ALWMTLKKKVLKAKAKALNAVLVGANA-NH₂, temporin SHa FLKGIKGMLGKLF—NH₂, piscidine, apidaecin isolate from a honey bee GNNRPVYIPQPRPPHPRI—OH, oreochromicin, microcin S, bactofensin LS1, APKAMKLLKKLLKLQKKGI (Arg rich synthetic peptides), glycopeptides, lipopeptides, lipoglycopeptides, and combinations thereof.

In some embodiments, the AMP comprises defensins and their derivatives. Defensins are the largest family of antibiotic peptides and are composed of 29 to 35 amino acid residues and constitute greater than 5% of total cellular protein in human neutrophils. For example, tracheal antibiotic peptide (TAP), is of the β-defensin class.

In some embodiments, the AMP is indolicidin, protegrin, prophenin, cecropin, magainin, lactoferricin, brvinin, tachyplesin, defensins, NK-lysin, or drosomycin.

In some embodiments, the particle has a loading amount of the antimicrobial agent that is measured by spectroscopic absorbance. In some embodiments, wherein the antimicrobial agent is present in an amount ranging from about 0.01 wt. % to about 99 wt. % by the total weight of the particle. In some embodiments, the antimicrobial agent loading amount is in a range from about 0.01 wt. % to about 95.0 wt. % by the total weight of the particle. In some embodiments, the antimicrobial agent loading amount is in a range from about 0.01 wt. % to about 20.0 wt. % by the total weight of the particle. In some embodiments, the particle has the antimicrobial agent loading amount in a range from about 1.0 wt. % to about 20.0 wt. % by the total weight of the particle. In some embodiments, the particle has the antimicrobial agent loading amount in a range from about 5.0 wt. % to about 20.0 wt. % by the total weight of the particle. In some embodiments, the particle has the antimicrobial agent loading amount in a range from about 10.0 wt. % to about 20.0 wt. % by the total weight of the particle. In some embodiments, the particle having the antimicrobial agent loading amount in a range from about 5.0 wt. % to about 15.0 wt. % by the total weight of the particle. In some embodiments, the particle has the antimicrobial agent loading amount in a range from about 10.0 wt. % to about 15.0 wt. % by the total weight of the particle. In some embodiments, the particle has the antimicrobial agent loading amount in a range from about 5.0 wt. % to about 12.5 wt. % by the total weight of the particle. In some embodiments, the antimicrobial agent loading amount is a value selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, and about 20.0 wt. % by the total weight of the particle. In some embodiments, the particle has the antimicrobial agent loading amount of about 12.5 wt. % by the total weight of the particle. In some embodiments, the antimicrobial agent loading amount is a value selected from the group of about 0.1 wt. %, about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 15.0 wt. %, about 20.0 wt. %, about 25.0 wt. %, about 30.0 wt. %, about 35.0 wt. %, about 40 wt. %, about 45 wt. %, about 50 wt. %, about 55.0 wt. %, about 60 wt. %, about 65.0 wt. %, about 70.0 wt. %, about 75.0 wt. %, about 80.0 wt. %, about 85.0 wt. %, about 90.0 wt. %, and about 95.0 wt. % by the total weight of the particle.

### Tumor Targeting Ligands

In some embodiments, the particle heater surface is further engineered to carry out additional functions (e.g., -localization of the particles in the tumor tissue) that improve the therapeutic efficiency. One such function is the targeted delivery of the particle heaters.

The systemic delivery of particle heaters to the tumor site is mainly based on “active” and “passive” mechanisms. Particle heaters with long systemic circulation properties tend to accumulate in the tumor interstitial space through a passive mechanism, where selective accumulation is mainly achieved by the enhanced permeability and retention (EPR) effect and is highly dependent on the leaky vasculature and impaired lymphatics intrinsic in fast-growing tumors. In the active targeting mode, the periphery of the particle heaters is conjugated or decorated with molecular ligands such as antibodies, peptides, biological proteins and cell-specific ligands, which may enhance the cellular uptake of particle heaters through receptor-mediated endocytosis. The active targeting of particle heaters with targeting ligands leads to increased material accumulation at the target tumor site, but the actual effect can be limited by various tumor microenvironmental factors such as tumor heterogeneity, hypoxia, endosomal escape and lysosomal degradation.

Ligand-mediated active targeting provides a way to increase the accuracy of localizing the particle heaters to the tumor site. There are numerous targets in tumors, both in the tumor blood vessels, and on the tumor and stromal cells that make up the tumor microenvironment. The targets within the tumors may include certain integrins, fibrin deposits, and tumor antigens, such as epidermal growth factor receptors (EGFR or HER2), folate receptors, prostate specific membrane antigen (PSMA), and carcinoembryonic antigen (CEA).

In some embodiments, the particle heater comprises a component capable of binding to a cancer protease of a target tumor type such that the particle heaters are localized to the targeted tumor site.

In some embodiments, the particles are conjugated with the corresponding tumor targeting ligands, such as proteins, peptides, aptamers, and small molecules through physical and chemical binding or covalent bonding. The targeting ligand used can be an antibody, a peptide or a natural ligand of a receptor preferentially expressed in tumors (e.g., folic acid to target the folate receptor). It is postulated that a particle heater coupled to a targeting ligand will preferentially accumulate in the tumor, resulting in greater thermal efficiency and fewer side effects elsewhere in the body.

In some embodiments, the particle heater comprises a targeting group on the particle surface selected from the group of tumor targeting folate, antibodies (e.g., Herceptin), antibody fragments, proteins, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, peptidomimetics, glycopeptides, peptoids, aptamer, claudin, HYNIC-(Ser)3-J18, FROP-1, and combinations thereof. In some embodiments, the targeting group is selected from the group of EGFR binding peptides, aptamers, claudin, HYNIC-(Ser)3-J18, FROP-1, and combinations thereof. In some embodiments, the targeting group is an EGRF binding peptide. In some embodiments, monoclonal antibodies (mAbs) are used to target B cells. The mAbs used to target B cells may include CD17 (acute lymphoma,) CD20 (mature) and CD22. Multiple myeloma is beyond the B cell phase. The mAbs used to target multiple myeloma is cD38 (only in plasma). In some embodiments, the targeting group selected will bind to CD17, CD20, CD22, and/or CD38 receptors.

EGFR mutations resulting in constitutive activation have been found in 10-35% of metastatic non-small cell lung cancer (NSCLC), and while EGFR inhibitors are effective for systemic disease, control of brain metastases remains limited by drug delivery. EGFR mutations are also found in 40-50% of primary glioblastoma multiforme (GBM) prevalent forms of brain cancer. While EGFR-tyrosine kinase inhibitors (TKIs), such as gefitinib, have shown promise in preclinical settings, they have demonstrated to be largely ineffective in brain cancer patients, likely due to poor tissue or central nervous system (CNS) penetration and dose-limiting toxicity.

Epidermal growth factor receptor (EGFR) is used for targeted therapy. In some embodiments, the targeting group is selected from the group of an EGFR binding antibody, an EGFR binding peptide, and combinations thereof. In some embodiments, the targeting group is an EGFR binding antibody selected from the group of cetuximab, panitumumab, and combinations thereof. In some embodiments, the targeting group is an EGFR peptide selected from the group of YHWYGYTPQNVI, YRWYGYTPQNVI, the L-AE (L amino acids in the sequence-FALGEA), D-AE (D-amino acids in the sequence-FALGEA), and combinations thereof. In some embodiments, the EGFR targeting group is covalently conjugated to the surface of the particle heater via a disulfide bond, the EGFR binding ligand as described above is release from the particle to impart therapeutic effects on killing cancer cells upon disulfide bond cleavage by the glutathione that is elevated in the tumor microenvironment.

In some embodiments, the targeting group is a cell membrane penetrating peptide (CPPs) including transferrin receptors and like peptides (CRGD, LyP-1 peptide). Tumor-penetrating peptides are particularly suitable for the targeted delivery of the material via particle to the tumor cells. First, internalization of the peptide and its payload into cells in the tumor makes tumor localizing more effective. Second, these CPP can take a particle payload into the cytoplasm, which is critical, for example, in the delivery of nucleic acid-based therapeutics. Third, tumor-penetrating capabilities can enhance particle extravasation and spreading in tumor tissue. Particles, because of their size, are particularly prone to be excluded from difficult-to-access parts of tumors and the CPP peptides can mitigate this problem. On the other hand, particles are a particularly favorable carrier for localizing peptides, including tumor-penetrating peptides, because multivalent presentation on the particle surface makes up for the relatively low affinity of the peptides through the avidity effect, enhancing receptor binding.

In some embodiments, the particle heaters are conjugated with tumor-penetrating peptides including, but not limited to, LyP-1 (sequence: vCGNKRTRGC (Cys-Gly-Asn-Lys-Arg-Thr-Arg-Gly-Cys), primarily accumulates in a myeloid cell/macrophage in tumors), i-LyP-1 (sequence: CGNKRTR (Cys-Gly-Asn-Lys-Arg-Thr-Arg)), TT1 (sequence: CKRGARSTC (Cys-Lys-Arg-Gly-Ala-Arg-Ser-Thr-Cys)), iNGR (sequence: CRNGRGPDC (Cys-Arg-Asn-Gly-Arg-Gly-Pro-Asp-Cys)), iRDG, a 9-amino acid cyclic peptide containing integrin-binding RDG motif (sequence: CRGDKGPDC (Cys-Arg-Gly-Asp-Lys-Gly-Pro-Asp-Cys)), F3 (sequence: KDEPQRRSARLSAKPAPPKPEPKPKKAPAKK (Lys-Asp-Glu-Pro-Gln-Arg-Arg-Ser-Ala-Arg-Leu-Ser-Ala-Lys-Pro-Ala-Pro-Pro-Lys-Pro-Glu-Pro-Lys-Pro-Lys-Lys-Ala-Pro-Ala-Lys-Lys)), CRGRRST (Cys-Arg-Gly-Arg-Arg-Ser-Thr), or a TAT peptide (sequence: YGRKKRRQRRR—COOH (Tyr-Gly-Arg-Lys-Lys-Arg-Arg-Gln-Arg-Arg-Arg-COOH)).

In this disclosure, the particle heaters are designed to have a peptide capable of binding to tumor protease between the surface of the particle heater and the targeting ligand. The cancer protease binding peptides used to conjugate the tumor-targeting group to the particle surface provides additional tumor type targeting by binding to tumor type specific protease elevated at the tumor microenvironment for precision in localizing particle heater to tumor sites.

In some embodiments, the particle surface targeting ligand modification further comprises a degradable spacer between the particle surface and the cancer protease binding peptide.

In some embodiments, the particle heater surface target ligand modifications comprises tumor targeting ligand-(amino-(spacer)x)y-peptide-carrier or tumor targeting ligand -(spacer)z-peptide-carrier, wherein the spacer has from 2 to 50 atoms, x, y and z are integers from 5 to 15.

In some embodiments, the degradable spacer is selected from the group of an ester bond, an amide bond, an imine bond, an acetal bond, a ketal bond, and combinations thereof.

In some embodiments, the spacer is selected from the group of polyethylene glycol having 2-50 repeating units, ε-Maleimidocaproic acid, para-aminobenzyloxy carbamater, and combinations thereof. In some embodiments, the spacer comprises polyamino acid having 2-30 amino acid residues. In some embodiments, the spacer comprises a linear polylysine, or polyglutamine

In terms of cancer protease binding peptides, overexpressed proteases have been identified in cancerous cells at elevated concentrations over healthy cells. It is reported that urokinase plasminogen activator (uPA), urokinase plasminogen activator (uPAR), cathepsin B, and membrane-type matrix metalloprotease (MMP) can initiate the activation of pro-MMPs. Then, extracellular matrix (ECM, collagen) degrading activities begin by extra-cellular serine proteases, like uPA, urokinase plasminogen activator receptor (uPAR), plasminogen, and MMPs to initiate cellular motility, invasiveness and a further cascade of tumor growth factors. It is reported that cathepsins, kallikreins, uPA, uPAR, caspase and MMPs are recognized as key proteases linked to cancer progression.

A protease substrate contains a recognition sequence for cancer type specific proteases. In certain cancers, particular proteases containing specific recognition peptide sequences have been identified. For example, MMP-1, MMP-8, MMP-13 (collagenase), and MMP-14 are overexpressed specifically in breast cancer; MMP-2 and MMP-9 (gelatinase) are overexpressed in colorectal, lung, gliomas, and ovarian cancer and the substrate recognition peptide sequence including e.g., Gly-Pro-Leu-Gly-Ile-Ala-Gly-Gln; prostate membrane specific antigen PSA (hK3) is overexpressed in prostate and ovarian cancers and the substrate recognition sequence including e.g., Arg-Arg-Ser-Ser-Tyr-Tyr-Ser-Gly; and uPA and uPAR are overexpressed in cervical, prostate, gastric and colorectal cancers.

In some embodiments, the particle heaters are constructed as multi-targeting nanoparticles with one or more targeting moieties aimed at the tumor cell-surface markers as well as tumor vascular markers. For example, one targeting unit (e.g., a mAb) will bind to the endothelium on intravenous administration and ensure particle transcytosis, whereas a second one (or sometimes the same mAb) on the same particle surface binds to the recipient tumor cell surface and achieves endosomal internalization of the particle heater. Examples of first targeting units that enable transcytosis are receptor ligands or antibodies that bind to the TfR or receptors for folate, leptin or insulin. The second targeting units will be directed to tumor-specific targets. Examples of tumor cell-surface marker proteins of choice are EGFR and HER-2.

The combination of the active targeting units can enable them to find tumor tissue/cell and molecular tumor markers with increased precision. Cell-surface targeting agents can localize the particle heater exclusively to the intended tissue/tumor cell.

In some embodiments, for any herein described particle heaters, drug delivery particles and particles suitable for antimicrobial treatment may further modified with the tumor-targeting group as described herein.

### Microbe-Targeting Groups

Many investigations have suggested that damage to the bacterial cell membrane (autolysis) is one of the main mechanisms of cell death. Differences between the cell wall ultra-structure of Gram-positive and Gram-negative bacteria play an important role in the susceptibility of bacteria to antibiotics delivered by the particulate carrier. Generally, neutral or anionic antibiotics bind efficiently to Gram-positive bacteria and inactivate them. In contrast, such antibiotics only bind to the outer membrane of Gram-negative bacteria, but do not photo-inactivate them. The high susceptibility of Gram-positive species is attributed to their cell wall structures, since a relatively porous layer of peptidoglycan surrounds their cytoplasmic membrane and lipoteichoic acid that allows organic antibiotics to diffuse to the target sites within the cell. The cell wall of Gram-negative bacteria consists of an inner cytoplasmic membrane and an outer membrane containing lipopolysaccharides (LPS) that are separated by the peptidoglycan-containing periplasm. The negatively charged LPS hinders the permeability of many molecules in the external environment into bacterial cell.

In some embodiments, the particle disclosed herein can be readily engineered to carry out additional functions (e.g. localizing of particles to the microbes). In some embodiments, the particle further comprises a microbial-targeting group on the particle surface. In some embodiments, the particle surface is modified with microbial targeting moieties for active targeting. In some embodiments, a microbial-targeting group is selected from the group of an antibody targeting a bacterial surface antigen; an antibody targeting a bacteria Toll Like Receptor (TLR); a cationic AMP; LPS binding compound; cell penetrating peptides, including apidaecin, tat, buforin, and magainin; and combinations thereof. In some embodiments, the microbial targeting group is a peptide, specifically a cyclic 9-amino acid peptide-CARGGLKSC (CARG). In some embodiments, the microbial targeting group is ubiquicidin (UBI₂₉₋₄₁).

In some embodiments, the microbial-targeting group is a group targeting MSCRAMM (microbial surface components recognizing adhesive matrix molecules), GADPH (surface enzyme), LPXTG domain, Lipid A, β-barrel proteins commonly called outer membrane proteins (OMPs), or combinations thereof.

In some embodiments, the particle surface is covalently conjugated with a positively charged moiety such as poly-lysine, chitosan etc. to localize the particle to the negatively charged bacterial membrane.

In some embodiments, the particle surface is labeled with a macrophage-targeting group selected from a group of dextran, tuftsin, mannose, hyaluronate, and combinations thereof.

In some embodiments, the microbial-targeting group is selected from the group of a ligand targeting pneumococcal surface protein A (PspA), putative proteinase maturation protein A (PpmA), pneumococcal surface adhesin A (PsaA), surface protein G, known as adhesin SasG, staphylococcal protein A (SpA), clumping factor B (ClfB), clumping factor A (clfA), collagen adhesin (CNA), SesL, SesB, SesC, SesK, SesM, Bam A (OMP), adhesin protein (intimin), Hsp90, FimH, OmpA, IROMPS (Iron Regulated Outer Membrane Proteins), M proteins (LPXTG conserved motif in strep), PGK (surface enzyme), TPI (surface enzyme), PGM (surface enzyme), C5a peptidase, SclA (Scl1), GRAB, pullulanase, Esp, Oprl (outer membrane protein I), PilY1, and combinations thereof.

In some embodiments, the microbial-targeting group is selected from the group of a microbial-binding portion of C-type lectins, Col-like lectins, ficolins, receptor-based lectins, lectins from the shrimp Marsupenaeus japonicas, non-C-type lectins, a lipopolysaccharide (LPS)-binding proteins, endotoxin-binding proteins, mannan-binding lectin (MBL), surfactant protein A, surfactant protein D, collectin 11, L-ficolin, ficolin A, DC-SIGN, DC-SIGNR, SIGNR1, macrophage mannose receptor 1, dectin-1, dectin-2, lectin A, lectin B, lectin C, wheat germ agglutinin, CD 14, MD2, lipopolysaccharide-binding protein (LBP), limulus anti-LPS factor (LAL-F), mammalian peptidoglycan recognition protein-1 (PGRP-1), PGRP-2, PGRP-3, PGRP-4, and combinations thereof. In some embodiments, the microbe-targeting group is a LPS binding protein. In some embodiments, the microbe-targeting group is an endotoxin-binding protein.

In some embodiments, AMP is the targeting group. AMP binds to negatively charged bacterial cell membranes via electrostatic interactions, disrupting their function, and resulting in the death of these prokaryotes.

In some embodiments, the microbial targeting group is a cyclic peptide antibiotic vancomycin and/or polymyxin (e.g., polymyxin B, polymyxin E).

In some embodiments, the microbial-targeting group is chemically conjugated to the surface of the particle by EDC-NHS chemistry where the primary amine groups of the targeting antibody/peptide are conjugated to the reactive —COOH groups on the particle surface, such as those from gelatin, collagen, or protein carrier.

In some embodiments, the particle surface is labeled with RGD sequences or a positively charged polymer, such as poly-lysine, chitosan etc., via covalent bonding to target the particle to the negatively charged bacteria membrane.

In some embodiments, the microbial-targeting group is the TAT (YGRKKRRQRRR) peptide that is covalently bound onto the particle surface. The TAT peptide is the shortest amino-acid sequence required for membrane translocation. The TAT peptide was found in the transcriptional activator TAT protein of the human immunodeficiency virus type-1 (HIV-1).

In some embodiments, for any herein described particle heaters, drug delivery particles and particles suitable for antimicrobial treatment may further modified with the microbe-targeting group as described herein.

The use of microbe-targeting group greatly improves the precision of the delivery of particle heaters to the desired infection site.

In some embodiments, the density of display of the targeting group on the particle surface is from about 1 ligand/nm² to about 50 ligands/nm². In some embodiments, the density of display of the targeting group (ligand) on the particle surface is selected from the group of about 1 ligand/nm², 2 ligands/nm², about 3 ligands/nm², about 4 ligands/nm², about 5 ligands/nm², about 6 ligands/nm², about 7 ligands/nm², about 8 ligands/nm², about 9 ligands/nm², about 10 ligands/nm², about 11 ligands/nm², about 12 ligands/nm², about 13 ligands/nm², about 14 ligands/nm², about 15 ligands/nm², about 16 ligands/nm², about 17 ligands/nm², about 18 ligands/nm², about 19 ligands/nm², about 20 ligands/nm², about 21 ligands/nm², about 22 ligands/nm², about 23 ligands/nm², about 24 ligands/nm², about 25 ligands/nm², about 26 ligands/nm², about 27 ligands/nm², about 28 ligands/nm², about 29 ligands/nm², about 30 ligands/nm², about 31 ligands/nm², about 32 ligands/nm², about 33 ligands/nm², about 34 ligands/nm², about 35 ligands/nm², about 36 ligands/nm², about 37 ligands/nm², about 38 ligands/nm², about 39 ligands/nm², about 40 ligands/nm², about 41 ligands/nm², about 42 ligands/nm², about 43 ligands/nm², about 44 ligands/nm², about 45 ligands/nm², about 46 ligands/nm², about 47 ligands/nm², about 48 ligands/nm², about 49 ligands/nm², about 4 ligands/nm², about 50 ligands/nm², about 60 ligands/nm², about 70 ligands/nm², about 80 ligands/nm², about 90 ligands/nm², about 100 ligands/nm², about 110 ligands/nm², about 120 ligands/nm², about 130 ligands/nm², about 140 ligands/nm², about 150 ligands/nm², about 160 ligands/nm², about 170 ligands/nm², about 180 ligands/nm², about 190 ligands/nm², and about 200 ligands/nm² of the particle surface area.

### Optional Additives

In some embodiments, the particle heater further includes thermal stabilizers. It should be noted that often the material that interacts with the exogenous source can be stable (low rate of degradation) at room temperature but when the particle comprising the material is inside body, at body temperature of 37.5° C., degradation of the material can be significantly accelerated. Examples of useful thermal stabilizers include phenolic antioxidants such as butylated hydroxytoluene (BHT), 2-t-butylhydroquinone, and 2-t-butylhydroxyanisole.

In some embodiments, the core of the particle heater may optionally comprise an additive. In some embodiments, the additive is an antioxidant, or a surfactant.

In some embodiments, the additive is an antioxidant. In some embodiments, the antioxidant is selected from the group of NADPH, uric acid, Vitamin A, Vitamin C (ascorbic acid), Vitamin E (tocopherol acetate), glutathione, beta-carotene and polyphenols, superoxide dismutase, glutathione oxidoreductase, thioredoxin disulfide reductase, and combinations thereof.

In some embodiments, the particles/compositions/medium may include inhibitors of enzymatic antioxidants such as superoxide dismutase (SOD), catalase (CAT), glutathione peroxidase (GPx) and thioredoxin (Trx). These inhibitors include but are not limited by: LCS-1 (4,5-dichloro-2-m-tolylpyridazin-3(2H)-one, salicylic acid, 6-Amino-5-nitroso-3-methyluracil, ATN-224 (bis-choline tetrathiomolybdate); 2-ME (2-methoxyoestradiol); N—N′-diethyldithiocarbamate, 3-Amino-1,2,4-Triazole, p-Hydroxybenzoic acid, misonidazole, d-penicillamine hydrochloride, 1-penicillamine hydantoin, dl-Buthionine-[S, R]-sulfoximine (BSO), and Au(I) thioglucose etc.

In some embodiments, the additive is an antioxidant for stabilizing the IR absorbing agents at human body temperature. In some embodiments, the antioxidants for stabilizing IR absorbing agents comprise sterically hindered phenols with para-propionate groups. In some embodiments, the antioxidant for stabilizing IR absorbing agents comprises pentaerythritol tetrakis(3,5-di-tert-butyl-4-hydroxyhydrocinnamate). In some embodiments, the antioxidant for stabilizing IR absorbing agents comprises a phosphite such as tris(2,4-di-tert-butylphenyl)phosphite. In some embodiments, the antioxidant for stabilizing IR absorbing agents comprises organosulfur compounds such as thioethers. In some embodiments, the antioxidant for stabilizing IR absorbing agents comprises 1,3,5-TRIS(4-tert-butyl-3-hydroxy-2,6-dimethylbenzyl)-1,3,5-triazine-2,4,6-(1H,3H,5H)-trione (Cyanox® 1790); wherein the Cyanox® 1790 is colorless.

In some embodiments, the additive is a surfactant. In some embodiments, the surfactant may include cationic, amphoteric, and non-ionic surfactants. In some embodiments, the surfactants comprise anionic surfactants selected from the group of fatty acid salts, bile salts, phospholipids, carnitines, ether carboxylates, succinylated monoglycerides, mono/diacetylated tartaric acid esters of mono- and diglycerides, citric acid esters of mono- and diglycerides, sodium oleate, sodium lauryl sulfate, sodium lauryl sarcosinate, sodium dioctyl sulfosuccinate (SDS), sodium cholate, sodium taurocholate, lauroyl carnitine, palmitoyl carnitine, myristoyl carnitine, lactylic esters of fatty acids, and combinations thereof. In some embodiments, anionic surfactants include di-(2-ethylhexyl) sodium sulfosuccinate. In some embodiments, the surfactants are non-ionic surfactants selected from the group of propylene glycol fatty acid esters, mixtures of propylene glycol fatty acid esters and glycerol fatty acid esters, triglycerides, sterol and sterol derivatives, sorbitan fatty acid esters and polyethylene glycol sorbitan fatty acid esters, sugar esters, polyethylene glycol alkyl ethers and polyethylene glycol alkyl phenol ethers, polyoxyethylene-polyoxypropylene block copolymers, lower alcohol fatty acid esters, and combinations thereof. In some embodiments, the surfactant may comprise fatty acids. Examples of fatty acids include caprylic acid, undecylic acid, lauric acid, tridecylic acid, myristic acid, palmitic acid, stearic acid, or oleic acid. In some embodiments, the surfactants comprise amphoteric surfactants including (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants.

In some embodiments, the surfactant comprises a hydrophilic amphiphilic surfactant polyoxyethylene (20) sorbitan monolaurate (TWEEN® 20) or polyvinyl alcohol that improves the distribution of the material in the polymeric carrier. In some embodiments, the surfactant comprises an amphiphilic surfactant if the IR absorbing agent is hydrophilic and the polymeric carrier is hydrophobic. In some embodiments, the surfactant is an anionic surfactant sodium bis(tridecyl) sulfosuccinate (Aerosol® TR-70). In some embodiments, the surfactant is sodium bis(tridecyl) sulfosuccinate, or sodium dodecyl sulfate (SDS).

In some embodiments, the use amount of the additive may be about 0.01 wt. % to about 10.0 wt. % by the total weight of the particle. In some embodiments, the use amount of the additive may be about 0.1 wt. % to about 10.0 wt. % by the total weight of the particle. In some embodiments, the use amount of the additive may be about 0.5 wt. % to about 10.0 wt. % by the total weight of the particle. In some embodiments, the use amount of the additive may be about 1.0 wt. % to about 10.0 wt. % by the total weight of the particle. In some embodiments, the use amount of the additive may be about 1.0 wt. % to about 9.0 wt. % by the total weight of the particle. In some embodiments, the use amount of the additive may be about 1.0 wt. % to about 8.0 wt. % by the total weight of the particle. In some embodiments, the use amount of the additive may be about 1.0 wt. % to about 7.0 wt. % by the total weight of the particle. In some embodiments, the use amount of the additive may be about 1.0 wt. % to about 6.0 wt. % by the total weight of the particle. In some embodiments, the use amount of the additive may be about 1.0 wt. % to about 5.0 wt. % by the total weight of the particle. In some embodiments, the use amount of the additive may be about 1.0 wt. % to about 4.0 wt. % by the total weight of the particle. In some embodiments, the use amount of the additive may be about 1.0 wt. % to about 3.0 wt. % by the total weight of the particle. In some embodiments, the use amount of the additive may be about 1.0 wt. % to about 2.5 wt. % by the total weight of the particle. In some embodiments, the use amount of the additive may be about 1.0 wt. % to about 2.0 wt. % by the total weight of the particle. In some embodiments, the use amount of the additive may be about 2.0 wt. % to about 10.0 wt. % by the total weight of the particle. In some embodiments, the use amount of the additive may be about 3.0 wt. % to about 10.0 wt. % by the total weight of the particle. In some embodiments, the use amount of the additive may be about 4.0 wt. % to about 10.0 wt. % by the total weight of the particle. In some embodiments, the use amount of the additive may be about 5.0 wt. % to about 10.0 wt. % by the total weight of the particle. In some embodiments, the use amount of the additive may be selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.1 wt. %, about 1.2 wt. %, about 1.3 wt. %, about 1.4 wt. %, about 1.5 wt. %, about 1.6 wt. %, about 1.7 wt. %, about 1.8 wt. %, about 1.9 wt. %, about 2.0 wt. %, about 2.25 wt. %, about 2.5 wt. %, about 2.75 wt. %, about 3.0 wt. %, about 3.25 wt. %, about 3.50 wt. %, about 3.75 wt. %, about 4.00 wt. %, about 4.25 wt. %, about 4.50 wt. %, about 4.75 wt. %, about 5.00 wt. %, about 5.25 wt. %, about 5.50 wt. %, about 5.75 wt. %, about 6.00 wt. %, about 6.25 wt. %, about 6.50 wt. %, about 6.75 wt. %, about 7.00 wt. %, about 7.25 wt. %, about 7.50 wt. %, about 7.75 wt. %, about 8.00 wt. %, about 8.25 wt. %, about 8.50 wt. %, about 8.75 wt. %, about 9.00 wt. %, about 9.25 wt. %, about 9.50 wt. %, about 9.75 wt. %, about 10.0 wt. %, about 10.25 wt. %, about 10.50 wt. %, about 10.75 wt. %, or about 11.00 wt. %.

In some embodiments, the particle comprises the carrier to the payload (e.g., active agent) in a weight ratio ranging from 1:10 to 10:1. In some embodiments, the weight ratio of the carrier to the payload ranges from 1:1 to 7:1. In some embodiments, the weight ratio of the carrier to the payload ranges from 2:1 to 7:1. In some embodiments, the weight ratio of the carrier to the payload ranges from 3:1 to 7:1. In some embodiments, the weight ratio of the carrier to the payload ranges from 4:1 to 7:1. In some embodiments, the weight ratio of the carrier to the payload ranges from 5:1 to 7:1. In some embodiments, the weight ratio of the carrier to the payload ranges from 6:1 to 7:1. In some embodiments, the weight ratio of the carrier to the payload ranges from 1:7 to 7:1. In some embodiments, the weight ratio of the carrier to the payload ranges from 1:5 to 5:1. In some embodiments, the weight ratio of the carrier to the payload ranges from 1:3 to 3:1. In some embodiments, the weight ratio of the carrier to the payload is a range selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1. In some embodiments, the weight ratio of the carrier to the payload is a range selected from the group of 1:1, 2:1, 3:1, 5:1, or 7:1. In some embodiments, the weight ratio of the carrier to the payload is 2:1. In some embodiments, the weight ratio of the carrier to the payload is 3:1. In some embodiments, the weight ratio of the carrier to the payload is 5:1. In some embodiments, the weight ratio of the carrier to the payload is 7:1.

### 2. Particle Properties

### 2(a) Particle Size and Particle Morphology

In vivo particle-based active agent delivery is fraught with a host of biophysical and biochemical challenges that can cause particle uptake (opsonization), excretion (kidneys) or non-specific loss (extravasation) and prevent the therapeutic payload from reaching the desired cells. One of the key parameters of a particle delivery construct is its physical size, where smaller particles (e.g., particles less than or equal to about 5 nm hydrodynamic diameter) can extravasate non-specifically, while much larger particles or aggregates (e.g., particles or aggregates greater than or equal to about 500 nm diameter) can become lodged in the microvasculature, rather than being trafficked to their intended targets.

For non-biodegradable materials, it is found that there is a preferable diameter range from 5 nm to 10 nm enabling renal filtration as a means of particle removal, while limiting the rate of renal clearance to enable the desired pharmacokinetics. Additionally, it was found that particles of this size range could also take advantage of the enhanced permeability and retention (EPR) effect that is, the passive accumulation of macromolecules in tumor microenvironments due to the leaky vasculature and impaired lymphatic drainage.

In some embodiments, the particles may be nanoparticles. In some embodiments, the particles may have spherical shape. In some embodiments, the particles may have a wide variety of non-spherical shapes. In some embodiments, the non-spherical particles may be in the shape of rectangular disks, high aspect ratio rectangular disks, rods, high aspect ratio rods, worms, oblate ellipses, prolate ellipses, elliptical disks, UFOs, circular disks, barrels, bullets, pills, pulleys, bi-convex lenses, ribbons, ravioli, flat pill, bicones, diamond disks, emarginated disks, elongated hexagonal disks, tacos, wrinkled prolate ellipsoids, wrinkled oblate ellipsoids, or porous elliptical disks. Additional shapes beyond those are also within the scope of the definition for “non-spherical” shapes.

In some embodiments, the particles have a PdI from about 0.05 to about 0.15, about 0.06 to about 0.14, about 0.07 to about 0.13, about 0.08 to about 0.12, or about 0.09 to about 0.11. In some embodiments, the particles have a PdI of about 0.05, about 0.06, about 0.07, about 0.08, about 0.09, about 0.10, about 0.11, about 0.12, about 0.13, about 0.14, or about 0.15.

In some embodiments, the particle has a median particle size less than 1000 nm. In some embodiments, the median particle size ranges from about 1 nm to about 1000 nm. In some embodiments, the median particle size ranges from about 1 nm to about 500 nm. In some embodiments, the median particle size ranges from about 1 nm to about 250 nm. In some embodiments, the median particle size ranges from about 1 nm to about 150 nm. In some embodiments, the median particle size ranges from about 1 nm to about 100 nm. In some embodiments, the median particle size ranges from about 1 nm to about 50 nm. In some embodiments, the median particle size ranges from about 1 nm to about 25 nm. In some embodiments, the median particle size ranges from about 1 nm to about 10 nm. In some embodiments, the particle has a median particle size selected from the group of about 1 nm, about 5 nm, about 10 nm, about 15 nm, about 20 nm, about 25 nm, about 30 nm, about 35 nm, about 40 nm, about 45 nm, about 50 nm, about 55 nm, about 60 nm, about 65 nm, about 70 nm, about 75 nm, about 80 nm, about 85 nm, about 90 nm, about 95 nm, about 100 nm, about 105 nm, about 110 nm, about 115 nm, about 120 nm, about 125 nm, about 130 nm, about 135 nm, about 140 nm, about 145 nm, about 150 nm, about 155 nm, about 160 nm, about 165 nm, about 170 nm, about 175 nm, about 180 nm, about 185 nm, about 190 nm, about 195 nm, about 200 nm, about 205 nm, about 210 nm, about 215 nm, about 220 nm, about 225 nm, about 230 nm, about 235 nm, about 240 nm, about 245 nm, about 250 nm, about 255 nm, about 260 nm, about 265 nm, about 270 nm, about 275 nm, about 280 nm, about 285 nm, about 290 nm, about 295 nm, about 300 nm, about 310 nm, about 320 nm, about 330 nm, about 340 nm, about 350 nm, about 360 nm, about 370 nm, about 380 nm, about 390 nm, about 400 nm, about 410 nm, about 420 nm, about 430 nm, about 440 nm, about 450 nm, about 460 nm, about 470 nm, about 480 nm, about 490 nm, about 500 nm, about 525 nm, about 550 nm, about 575 nm, about 600 nm, about 625 nm, about 650 nm, about 675 nm, about 700 nm, about 725 nm, about 750 nm, about 775 nm, about 800 nm, about 825 nm, about 850 nm, about 875 nm, about 900 nm, about 925 nm, about 950 nm, about 975 nm, and about 1000 nm. In some embodiments, the particle has a median particle size of 500 nm. In some embodiments, the particle has a median particle size of 250 nm. In some embodiments, the particle has a median particle size of 750 nm. In some embodiments, the particle has a median particle size of about 250 nm. In some embodiments, the particle has a median particle size of about 150 nm. In some embodiments, the particle has a median particle size of about 125 nm. In some embodiments, the particle has a median particle size of about 100 nm. In some embodiments, the particle has a median particle size of about 75 nm. In some embodiments, the particle has a median particle size of 50 nm. In some embodiments, the particle has a median particle size ranges from about 1 nm to about 50 nm.

In some embodiments, the particles are microparticles having a median particle size equal or greater than 1000 nm (1 micron). In some embodiments, the particles have a median particle size selected from the group of about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 25 μm, about 30 μm, about 35 μm, about 40 μm, about 45 μm, about 50 μm, about 55 μm, about 60 μm, about 65 μm, about 70 μm, about 75 μm, about 80 μm, about 85 μm, about 90 μm, about 95 μm, about 100 μm, about 105 μm, about 110 μm, about 115 μm, about 120 μm, about 125 μm, about 130 μm, about 140 μm, about 145 μm, about 150 μm, about 155 μm, about 160 μm, about 165 μm, about 170 μm, about 175 μm, about 180 μm, about 185 μm, about 190 μm, about 195 μm, about 200 μm, about 205 μm, about 210 μm, about 215 μm, about 220 μm, about 225 μm, about 230 μm, about 235 μm, about 240 μm, about 245 μm, about 250 μm, about 255 μm, about 260 μm, about 265 μm, about 270 μm, about 275 μm, about 280 μm, about 285 μm, about 290 μm, about 295 μm, about 300 μm, about 310 μm, about 320 μm, about 330 μm, about 340 μm, about 350 μm, about 360 μm, about 370 μm, about 380 μm, about 390 μm, about 400 μm, about 410 μm, about 420 μm, about 430 μm, about 440 μm, about 450 μm, about 460 μm, about 470 μm, about 480 μm, about 490 μm, and about 500 μm. In some embodiments, the particle has a median particle size in a range from about 1 μm to about 500 μm. In some embodiments, the particle has a median particle size in a range from about 1 μm to about 250 μm. In some embodiments, the particle has a median particle size in a range from about 1 μm to about 100 μm. In some embodiments, the particle has a median particle size in the range from about 1 μm to about 50 μm. In some embodiments, the particle has a median particle size in a range from about 1 μm to about 25 μm. In some embodiments, the particle has a median particle size in a range from about 1 μm to about 10 μm. In some embodiments, the particle has a median particle size in a range from about 1 μm to about 6 μm. In some embodiments, the particle has a median particle size in a range from about 1 μm to about 5 μm. In some embodiments, the particle has a median particle size in a range from about 1 μm to about 3 μm. In some embodiments, the particle has a median particle size in a range from about 1 μm to about 2 μm. In some embodiments, the particle has a median particle size in a range from about 2 μm to about 5 μm. In some embodiments, the particle has a median particle size in a range from about 2 μm to about 4 μm. In some embodiments, the particle has a median particle size in a range from about 2 μm to about 3 μm. In some embodiments, the particle has a median particle size in a range from about 3 μm to about 5 μm. In some embodiments, the particle has a median particle size in a range from about 3 μm to about 4 μm. In some embodiments, the particle has a median particle size in a range from about 4 μm to about 5 μm. In some embodiments, the particle has a median particle size from about 1 μm, about 2 μm, about 3 μm, about 4 μm, about 5 μm, or about 6 μm. In some embodiments, the particle has a median particle size in the range from about 1 μm to about 2 μm. In some embodiments, the particle has a median particle size in the range from about 1 μm to about 3 μm. In some embodiments, the particle has a median particle size in the range from about 1 μm to about 4 μm.

### 2(b) Particle Surface Modification

In some embodiments, the particle surface further comprises a hydrophilic polymer that promotes prolonged blood circulation (known as “stealth”). Examples of the hydrophilic polymer include, but are not limited to, polyethylene glycol (PEG); PEG containing block copolymer; polyalkylene oxide, including polypropylene oxide, polybutylene oxide; block copolymer of PEG and polypropylene oxide; polyoxyethylene-polyoxypropylene block copolymer (Pluronic® F-68, F-127), polyxamer (polyethylene oxide block copolymer); hyperbranched polyglycerol; hyaluronic acid; or combinations thereof.

The presence of the hydrophilic polymer on the particle surface can affect the zeta-potential of the particle. In one embodiment, the zeta potential of the particle is from about −60 mV to about 60 mV, from about −50 mV to about 50 mV, from about −30 mV to about 30 mV, from about −25 mV to about 25 mV, from about −20 mV to about 20 mV, from about −10 mV to about 10 mV, from about −10 mV to 5 mV, from about −5 mV to about 5 mV, or from about −2 mV to about 2 mV. In some embodiments, the zeta potential of the particle is in a range selected from the group of about −10 mV to about 10 mV, from about −5 mV to about 5 mV, and from about −2 mV to about 2 mV. In some embodiments, the particle surface charge is neutral or near-neutral (i.e., zeta potential is from about −10 mV to about 10 mV).

In some embodiments, the hydrophilic polymer is a polyethylene glycol. In some embodiments, the hydrophilic polymer on the particle surface is polyethylene glycol having a number average molecular weight ranging from about 300 Da to about 100,000 Da. In some embodiments, the polyethylene glycol has a number average molecular weight selected from the group of 300 Da, 600 Da, 1 kDa, 2 kDa, 3 kDa, 4 kDa, 6 kDa, 8 kDa, 10 kDa, 15 kDa, 20 kDa, 30 kDa, 50 kDa, 100 kDa, 200 kDa, and 500 kDa. Polyethylene glycol of any given molecular weight may vary in other characteristics such as length, density, and branching. In some embodiments, the particle surface modifier is a PEG having a number average molecular weight ranging from 2000 Da to 80,000 Da. In some embodiments, the particle surface modifier is a PEG having a number average molecular weight ranging from 2000 Da to 70,000 Da. In some embodiments, the particle surface modifier is a PEG having a number average molecular weight ranging from 2000 Da to 60,000 Da. In some embodiments, the particle surface modifier is a PEG having a number average molecular weight ranging from 2000 Da to 50,000 Da. In some embodiments, the particle surface modifier is a PEG having a number average molecular weight ranging from 2000 Da to 40,000 Da. In some embodiments, the particle surface modifier is a PEG having a number average molecular weight ranging from 2000 Da to 30,000 Da. In some embodiments, the particle surface modifier is a PEG having a number average molecular weight ranging from 2000 Da to 20,000 Da. In some embodiments, the particle surface modifier is a PEG having a number average molecular weight ranging from 2000 Da to 10,000 Da. In some embodiments, the particle surface modifier is a PEG having a number average molecular weight ranging from 2000 Da to 9,000 Da. In some embodiments, the particle surface modifier is a PEG having a number average molecular weight ranging from 2000 Da to 8,000 Da. In some embodiments, the particle surface modifier is a PEG having a number average molecular weight ranging from 5000 Da to 10,000 Da. In some embodiments, the particle surface modifier is a PEG having a number average molecular weight ranging from 7000 Da to 10,000 Da.

In some embodiments, the particle surface modifier is a PEG having a number average molecular weight selected from the group of 2000 Da, 3000 Da, 4000 Da, 5000 Da, 6000 Da, 7000 Da, 8000 Da, 9000 Da, 10,000 Da, 11,000 Da, 12,000 Da, 13,000 Da, 14,000 Da, 15,000 Da, 16,000 Da, 17,000 Da, 18,000 Da, 19,000 Da, 20,000 Da, 21,000 Da, 22,000 Da, 23,000 Da, 24,000 Da, 25,000 Da, 26,000 Da, 27,000 Da, 28,000 Da, 29,000 Da, 30,000 Da, 31,000 Da, 32,000 Da, 33,000 Da, 34,000 Da, 35,000 Da, 36,000 Da, 37,000 Da, 38,000 Da, 39,000 Da, 40,000 Da, 41,000 Da, 42,000 Da, 43,000 Da, 44,000 Da, 45,000 Da, 46,000 Da, 47,000 Da, 48,000 Da, 49,000 Da, 50,000 Da, 51,000 Da, 52,000 Da, 53,000 Da, 54,000 Da, 55,000 Da, 56,000 Da, 57,000 Da, 58,000 Da, 59,000 Da, 60,000 Da, 61,000 Da, 62,000 Da, 63,000 Da, 64,000 Da, 65,000 Da, 66,000 Da, 67,000 Da, 68,000 Da, 69,000 Da, 70,000 Da, 71,000 Da, 72,000 Da, 73,000 Da, 74,000 Da, 75,000 Da, 76,000 Da, 77,000 Da, 78,000 Da, 79,000 Da, 80,000 Da, 81,000 Da, 82,000 Da, 83,000 Da, 84,000 Da, 85,000 Da, 86,000 Da, 87,000 Da, 88,000 Da, 89,000 Da, 90,000 Da, 91,000 Da, 92,000 Da, 93,000 Da, 94,000 Da, 95,000 Da, 96,000 Da, 97,000 Da, 98,000 Da, 99,000 Da, and 100,000 Da.

In some embodiments, the amount of the hydrophilic polymer attached to the particle surface is expressed as a percentage by the total weight of the uncoated particle. In some embodiments, the weight ratio of the hydrophilic polymer to the uncoated particle is at least 1/10,000, 1/7500, 1/5000, 1/4000, 1/3400, 1/2500, 1/2000, 1/1500, 1/1000, 1/750, 1/500, 1/250, 1/200, 1/150, 1/100, 1/75, 1/50, 1/25, 1/20, 1/5, 1/2, or 9/10 by the weight of the uncoated particle. In some embodiments, the weight ratio of the hydrophilic polymer to the uncoated particle is in a range from 1/10,000 to 9/10 by the weight of the uncoated particle. In some embodiments, the hydrophilic polymer on the particle surface has a weight percent by the weight of the uncoated particle is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100%. In some embodiments, the hydrophilic polymer covers at least 90% of the particle surface area. In some embodiments, the hydrophilic polymer covers about 100% of the particle surface area. In some embodiments, the hydrophilic polymer covers at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% of the particle surface area.

### (c) Cytotoxicity and Porosity, Active Agent Stability

In some embodiments, the particle has a substantially low leakage of active agent such that the particle has low cytotoxicity. In some embodiments, the substantial low leakage of active agent refers to an active agent leakage being less than about 20.0%. In some embodiments, the leakage of active agent is less than about 15.0%. In some embodiments, the leakage of active agent is less than about 10.0%. In some embodiments, the leakage of active agent is less than about 5.0%. In some embodiments, the leakage of active agent is less than about 4.0%. In some embodiments, the leakage of active agent is less than about 3.0%. In some embodiments, the leakage of active agent is less than about 2.0%. In some embodiments, the leakage of the active agent is less than about 1.0%. In some embodiments, the leakage of active agent is less than about 0.1%. In some embodiments, the leakage of active agent is less than about 0.01%. In some embodiments, the leakage of the active agent is 0%. In some embodiments, the leakage of the active agent is less than a percentage value selected from the group of: about 0.01%, 0.1%, 0.5%, 1.0%, 1.5%, 2.0%, 2.5%, 3.0%, 3.5%, 4.0%, 4.5%, 5.0%, 5.5%, 6.0%, 6.5%, 7.0, 7.5%, 8.0%, 8.5%, 9.0%, 9.5%, 10.0%, 10.5%, 11.0%, 11.5%, 12.0%, 12.5%, 13.0%, 13.5%, 14.0%, 14.5%, 15.0%, 15.5%, 16.0%, 16.5%, 17.0%, 17.5%, 18.0%, 18.5%, 19.0%, 19.5%, 20.0%, 20.5%, 21.0%, 21.5%, 22.0%, 22.5%, 23.0%, 23.5%, 24.0%, 24.5%, or 25.0%. In some embodiments, the leakage of the active agent ranging from about 0.01% to about 5.0%. In some embodiments, the leakage of the active agent ranging from about 0.01% to about 4.0%. In some embodiments, the leakage of the active agent ranging from about 0.01% to about 3.0%. In some embodiments, the leakage of the active agent ranging from about 0.01% to about 2.0%. In some embodiments, the leakage of the active agent ranging from about 0.01% to about 1.0%. In some embodiments, the leakage of the active agent ranging from about 0.01% to about 0.1%. In some embodiments, the leakage of the active agent ranging from about 0.1% to about 5.0%. In some embodiments, the leakage of the active agent ranging from about 0.1% to about 4.0%. In some embodiments, the leakage of the active agent ranging from about 0.1% to about 3.0%. In some embodiments, the leakage of the active agent ranging from about 0.1% to about 2.0%. In some embodiments, the leakage of the active agent ranging from about 0.1% to about 1.0%.

### 3. Remotely-Triggered Thermal Therapy

Photothermal therapy (PTT), a minimally invasive therapeutic strategy in which photon energy is converted into heat sufficient to destroy unwanted cells. Heating sources including near infrared or visible light, radiofrequency waves, microwaves, and ultrasound waves are used to induce moderate temperature rise in a specific target region to destroy the unwanted cells, clinically termed as hyperthermia. Synthetic organic IR absorbing agent molecules, such as indocyanine green, pthalocyanines, naphthalocyanines and porphyrins coordinated with transition metals, are externally administrated into the tissue sites to enhance the photothermal effects.

Molecules and materials that can absorb energy from an exogenous source to generate heat for controlled and localized temperature increments are potentially valuable for numerous applications in remotely triggered thermal therapies. Unlike conventional chemotherapies, there are no known resistance mechanisms to thermal therapies. Remotely triggered thermal therapies are therefore highly attractive for inducing toxicity in drug-resistant cells.

One of the challenges associated with the applications of the photothermal materials in PTT is the non-uniform and inefficient heating during and after the irradiation of the photo-absorbing chromophores such like indocyanine green, vital blue, and carbon black with an exogenous light source supplied in situ due to the poor penetration of the radiation through the tissue. Additionally, production of sufficient and uniform heat using this technique remains a challenge. Some of these chromophores may cause toxicity to the body. Furthermore, the chromophores may be degraded by the body into unwanted chemicals that are toxic to the body. Degradation of the chromophores by the body may also lead to insufficient heating at the site of action and thereby increase the dose required for effective heating which can compound toxicity to the body. Thermal cytotoxicity due to the heat generated following the irradiation of the photothermal materials can also be a problem that has not be adequately addressed in the prior art on photothermal materials.

Remotely triggering a molecule to generate localized heat for selective killing of certain unwanted cells (e.g., tumor cells or microbes) is of great interest in the medical field. Light has been extensively explored as a remote trigger to generate localized hyperthermia for achieving cell killing and is referred to as Photothermal Therapy (PTT). PTT employing near-infrared light absorbing particles to generate heat from optical energy to kill cancer cells has gained great attention in recent years. Most photothermal conversion agents are based on various gold (Au) nanostructures. Despite the overwhelming potential of particle-mediated photothermal therapy to improve cancer treatment, the only particle-mediated PTT that has advanced to clinical trial is Aurolase™ therapy, consisting of Au nanoshells of 150 nm diameter with a silica core. The therapeutic potential of Aurolase™ therapy is limited by its low photothermal conversion efficiency, potential long-term toxicity due to non-biodegradability, and lack of photostability due to melting of the Au nanostructure by the heat generated from laser irradiation. Organic molecules are also being investigated for PTT applications. But these small molecules are rapidly cleared from the body and can cause unwanted toxicity to the body. Particles encapsulating these small molecule IR absorbing agents are being researched for PTT applications, but these particles are leaky enough for the IR absorbing agents to be released prior to laser irradiation increasing cytotoxicity and reducing their efficacy. Body chemicals can also penetrate these particles and degrade the organic IR absorbing agents, again reducing the efficacy of photothermal killing. Often the photothermal effects can also lead to thermal cytotoxicity. There is therefore a need for particles that are designed to improve the remotely triggered, thermal killing of unwanted cells while limiting collateral, chemical, and thermal toxicities to neighboring cells and tissues.

### 3(a). Photothermal Cancer Therapy

Hyperthermia is a type of cancer treatment in which body tissue is exposed to high temperatures (up to 113° F., 45° C.). Research has shown that high temperatures can damage and kill cancer cells, usually with minimal injury to normal tissues (van der Zee J. Heating the patient: a promising approach? Annals of Oncology 2002; 13(8):1173-1184). By killing cancer cells and damaging proteins and structures within cells (Hildebrandt et al. The cellular and molecular basis of hyperthermia. Critical Reviews in Oncology/Hematology 2002; 43(1):33-56), hyperthermia may shrink tumors.

Hyperthermia may be used with other forms of cancer therapy, such as radiation therapy and chemotherapy (Wust et al. Hyperthermia in combined treatment of cancer. The Lancet Oncology 2002; 3(8):487-497). Hyperthermia may make some cancer cells more sensitive to radiation or harm other cancer cells that radiation cannot damage. When hyperthermia and radiation therapy are combined, they are often given within an hour of each other. Hyperthermia can also enhance the effects of certain anticancer drugs.

Conventional chemotherapies for cancer treatment have their inherent drawbacks due to severe toxic side effects to the body. Using relatively non-toxic agents that can be triggered exogenously only in the tumor tissue to cause cancer cell death is a very attractive way to treat cancers with reduced collateral damage to the body. Light-triggered therapies like photodynamic therapy (PDT) and photothermal therapy (PTT) have been explored for cancer treatment. PDT involves the generation of reactive molecular species like singlet oxygen to localize the destruction of cells. PDT is approved for treating cancers.

Many inorganic photothermal agents, e.g., gold, silver, platinum and transitional metal sulfide or oxide nanoparticles, have been used for PTT. These inorganic photothermal agents achieve high therapeutic efficacy in many preclinical animal models, however, the clinical application is significantly limited due to their non-biodegradability and potential long-term toxicities.

Organic molecules can also be used as PTT agents but usually suffer from poor bioavailability and non-specific toxicity. Encapsulation of organic PTT agents into particles has been explored and these particles can overcome some of these shortcomings of the small organic molecules. Indocyanine green (ICG) is a clinically used diagnostic contrast agent that can also produce heat following laser irradiation. The use of particles encapsulating ICG for PTT has been explored for cancer, but these particles tend to be leaky, thus reducing the PTT efficacy, and causing unwanted cytotoxicity. Moreover, a large amount of ICG is needed for the desired efficacy because of body chemicals breaking down the ICG in the leaky particles. Further, the clinical application of the ICG based particle heater is also limited due to their lack of targeting abilities.

Therefore, there exists a need for a clinically effective thermotherapy with low toxicity and low collateral damage to non-cancer cells. The present invention provides a particle heater meeting such needs with high energy-to-heat conversion efficiency, improved biocompatibility, and lowered cytotoxicity.

In an embodiment, this disclosure provides a particle heater for use in the remotely-triggered thermotherapy of a cancer comprising: the material described herein admixed with the carrier described herein, wherein the material in the particle heater exhibits stability such that the particle is considered passing the Efficacy Determination Protocol; wherein the particle is constructed such that it passes the Extractable Cytotoxicity Test; wherein the particle and specific dose(s) of the exogenous source pass the Thermal Cytotoxicity Test; wherein the material absorbs the energy from the exogenous source and converts the energy into heat; and then the heat travels outside the particle to induce localized hyperthermia sufficient to selectively kill the cancer cells.

In some embodiments, the material exhibits at least 20% efficiency of conversion of the energy from the exogenous source to heat. In some embodiments, the material exhibits at least 20% photothermal conversion efficiency.

In some embodiments, at least a portion of the exterior surface of the particle has a modification that is polar, non-polar, charged, ionic, basic, acidic, reactive, hydrophobic, or hydrophilic.

In some embodiments, the particle further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof.

In some embodiments, the shell results from the use of an alkyltrimethoxysilane reagent (CnTMS, n is an integer ranging from 1 to 12) in the Stöber synthesis. In some embodiments, the shell results from the use of C1-C7 alkyl trimethoxysilane reagent in the Stöber synthesis. In some embodiments, the shell results from the use of C1-C7 alkenyl trimethoxysilane reagent in the Stöber synthesis. In some embodiments, the shell results from the use of C1-C7 alkynyl trimethoxysilane reagent in the Stöber synthesis. In some embodiments, the C1-C7 alkyl group, the C1-C7 alkenyl group, or the C1-C7 alkynyl group may be linear or branched. In some embodiments, the shell results from the use of C2-C6 linear alkyl trimethoxysilane reagent in the Stöber synthesis. In some embodiments, the shell results from the use of C2-C4 linear alkyl trimethoxysilane reagent in the Stöber synthesis. In some embodiments, the shell results from the use of ethyl (C2) trimethoxysilane reagent in Stöber synthesis. In some embodiments, the shell results from the use of vinyltrimethoxysilane (VTMS) reagent in Stöber synthesis. In some embodiments, the shell results from the condensation reaction of hydroxymethylsilanetriol prepared by the hydrolysis of hydroxymethyltrichlorosilane. In some embodiments, the shell results from the condensation reaction of (3-mercaptopropyl)silanetriol prepared by the hydrolysis of (3-mercaptopropyl)trimethoxysilane. The silicate shell having hydroxymethyl and 3-mercaptopropyl modification on the surface provides reactive functional group for further engineering of the particle with targeting groups and other functional surface modifications.

In some embodiments, the shell layer is present in an amount of greater than 10.0 wt. % of the total weight of the uncoated particles. In some embodiments, the shell layer is present in an amount of greater than 20.0 wt. % of the total weight of the uncoated particles. In some embodiments, the shell layer is present in an amount of greater than 30.0 wt. % of the total weight of the uncoated particles. In some embodiments, the shell layer is present in an amount of greater than 40.0 wt. % of the total weight of the uncoated particles. In some embodiments, the shell layer is present in an amount of greater than 50.0 wt. % of the total weight of the uncoated particles. In some embodiments, the shell layer is present in an amount of greater than 60.0 wt. % of the total weight of the uncoated particles.

In some embodiments, the amount of shell is at a weight percentage by the total weight of the shell and the uncoated particle ranging from about 5 wt. % to about 40 wt. %. In some embodiments, the amount of shell is at a weight percentage by the total weight of the shell and the uncoated particle ranging from about 6 wt. % to about 40 wt. %. In some embodiments, the amount of shell is at a weight percentage by the total weight of the shell and the uncoated particle ranging from about 7 wt. % to about 40 wt. %. In some embodiments, the amount of shell is at a weight percentage by the total weight of the shell and the uncoated particle ranging from about 8 wt. % to about 40 wt. %. In some embodiments, the amount of shell is at a weight percentage by the total weight of the shell and the uncoated particle ranging from about 9 wt. % to about 40 wt. %. In some embodiments, the amount of shell is at a weight percentage by the total weight of the shell and the uncoated particle ranging from about 10 wt. % to about 40 wt. %. In some embodiments, the amount of shell is at a weight percentage by the total weight of the shell and the uncoated particle ranging from about 15 wt. % to about 40 wt. %. In some embodiments, the amount of shell is at a weight percentage by the total weight of the shell and the uncoated particle ranging from about 25 wt. % to about 40 wt. %. In some embodiments, the amount of shell is at a weight percentage by the total weight of the shell and the uncoated particle ranging from about 30 wt. % to about 40 wt. %. In some embodiments, the amount of shell is at a weight percentage by the total weight of the shell and the uncoated particle ranging from about 35 wt. % to about 40 wt. %. In some embodiments, the amount of shell is at a weight percentage by the total weight of the shell and the uncoated particle ranging from about 12.5 wt. % to about 40 wt. %. In some embodiments, the amount of shell is at a weight percentage by the total weight of the shell and the uncoated particle ranging from about 15 wt. % to about 40 wt. %. In some embodiments, the amount of shell is at a weight percentage by the total weight of the shell and the uncoated particle ranging from about 17.5 wt. % to about 40 wt. %. In some embodiments, the amount of shell is at a weight percentage by the total weight of the shell and the uncoated particle ranging from about 20 wt. % to about 40 wt. %. In some embodiments, the amount of shell is at a weight percentage by the total weight of the shell and the uncoated particle ranging from about 22.5 wt. % to about 40 wt. %. In some embodiments, the amount of shell is at a weight percentage by the total weight of the shell and the uncoated particle ranging from about 25 wt. % to about 40 wt. %. In some embodiments, the amount of shell is at a weight percentage by the total weight of the shell and the uncoated particle ranging from about 27.5 wt. % to about 40 wt. %. In some embodiments, the amount of shell is at a weight percentage by the total weight of the shell and the uncoated particle ranging from about 30.0 wt. % to about 40 wt. %. In some embodiments, the amount of shell is at a weight percentage by the total weight of the shell and the uncoated particle ranging from about 35 wt. % to about 40 wt. %. In some embodiments, the amount of shell is at a weight percentage by the total weight of the shell and the uncoated particle ranging from about 37.5 wt. % to about 40 wt. %.

In some embodiments, the amount of shell is at a weight percentage by the total weight of the shell and the uncoated particle ranging from about 5 wt. % to about 35 wt. %. In some embodiments, the amount of shell is at a weight percentage by the total weight of the shell and the uncoated particle ranging from about 6 wt. % to about 35 wt. %. In some embodiments, the amount of shell is at a weight percentage by the total weight of the shell and the uncoated particle ranging from about 7 wt. % to about 35 wt. %. In some embodiments, the amount of shell is at a weight percentage by the total weight of the shell and the uncoated particle ranging from about 8 wt. % to about 35 wt. %. In some embodiments, the amount of shell is at a weight percentage by the total weight of the shell and the uncoated particle ranging from about 9 wt. % to about 35 wt. %. In some embodiments, the amount of shell is at a weight percentage by the total weight of the shell and the uncoated particle ranging from about 10 wt. % to about 35 wt. %. In some embodiments, the amount of shell is at a weight percentage by the total weight of the shell and the uncoated particle ranging from about 15 wt. % to about 35 wt. %. In some embodiments, the amount of shell is at a weight percentage by the total weight of the shell and the uncoated particle ranging from about 25 wt. % to about 35 wt. %. In some embodiments, the amount of shell is at a weight percentage by the total weight of the shell and the uncoated particle ranging from about 30 wt. % to about 35 wt. %. In some embodiments, the amount of shell is at a weight percentage by the total weight of the shell and the uncoated particle ranging from about 12.5 wt. % to about 35 wt. %. In some embodiments, the amount of shell is at a weight percentage by the total weight of the shell and the uncoated particle ranging from about 15 wt. % to about 35 wt. %. In some embodiments, the amount of shell is at a weight percentage by the total weight of the shell and the uncoated particle ranging from about 17.5 wt. % to about 35 wt. %. In some embodiments, the amount of shell is at a weight percentage by the total weight of the shell and the uncoated particle ranging from about 20 wt. % to about 35 wt. %. In some embodiments, the amount of shell is at a weight percentage by the total weight of the shell and the uncoated particle ranging from about 22.5 wt. % to about 35 wt. %. In some embodiments, the amount of shell is at a weight percentage by the total weight of the shell and the uncoated particle ranging from about 25 wt. % to about 35 wt. %. In some embodiments, the amount of shell is at a weight percentage by the total weight of the shell and the uncoated particle ranging from about 27.5 wt. % to about 35 wt. %. In some embodiments, the amount of shell is at a weight percentage by the total weight of the shell and the uncoated particle ranging from about 30.0 wt. % to about 35 wt. %.

In some embodiments, the amount of shell is at a weight percentage by the total weight of the shell and the uncoated particle ranging from about 5 wt. % to about 30 wt. %. In some embodiments, the amount of shell is at a weight percentage by the total weight of the shell and the uncoated particle ranging from about 6 wt. % to about 30 wt. %. In some embodiments, the amount of shell is at a weight percentage by the total weight of the shell and the uncoated particle ranging from about 7 wt. % to about 30 wt. %. In some embodiments, the amount of shell is at a weight percentage by the total weight of the shell and the uncoated particle ranging from about 8 wt. % to about 30 wt. %. In some embodiments, the amount of shell is at a weight percentage by the total weight of the shell and the uncoated particle ranging from about 9 wt. % to about 30 wt. %. In some embodiments, the amount of shell is at a weight percentage by the total weight of the shell and the uncoated particle ranging from about 10 wt. % to about 30 wt. %. In some embodiments, the amount of shell is at a weight percentage by the total weight of the shell and the uncoated particle ranging from about 12.5 wt. % to about 30 wt. %. In some embodiments, the amount of shell is at a weight percentage by the total weight of the shell and the uncoated particle ranging from about 15 wt. % to about 30 wt. %. In some embodiments, the amount of shell is at a weight percentage by the total weight of the shell and the uncoated particle ranging from about 17.5 wt. % to about 30 wt. %. In some embodiments, the amount of shell is at a weight percentage by the total weight of the shell and the uncoated particle ranging from about 20 wt. % to about 30 wt. %. In some embodiments, the amount of shell is at a weight percentage by the total weight of the shell and the uncoated particle ranging from about 22.5 wt. % to about 30 wt. %. In some embodiments, the amount of shell is at a weight percentage by the total weight of the shell and the uncoated particle ranging from about 25 wt. % to about 30 wt. %. In some embodiments, the amount of shell is at a weight percentage by the total weight of the shell and the uncoated particle ranging from about 27.5 wt. % to about 30 wt. %.

In some embodiments, the amount of shell is at a weight percentage by the total weight of the shell and the uncoated particle ranging from about 21.0 wt. % to about 29.0 wt. %. In some embodiments, the amount of shell is at a weight percentage by the total weight of the shell and the uncoated particle ranging from about 22.0 wt. % to about 26.0 wt. %. In some embodiments, the amount of shell is at a weight percentage by the total weight of the shell and the uncoated particle ranging from about 23.0 wt. % to about 26.0 wt. %. In some embodiments, the amount of shell is at a weight percentage by the total weight of the shell and the uncoated particle ranging from about 24.0 wt. % to about 26.0 wt. %.

In some embodiments, the amount of shell is at a weight percentage by the total weight of the shell and the uncoated particle ranging from about 5 wt. % to about 25 wt. %. In some embodiments, the amount of shell is at a weight percentage by the total weight of the shell and the uncoated particle ranging from about 7.5 wt. % to about 25 wt. %. In some embodiments, the amount of shell is at a weight percentage by the total weight of the shell and the uncoated particle ranging from about 6 wt. % to about 25 wt. %. In some embodiments, the amount of shell is at a weight percentage by the total weight of the shell and the uncoated particle ranging from about 7 wt. % to about 25 wt. %. In some embodiments, the amount of shell is at a weight percentage by the total weight of the shell and the uncoated particle ranging from about 8 wt. % to about 25 wt. %. In some embodiments, the amount of shell is at a weight percentage by the total weight of the shell and the uncoated particle ranging from about 9 wt. % to about 25 wt. %. In some embodiments, the amount of shell is at a weight percentage by the total weight of the shell and the uncoated particle ranging from about 10 wt. % to about 25 wt. %. In some embodiments, the amount of shell is at a weight percentage by the total weight of the shell and the uncoated particle ranging from about 15 wt. % to about 25 wt. %. In some embodiments, the amount of shell is at a weight percentage by the total weight of the shell and the uncoated particle ranging from about 12.5 wt. % to about 25 wt. %. In some embodiments, the amount of shell is at a weight percentage by the total weight of the shell and the uncoated particle ranging from about 15 wt. % to about 25 wt. %. In some embodiments, the amount of shell is at a weight percentage by the total weight of the shell and the uncoated particle ranging from about 17.5 wt. % to about 25 wt. %. In some embodiments, the amount of shell is at a weight percentage by the total weight of the shell and the uncoated particle ranging from about 20 wt. % to about 25 wt. %.

In some embodiments, the amount of shell is at a weight percentage by the total weight of the shell and the uncoated particle selected from the group of about 5.0 wt. %, about 5.5 wt. %, about 6.0 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, about 25.0 wt. %, about 25.5 wt. %, about 26.0 wt. %, about 26.5 wt. %, about 27.0 wt. %, about 27.5 wt. %, about 28.0 wt. %, about 28.5 wt. %, about 29.0 wt. %, about 29.5 wt. %, about 30.0 wt. %, about 30.5 wt. %, about 31.0 wt. %, about 31.5 wt. %, about 32.0 wt. %, about 32.5 wt. %, about 33.0 wt. %, about 33.5 wt. %, about 34.0 wt. %, about 34.5 wt. %, about 35.0 wt. %, about 35.5 wt. %, about 36.0 wt. %, about 36.5 wt. %, about 37.0 wt. %, about 37.5 wt. %, about 38.0 wt. %, about 38.5 wt. %, about 39.0 wt. %, about 39.5 wt. %, or 40.0 wt. %. In an embodiment, the amount of shell is about 7.5 wt. % by the total weight of the shell and the uncoated particle. In an embodiment, the amount of shell is about 10.0 wt. % by the total weight of the shell and the uncoated particle. In an embodiment, the amount of shell 1 is about 15.0 wt. % by the total weight of the shell and the uncoated particle. In an embodiment, the amount of shell is about 20.0 wt. % by the total weight of the shell and the uncoated particle. In an embodiment, the amount of shell is about 25.0 wt. % by the total weight of the shell and the uncoated particle. In an embodiment, the amount of shell is about 30.0 wt. % by the total weight of the shell and the uncoated particle.

In some embodiments, the shell layer is present in an amount in a range from about 10.0 wt. % to about 200 wt. % of the total weight of the uncoated particles. In some embodiments, the shell layer is present in an amount ranging from about 20.0 wt. % to about 100 wt. % of the total weight of the uncoated particles. In some embodiments, the shell layer is present in an amount ranging from about 20.0 wt. % to about 120 wt. % of the total weight of the uncoated particles. In some embodiments, the shell layer is present in an amount ranging from about 20.0 wt. % to about 130 wt. % of the total weight of the uncoated particles. In some embodiments, the shell layer is present in an amount ranging from about 20.0 wt. % to about 140 wt. % of the total weight of the uncoated particles. In some embodiments, the shell layer is present in an amount ranging from about 20.0 wt. % to about 150 wt. % of the total weight of the uncoated particles. In some embodiments, the shell layer is present in an amount ranging from about 20.0 wt. % to about 200 wt. % of the total weight of the uncoated particles. In some embodiments, the shell layer is present in an amount ranging from about 30.0 wt. % to about 100 wt. % of the total weight of the uncoated particles. In some embodiments, the shell layer is present in an amount ranging from about 40.0 wt. % to about 100 wt. % of the total weight of the uncoated particles. In some embodiments, the shell layer is present in an amount ranging from about 60.0 wt. % to about 100 wt. % of the total weight of the uncoated particles. In some embodiments, the shell layer is present in an amount ranging from about 70.0 wt. % to about 100 wt. % of the total weight of the uncoated particles. In some embodiments, the shell layer is present in an amount (e.g., 10 wt. % of the total weight of the uncoated particles) that forms an imperfect shell that is unable to completely prevent leakage of components or that meets the cytotoxicity IC₃₀ criteria as set forth above. In some embodiments, the shell layer is present in an amount of about 100 wt. % of the total weight of the uncoated particles. In some embodiments, the shell layer is present in an amount of about 200 wt. % of the total weight of the uncoated particles. In some embodiments, the shell layer is present in an amount in selected from the group of about 10.0 wt. %, about 15.0 wt. %, about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, about 100 wt. %, about 110 wt. %, about 115 wt. %, about 120 wt. %, about 125 wt. %, about 130 wt. %, about 135 wt. %, about 140 wt. %, about 145 wt. %, about 150 wt. %, about 155 wt. %, about 160 wt. %, about 165 wt. %, about 170 wt. %, about 175 wt. %, about 180 wt. %, about 185 wt. %, about 190 wt. %, about 195 wt. %, about 200 wt. % of the total weight of the uncoated particles. In some embodiments, the shell layer is present in an amount in a range from 10.0 wt. % to about 35.0 wt. % of the total weight of the uncoated particles. In some embodiments, the shell is present in an amount of about 35.0 wt. % of the total weight of the uncoated particles.

In some embodiments, the exogenous source is electromagnetic radiation, microwaves, radio waves, sound waves, electrical, or magnetic field. Currently, several energy sources (e.g. laser light, focused ultrasound and microwave) have been employed in thermal cancer therapy.

In some embodiments, the exogenous source may be electromagnetic radiation (EMR). In some embodiments, the exogenous source comprises a laser light. In some embodiments, the exogenous source comprises a LED light. In some embodiments, the laser light is a pulsed laser light. In some embodiments, the laser pulse duration is in a range from milliseconds to nanoseconds, and the laser has an oscillation wavelength at either 805 nm, 808 nm or 1064 nm. In some embodiments, the laser pulse duration is in a range from milliseconds to femtoseconds and the laser has an oscillation wavelength at 805 nm, 808 nm or 1064 nm. In some embodiments, the laser emits light at 808 nm. In some embodiments, the laser emits light at 805 nm.

In some embodiments, the exogenous source may have a cold tip to cool the target tissue area before, during and after application of the exogenous energy. In some embodiments the cold tip may be a temperature from 2-8° C.

In some embodiments, the material interacting with the exogenous source produces heat that performs a function, like inducing cytotoxicity by raising the temperature to above normal body temperature.

In some embodiments, the material is an IR-absorbing agent selected from the group of phthalocyanines, naphthalocyanines, and combinations thereof. In some embodiments, the IR absorbing agent is selected from the group of a tris-aminium dye, a tetrakis aminium dye, a squarylium dye, a cyanine dye, zinc copper phosphate pigment, a palladate compound, a platinate compound, and combinations thereof. In some embodiments, the IR absorbing agent comprises cyanine dyes selected from the group of indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof.

In some embodiments, the IR absorbing agent is indocyanine green (ICG).

In some embodiments, the squarylium dye is a benzopyrylium squarylium dye having formula (III)

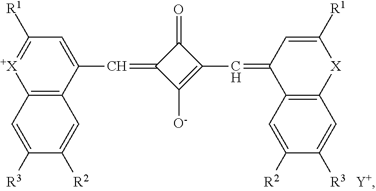

wherein each X is independently 0, S, Se; Y⁺ is a counterion selected from the group of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof each R¹ is a non-aromatic organic substituent, each R²═H or OR³, R³=cycloalkyl, alkenyl, acyl, silyl; each R³═—NR⁴R⁵, each R⁴, R⁵ is independently H, C1-8 alkyl. In some embodiments, the squarylium dye of formula (III) is a compound when R¹═—CMe₃, R²═OCHMeEt, X═O with a strong absorption at 788 nm. In some embodiments, the squarylium dye of formula (III) is a compound when R₁═—CMe₃, R²═H, R³═—NEt₂, X═O with a strong absorption at 808 nm (IR 193 dye).

In some embodiments, the IR absorbing agent may include a squarylium dye. In some embodiments, the IR absorbing agent may include a squaraine dye. In some embodiments, the IR absorbing agent may include IR 193 dye.

In some embodiments, the IR absorbing agent is selected from the group of a tris-aminium dye, a tetrakis aminium dye, a squarylium dye, a cyanine dye, zinc copper phosphate pigment, gold nanostructure, iron oxide, a palladate compound, a platinate compound, and combinations thereof.

In some embodiments, the inorganic IR absorbing agent comprises iron oxide nanoparticle (also known to function as MRI contrast agent, magnetic energy absorbing agent).

In some embodiments, the material is selected from the group of a tetrakis aminium dye, a cyanine dye, a squarylium dye, indocyanine green (ICG), new ICG (IR 820), squaraine dye, IR 780 dye, IR 193 dye, Epolight™ IR 1117, Epolight™ 1175, iron oxide, zinc iron phosphate pigment, and combinations thereof.

In some embodiments, the IR absorbing agent is a tetrakis aminium dye. In some embodiments, the tetrakis aminium dye is a narrow band absorber including commercially available IR absorbing agents sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broadband absorber including commercially available IR absorbing agents sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the IR absorbing agent is admixed within the carrier to form a homogeneous dispersion or a solid solution. In some embodiments, the IR absorbing agent and the carrier may have oppositely charged functional group(s) (e.g., IR absorbing agent is positively charged tetrakis aminium dye, and the carrier has negatively charged functional group such as carboxylate anion of polymethacrylate polymers) such that the IR absorbing agent attaches to the carrier via hydrogen bond or via ionic electrostatic interactions.

In some embodiments, the material interacting with the exogenous source also comprises plasmonic absorbers or iron oxide. In some embodiments, the material comprises plasmonic absorber. In some embodiments, the material comprises iron oxide.

In some embodiments, the shell comprises a plasmonic absorber or iron oxide. In some embodiments, iron oxide is in the form of iron oxide nanoparticle or iron oxide coating layer. In some embodiments, the shell is formed of plasmonic absorber only. In some embodiments, the shell is composed of the crosslinked inorganic polymer doped with the plasmonic absorber.

In some embodiments, the plasmonic absorber is selected from the group of gold nanostructures, silver nanoparticles, graphene oxide nanomaterials and combinations thereof.

In some embodiments, the plasmonic absorbers comprise plasmonic nanomaterials of noble metal nanostructures including gold (Au) nanostructures, silver (Ag) nanoparticles, and copper (Cu) nanoparticles doped with sulfur (S), selenium (Se) or tellurium (Te) having a plasmonic resonance at NIR wavelength. In some embodiments, the plasmonic absorbers comprise gold nanostructures such as nanoporous gold thin films, or gold nanospheres, gold nanorods, gold nanoshells, gold nanocages, silver nanoparticles, and Cu₉S₅ nanoparticle. In some embodiments, the plasmonic absorbers comprise gold nanostructures.

Compared to non-metallic nanoparticles, plasmonic nanomaterials hold a unique photophysical phenomenon, called localized surface plasmon resonance (LSPR) because of the absorption of light of resonant frequency. The plasmonic nanomaterials (e.g., noble metal nanostructures) show superior light absorption efficiency over conventional IR absorbing agent molecules. Upon irradiation with electromagnetic radiation, strong surface fields are induced due to the coherent excitation of the electrons in the metallic nanoparticles. The rapid relaxation of these excited electrons produces strong localized heat capable of destroying the surrounding tumor cells via hyperthermia or other cytotoxic effects (e.g., cell killing effects of the radicals). By changing the structure (e.g., size) and shape, the LSPR frequency of the noble metal nanostructures can be tuned for the resulting plasmonic resonance wavelength in the NIR therapeutic window (750-1300 nm), where light penetration in the tissue is optimal. The endogenous absorption coefficient of the tissue is nearly two orders of magnitude lower than that in the visible part of electromagnetic spectrum. In some embodiments, the plasmonic absorbers may have an LSPR ranging from about 700 nm to about 900 nm. In some embodiments, the plasmonic absorbers may have an LSPR raging from about 900 nm to about 1064 nm.

In some embodiments, the particle heater has a loading amount of the material interacting with exogenous source that is measured by spectroscopic absorbance. In some embodiments, the particle heater has a loading amount of the material that is measured by known analytical technology in the art, like UV-VIS-NIR, NMR, HPLC, LCMS, etc. In some embodiments, the loading amount of the material is in a range from about 0.01 wt. % to about 20.0 wt. % by the total weight of the particle heater. In some embodiments, the loading amount of the material in a range from about 1.0 wt. % to about 20.0 wt. % by the total weight of the particle heater. In some embodiments, the loading amount of the material ranges from about 5.0 wt. % to about 20.0 wt. % by the total weight of the particle heater. In some embodiments, the loading amount of the material ranges from about 10.0 wt. % to about 20.0 wt. % by the total weight of the particle heater. In some embodiments, the loading amount of the material ranges from about 5.0 wt. % to about 15.0 wt. % by the total weight of the particle heater. In some embodiments, the loading amount of the material ranges from about 10.0 wt. % to about 15.0 wt. % by the total weight of the particle heater. In some embodiments, the loading amount of the material ranges from about 5.0 wt. % to about 12.5 wt. % by the total weight of the particle heater. In some embodiments, the loading amount of the material is selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, and about 20.0 wt. % by the total weight of the particle heater.

In some embodiments, the material interacting with an exogenous source is an IR absorbing agent and the particle heater has the IR absorbing agent in an amount ranging from about 0.1 wt. % to about 15.0 wt. % by the total weight of the particle heater. In some embodiments, the particle heater has the IR absorbing agent in an amount selected from the group of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, and about 15.0 wt. % by the total weight of the particle heater. In some embodiments, the particle heater has the IR absorbing agent in an amount selected from the group of about 0.1% wt. %, about 0.2% wt. %, about 0.3% wt. %, about 0.4% wt. %, about 0.5% wt. %, about 0.6% wt. %, about 0.7% wt. %, about 0.8% wt. %, about 0.9% wt. %, about 1.0% wt. %, about 1.1% wt. %, about 1.2% wt. %, about 1.3% wt. %, about 1.4% wt. %, about 1.5% wt. %, about 1.6% wt. %, about 1.7% wt. %, about 1.8% wt. %, about 1.9% wt. %, about 2.0% wt. %, about 2.1% wt. %, about 2.2% wt. %, about 2.3% wt. %, about 2.4% wt. %, about 2.5% wt. %, about 2.6% wt. %, about 2.7% wt. %, about 2.8% wt. %, about 2.9% wt. %, about 3.1% wt. %, about 3.1% wt. %, about 3.2% wt. %, about 3.3% wt. %, about 3.4% wt. %, about 3.5% wt. %, about 3.6% wt. %, about 3.7% wt. %, about 3.8% wt. %, about 3.9% wt. %, about 4.0% wt. %, about 4.1% wt. %, about 4.2% wt. %, about 4.3% wt. %, about 4.4% wt. %, about 4.5% wt. %, about 4.6% wt. %, about 4.7% wt. %, about 4.8% wt. %, about 4.9% wt. %, and about 5.0% wt. % by the total weight of the particle heater.

In some embodiments, the particle has a weight ratio of the carrier to the material ranging from 1:1 to 7:1. In some embodiments, the particle has a weight ratio of the carrier to the material selected from the group of 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and 7.0:1.

In some embodiments, the particle has a weight ratio of the material in the core to the plasmonic absorber in the shell ranging from 5:1 to 1:5. In some embodiments, the particle has a weight ratio of the material in the core to the plasmonic absorber in the shell selected from the group of 5.0:1, 4.9:1, 4.8:1, 4.7:1, 4.6:1, 4.5:1, 4.4:1, 4.3:1, 4.2:1, 4.1:1, 4.0:1, 3.9:1, 3.8:1, 3.7:1, 3.6:1, 3.5:1, 3.4:1, 3.3:1, 3.2:1, 3.1:1, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, and 1:5.

In some embodiments, the particle heater exhibits stability such that the degradation of the material by body chemicals is less than 20% as measured by the Efficacy Determination Protocol after incubating the particles in the extraction medium (serum) for 24 hours at 37° C. In some embodiments, the particle exhibits stability such that material has a degree of degradation selected from the group of about 5.0%, about 10%, about 15%, and about 20% as measured by Efficacy Determination Protocol. In some embodiments, the material has a degree of degradation in a range selected from the group of less than about 20.0%, less than about 15.0%, less than about 10.0%, less than about 5.0%, less than about 1.0%, less than about 0.5%, less than about 0.1%, and less than about 0.01% as determined by Efficacy Determination Protocol. In some embodiments, the material has a degree of degradation less than about 10.0% as determined by Efficacy Determination Protocol. In some embodiments, the material has a degree of degradation less than about 5.0% as measured by Efficacy Determination Protocol. In some embodiments, the material has a degree of degradation less than about 1.0% as measured by Efficacy Determination Protocol. In some embodiments, the material has a degree of degradation less than about 0.1% as measured by Efficacy Determination Protocol.

In some embodiments, the particle exhibits energy-to-heat conversion stability such that the loss in absorbance of the material is less than 50% as measured by the Material Process Stability Test after exposure to a pulsed laser light.

In some embodiments, the carrier is selected based on the specific material to be encapsulated, e.g., carrier is chemically compatible with the material. In some embodiments, the carrier comprises organic or inorganic polymer. In some embodiments, the carrier is an organic polymer. In some embodiments, the carrier comprises polymer or copolymer of methylmethacrylate. In some embodiments, the carrier comprises mesoporous silica. In some embodiments, the carrier comprises a biodegradable and/or biocompatible polymer. In some embodiments, the biodegradable and/or biocompatible polymer may include, but is not limited to, a polyester, a polyurea, a polyanhydride, a polysaccharide, a polyphosphoester, a poly(ortho ester), a poly(amino acid), a protein, polyurea, and combinations thereof.

In some embodiments, the biodegradable and/or biocompatible polymer may include, but are not limited to: polymethyl methacrylate, polyester, poly caprolactone (PCL), poly(trimethylene carbonate) or other poly (alpha-esters), polyurethanes, poly(allylamine hydrochloride), poly(ester amides), poly (ortho esters), polyanyhydrides, poly (anhydride-co-imide), cross linked polyanhydrides, pseudo poly(amino acids), poly (alkylcyanoacrylates), polyphosphoesters, polyphosphazenes, chitosan, collagen, natural or synthetic poly(amino acids), elastin, elastin-like polypeptides, albumin, fibrin, polysiloxanes, polycarbosiloxanes, polysilazanes, polyalkoxysiloxanes, polysaccharides, cross-linkable polymers, thermo-responsive polymers, thermo-thinning polymers, thermo-thickening polymers, or block co-polymers of the above polymers with polyethylene glycol, and combinations thereof.

In some embodiments, the carrier comprises a hydrophobic polymer or copolymer of polymethacrylates, polycarbonate, or combinations thereof. In some embodiments, the carrier comprises polymethylmethacrylate (PMMA, Neocryl® 728 sold by DSM, T_{g}=111° C.). In some embodiments, the polymethacrylate copolymer is MMA/BMA copolymer and the weight ratio of MMA to BMA is 96:4 (e.g. Neocryl® 805 by DSM, acid value less than 1).

In some embodiments, the particle is amorphous or partially amorphous or partially crystalline.

In an embodiment, this disclosure provides a particle heater for use in the remotely-triggered thermal treatment of a cancer comprising:

a material that interacts with an exogenous source, wherein the material is an IR absorbing agent selected from the group of a tris-aminium dye, a di-imonium dye, a tetrakis aminium dye, a cyanine dye, a squaraine dye, a zinc iron phosphate pigment, and combinations thereof,

a carrier comprising a polymer selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof;

wherein the particle heater has a median particle size less than 5 μm,

wherein the material interacting with an exogenous source is encapsulated by the carrier to form a particle,

wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells. In some embodiments, the particle further passes the Thermal Cytotoxicity Test.

In some embodiments, the particle heater further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof.

In some embodiments, the particle surface further comprises a targeting group selected from the group of an EGFR binding antibodies including cetuximab, and panitumumab; an EGFR binding peptides selected from the group of YHWYGYTPQNVI, YRWYGYTPQNVI, L-AE (L amino acids in the sequence-FALGEA), D-AE (D-amino acids in the sequence-FALGEA), and combinations thereof.

In some embodiments, the particle surface is further modified with a hydrophilic polymer selected from the group of polyethylene glycols, hyperbranched polyglycerol, hyaluronic acid, and combinations thereof.

In an embodiment, this disclosure provides a particle heater for use in the remotely-triggered thermal treatment of a cancer comprising: (a) a material that interacts with an exogenous source, wherein the material is a tetrakis aminium dye, a cyanine dye, a squaraine dye or combinations thereof, (b) a carrier comprising a blend of PLGA 75:25 (weight ratio of lactic acid:glycolic acid=75:25) with PLGA 75:25-polyethylene glycol block copolymer (PLGA 75:25-b-PEG) (weight ratio of lactic acid:glycolic acid=75:25); wherein the particle heater has a median particle size ranging from 1 nm to 250 nm, wherein the tetrakis aminium dye, or the squaraine dye, or the cyanine dye is encapsulated by the carrier to form a particle.

In some embodiments, the targeting group is conjugated to the particle heater surface via a linking segment comprising a specific type cancer protease binding peptide for enhancing the precision of the delivery of the particle heater to the tumor site.

### 3(b). Photothermal Antimicrobial Therapy

Microbes are more susceptible to low hyperthermal effects than normal tissues; therefore both systemic and localized hyperthermia regimes have been successful in treating microbial infections. Many natural light absorbers in tissues (e.g. water, hemoglobin, oxyhemoglobin and melanin) can convert light energy into heat for causing hyperthermia damage to both microbes and healthy tissues. However, near infrared light (NIR) induces minimal photothermal heating in both microbes and healthy tissues as the absorption of biological tissues is lowest in a NIR region (700 nm to 1400 nm). Microbial damage is evident within minutes when the temperature of the infected tissue reaches 55-95° C.

Antimicrobial thermal therapy is based on the ability to convert light into heat to destroy microorganisms such as bacteria thermally. Heating sources including near infrared or visible light, radiofrequency waves, microwaves, and ultrasound waves are used to induce moderate temperature rise in a specific target region to destroy the pathogenic microbes, clinically termed as hyperthermia. Due to the low absorption efficiency of the natural tissue absorbents, synthetic organic IR absorbing agent, such as indocyanine green, naphthalocyanines and porphyrins coordinated with transition metals are externally administered into the tissue sites to enhance the thermal effects.

Molecules and materials that can absorb energy from an exogenous source to generate heat for controlled and localized temperature increments are potentially valuable for numerous applications in remotely triggered thermal therapies. Unlike conventional chemotherapies, there are no known resistance mechanisms to thermal therapies. Remotely triggered thermal therapies are therefore highly attractive for inducing toxicity in drug-resistant pathogenic microbes.

PTT triggered by near-infrared (NIR) light usually requires a temperature of 50° C. or higher to denature proteins and kill microbes because cell damage such as apoptosis at a lower temperature (e.g. 45° C.) can be repaired, but the high temperature may cause inflammation and thermal damage to nearby host tissues. Therefore, a bactericidal strategy that minimizes collateral damage is more desirable, for example, at moderate hyperthermia temperature range of about 41.1° C. to about 45° C.

Photothermal therapy (PTT) employs NIR light induced localized hyperthermia to cause cytotoxic effects on microbes (e.g. apoptosis or necrosis depending on the laser dosage, type and irradiation duration). Hyperthermia can lead to cell death via protein denaturation or rupture of the cellular membrane (autolysis) and subsequently result in the removal of microbes by macrophages, which achieve numerous potential benefits over conventional antimicrobial therapies. Compared with traditional chemotherapy, PTT exhibits unique advantages such as higher specificity, minimal invasiveness and higher efficacy.

Numerous cyanine dyes have been employed as photothermal conversion agents due to its strong NIR absorbance and the conversion of the absorbed photonic energy to heat. However, the direct use of free cyanine dyes in PTT is severely limited by their poor aqueous solubility, rapid body clearance, poor cellular uptake, and lack of targeting ability. Indocyanine green (ICG) is approved by FDA for clinical imaging and diagnosis. In some embodiments, this disclosure provides particle heaters for antimicrobial thermal therapy comprising a carrier for encapsulating a material that interacts with an exogenous source. Upon interaction with the exogenous source, the material produces heat, which is then used to kill the pathogenic microbial cells at the infection site. Particle heaters may further include a diagnostic agent that remains colorless unless there are specific antimicrobial drug-resistant microbes present at the infection site in which case the diagnostic agent changes to a colored state that can be visually seen by the physician. This color change can be caused in a few minutes to up to two hours following application of the particles to the surgical site.

The particle structure is designed using three tests: 1. Extractable Cytotoxicity Test, which evaluates the ability of body chemicals (like serum) to extract the material that interacts with the exogenous source and/or the diagnostic agent and tests the ability of these extracts to kill normal host cells. Particle structure that limits leakage of the material encapsulated within the particle such that no more than 30% of the normal host cells are killed are considered safe for further use. 2. Efficacy Determination Protocol, which evaluates the ability of the particle structure to protect chemical components within the particle. In this assay, particles are incubated with physiologically relevant media (e.g. cell culture media containing serum proteins) such that chemicals present in these media may enter the particle and breakdown or reduce the efficacy of the material to absorb exogenous energy and convert it to heat. The particle structure is iteratively modified such that the chemicals break down no more than 25% of the material. 3. Thermal Cytotoxicity Test, which is an in vitro test specifically designed to test the particles and the specific exogenous source(s) for their ability to kill the pathogenic microbial cells while sparing the normal host cells. The thermal cytotoxicity test is a trans-well assay wherein two different cells types, one being the microbial cells with the other type being the normal, host cells, are grown in the same well and exposed to different doses of the particles and the exogenous source (see FIG. 6). Viabilities of the two cells types are assessed a day after exposure of the cells to the compositions and exogenous source using standard colorimetric assays. Different types of pathogenic microbial or normal host cells can be selected for this test for different antimicrobial applications. The particle and exogenous source (e.g. light) dose(s) that do not kill any more than 30% of the healthy host cells but kill at least 70% of the pathogenic microbial cells are considered passing the thermal cytotoxicity test. Use of any of these rigid tests to improve particle structural design through a feedback loop is not explored in the prior art.

In some embodiments, the degradation for the material encapsulated within the particle can be determined using the material loading determination protocol as set forth in Example 3 The degradation of non-encapsulated material can also be compared to that of the encapsulated material to evaluate the effect of encapsulation in particles. Depending on the application, different biological agents can be added to the cell culture media to simulate conditions that occur in vivo. This protocol in conjunction with the Extractable Cytotoxicity Test and/or Thermal Cytotoxicity Test will provide feedback (Feedback Loop 1A or Feedback Loop 1B) to design the particle structure such that the material (e.g., the IR absorbing agent) can be protected from the degradation by body chemicals. The Extractable Cytotoxicity Test is conducted according to the protocols set forth below (See FIGS. 1A-B). The particle structure characteristics (e.g. carrier material selection, particle size, morphology, adding a shell, particle surface modification etc.) and the exogenous source characteristics (e.g. laser wavelength, pulse duration and energy efficiency) are designed sequentially based on the structure-property relationship feedbacks provides from the tests in the flow chart of FIGS. 1A-B including Extractable Cytotoxicity Test, Efficacy Determination Test and/or Thermal Cytotoxicity Test. The ideal particle heaters possess the characteristics of high energy-to-heat conversion efficiency, thermal stability, and low collateral damage.

In some embodiments, the material interacting with the exogenous source produces heat that performs a function, like inducing cytotoxicity by raising the temperature to above normal body temperature.

In some embodiments, the material is an IR-absorbing agent selected from the group of phthalocyanines, naphthalocyanines, and combinations thereof. In some embodiments, the IR absorbing agent is selected from the group of a tris-aminium dye, a tetrakis aminium dye, a squarylium dye, a cyanine dye, zinc copper phosphate pigment, a palladate compound, a platinate compound, and combinations thereof. In some embodiments, the IR absorbing agent comprises cyanine dyes selected from the group of indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof.

In some embodiments, the IR absorbing agent is indocyanine green (ICG).

In some embodiments, the squarylium dye is a benzopyrylium squarylium dye having formula (III)

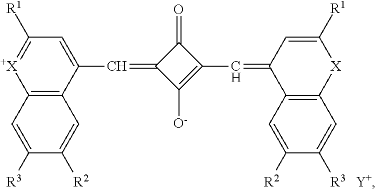

wherein each X is independently 0, S, Se; Y⁺ is a counterion selected from the group of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof each R¹ is a non-aromatic organic substituent, each R²═H or OR³, R³=cycloalkyl, alkenyl, acyl, silyl; each R³═—NR⁴R⁵, each R⁴, R⁵ is independently H, C1-8 alkyl. In some embodiments, the squarylium dye of formula (III) is a compound when R¹═—CMe₃, R²═OCHMeEt, X═O with a strong absorption at 788 nm. In some embodiments, the squarylium dye of formula (III) is a compound when R¹═—CMe₃, R²═H, R³═—NEt₂, X═O with a strong absorption at 808 nm (IR 193 dye).

In some embodiments, the IR absorbing agent may include a squarylium dye. In some embodiments, the IR absorbing agent may include a squaraine dye. In some embodiments, the IR absorbing agent may include IR 193 dye.

In some embodiments, the IR absorbing agent is selected from the group of a tris-aminium dye, a tetrakis aminium dye, a squarylium dye, a cyanine dye, zinc copper phosphate pigment, gold nanostructure, iron oxide, a palladate compound, a platinate compound, and combinations thereof.

In some embodiments, the inorganic IR absorbing agent comprises iron oxide nanoparticle (also known to function as MRI contrast agent, magnetic energy absorbing agent).

In some embodiments, the material is selected from the group of a tetrakis aminium dye, a cyanine dye, a squarylium dye, indocyanine green (ICG), new ICG (IR 820), squaraine dye, IR 780 dye, IR 193 dye, Epolight™ 1117 dye, Epolight™ 1175, iron oxide, zinc iron phosphate pigment, and combinations thereof.

In some embodiments, the IR absorbing agent is a tetrakis aminium dye. In some embodiments, the tetrakis aminium dye is a narrow band absorber including commercially available dyes sold under the trademark names Epolight™ 1117 (peak absorption, 1071 nm), Epolight™ 1151 (peak absorption, 1070 nm), or Epolight™ 1178 (peak absorption, 1073 nm). In some embodiments, the tetrakis aminium dyes is a broadband absorber including commercially available IR absorbing agents sold under the trademark names Epolight™ 1175 (peak absorption, 948 nm), Epolight™ 1125 (peak absorption, 950 nm), and Epolight™ 1130 (peak absorption, 960 nm). In some embodiments, the tetrakis aminium dye is Epolight™ 1178.

In some embodiments, the IR absorbing agent is admixed within the carrier to form a homogeneous dispersion or a solid solution. In some embodiments, the IR absorbing agent and the carrier may have oppositely charged functional group(s) (e.g., IR absorbing agent is positively charged tetrakis aminium dye, and the carrier has negatively charged functional group such as carboxylate anion of polymethacrylate polymers) such that the IR absorbing agent attaches to the carrier via hydrogen bond or via ionic electrostatic interactions.

In some embodiments, the material interacting with the exogenous source also comprises plasmonic absorbers or iron oxide. In some embodiments, the material comprises plasmonic absorber. In some embodiments, the material comprises iron oxide.

In some embodiments, the shell comprises a plasmonic absorber or iron oxide. In some embodiments, iron oxide is in the form of iron oxide nanoparticle or iron oxide coating layer. In some embodiments, the shell is formed of plasmonic absorber only. In some embodiments, the shell is composed of the crosslinked inorganic polymer doped with the plasmonic absorber.

In some embodiments, the plasmonic absorber is selected from the group of gold nanostructures, silver nanoparticles, graphene oxide nanomaterials and combinations thereof.

In some embodiments, the plasmonic absorbers comprise plasmonic nanomaterials of noble metal nanostructures including gold (Au) nanostructures, silver (Ag) nanoparticles, and copper (Cu) nanoparticles doped with sulfur (S), selenium (Se) or tellurium (Te) having a plasmonic resonance at NIR wavelength. In some embodiments, the plasmonic absorbers comprise gold nanostructures such as nanoporous gold thin films, or gold nanospheres, gold nanorods, gold nanoshells, gold nanocages, silver nanoparticles, and Cu₉S₅ nanoparticle. In some embodiments, the plasmonic absorbers comprise gold nanostructures.

Compared to non-metallic nanoparticles, plasmonic nanomaterials hold a unique photophysical phenomenon, called localized surface plasmon resonance (LSPR) because of the absorption of light of resonant frequency. The plasmonic nanomaterials (e.g., noble metal nanostructures) show superior light absorption efficiency over conventional IR absorbing agent. Upon irradiation with electromagnetic radiation, strong surface fields are induced due to the coherent excitation of the electrons in the metallic nanoparticles. The rapid relaxation of these excited electrons produces strong localized heat capable of destroying the surrounding tumor cells via hyperthermia or other cytotoxic effects (e.g., cell killing effects of the radicals). By changing the structure (e.g., size) and shape, the LSPR frequency of the noble metal nanostructures can be tuned for the resulting plasmonic resonance wavelength in the NIR therapeutic window (750-1300 nm), where light penetration in the tissue is optimal. The endogenous absorption coefficient of the tissue is nearly two orders of magnitude lower than that in the visible part of electromagnetic spectrum. In some embodiments, the plasmonic absorbers may have an LSPR ranging from about 700 nm to about 900 nm. In some embodiments, the plasmonic absorbers may have an LSPR raging from about 900 nm to about 1064 nm.

In some embodiments, the particle heater has a loading amount of the material interacting with exogenous source that is measured by spectroscopic absorbance. In some embodiments, the particle heater has a loading amount of the material that is measured by known analytical technology in the art, like UV-VIS-NIR, NMR, HPLC, LCMS, etc. In some embodiments, the loading amount of the material is in a range from about 0.01 wt. % to about 20.0 wt. % by the total weight of the particle heater. In some embodiments, the loading amount of the material in a range from about 1.0 wt. % to about 20.0 wt. % by the total weight of the particle heater. In some embodiments, the loading amount of the material ranges from about 5.0 wt. % to about 20.0 wt. % by the total weight of the particle heater. In some embodiments, the loading amount of the material ranges from about 10.0 wt. % to about 20.0 wt. % by the total weight of the particle heater. In some embodiments, the loading amount of the material ranges from about 5.0 wt. % to about 15.0 wt. % by the total weight of the particle heater. In some embodiments, the loading amount of the material ranges from about 10.0 wt. % to about 15.0 wt. % by the total weight of the particle heater. In some embodiments, the loading amount of the material ranges from about 5.0 wt. % to about 12.5 wt. % by the total weight of the particle heater. In some embodiments, the loading amount of the material is selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, and about 20.0 wt. % by the total weight of the particle heater.

In some embodiments, the material interacting with an exogenous source is an IR absorbing agent and the particle heater has the IR absorbing agent in an amount ranging from about 0.1 wt. % to about 15.0 wt. % by the total weight of the particle heater. In some embodiments, the particle heater has the IR absorbing agent in an amount selected from the group of about 5.0 wt. %, about 5.25 wt. %, about 5.5 wt. %, about 5.75 wt. %, about 6.0 wt. %, 6.25 wt. %, about 6.5 wt. %, about 6.75 wt. %, about 7.0 wt. %, 7.25 wt. %, about 7.5 wt. %, about 7.75 wt. %, about 8.0 wt. %, about 8.25 wt. %, about 8.5 wt. %, about 8.75 wt. %, about 9.0 wt. %, about 9.25 wt. %, about 9.5 wt. %, about 9.75 wt. %, about 10.0 wt. %, about 10.25 wt. %, about 10.5 wt. %, about 10.75 wt. %, about 11.0 wt. %, about 11.25 wt. %, about 11.5 wt. %, about 11.75 wt. %, about 12.0 wt. %, about 12.25 wt. %, about 12.5 wt. %, about 12.75 wt. %, about 13.0 wt. %, about 13.25 wt. %, about 13.5 wt. %, about 13.75 wt. %, about 14.0 wt. %, about 14.25 wt. %, about 14.5 wt. %, about 14.75 wt. %, and about 15.0 wt. % by the total weight of the particle heater. In some embodiments, the particle heater has the IR absorbing agent in an amount selected from the group of about 0.1% wt. %, about 0.2% wt. %, about 0.3% wt. %, about 0.4% wt. %, about 0.5% wt. %, about 0.6% wt. %, about 0.7% wt. %, about 0.8% wt. %, about 0.9% wt. %, about 1.0% wt. %, about 1.1% wt. %, about 1.2% wt. %, about 1.3% wt. %, about 1.4% wt. %, about 1.5% wt. %, about 1.6% wt. %, about 1.7% wt. %, about 1.8% wt. %, about 1.9% wt. %, about 2.0% wt. %, about 2.1% wt. %, about 2.2% wt. %, about 2.3% wt. %, about 2.4% wt. %, about 2.5% wt. %, about 2.6% wt. %, about 2.7% wt. %, about 2.8% wt. %, about 2.9% wt. %, about 3.1% wt. %, about 3.1% wt. %, about 3.2% wt. %, about 3.3% wt. %, about 3.4% wt. %, about 3.5% wt. %, about 3.6% wt. %, about 3.7% wt. %, about 3.8% wt. %, about 3.9% wt. %, about 4.0% wt. %, about 4.1% wt. %, about 4.2% wt. %, about 4.3% wt. %, about 4.4% wt. %, about 4.5% wt. %, about 4.6% wt. %, about 4.7% wt. %, about 4.8% wt. %, about 4.9% wt. %, and about 5.0% wt. % by the total weight of the particle heater.

In some embodiments, the particle has a weight ratio of the carrier to the material ranging from 1:1 to 7:1. In some embodiments, the particle has a weight ratio of the carrier to the material selected from the group of 1.0:1, 1.1:1, 1.2:1, 1.3:1, 1.4:1, 1.5:1, 1.6:1, 1.7:1, 1.8:1, 1.9:1, 2.0:1, 2.1:1, 2.2:1, 2.3:1, 2.4:1, 2.5:1, 2.6:1, 2.7:1, 2.8:1, 2.9:1, 3.0:1, 3.1:1, 3.2:1, 3.3:1, 3.4:1, 3.5:1, 3.6:1, 3.7:1, 3.8:1, 3.9:1, 4.0:1, 4.1:1, 4.2:1, 4.3:1, 4.4:1, 4.5:1, 4.6:1, 4.7:1, 4.8:1, 4.9:1, 5.0:1, 5.1:1, 5.2:1, 5.3:1, 5.4:1, 5.5:1, 5.6:1, 5.7:1, 5.8:1, 5.9:1, 6.0:1, 6.1:1, 6.2:1, 6.3:1, 6.4:1, 6.5:1, 6.6:1, 6.7:1, 6.8:1, 6.9:1, and 7.0:1.

In some embodiments, the particle has a weight ratio of the material in the core to the plasmonic absorber in the shell ranging from 5:1 to 1:5. In some embodiments, the particle has a weight ratio of the material in the core to the plasmonic absorber in the shell selected from the group of 5.0:1, 4.9:1, 4.8:1, 4.7:1, 4.6:1, 4.5:1, 4.4:1, 4.3:1, 4.2:1, 4.1:1, 4.0:1, 3.9:1, 3.8:1, 3.7:1, 3.6:1, 3.5:1, 3.4:1, 3.3:1, 3.2:1, 3.1:1, 3.0:1, 2.9:1, 2.8:1, 2.7:1, 2.6:1, 2.5:1, 2.4:1, 2.3:1, 2.2:1, 2.1:1, 2.0:1, 1.9:1, 1.8:1, 1.7:1, 1.6:1, 1.5:1, 1.4:1, 1.3:1, 1.2:1, 1.1:1, 1.0:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, and 1:5.

In some embodiments, the particle heater exhibits stability such that the degradation of the material by body chemicals is less than 20% as measured by the Efficacy Determination Protocol after incubating the particles in the extraction medium (serum) for 24 hours at 37° C. In some embodiments, the particle exhibits stability such that material has a degree of degradation selected from the group of about 5.0%, about 10%, about 15%, and about 20% as measured by Efficacy Determination Protocol. In some embodiments, the material has a degree of degradation in a range selected from the group of less than about 20.0%, less than about 15.0%, less than about 10.0%, less than about 5.0%, less than about 1.0%, less than about 0.5%, less than about 0.1%, and less than about 0.01% as determined by Efficacy Determination Protocol. In some embodiments, the material has a degree of degradation less than about 10.0% as determined by Efficacy Determination Protocol. In some embodiments, the material has a degree of degradation less than about 5.0% as measured by Efficacy Determination Protocol. In some embodiments, the material has a degree of degradation less than about 1.0% as measured by Efficacy Determination Protocol. In some embodiments, the material has a degree of degradation less than about 0.1% as measured by Efficacy Determination Protocol.

In some embodiments, the particle exhibits material process stability that the particle heater preserves greater than 50% of absorbance after being subject to the exogenous source process conditions.

In some embodiments, the carrier is selected based on the specific material to be encapsulated, e.g., carrier is chemically compatible with the material. In some embodiments, the carrier comprises organic or inorganic polymer. In some embodiments, the carrier is an organic polymer. In some embodiments, the carrier comprises polymer or copolymer of methylmethacrylate. In some embodiments, the carrier comprises mesoporous silica. In some embodiments, the carrier comprises a biodegradable and/or biocompatible polymer. In some embodiments, the biodegradable and/or biocompatible polymer may include, but is not limited to, a polyester, a polyurea, a polyanhydride, a polysaccharide, a polyphosphoester, a poly(ortho ester), a poly(amino acid), a protein, polyurea, and combinations thereof.

In some embodiments, the biodegradable and/or biocompatible polymer may include, but are not limited to: polymethyl methacrylate, polyester, poly caprolactone (PCL), poly(trimethylene carbonate) or other poly (alpha-esters), polyurethanes, poly(allylamine hydrochloride), poly(ester amides), poly (ortho esters), polyanyhydrides, poly (anhydride-co-imide), cross linked polyanhydrides, pseudo poly(amino acids), poly (alkylcyanoacrylates), polyphosphoesters, polyphosphazenes, chitosan, collagen, natural or synthetic poly(amino acids), elastin, elastin-like polypeptides, albumin, fibrin, polysiloxanes, polycarbosiloxanes, polysilazanes, polyalkoxysiloxanes, polysaccharides, cross-linkable polymers, thermo-responsive polymers, thermo-thinning polymers, thermo-thickening polymers, or block co-polymers of the above polymers with polyethylene glycol, and combinations thereof.

In some embodiments, the carrier comprises a hydrophobic polymer or copolymer of polymethacrylates, polycarbonate, or combinations thereof. In some embodiments, the carrier comprises polymethylmethacrylate (PMMA, Neocryl® 728 sold by DSM, T_{g}=111° C.). In some embodiments, the polymethacrylate copolymer is MMA/BMA copolymer and the weight ratio of MMA to BMA is 96:4 (e.g. Neocryl® 805 by DSM, acid value less than 1).

### 4. Remotely-Triggered Drug Delivery Particles for Chemotherapy

### 4(a). Remotely-Triggered Drug Delivery Particles for Cancer Chemotherapy

In an embodiment, this disclosure provides an externally controlled anticancer drug delivery system. Such delivery system can effectively reduce the high toxicities associated with anticancer drugs, and also improve their bioavailability.

One of the hallmarks of cancer is characterized by the uncontrolled growth of abnormal or neoplastic cells that form a tumor mass and invade adjacent tissues. Malignant cells spread by way of the blood system, by the lymphatic system to lymph nodes, by migration of cancer cells within the fluids of the peritoneal cavity, and to distant sites through a process known as metastasis.

Many compounds have been developed for the prevention and treatment of various types of cancer. However, the clinical applications of many of these anticancer therapies are limited by their unfavorable pharmacokinetic/pharmacodynamics properties such as poor water solubility, short plasma half-life (e.g. less than 30 minutes), low bioavailability, as well as difficulties in clinical administration.

For example, paclitaxel (Taxol®) is one of the known anticancer drugs and is active against a wide spectrum of cancers, including breast cancer, ovarian cancer, colon cancer, small and non-small cell lung cancer, and neck cancer. However, the clinical application of paclitaxel is limited by its limited natural source and its low solubility in water and most pharmaceutical solvents. One of the current clinical paclitaxel formulations contains solubility adjuvant Cremophor EL®. But Cremophor EL® is known to be associated with various severe side effects including hypersensitivity reaction, nephrotoxicity, neurotoxicity and cardiotoxicity.

All anticancer agents have a specific minimum dose or concentration to impart functional activity at the tumor site. Following administration, the body's natural defense mechanisms clear a large percent of the anticancer agents. Therefore, the dose or amount of the anticancer agents often are administered at an excess amount to achieve the desired functional effects at the targeted tumor site. Anticancer agents generally have various degrees of toxicity to the body. Sometimes such anticancer agents are encapsulated to minimize toxicity to the body, like Abraxane®. Even with such encapsulation, in general, there can be some leakage of the anticancer agent out of the particle which can cause toxicity. Accordingly, there exists a need to reduce the toxic effects of such anticancer agents even when they are encapsulated. The present invention provides an externally controlled anticancer drug delivery system. Such delivery system can effectively reduce the high toxicities associated with the anticancer agents and increase their efficacies, thereby increasing the therapeutic index of the agent.

Early generation polymer-drug conjugates, which aim to maximize tumor delivery, have to date demonstrated only moderate clinical benefits due to drug release prior to reaching the tumor site, and drug degradation by the body chemicals resulting from the body's defense mechanisms. For example, N-(2-hydroxypropyl)methacrylamide (HPMA) copolymer-doxorubicin and poly(ethylene glycol) (PEG)-camptothecin have not obtained the same success in the clinic as other medicines such as Doxil® (liposome-doxorubicin) and Abraxane® (nanoparticle albumin bound-paclitaxel, or nab-paclitaxel). While much of this may be related to other variables such as drug release kinetics, the lack of enough delivery of the drug to the tumor (<15% of the injected dose) represents the primary barrier to success. Recent efforts to improve this type of delivery device, such as using high molecular weight polymers which exhibit prolonged blood circulation as well as using polymers with different architectures (i.e. dendrimers and branched polymers), have achieved limited success.

Many potential anticancer drugs failed clinical trials due to their high toxicities. There is only a 3.4% chance of success for an anticancer drug to complete all the required clinical trials and reach the market. (Wong, et al, Biostatistics, 2018, pages 1-4). Many of the toxicities are caused by the high systemic concentrations inside the body. Such high concentration is necessary for a therapeutically effective amount of the anticancer drug to reach the targeted tissue.

The present invention provides a new and effective anticancer drug delivery system, namely an externally controlled anticancer drug delivery system. Such delivery system uses a particle as a vehicle. The particle comprises the herein described anticancer agent, the carrier, and the material that interacts with an exogenous source, wherein the anticancer agent is encapsulated in the carrier, and the particle optionally further comprises a shell to enclose the particle.

Various embodiments of the invention are directed to pharmaceutical compositions comprising particles, including microparticles and/or nanoparticles, for externally controlled release of the anticancer agents and method for using such pharmaceutical compositions. The pharmaceutical compositions are capable of delivering therapeutic levels of the anticancer agent to diseased tissues over the desired extended time frame, and in some embodiments, the particles may have different sizes and degradation profiles. Such pharmaceutical compositions may allow for continuous delivery of therapeutically effective amounts of the anticancer agent for a time period ranging from one day to one month in a single dose.

In some embodiments, the invention in this disclosure provides particles comprising an anticancer agent and an IR absorbing agent such that the release of the anticancer drug is accelerated by the heat generated by the IR absorbing agent after the activation by an exogenous source.

In one embodiment, the present invention provides a pharmaceutical composition comprising a particle for use in treating a cancer comprising:

an anticancer agent as described herein,
a carrier,
a material that interacts with an exogenous source,
wherein the anticancer agent is encapsulated by the carrier,
wherein the anticancer agent and the material in the particle exhibit stability such that the particle is considered passing the Efficacy Determination Protocol; wherein the particle structure is constructed such that it passes the Extractable Cytotoxicity Test; wherein the material absorbs the energy from the exogenous source and converts the energy into heat; and then the anticancer agent is released outside the particle.

In some embodiments, the present invention provides a method for treating a cancer in a patient in need thereof comprising: (1) administering to the patient a pharmaceutical composition comprising the particle of the invention, and (2) activating the particle with the exogenous source, wherein the material absorbs the energy from the exogenous source and converts the energy into heat; and wherein the heat causes the degradation of the carrier, and then the anticancer agent is released outside the particle. In an embodiment, the carrier is degraded via hydrolysis. In some embodiments, the carrier is degraded by random-chain/end-chain depolymerization.

The particles, once administered, can be activated by an exogenous source outside a human body. Once the exogenous source (e.g. an IR laser) is applied, the material that interacts with the exogenous source absorbs the energy from the exogenous source, and converts the energy into heat; and wherein the heat causes the degradation of the carrier, and then the anticancer agent is released to the targeted cancer site to impart therapeutic effects against cancer cells. Such a drug delivery system can effectively reduce the high toxicity associated with majority of the anticancer drugs and improve their bioavailability. In an embodiment, the carrier is degraded via hydrolysis. In some embodiments, the carrier is degraded by random-chain/end-chain depolymerization.

In some embodiments, the exogenous source is selected from the group of electromagnetic radiation, an electrical field, microwaves, radiowaves, an ultrasonic field, a magnetic field, and combinations thereof. In some embodiments, the exogenous source comprises near infrared radiation. In some embodiments, the exogenous source comprises a laser light that has oscillation wavelength in the near infrared region. In some embodiments, the laser light is a pulsed laser light.

In some embodiments, activation of the particle by the exogenous source (e.g. laser irradiation) in this disclosure creates a photothermal effect; that is the conversion of photonic energy into heat. The photothermal effect is highly selective being dependent upon both the location of the particles and the wavelength of the excitation source (a property which can be tuned by altering the composition of the particles).

In some embodiments, the exogenous source is a laser. In some embodiments, the material encapsulated in the particle absorbs the photons of the laser to generate heat. Such heat is localized inside the particle and causes the degradation of the carrier. In one embodiment, the carrier is degraded via hydrolysis. In some embodiments, the carrier is degraded by random-chain/end-chain depolymerization.

The advantages of the efficient localized heating achieved by the particles in this disclosure is immediately evident because the temperature increase is primary limited to the interior of particles.

In some embodiments, a wavelength of the laser irradiation is absorbed by the material contained in the particles. In some embodiments, the material has strong absorption of photons at wavelengths overlapping with the output of the various commercially available lasers. In some embodiments, the laser irradiation is delivered in a pulse duration shorter than the TRT of the particles such that the heat energy generated in the particle stays inside the particle. In some embodiments, the flow of the heat delivered to the interior of the particles can be achieved by manipulating the wavelength of the laser irradiation, pulse duration, particle size and the density of the particles at the targeted heat delivery site.

In an embodiment, the particle can be monitored after administration by an incorporated imaging agent such as fluorescent dye, a computed tomography (CT) contrast agent (like iodine) or magnetic nanoparticles. Once it travels to the targeted tissue as indicated by the imaging agent, an exogenous source is applied, then causing the anticancer agent encapsulated in the particle to be released from the particle. In one embodiment, the target tissue is selected from the group of malignant tumors, benign tissue, ulcers, polyps, fibroids, nodules, and dysplasia.

In an embodiment, the targeted drug delivery particle can be activated with an IR laser to localize the anticancer drugs to the site of the tumor with a concomitant reduction in the off-target adverse events and drug dose limiting toxicities. The carrier is sensitive to the heat generated by exciting a near infrared spectrum region (NIR) light absorbing agent encapsulated in the particle. The particle is also conjugated with cancer targeting ligands selected from the group of nucleic acids, vitamins, carbohydrates, proteins, monoclonal antibodies, peptides, and combinations thereof. Such cancer targeting ligands lead to the particle preferentially traveling to the targeted cancer site. Once the particle arrives at the targeted cancer site, the IR laser is applied, causing the material to absorb the energy from the IR laser and convert the energy into heat; and wherein the heat causes the degradation of the carrier, and then the anticancer agent is released to the targeted cancer site. In one embodiment, the carrier is degraded via hydrolysis. In some embodiments, the carrier is degraded by random-chain/end-chain depolymerization.

The anticancer drug is either fully encapsulated within the carrier, tethered to the carrier via a covalent bond, or has a high affinity for the highly charged or hydrophobic groups in a porous particle matrix.

In some embodiments, this disclosure provides remotely-triggered anticancer drug delivery particles comprising a herein described anticancer agent admixed with a material that interacts with an exogenous source. Such particles minimize the exposure of the healthy cells to the toxic effects of any anticancer agent and the material that interacts with the exogenous source which have leaked out of the particle into the body as well as minimize the entry of body fluids into the particle at concentrations that can degrade both the anticancer agent and the material inside the particle.

The encapsulation of the anticancer agent and/or the material within a carrier may reduce the degradation and the leakage mentioned above, but only to some extent due to the inherent porosity of the polymer particle.

Therefore, it is desirable that the anticancer agent is not only shielded from the attack of the body chemicals until the activation by the exogenous source, and but also the leakage of the anticancer agent is minimized until there is remote activation by the exogenous source.

To this end, the present disclosure provides a method of making a particle such that the anticancer agent encapsulated therein is stable and has minimum leakage until the activation by an exogenous source (e.g. an IR laser). The present disclosure also provides a method of designing a particle of the desired specific properties (stability and controlled release) by the feedback loop protocols.

Conventional liposomes are commonly used for anticancer drug delivery (e.g. FDA approved liposome doxorubicin formulation), however the conventional liposome vesicles tend to be very fragile and leaky thereby making them less-than ideal carrier of most cancer drug therapies. To overcome the deficiencies of the conventional liposome, in an embodiment, particles comprising a carrier are developed to encapsulate the anticancer agent such that to prevent the attack by the body chemicals. Such polymer is susceptible to hydrolysis degradation initiated by an exogenous source. Once the exogenous source (e.g. an IR laser) is applied or activated, the material that interacts with the exogenous source absorbs the energy from the exogenous source, and converts the energy into heat; and wherein the heat causes acceleration of the degradation of the carrier, and then causes the anticancer agent to be released at the targeted cancer site. The externally controlled drug delivery particles described herein improve the therapeutic index of the anticancer agent. In an embodiment, the carrier is degraded via hydrolysis. In some embodiments, the carrier is degraded by random-chain/end-chain depolymerization.

In an embodiment, the material that interacts with the exogenous source is an IR absorbing agent.

In some embodiments, the IR absorbing agent is admixed within the carrier to form a homogeneous dispersion or a solid solution. In some embodiments, the IR absorbing agent and the carrier may have oppositely charged functional group(s) (e.g. IR absorbing agent is positively charged tetrakis aminium dye, and the carrier has negatively charged functional group such as carboxylate anion of polymethacrylate polymers) such that the IR absorbing agent attaches to the carrier via ionic electrostatic interactions.

In some embodiments, the IR absorbing agent induces photothermal heating inside the particle to rapidly raise the temperature above 100° C. to enhance the delivery of the anticancer agent by accelerating the degradation of the carrier. In some embodiments, the carrier is degraded via hydrolysis. In some embodiments, the carrier is degraded by random-chain/end-chain depolymerization.

In some embodiments, the material interacting with the exogenous source has significant absorption at wavelengths ranging from 700 nm to 1500 nm, and little or no absorption in the visible region of light at wavelengths from 400 nm to 700 nm. In some embodiments, the material interacting with the exogenous source has significant absorption in the NIR wavelengths ranging from 750 nm to 1100 nm. In some embodiments, the material interacting with the exogenous source has significant absorption in the NIR wavelengths ranging from 1000 nm to 1100 nm. In some embodiments, the material interacting with the exogenous source has significant absorption in the NIR wavelengths ranging from 1000 nm to 1075 nm. In some embodiments, the material interacting with the exogenous source has significant absorption at a wavelength selected from the group of 700 nm, 766 nm, 777 nm, 780 nm, 783 nm, 785 nm, 800 nm, 808 nm, 810 nm, 820 nm, 825 nm, 900 nm, 948 nm, 950 nm, 960 nm, 980 nm, 1000 nm, 1064 nm, 1070 nm, 1071 nm, 1073 nm, 1098 nm, and 1100 nm. In some embodiments, the material interacting with the exogenous source has significant absorption at 1064 nm wavelength.

In some embodiments, the material is an IR-absorbing agent selected from the group of phthalocyanines, naphthalocyanines, and combinations thereof. In some embodiments, the IR absorbing agent is selected from the group of a tris-aminium dye, a tetrakis aminium dye, a squarylium dye, a cyanine dye, zinc copper phosphate pigment, a palladate compound, a platinate compound, and combinations thereof. In some embodiments, the IR absorbing agent comprises cyanine dyes selected from the group of indocyanine dye (ICG), 2-[2-[2-chloro-3-[[1,3-dihydro-1,1-dimethyl-3-(4-sulfobutyl)-2H-benzo[e]indol-2-ylidene]-ethylidene]-1-cyclohexen-1-yl]-ethenyl]-1,1-dimethyl-3-(4-sulfobutyl)-1H-benzo[e]indolium hydroxide inner salt, sodium salt (IR820, new ICG dye), heptamethine cyanine (IR825), heptamethine cyanine (IR780), and combinations thereof.

In some embodiments, the material interacting with the exogenous source is an IR absorbing agent. In some embodiments, the IR absorbing agent is a tetrakis aminium dye. In some embodiments, the tetrakis aminium dye is a narrow band absorber including commercially available IR absorbing agents sold under the trademark names Epolight® 1117 (peak absorption, 1071 nm), Epolight® 1151 (peak absorption, 1070 nm), or Epolight® 1178 (peak absorption, 1073 nm). In some embodiments, the tetrakis aminium dye is a broad band absorber including commercially available IR absorbing agents sold under the trademark names Epolight® 1175 (peak absorption, 948 nm), Epolight® 1125 (peak absorption, 950 nm), and Epolight® 1130 (peak absorption, 960 nm). In some embodiments, the tetrakis aminium dye is Epolight™ 1178.

In some embodiments, the IR absorbing agent is indocyanine green (ICG).

In some embodiments, the squarylium dye is a benzopyrylium squarylium dye having formula (III)

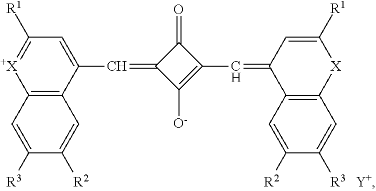

wherein each X is independently O, S, Se; Y⁺ is a counterion selected from the group of hexafluoroarsenate (AsF₆⁻), hexafluoroantimonate (SbF₆⁻), hexafluorophosphate (PF₆⁻), (C₆F₅)₄B⁻, tetrafluoroborate (BF₄⁻), and combinations thereof; each R¹ is a non-aromatic organic substituent, each R²═H or OR³, R³=cycloalkyl, alkenyl, acyl, silyl; each R³═—NR⁴R⁵, each R⁴, R⁵ is independently H, C1-8 alkyl. In some embodiments, the squarylium dye of formula (III) is a compound when R¹═—CMe₃, R²═OCHMeEt, X═O with a strong absorption at 788 nm. In some embodiments, the squarylium dye of formula (III) is a compound when R¹═—CMe₃, R²═H, R³═—NEt₂, X═O with a strong absorption at 808 nm (IR 193 dye).

In some embodiments, the IR absorbing agent may include a squarylium dye. In some embodiments, the IR absorbing agent may include a squaraine dye. In some embodiments, the IR absorbing agent may include IR 193 dye.

In some embodiments, the material interacting with the exogenous source is an inorganic IR absorbing agent. In some embodiments, the inorganic IR absorbing agent comprises one or more transition metal elements in the form of an ion such as a titanium(III), a vanadium(IV), a chromium(V), an iron(II), a nickel(II), a cobalt(II) or a copper(II) ion (corresponding to the chemical formulas Ti³⁺, VO²⁺, Cr⁵⁺, Fe²⁺, Ni²⁺, Co²⁺, and Cu²⁺). In some embodiments, the material interacting with the exogenous source is an inorganic IR absorbing agent with near-infrared absorbing properties selected from the group of zinc copper phosphate pigment ((Zn,Cu)₂P₂O₇), zinc iron phosphate pigment ((Zn,Fe)₃(PO₄)₂), magnesium copper silicate ((Mg,Cu)₂Si₂O₆ solid solutions), and combinations thereof. In some embodiments, the inorganic IR absorbing agents is a zinc iron phosphate pigment. In some embodiments, the inorganic IR absorbing agent comprises palladates or platinates.

In some embodiments, the material is a plasmonic absorber or iron oxide. In some embodiments, the plasmonic absorber is selected from the group of gold nanostructures, silver nanoparticles, graphene oxide nanomaterials and combinations thereof.

The preferred concentration of the material responsive to the exogenous source depends on the specific application. For example, in the case of an IR absorbing agent needed to absorb incident IR radiation, too little amount of IR absorbing agent can limit the temperature rise that would be desired. Likewise, too high a concentration can lead to IR absorbing agent aggregation, which can shift the absorption and reduce its absorptivity, such that the IR absorbing agent no longer absorbs the specific wavelength of light provided by the laser. In some embodiments, the material responsive to the exogenous source is present in an amount ranging from about 0.01 wt. % to about 25.0 wt. % by the total weight of the particle. In some embodiments, the material responsive to the exogenous source is present in an amount ranging from about 1.0 wt. % to about 20.0 wt. % by the total weight of the particle. In some embodiments, the material responsive to the exogenous source is present in an amount ranging from about 5.0 wt. % to about 20.0 wt. % by the total weight of the particle. In some embodiments, the material responsive to the exogenous source is present in an amount ranging from about 5.0 wt. % to about 15.0 wt. % by the total weight of the particle. In some embodiments, the material responsive to the exogenous source is present in an amount ranging from about 10.0 wt. % to about 15.0 wt. % by the total weight of the particle. In some embodiments, the material responsive to the exogenous source is present in a weight percentage by the total weight of the particle selected from the group of about 0.01 wt. %, about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. %. In some embodiments, the material responsive to the exogenous source is present in a weight percentage by the total weight of the particle selected from the group of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. %. In some embodiments, the material responsive to the exogenous source is present in a weight percentage by the total weight of the particle selected from the group of about 1.0 wt. %, about 5.0 wt. %, about 10.0 wt. %, and about 15.0 wt. %.

In some embodiments, the particle has a weight ratio of the material responsive to the exogenous source to the anticancer agent of 10:1 to 1:10. In some embodiments, the weight ratio of the material responsive to the exogenous source to the anticancer agent is 10:1, 9:1, 8:1, 7:1, 6:1, 5:1, 4:1, 3:1, 2:1, 1:1, 1;2, 1:3, 1:4, 1:5, 1:6, 1:7, 1:8, 1:9, or 1:10. In some embodiments, the weight ratio of the material responsive to the exogenous source to the anticancer agent is 1:1.

In some embodiments, the particle comprises the carrier to the material interacting with the exogenous source in a weight ratio ranging from 1:10 to 10:1. In some embodiments, the weight ratio of the carrier to the material ranges from 1:1 to 7:1. In some embodiments, the weight ratio of the carrier to the material is selected from the group of 1:10, 1:9, 1:8, 1:7, 1;6, 1:5, 1:4, 1:3, 1:2, 1:1, 2:1, 3:1, 4:1, 5:1, 6:1, 7:1, 8:1, 9:1, and 10:1. In some embodiments, the weight ratio of the carrier to the material is selected from the group of 1:1, 2:1, 3:1, 5:1, and 7:1. In some embodiments, the weight ratio of the carrier to the material is 2:1. In some embodiments, the weight ratio of the carrier to the material is 3:1. In some embodiments, the weight ratio of the carrier to the material is 4:1. In some embodiments, the weight ratio of the carrier to the material is 4.4:1. In some embodiments, the weight ratio of the carrier to the material is 5:1. In some embodiments, the weight ratio of the carrier to the material is 7:1.

In some embodiments, the carrier comprises a biocompatible and/or biodegradable polymer.

In some embodiments, the carrier comprises organic or inorganic polymer. In some embodiments, the carrier is an organic polymer. In some embodiments, the carrier comprises polymer or copolymer of methylmethacrylate. In some embodiments, the carrier comprises mesoporous silica.

In some embodiments, the polymers may include, but are not limited to: polymethyl methacrylate, polyester, poly caprolactone (PCL), poly(trimethylene carbonate) or other poly (alpha-esters), polyurethanes, poly(allylamine hydrochloride), poly(ester amides), poly (ortho esters), polyanyhydrides, poly (anhydride-co-imide), cross linked polyanhydrides, pseudo poly(amino acids), poly (alkylcyanoacrylates), polyphosphoesters, polyphosphazenes, chitosan, collagen, natural or synthetic poly(amino acids), elastin, elastin-like polypeptides, albumin, fibrin, polysiloxanes, polycarbosiloxanes, polysilazanes, polyalkoxysiloxanes, polysaccharides, cross-linkable polymers, thermo-responsive polymers, thermo-thinning polymers, thermo-thickening polymers, or block co-polymers of the above polymers with polyethylene glycol, and combinations thereof.

In some embodiments, the carrier comprises a hydrophobic polymer or copolymer of polymethacrylates, polycarbonate, or combinations thereof. In some embodiments, the carrier comprises polymethylmethacrylate (PMMA, Neocryl® 728 sold by DSM, T_{g}=111° C.). In some embodiments, the polymethacrylate copolymer is MMA/BMA copolymer and the weight ratio of MMA to BMA is 96:4 (e.g. Neocryl® 805 by DSM, acid value less than 1).

In one embodiment, the carrier is a polyester. Polyesters are a class of polymers characterized by ester linkages in the backbone, such as poly (lactic acid) (PLA), poly (glycolic acid) (PGA), PLGA, etc. PLGA is one of the commonly used polymers in developing particulate drug delivery systems. PLGA degrades via hydrolysis of its ester linkages in the presence of water. Due to the hydrophobic nature of PLGA, PLGA particles with core-shell structures are prepared through various emulsification processes and hydrophilic drugs could be encapsulated in the hydrophilic shell of the particles, while hydrophobic drugs tend to distribute in the hydrophobic core.

Surface degradation and bulk degradations are two typical modes of polymer degradation. In a surface degrading polymer, degradation is confined to the outer surface of the particle. In a bulk degrading polymer, however, degradation occurs homogeneously throughout the particle. Water intrusion into the interior of the particle during hydrolysis is of significant importance for the polymer degradation kinetics as well as drug release kinetics.

Upon contact with biological fluids, PLGA is degraded into shorter chain acids. PLGA particles are known to be bulk eroding material (degradation takes place throughout the particle) because the diffusion of biological fluids into PLGA particles is much more rapid than the subsequent ester hydrolysis. Due to the concentration gradient and slow diffusion process, an accumulation of the carboxylic acid resulting from hydrolysis can lead to a significant drop in local-pH and subsequently accelerates the polymer degradation. The ester bond cleavage during PLGA degradation is accelerated due to the auto-catalysis by acidic protons.

Because these polymers generally cannot intrinsically absorb light in the NIR region, a near infrared absorbing compound (i.e. IR absorbing agent) is incorporated into the polymer particle matrix in order to enhance the absorption in the NIR region. After the activation by an exogenous source (e.g. laser irradiation), the heat generated by the incorporated IR absorbing agent inside the particle would be expected to raise the temperature rapidly within the PLGA particle, such that the degradation of the PLGA carrier would be accelerated. In an embodiment, the PLGA carrier is degraded via hydrolysis. In some embodiments, the PLGA is degraded by random-chain/end-chain depolymerization.

The release of paclitaxel from PLGA (lactide/glycolide=75:25) particles was reported to be extremely slow. This is because the drug is highly hydrophobic and PLGA is also hydrophobic. It was reported that only 50% of the paclitaxel could be released within 3 months. However, the continuous release of an anticancer drug from a controlled release delivery device for one week to one month is usually required for effective treatment of cancer. Therefore, it is desirable to have particles comprising an IR absorbing agent and a herein described anticancer agent. Once the IR absorbing agent is activated by an exogenous source (e.g., laser), the IR absorbing agent will cause the release acceleration of the anticancer agent.

The particles in this disclosure are designed to address the above described challenges associated with anticancer drug delivery particles based on biocompatible carrier.

In some embodiments, the carrier for the particle comprises a lipid, an inorganic polymer, organic polymer, or combinations thereof.

In some embodiments, the carrier may include, but are not limited to, a polyester, a polyurea, a polyanhydride, a polysaccharide, a polyphosphoester, a poly(ortho ester), a poly(amino acid), a protein, and combinations thereof.

In some embodiments, the carrier is a biodegradable polymer selected from the group of poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (ε-lysine), poly-L-lysine (PLL), poly(valeric acid), and poly-L-glutamic acid, poly(ester amide), poly(ester ether) diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

In some embodiments, the carrier comprises polyester selected from the group of poly(lactic acid) (PLA), poly(glycolic acid) (PGA), PLGA, and combinations thereof.

Copolymers of PEG or derivatives thereof with any of the polymers described above may be used to make the polymeric particles. In certain embodiments, the PEG or derivatives may locate in the interior positions of the triblock copolymer (e.g. PLA-PEG-PLA). Alternatively, the PEG or derivatives may locate near or at the terminal positions of the block copolymer. In certain embodiments, the microparticles or nanoparticles are formed under conditions that allow regions of PEG to phase separate or otherwise to reside on the surface of the particles.

In some embodiments, the carrier comprises PLGA. PLGA denotes a copolymer (or co-condensate) of lactic acid and glycolic acid. The PLGA copolymers for use in the present invention are preferably biodegradable, i.e. they degrade in an organism over time by enzymatic or hydrolytic action or by similar mechanisms, thereby producing pharmaceutically acceptable degradation products, and biocompatible, i.e. that do not cause toxic or irritating effects or immunological rejection when brought into contact with a body fluid. The lactic acid units may be L-lactic acid, D-lactic acid or a mixture of both.

In some embodiments, the anticancer agent is hydrophobic. The hydrophobic anticancer agent release characteristics may be modulated by varying the molar ratio of the hydrophobic repeating unit polyglycolide to the hydrophilic repeating unit polylactide in a PLGA copolymer. In some embodiments, the proportion of lactic acid units and glycolic acids units within the copolymer may be in a range selected from the group of 10:90 to 90:10, from 15:85 to 85:15, from 20:80 to 80:20, from 25:75 to 75:25, from 30:70 to 70:30, from 35:65 to 65:35, from 40:60 to 60:40, and from 45:55 to 55:45 and the PLGA has a number average molecular weight ranging from 450 Da to 15,000 Da. In some embodiments, the polymer comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45, and has a number average molecular weight ranging from 450 Da to 10,000 Da. In some embodiments, the polymer comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45, and has a number average molecular weight ranging from 10,000 Da to 15,000 Da.

In some embodiments, the carrier comprises a PLGA having a lactide:glycolide molar ratio from 15:85 to 85:15, 25:75 to 75:25, 40:60 to 60:40, or 45:55 to 55:45, and has a number average molecular weight ranging from 450 Da to 15,000 Da. In some embodiments, the polymer comprises a PLGA having a lactide:glycolide molar ratio from 15:85 to 85:15, 25:75 to 75:25, 40:60 to 60:40, or 45:55 to 55:45, and has a number average molecular weight ranging from 570 Da to 8000 Da. In some embodiments, the polymer comprises a PLGA having a lactide:glycolide molar ratio from 25:75 to 75:25, 40:60 to 60:40, or 45:55 to 55:45 and has a number average molecular weight ranging from 570 Da to 3000 Da. In some embodiments, the polymer comprises a PLGA having a lactide:glycolide molar ratio from 25:75 to 75:25, 40:60 to 60:40, or 45:55 to 55:45 and has a number average molecular weight ranging from 1000 Da to 10,000 Da. In some embodiments, the polymer comprises a PLGA having a lactide:glycolide molar ratio from 25:75 to 75:25, 40:60 to 60:40, or 45:55 to 55:45 and has a number average molecular weight selected from the group of 5000 Da, 6000 Da, 7000 Da, 8000 Da, 9000 Da, 10,000 Da, 11, 000 Da, 12,000 Da, 13,000 Da, 14,000 Da, and 15,000 Da.

In some embodiments, the PLGA has a lactide:glycolide monomer ratio ranging from 70:30 to 30:70 and an average molecular weight of 4,000 Da, or 11,000 Da. In some embodiments, the PLGA has a 70:30 lactide:glycolide monomer ratio and a number average molecular weight of 1500 Da, or 4500 Da (PLG 1600HL™). In some embodiments, the PLGA has a 75:25 lactide:glycolide monomer ratio and a weight average molecular weight of 90,000 Da to 126,000 Da (PLGA 7525). In some embodiments, the PLGA has a 50:50 lactide:glycolide monomer ratio and a number average molecular weight 2515 Da (Resomer RG® 502H).

In some embodiments, copolymer of D, L isomer of lactic acids is applied to modulate the polymer water solubility property and cancer drug release characteristics. In some embodiments, the polymer is a poly(L-co-D,L-lactic acid (PLDLA) in a L-LA to D,L-LA monomer ratio selected from the group of 90:10, 85:15, 80:20, 75:25, 70:30, 65:35, 60:40, and 55:45 to form micro/nanoparticle to encapsulate hydrophobic anticancer drug such as paclitaxel. In some embodiments, PLDLA has a number average molecular weight ranging from 2000 Da to 50,000 Da (or a weight average molecular weight Mw ranging from 3400 Da to 85,000 Da, polydispersity 1.7 (Mw/Mn)). In some embodiments, the carrier comprises a poly(L-co-D,L-lactic acid (PLDLA) in the 70:30 L-LA to D,L-LA monomer ratio and has a number average molecular weight ranging from 2000 Da to 50,000 Da (or a weight average molecular weight Mw ranging from 3400 Da to 85,000 Da, polydispersity 1.7 (Mw/Mn)). The PLDLA in 70:30 monomer ratio is an amorphous polymer that facilitates the degradation. The PLDLA polymer has excellent biodegradability, biocompatibility and controlled degradation characteristics. The PLDLA particles are localized to the tumor after administration, and excited with laser such that the drug releases outside of the particle in controlled fashion, for example, the release greater than 90% of the hydrophobic anticancer agent is over a period of time selected from the group of 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, and 30 days. The PLDLA particles release greater than 50%, 55%, 60%, 65%, 70%, 75%, 80%, 85%, 90%, 95% or 100% of the hydrophobic anticancer agent over a 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30-day period.

In some embodiments, the hydrophilic polymer segment is incorporated into the hydrophobic PLGA or PLA polymer backbone to modulate the hydrophobic anticancer drug release characteristics. In some embodiments, the hydrophilic segment comprises polyethylene glycol (PEG), polyalkyleneoxide, block copolymer of polyalkyleneoxide, or dendritic polyglycerol. In some embodiments, the hydrophilic segment is polyethylene glycol having a number average molecular weight ranging from 500 Da to 10,000 Da. In some embodiments, the PEG segments has a number average molecular weight selected from the group of 500 Da, 550 Da, 600 Da, 650 Da, 700 Da, 750 Da, 800 Da, 850 Da, 900 Da, 950 Da, 1000 Da, 1050 Da, 1100 Da, 1150 Da, 1200 Da, 1250 Da, 1300 Da, 1350 Da, 1400 Da, 1450 Da, 1500 Da, 1550 Da, 1600 Da, 1650 Da, 1700 Da, 1750 Da, 1800 Da, 1850 Da, 1900 Da, 1950 Da, 2000 Da, 2050 Da, 2100 Da, 2105 Da, 2200 Da, 2250 Da, 2300 Da, 2350 Da, 2400 Da, 2450 Da, 2500 Da, 2550 Da, 2600 Da, 2700 Da, 2800 Da, 2900 Da, 3000 Da, 3100 Da, 3200 Da, 3300 Da, 3400 Da, 3500 Da, 3600 Da, 3700 Da, 3800 Da, 3900 Da, 4000 Da, 4100 Da, 4200 Da, 4300 Da, 4400 Da, 4500 Da, 4600 Da, 4700 Da, 4800 Da, 4900 Da, 5000 Da, 5100 Da, 5200 Da, 5300 Da, 5400 Da, 5500 Da, 5600 Da, 5700 Da, 5800 Da, 5900 Da, 6000 Da, 6100 Da, 6200 Da, 6300 Da, 6400 Da, 6500 Da, 6600 Da, 6700 Da, 6800 Da, 6900 Da, 7000 Da, 7100 Da, 7200 Da, 7300 Da, 7400 Da, 7500 Da, 7600 Da, 7700 Da, 7800 Da, 7900 Da, 8000 Da, 8100 Da, 8200 Da, 8300 Da, 8400 Da, 8500 Da, 8600 Da, 8700 Da, 8800 Da, 8900 Da, 9000 Da, 9100 Da, 9200 Da, 9300 Da, 9400 Da, 9500 Da, 9600 Da, 9700 Da, 9800 Da, 9900 Da, and 10,000 Da. The advantages provided by the hydrophilic segments such as PEG would be improvement of the biocompatibility of the particle due to the fact that most biological environments are hydrophilic in nature and biocompatibility is correlated directly with the degree of hydrophilicity of the particle surface. In some embodiments, the PEG, PLGA, and PLA block copolymer is a triblock polymer is PLA-PEG-PLA or PLGA-PEG-PLGA. In some embodiments, the triblock copolymer is PLA-PEG-PLA, wherein the PLA block has a number average molecular weight of 450 Da to 9000 Da, and the PEG block has a number average molecular weight of 200 Da to 9000 Da. In some embodiments, the triblock copolymer is PLA-PEG-PLA, wherein the PLA block has a number average molecular weight of 450 Da to 5000 Da, and the PEG block has a number average molecular weight of 200 Da to 7500 Da. In some embodiments, the triblock copolymer is PLA-PEG-PLA, wherein the PLA block has a number average molecular weight of 500 Da to 3000 Da, and the PEG block has a number average molecular weight of 200 Da to 3500 Da. In some embodiments, the triblock copolymer is PLA-PEG-PLA, wherein the PLA block has a number average molecular weight of 2000 Da to 3000 Da, and the PEG block has a number average molecular weight of 3000 Da to 3500 Da. In some embodiments, the triblock copolymer is PLA-PEG-PLA, wherein the PLA block has a number average molecular weight of 2000 Da, and the PEG block has a number average molecular weight of 10,000 Da (PLA(2K)-b-PEG(10K)-b-PLA(2K).

In some embodiments, the PEG modified polyester polymer is di-block copolymer of poly(sebacic acid) and polyethylene glycol (PSA-PEG), wherein the PSA has a number average molecular weight ranging from 500 Da to 15,000 Da and the PEG segment has a number average molecular weight ranging from 450 Da to 15,000 Da. In some embodiments, the carrier is a PSA-PEG diblock copolymer, wherein the PSA segment of the diblock copolymer PSA-PEG has a number average molecular weight ranging from 500 Da to 10,000 Da and the PEG segment of the diblock copolymer PSA-PEG has a number average molecular weight ranging from 450 Da to 10,000 Da. In some embodiments, the carrier is a PSA-PEG diblock copolymer, wherein the PSA segment of the diblock copolymer PSA-PEG has a number average molecular weight ranging from 500 Da to 10,000 Da and the PEG segment of the diblock copolymer PSA-PEG has a number average molecular weight ranging from 450 Da to 5,000 Da.

In some embodiments, the blending of two different polyesters having different number average molecular weight and different hydrophobicity is applied to modulate the polymer water solubility property and cancer drug release characteristics.

In some embodiments, the carrier comprises a mixture of poly(aspartic acid-co-1-lactide)(PAL) and polyethylene glycol such that the particle formed thereof comprises PEG in its shell to enclose the hydrophobic core. In some embodiments, the carrier comprises poly(aspartic acid-co-1-lactide) and PEG having a weight ratio of poly(aspartic acid-co-1-lactide) to PEG ranging from 1:10 to 10:1. In some embodiments, the weight ratio of poly(aspartic acid-co-1-lactide) to PEG in the particle ranges from 1:1 to 7:1. In some embodiments, the weight ratio of poly(aspartic acid-co-1-lactide) to PEG in the particle is selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1. In some embodiments, the weight ratio of poly(aspartic acid-co-1-lactide) to PEG in the particle is selected from the group of 1:1, 2:1, 3:1, 5:1, and 7:1. In some embodiments, the weight ratio of poly(aspartic acid-co-1-lactide) to PEG in the particle is 1:1.

In some embodiments, the weight ratio of poly(aspartic acid-co-1-lactide) to PEG in the particle is 3:1. In some embodiments, the weight ratio of poly(aspartic acid-co-1-lactide) to PEG in the particle is 5:1. In some embodiments, the weight ratio of poly(aspartic acid-co-1-lactide) to PEG in the particle is 7:1. In some embodiments, the weight ratio of poly(aspartic acid-co-1-lactide) to PEG in the particle is 1:2. In some embodiments, the weight ratio of poly(aspartic acid-co-1-lactide) to PEG in the particle is 1:3. In some embodiments, the weight ratio of poly(aspartic acid-co-1-lactide) to PEG in the particle is 1:4. In some embodiments, the weight ratio of poly(aspartic acid-co-1-lactide) to PEG in the particle is 1:5. In some embodiments, the weight ratio of poly(aspartic acid-co-1-lactide) to PEG in the particle is 1:7.

In some embodiments, the carrier comprises a mixture of poly(l-lactic acid) (PLLA) and poly(aspartic acid-co-1-lactide) (PAL). The degradation rate becomes higher for the carrier containing PAL with higher molar ratios of lactide to aspartic acid units [LA]/[Asp].

In some embodiments, the polymer comprises a mixture of (i) a first PLGA having a first number average molecular weight Mn1 ranging from 2000 Da to 3000 Da, and (ii) a second PLGA having a second number average molecular weight Mn2 ranging from 3600 Da to 10500 Da. In some embodiments, the polymer comprises a mixture of (i) a first PLGA having a first number average molecular weight Mn1 ranging from 2000 Da to 3000 Da, and (ii) a second PLGA having a second number average molecular weight Mn2 ranging from 667 Da to 9,000 Da. In some embodiments, the polymer comprises a mixture of (i) a first PLGA having a first number average molecular weight Mn1 ranging from 2000 Da to 3000 Da, and (ii) a second PLGA having a second number average molecular weight Mn2 ranging from 667 Da to 4500 Da. In some embodiments, the polymer comprises a mixture of (i) a first PLGA having a first number average molecular weight Mn1 ranging from 2000 Da to 3000 Da, and (ii) a second PLGA having a second number average molecular weight Mn2 ranging from 570 Da to 1667 Da. In some embodiments, the first and second PLGA have a lactide:glycolide molar ratio from 5:95 to 95:5, 10:90 to 90:10, 15:85 to 85:15, 25:75 to 75:25, 40:60 to 60:40, or 45:55 to 55:45.

In general, both the first PLGA and the second PLGA are monodisperse copolymers. A molecular weight distribution centered around an average value is meant to define the essentially monomodal molecular weight distribution associated with the number average value. In general, the poly(lactide-co-glycolide) has a polydispersity (which is the quotient of the weight average molecular weight over the number average weight) of not more than 3.2. Molecular weights of polymers can be measured by size exclusion chromatography (SEC). Waters HPLC equipment (Waters 515) fitted with 4 coupled Waters Styragel columns as the stationary phase, tetrahydrofuran at 1 mL/min flow rate as the mobile phase, and a Waters 410 refractometer as the detector is used. Molecular weights are expressed as number-average molecular weights (Mn) and weight-average molecular weight (Mw) with polydispersity (PD=Mw/Mn). Molecular weight is calculated by the system calibration software using polystyrene standards of known molecular weights.

In some embodiments, the PLGA has a polydispersity (PD=Mw/Mn) ranging from 1.0 to 10.0. In some embodiments, the PLGA has a polydispersity ranging from 1.0 to 3.0. In some embodiments, the PLGA has a polydispersity ranging from 2.0 to 3.0. In some embodiments, the PLGA has a polydispersity selected from the group of 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, and 3.0. In some embodiments, the PLGA has a polydispersity ranging from 1.0 to 2.0. In some embodiments, the PLGA has a polydispersity of about 1.2.

In some embodiments, the first PLGA is Resomer® Condensate RG 50:50, Mn 2300 g/mol (Boehringer Ingelheim Pharma GmbH & Co. KG, Ingelheim, Germany (also referred to as Resomer Mn 2300 herein) which is a copolymer of D,L-lactate and glycolate in a molar ratio from 45:55 to 55:45 and has a molecular weight Mn of 2000-2500 g/mol; and a second PLGA is Resomer® Condensate RG 50:50, Mn 800 g/mol, obtained from Boehringer Ingelheim Pharma GmbH & Co. KG (also referred to as Resomer Mn 800 herein) which is a copolymer of D,L-lactate and glycolate in a molar ratio from 45:55 to 55:45 and has a molecular weight Mn of 800 g/mol.

In some embodiments, the mixture comprises the first PLGA and the second PLGA in a weight ratio of first PLGA to second PLGA ranging from 10:1 to 1:10. In some embodiments, the mixture comprises the first PLGA and the second PLGA in a weight ratio of first PLGA to second PLGA in a ratio selected from the group of 10:1, 9.9:1, 9.8:1, 9.7:1, 9.6:1, 9.5:1, 9.4:1, 9.3:1, 9.2:1, 9.1:1, 9:1, 8.9:1, 8.8:1, 8.7:1, 8.6:1, 8.5:1, 8.4:1, 8.3:1, 8.2:1, 8.1:1, 8:1, 7.9:1, 7.8:1, 7.7:1, 7.6:1, 7.5:1, 7.4:1, 7.3:1, 7.2:1, 7.1:1, 7:1, 6.9:1, 6.8:1, 6.7:1, 6.6:1, 6.5:1, 6.4:1, 6.3:1, 6.2:1, 6.1:1, 6:1, 5.9:1, 5.8:1, 5.7:1, 5.6:1, 5.5:1, 5.4:1, 5.3:1, 5.2:1, 5.1:1, 5:1, 4.9:1, 4.8:1, 4.7:1, 4.6:1, 4.5:1, 4.4:1, 4.3:1, 4.2:1, 4.1:1, 4:1, 3.9:1, 3.8:1, 3.7:1, 3.6:1, 3.5:1, 3.4:1, 3.3:1, 3.2:1, 3.1:1, 3:1, 2.9:1, 2.8:1, 2.7:1, 2.6:1, 2.5:1, 2.4:1, 2.3:1, 2.2:1, 2.1:1, 2:1, 1.9:1, 1.8:1, 1.7:1, 1.6:1, 1.5:1, 1.4:1, 1.3:1, 1.2:1, 1.1:1, 1:1, 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.8, 1:9.9, and 1:10.

In some embodiments, the carrier comprises PLGA 75:25 having a number average molecular weight (Mn) of 2,000 Da. In some embodiments, the carrier comprises a polymer mixture containing about 80 wt. % of PLGA (75:25, Mn 8000 Da) and 20 wt. % of PLGA (75:25)-PEG (Mn 8,000 Da (PLGA)-Mn 2,000 Da (PEG)). In some embodiments, the carrier comprises Poly(sebacic anhydride) having a number average molecular weight (Mn) of 10,000 Da. In some embodiments, the carrier comprises a polymer mixture containing 70 wt. % PLGA 90:10 (Mn 6,000 Da), 20 wt. % poly (malic acid-co-glycolide) 90:10 (Mn 6000 Da) and 10 wt. % PLGA (75:25)-PEG (Mn 4000 Da (PLGA)-Mn 2000 Da (PEG)). In some embodiments, the carrier comprises 70 wt. % PLGA 85:15 (Mn 5000 Da), 20 wt. % poly (aspartic acid-co-glycolide) 90:10 (Mn 5000 Da) and 10 wt. % PLGA (75:25)-PEG (Mn 3000 Da (PLGA)-Mn 2000 Da (PEG)]). In some embodiments, the carrier comprises 60 wt. % of PLGA (75:25, Mn 5000 Da) to 40 wt. % of PLGA (75:25)-PEG (Mn 5000 Da (PLGA)-Mn 2000 Da (PEG)].

In some embodiments, the carrier comprises 2 parts of 1,2-distearoyl-sn-glycero-3-phosphoglycerol (DSPG), 1 part of cholesterol, and 0.2 part of 1,2-distearoyl-sn-glycero-3-phosphoethanolamine (DSPE-PEG2000). In some embodiments, the carrier comprises 2 parts sphingomyelin (egg), 1 part cholesterol and 0.2 parts of 1,2-distearoyl-sn-glycero-3-phosphoethanolamine (DSPE-PEG2000).

In some embodiments, the blending of a hydrophobic polyester with a hydrophilic polymer is applied to modulate the release characteristics of a hydrophobic anticancer drug.

In some embodiments, the particle comprises the carrier to the anticancer agent in a weight ratio ranging from 1:10 to 10:1. In some embodiments, the weight ratio of the carrier to the anticancer agent ranges from 1:1 to 7:1. In some embodiments, the weight ratio of the carrier to the anticancer agent is selected from the group of 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, and 10:1. In some embodiments, the weight ratio of the carrier to the anticancer agent is selected from the group of 1:1, 2:1, 3:1, 5:1, and 7:1. In some embodiments, the weight ratio of the carrier to the anticancer agent is 7:3. In some embodiments, the weight ratio of the carrier to the anticancer agent is 2:1. In some embodiments, the weight ratio of the carrier to the anticancer agent is 3:1. In some embodiments, the weight ratio of the carrier to the anticancer agent is 4:1. In some embodiments, the weight ratio of the carrier to the anticancer agent is 5:1. In some embodiments, the weight ratio of the carrier to the anticancer agent is 7:1. In some embodiments, the weight ratio of the carrier to the anticancer agent is 9:1.

In some embodiments, the amount of the carrier is present in a weight percentage by the total weight of the particle ranging from about 5 wt. % to about 95 wt. %. In some embodiments, the amount of the carrier is present in a weight percentage selected from the group of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, about 15.0 wt. %, about 20.0 wt. %, about 25.0 wt. %, about 30.0 wt. %, about 35.0 wt. %, about 40.0 wt. %, about 45.0 wt. %, about 50.0 wt. %, about 55.0 wt. %, about 60.0 wt. %, about 65.0 wt. %, about 70.0 wt. %, about 75.0 wt. %, about 80.0 wt. %, about 85.0 wt. %, about 90.0 wt. %, about 95.0 wt. %, about 96.0 wt. %, about 97 wt. %, about 98.0 wt. %, about 99.0 wt. %, about 99.9 wt. %, and about 99.99 wt. % by the total weight of the particle. In some embodiments, the carrier is present in a weight percentage by the total weight of the particle ranging from about 85.0 wt. % to about 98.0 wt. %. In some embodiments, the carrier is present in a weight percentage by the total weight of the particle ranging from about 50.0 wt. % to about 95.0 wt. %. In some embodiments, the carrier is present in a weight percentage by the total weight of the particle ranging from about 5.0 wt. % to about 50 wt. %.

In some embodiments, the anticancer agent is covalently bonded to the carrier via a thermally activatable fragmentation linker. In some embodiments, the thermally activatable fragmentation linker comprises substituted and unsubstituted carbonates, carbamates, esters, lactams, lactones, amides, imides, oximes, sulfonates, phosphates, or phosphonates. Upon exposure to the exogenous laser irradiation, the IR absorbing agent encapsulated within the particle absorbs the near infrared irradiation from the laser and converts it to heat such that the temperature inside the particle is raised rapidly. Accordingly, the anticancer drug is released from the carrier due to the degradation of the thermally activatable fragmentation linker that anchors the anticancer agent to the carrier.

In some embodiments, at least a portion of the exterior surface of the particle has a modification that is polar, non-polar, charged, ionic, basic, acidic, reactive, hydrophobic, or hydrophilic. In some embodiments, the particle may further comprise a shell to enclose the particle.

In some embodiments, the particle further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof.

In some embodiments, the shell results from the use of an alkyltrimethoxysilane reagent (CnTMS, n is an integer ranging from 1 to 12) in the Stöber synthesis. In some embodiments, the shell results from the use of C1-C7 alkyl trimethoxysilane reagent in the Stöber synthesis. In some embodiments, the shell results from the use of C1-C7 alkenyl trimethoxysilane reagent in the Stöber synthesis. In some embodiments, the shell results from the use of C1-C7 alkynyl trimethoxysilane reagent in the Stöber synthesis. In some embodiments, the C1-C7 alkyl group, the C1-C7 alkenyl group, or the C1-C7 alkynyl group may be linear or branched. In some embodiments, the shell results from the use of C2-C6 linear alkyl trimethoxysilane reagent in the Stöber synthesis. In some embodiments, the shell results from the use of C2-C4 linear alkyl trimethoxysilane reagent in the Stöber synthesis. In some embodiments, the shell results from the use of ethyl (C2) trimethoxysilane reagent in Stöber synthesis. In some embodiments, the shell results from the use of vinyltrimethoxysilane (VTMS) reagent in Stöber synthesis. In some embodiments, the shell results from the condensation reaction of hydroxymethylsilanetriol prepared by the hydrolysis of hydroxymethyltrichlorosilane. In some embodiments, the shell results from the condensation reaction of (3-mercaptopropyl)silanetriol prepared by the hydrolysis of (3-mercaptopropyl)trimethoxysilane. The silicate shell having hydroxymethyl and 3-mercaptopropyl modification on the surface provides reactive functional group for further engineering of the particle with targeting groups and other functional surface modifications.

In an embodiment, this disclosure provides a particle for use in the remotely-triggered delivery of anticancer agent to tumor site comprising:

a material that interacts with an exogenous source, wherein the material is an IR absorbing agent selected from the group of a tris-aminium dye, a di-imonium dye, a tetrakis aminium dye, a cyanine dye, a squaraine dye, a zinc iron phosphate pigment, and combinations thereof,
a carrier comprising a polymer selected from the group of PLGA, polycarbonate, polymethylmethacrylate (PMMA, Neocryl® 728), MMA/BMA copolymer having the weight ratio of MMA to BMA at 96:4 (e.g. Neocryl® 805) (e.g. Neocryl® 805), and combinations thereof;
an anticancer agent is curcumin or paclitaxel;
wherein the particle heater has a median particle size less than 1 μm,
wherein the material interacting with an exogenous source is encapsulated by the carrier to form a particle,
wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells. In some embodiments, the particle further passes the Thermal Cytotoxicity Test.

In some embodiments, this disclosure provides a particle for use in the remotely-triggered delivery of anticancer agent to tumor site comprising: (a) a tetrakis aminium dye Epolight™ 1117, (b) PLGA; (c) an anticancer agent is curcumin or paclitaxel; wherein the particle heater has a median particle size less than 1 μm.

In some embodiments, this disclosure provides a particle for use in the remotely-triggered delivery of anticancer agent to tumor site comprising:

a material that interacts with an exogenous source, wherein the material is an IR absorbing agent selected from the group of a tris-aminium dye, a di-imonium dye, a tetrakis aminium dye, a cyanine dye, a squaraine dye, a zinc iron phosphate pigment, and combinations thereof,
a carrier comprising a polymer selected from the group of polymethylmethacrylate (PMMA, Neocryl® 728), MMA/BMA copolymer having the weight ratio of MMA to BMA at 96:4 (e.g. Neocryl® 805), and combinations thereof;
an anticancer agent is curcumin or paclitaxel;
wherein the particle heater has a median particle size less than 1 μm,
wherein the material interacting with an exogenous source is encapsulated by the carrier to form a particle,
wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells. In some embodiments, the particle further passes the Thermal Cytotoxicity Test.

In some embodiments, this disclosure provides a particle for use in the remotely-triggered delivery of anticancer agent to tumor site comprising:

a tetrakis aminium dye,
MMA/BMA copolymer having the weight ratio of MMA to BMA at 96:4 (e.g. Neocryl® 805) (e.g. Neocryl® 805);
an anticancer agent is curcumin or paclitaxel;
wherein the particle heater has a median particle size less than 1 μm,
wherein the material interacting with an exogenous source is encapsulated by the carrier to form a particle,
wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells. In some embodiments, the particle further passes the Thermal Cytotoxicity Test.

In some embodiments, this disclosure provides a particle for use in the remotely-triggered delivery of anticancer agent to tumor site comprising:

a tetrakis aminium dye Epolight™ 1117,
MMA/BMA copolymer (PMMA/BMA) having the weight ratio of MMA to BMA at 96:4 (e.g. Neocryl® 805) (e.g. Neocryl® 805);
an anticancer agent is curcumin or paclitaxel;
wherein the particle heater has a median particle size less than 1 μm,
wherein the material interacting with an exogenous source is encapsulated by the carrier to form a particle,
wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells. In some embodiments, the particle further passes the Thermal Cytotoxicity Test. In some embodiments, the PMMA/BMA comprises Epolight™ IR 1117 and curcumin. In some embodiments, the PMMA/BMA particles as described herein comprise Epolight™ IR 1117 and paclitaxel.

In some embodiments, the PMMA/BMA-Epolight™ IR 1117-curcumin particles as described herein contain about 70.0 wt. % to 95.0% of Neocryl® 805 PMMA/BMA, about 1.0 wt. % to about 9.0 wt. % of Epolight™ IR 1117, and about 1.0 wt. % to about 5.0 wt. % of curcumin.

In some embodiments, the PMMA/BMA-Epolight™ IR 1117-curcumin particles as described herein contain about 85.0 wt. % to 95.0% of Neocryl® 805 PMMA/BMA, about 5.0 wt. % to about 9.0 wt. % of Epolight™ IR 1117, and about 1.0 wt. % to about 5.0 wt. % of curcumin.

In some embodiments, the PMMA/BMA-Epolight™ IR 1117-curcumin particles as described herein contain about 1.0 wt. %, 1.1 wt. %, 1.2 wt. %, 1.3 wt. %, 1.4 wt. %, 1.5 wt. %, 1.6 wt. %, 1.7 wt. %, 1.8 wt. %, 1.9 wt. %, 2.0 wt. %, 2.1 wt. %, 2.2 wt. %, 2.3 wt. %, 2.4 wt. %, 2.5 wt. %, 2.6 wt. %, 2.7 wt. %, 2.8 wt. %, 2.9 wt. %, 3.0 wt. %, 3.1 wt. %, 3.2 wt. %, 3.3 wt. %, 3.4 wt. %, 3.5 wt. %, 3.6 wt. %, 3.7 wt. %, 3.8 wt. %, 3.9 wt. %, 4.0 wt. %, 4.1 wt. %, 4.2 wt. %, 4.3 wt. %, 4.4 wt. %, 4.5 wt. %, 4.6 wt. %, 4.7 wt. %, 4.8 wt. %, 4.9 wt. %, and 5.0 wt. % of curcumin. In some embodiments, the PMMA/BMA-Epolight™ IR 1117-curcumin particles as described herein contain about 2.0 wt. % of curcumin.

In some embodiments, the PMMA/BMA-Epolight™ IR 1117-curcumin particles as described herein contain about 90.0 wt. % of Neocryl® 805 PMMA/BMA, about 8.0 wt. % of Epolight™ IR 1117, and about 2.0 wt. % of curcumin.

In some embodiments, the PMMA/BMA-Epolight™ IR 1117-curcumin particles as described here further comprise a silica shell to encase the curcumin core particle to form core-shell particle. In some embodiments, the shell comprises a cross-linked inorganic polymer selected from the group of mesoporous silica, organo-modified silicate polymer derived from condensation of organotrisilanol or halotrisilanol, and combinations thereof.

In some embodiments, the shell encasing the PMMA/BMA-Epolight™ IR 1117-curcumin particles results from the use of an alkyltrimethoxysilane reagent (CnTMS, n is an integer ranging from 1 to 12) in the Stöber synthesis. In some embodiments, the shell results from the use of C1-C7 alkyl trimethoxysilane reagent in the Stöber synthesis. In some embodiments, the shell results from the use of C1-C7 alkenyl trimethoxysilane reagent in the Stöber synthesis. In some embodiments, the shell results from the use of C1-C7 alkynyl trimethoxysilane reagent in the Stöber synthesis. In some embodiments, the C1-C7 alkyl group, the C1-C7 alkenyl group, or the C1-C7 alkynyl group may be linear or branched. In some embodiments, the shell results from the use of C2-C6 linear alkyl trimethoxysilane reagent in the Stöber synthesis. In some embodiments, the shell results from the use of C2-C4 linear alkyl trimethoxysilane reagent in the Stöber synthesis. In some embodiments, the shell results from the use of ethyl (C2) trimethoxysilane reagent in Stöber synthesis. In some embodiments, the shell results from the use of vinyltrimethoxysilane (VTMS) reagent in Stöber synthesis. In some embodiments, the shell results from the condensation reaction of hydroxymethylsilanetriol prepared by the hydrolysis of hydroxymethyltrichlorosilane. In some embodiments, the shell results from the condensation reaction of (3-mercaptopropyl)silanetriol prepared by the hydrolysis of (3-mercaptopropyl)trimethoxysilane. The silicate shell having hydroxymethyl and 3-mercaptopropyl modification on the surface provides reactive functional group for further engineering of the particle with targeting groups and other functional surface modifications.

In some embodiments, the shell encased the PMMA/BMA-Epolight™ IR 1117-curcumin particles results from vinyltrimethoxysilane (VTMS) via Stöber synthesis.

In some embodiments, the VTMS encased PMMA/BMA-Epolight™ IR 1117-curcumin particles passes the Extractable Cytotoxicity Test. In some embodiments, the VTMS encased PMMA/BMA-Epolight™ IR 1117-curcumin particles further passes the Efficacy Determination Protocol.

In some embodiments, the PMMA/BMA-Epolight™ IR 1117-curcumin particles have a median particle size ranging from about 410 nm to about 460 nm. In some embodiments, the VTMS encased PMMA/BMA-Epolight™ IR 1117-curcumin particles have a median particle size of about 500 nm.

In some embodiments, the VTMS encased PMMA/BMA-Epolight™ IR 1117-curcumin particles have about 78% curcumin encapsulation rate and 76% Epolight™ IR 1117 encapsulation rate.

Various particles with different anticancer agents and carriers are prepared using the methods described in Example 1 below. The particle compositions are summarized in Table 2 below.

TABLE 2
Particle Composition

| | Anti cancer | | Carrier/Anticancer | | | The |
|---|---|---|---|---|---|---|
| Entry | agent | Carrier | agent weight ratio | Size | | material |
| 1 | doxorubicin | PLGA 75:25, Mn 2000 | 9:1 | 150 | nm | Tetrakis |
| | | | | | | aminium |
| 2 | paclitaxel | 80% of PLGA (75:25, Mn | 4:1 | 120 | nm | Tetrakis |
| | | 8000) to 20% of PLGA | | | | aminium |
| | | (75:25)-PEG, | | | | |
| | | [Mn 8000 (PLGA) - Mn | | | | |
| | | 2000 (PEG)] | | | | |
| 3 | gefitinib | Poly(sebacic anhydride), | 7:3 | 90 | nm | Tetrakis |
| | | Mn 10,000 | | | | aminium |
| 4 | paclitaxel & | 75% of PLGA (55:45, Mn | 7:1:2 | 100 | nm | Tetrakis |
| | gefitinib | 10000) to 25% of PLGA | | | | aminium |
| | | (75:25)-PEG, | | | | |
| | | [Mn 10000 (PLGA) - Mn | | | | |
| | | 2000 (PEG)] | | | | |
| 5 | cisplatin | 2 parts of 1,2-distearoyl-sn- | 4:1 | 100 | nm | Tetrakis |
| | | glycero-3 -phosphoglycerol | | | | aminium |
| | | (DSPG), 1 part of | | | | |
| | | cholesterol, 0.2 part of 1,2- | | | | |
| | | distearoyl-sn-glycero-3- | | | | |
| | | phosphoethanolamine | | | | |
| | | (DSPE-PEG2000) | | | | |
| 6 | 5-FU | 70% PLGA 90:10, Mn | 7:3 | 180 | nm | Tetrakis |
| | | 6000, 20% Poly (malic | | | | aminium |
| | | acid-co-glycolide), 90:10 | | | | |
| | | Mn 6000 & 10% PLGA | | | | |
| | | (75:25)-PEG [Mn 4000 | | | | |
| | | (PLGA) - Mn 2000 (PEG)] | | | | |
| 7 | GSK461364 | 75% of PLGA (75:25, Mn | 9:1 | 125 | nm | Tetrakis |
| | | 8000) to 25% of PLGA | | | | aminium |
| | | (75:25)-PEG, | | | | |
| | | [Mn 8000 (PLGA) - Mn | | | | |
| | | 2000 (PEG)] | | | | |
| 8 | Etoposide | 70% PLGA 85:15, Mn | 2:1 | 200 | nm | Tetrakis |
| | | 5000, 20% Poly (aspartic | | | | aminium |
| | | acid-co-glycolide), 90:10 | | | | |
| | | Mn 5000 & 10% PLGA | | | | |
| | | (75:25)-PEG [Mn 3000 | | | | |
| | | (PLGA) - Mn 2000 (PEG)] | | | | |
| 9 | Vincristine | 2 parts sphingomyelin | 3:1 | 150 | nm | Tetrakis |
| | sulphate | (egg), 1 part cholesterol and | | | | aminium |
| | | 0.2 parts of 1,2-distearoyl- | | | | |
| | | sn-glycero-3- | | | | |
| | | phosphoethanolamine | | | | |
| | | (DSPE-PEG2000) | | | | |
| 10 | Iriinotecan | 50% HSPC, 20% DSPG | 2:1 | 130 | nm | Tetrakis |
| | | 20% Cholesterol and 10% | | | | aminium |
| | | Alpha-tocopherol | | | | |
| 11 | Gemcitabine | 60% of PLGA (75:25, Mn | 2:1 | 200 | nm | Tetrakis |
| | | 5000) to 40% of PLGA | | | | aminium |
| | | (75:25)-PEG, | | | | |
| | | [Mn 5000 (PLGA) - Mn | | | | |
| | | 2000 (PEG)] | | | | |

### 4(b). Remotely-Triggered Drug Delivery Particles for Antimicrobial Chemotherapy

Infectious diseases remain one of the leading causes of morbidity and mortality worldwide. Additionally, multidrug resistance has been increasing for last decades. Particles (e.g., metallic, organic, carbon nanotubes, etc.) have been developed to deliver antimicrobials to fight microbial infections including multidrug resistant infections, since particles may circumvent drug resistance mechanisms in bacteria and inhibit biofilm formation or other important processes.

Some of the advantages of using particle based antimicrobial therapies are due to their small and controllable size; their protective action against the pathogen and host enzymes that would otherwise destroy the antimicrobial compounds; their ability to actively deliver antibiotics; and their capability to combine several therapeutic modalities onto a single particle (e.g., several antibiotics/compounds into the same particle for combined action; combining silencing agents and drugs, etc.).

Various embodiments of the invention are directed to pharmaceutical compositions comprising particles, including microparticles and/or nanoparticles, for the externally controlled release of antimicrobial agents to treat localized microbial infections and methods for using such pharmaceutical compositions. The pharmaceutical compositions are capable of delivering therapeutic levels of the antimicrobial agent to the infection over the desired extended time frame, and, in some embodiments, the particles may have different sizes and degradation profiles. In some embodiments, the particles of the invention have been developed for encapsulating antimicrobial agents and allowing for their targeted, controlled and sustained delivery. The antimicrobial agents are released outside the particle when an exogenous source is applied to the particle.

The present invention also provides an externally controlled antimicrobial drug delivery system. Such a delivery system allows the precise dosing of an antibiotic while minimizing the potential for misuse or abuse of the antibiotics (e.g. save the antibiotic for use by the future generations).

In some embodiments, this disclosure provides compositions and methods that allow a highly controlled, targeted treatment of localized microbial infections through the externally controlled release of antimicrobial agents. The disclosure also provides an externally controlled antimicrobial drug delivery system. Such a delivery system provides a novel means to treat localized microbial infections including multidrug resistant infections, and Gram-positive and Gram-negative microbial infections.

The disclosure also provides particles comprising an antimicrobial agent and a material that interacts with an exogenous source. Such particles minimize the exposure of healthy cells to the toxic effects of an antimicrobial agent and the material that interacts with the exogenous source which have leaked out of the particle into the body. The particles also minimize the entry of body fluids inside the particle at concentrations that can degrade both the antimicrobial agent and the material. Furthermore, the externally controlled drug delivery system described herein improves the therapeutic index of the antimicrobial agent.

In some embodiments, this disclosure provides a remotely-triggered particle for delivery of antimicrobial chemoactive agent comprising (a) an antimicrobial agent, (b) a carrier, (c) a material that interacts with an exogenous source, wherein the antimicrobial agent is encapsulated by the carrier, wherein the antimicrobial agent and the material in the particle exhibit stability such that the particle is considered passing the Efficacy Determination Protocol; wherein the particle structure is constructed such that it passes the Extractable Cytotoxicity Test; and wherein the antimicrobial agent is released outside the particle when the exogenous source is applied. In some embodiments, the particle is amorphous or partially amorphous or partially crystalline.

In some embodiments, the antimicrobial drug delivery system can be activated with an infrared laser, which acts as the exogenous source to localize the antimicrobial agent to the site of the infection with a concomitant reduction in the off-target adverse events. In one embodiment, the carrier also is conjugated with a microbial targeting ligand selected from the group of a nucleic acid, a protein, a monoclonal antibody, antibody fragment, and a peptide. The microbial targeting ligand leads the particle to the targeted infection site. Once the particle arrives at the targeted infection site, the IR laser is applied, causing the material to absorb the energy from the IR laser and to convert the energy into heat; and wherein the heat causes acceleration of the degradation of the carrier, and then causes the antimicrobial agent to be released at the targeted infection site. The antimicrobial drug is either fully encapsulated within the carrier or has a high affinity for the highly charged or hydrophobic groups in a porous particle matrix.

In some embodiments, the particle further comprises a shell enclosing the particle to form a core-shell particle.

In some embodiments, the particle further comprises a microbial targeting group on the particle surface. In some embodiments, the bacteria targeting group is selected from the group of an antibody targeting the surface antigen of the bacteria, a cationic antimicrobial peptide, cell penetrating peptides including apidaecin, tat, buforin, magainin, and combinations thereof.

In some embodiments, the particle can be monitored after administration by an incorporated imaging agent such as fluorescent dye, a computed tomography (CT) contrast agent (like iodine) or magnetic particles. Once the particle travels to the targeted tissue as indicated by the imaging agent, an exogenous source is applied, then causing the antimicrobial agent encapsulated in the particle to be released from the particle.

In some embodiments, the energy-to-heat conversion agent is an IR absorbing agent. Upon contacting with the exogenous source, the IR absorbing agent absorbs the photonic energy from the electromagnetic radiation and converts the energy to heat to induce localized hyperthermia at a temperature range that is sufficient to kill the microbes in the infected tissue. Particles may have hydrophilic polymer surface modifications as described above that allow them to circulate in the body's vascular system for a prolonged period to get high accumulation in the infected tissue. In some embodiments, the antimicrobial agent and the material may be co-encapsulated within the same particle. In some embodiments, the antimicrobial agent and the material are in two discrete population of particles. In some embodiments, the antimicrobial agent is not encapsulated and combined with the particle heaters concurrently or sequentially.

The particle heaters may further be surface modified with a microbe-targeting group to help localize the particles to the infection site. In some embodiments, the bacteria targeting group is selected from the group of an antibody targeting the surface antigen of the bacteria, a cationic antimicrobial peptide, cell penetrating peptides including apidaecin, tat, buforin, magainin, and combinations thereof. In some embodiments, the microbial targeting group is selected from the group of a group targeting MSCRAMM (microbial surface components recognizing adhesive matrix molecules), GADPH (surface enzyme), LPXTG domain, Lipid A, β-barrel proteins commonly called outer membrane proteins (OMPs), CARGGLKSC (CARG), and combinations thereof. In some embodiments, the microbial targeting group is ubiquicidin (UBI₂₉₋₄₁). In some embodiments, the particle surface is labeled with a positively charged moiety such as poly-lysine, chitosan etc. via covalent bonding to localize the particle to the negatively charged bacteria membrane. In some embodiments, the particle surface is labeled with a macrophage-targeting group selected from a group of dextran, tuftsin, mannose, hyaluronate, and combinations thereof. The use of microbe targeting ligands greatly improves the precision of the delivery of particle heaters to the desired infection site.

This particle design provides the synergistic benefits of enhanced concentration of particle heater at the infection site via microbe cells membrane receptor targeting and the on-demand induction of localized hyperthermia via the energy-to-heat conversion by the material. The encapsulation of the material by the carrier minimizes the cytotoxicity caused by leakage of material or toxic degradation component of the material outside the particle after exposure to the laser light.

In some embodiments, the antimicrobial agent encapsulated with the herein described remotely-triggered drug delivery particle is an antibiotic selected from the group of ampicillin, sulbactam, cefotaxime, telithromycin, temafloxacin, temafloxacin, trovafloxacin, praziquantel, amikacin, ciprofloxacin, vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combination thereof.

In some embodiments, the antimicrobial agent is a thermal-stable antibiotic. In some embodiments, the thermal-stable antibiotic is gentamicin, curcumin, or vanvomycin. In some embodiments, the thermal-stable antibiotic is curcumin. In some embodiments, the thermal-stable antibiotic is vanvomycin. In some embodiments, the thermal-stable antibiotic is gentamicin.

Some embodiments of the remotely-triggered drug delivery particles described herein are listed in the Table 3 below, which can be made according to the method described in this disclosure.

TABLE 3
Remotely-Triggered Antimicrobial Drug Delivery Particles

| | Antimicrobial | | Carrier/Antimicrobial | | | The |
|---|---|---|---|---|---|---|
| # | agent | Carrier | agent weight ratio | Size | | material |
| 1 | streptomycin | PLGA 75:25, Mn 2000 | 7:1 | 150 | nm | Tetrakis |
| | | | | | | aminium |
| 2 | ampicillin | 80% of PLGA (75:25, Mn | 4:1 | 120 | nm | Tetrakis |
| | | 8000) to 20% of PLGA | | | | aminium |
| | | (75:25)-PEG, | | | | |
| | | [Mn 8000 (PLGA) - Mn 2000 | | | | |
| | | (PEG)] | | | | |
| 3 | sulbactam | Poly(sebacic anhydride), Mn | 7:3 | 90 | nm | Tetrakis |
| | | 10,000 | | | | aminium |
| 4 | ampicillin & | 75% of PLGA (55:45, Mn | 7:1:2 | 100 | nm | Tetrakis |
| | sulbactam | 10000) to 25% of PLGA | | | | aminium |
| | | (75:25)-PEG, | | | | |
| | | [Mn 10000 (PLGA) - Mn 2000 | | | | |
| | | (PEG)] | | | | |
| 5 | amphotericin | 2 parts of 1,2-distearoyl-sn- | 4:1 | 100 | nm | Tetrakis |
| | B | glycero-3 -phosphoglycerol | | | | aminium |
| | | (DSPG), 1 part of cholesterol, | | | | |
| | | 0.2 part of 1,2-distearoyl-sn- | | | | |
| | | glycero-3- | | | | |
| | | phosphoethanolamine (DSPE- | | | | |
| | | PEG2000) | | | | |
| 6 | cefotaxime | 70% PLGA 90:10, Mn 6000, | 7:3 | 180 | nm | Tetrakis |
| | | 20% Poly (malic acid-co- | | | | aminium |
| | | glycolide), 90:10 Mn 6000 & | | | | |
| | | 10% PLGA (75:25)-PEG [Mn | | | | |
| | | 4000 (PLGA) - Mn 2000 | | | | |
| | | (PEG)] | | | | |
| 7 | gentamicin | 75% of PLGA (75:25, Mn | 9:1 | 125 | nm | Tetrakis |
| | | 8000) to 25% of PLGA | | | | aminium |
| | | (75:25)-PEG, | | | | |
| | | [Mn 8000 (PLGA) - Mn 2000 | | | | |
| | | (PEG)] | | | | |
| 8 | telithromycin | 70% PLGA 85:15, Mn 5000, | 2:1 | 200 | nm | Tetrakis |
| | | 20% Poly (aspartic acid-co- | | | | aminium |
| | | glycolide), 90:10 Mn 5000 & | | | | |
| | | 10% PLGA (75:25)-PEG [Mn | | | | |
| | | 3000 (PLGA) - Mn 2000 | | | | |
| | | (PEG)] | | | | |
| 9 | temafloxacin | 2 parts sphingomyelin (egg), 1 | 3:1 | 150 | nm | Tetrakis |
| | | part cholesterol and 0.2 parts of | | | | aminium |
| | | 1,2-distearoyl-sn-glycero-3- | | | | |
| | | phosphoethanolamine (DSPE- | | | | |
| | | PEG2000) | | | | |
| 10 | trovafloxacin | 50% HSPC, 20% DSPG | 2:1 | 130 | nm | Tetrakis |
| | | 20% Cholesterol and 10% | | | | aminium |
| | | Alpha-tocopherol | | | | |
| 11 | praziquantel | 60% of PLGA (75:25, Mn | 2:1 | 200 | nm | Tetrakis |
| | | 5000) to 40% of PLGA | | | | aminium |
| | | (75:25)-PEG, | | | | |
| | | [Mn 5000 (PLGA) - Mn 2000 | | | | |
| | | (PEG)] | | | | |

In some embodiments, the present invention provides a method for treating a localized microbial infection in a patient in need thereof comprising: (1) administering to the patient a pharmaceutical composition comprising the herein described remotely-triggered antimicrobial drug delivery particle; (2) irradiating the drug delivery particles with pulsed laser.

In some embodiments, the localized microbial infection is caused by multidrug resistant bacteria. In an embodiment, the multidrug resistant bacteria comprise Gram positive bacteria. In an embodiment, the multidrug resistant bacteria comprise Gram negative bacteria. In an embodiment, the multidrug resistant bacteria comprise both Gram positive and Gram negative bacteria. In an embodiment, the multidrug resistant bacteria comprise one or more species selected from the group of E. coli, K. pneumonia, M tuberculosis, Streptococcus aureus, P. aeruginosa, Streptococcus epidermidis, and Streptococcus haemolyticus.

### 5(a). Remotely-Triggered Particles for Combination Therapy

Primary treatment for many solid cancers includes surgical excision or radiation therapy, with or without the use of adjuvant therapy. This can include the addition of radiation, chemotherapy and/or thermal after primary surgical therapy, or the addition of chemotherapy and salvage surgery to primary radiation therapy. Both primary therapies, thermotherapy, surgery and radiation, require precise anatomic localization of tumor. If tumor is not targeted adequately with initial treatment, disease recurrence may ensue, and if targeting is too broad, unnecessary morbidity may occur to nearby structures or remaining normal tissue.

Combinations of various therapeutic modalities with non-overlapping toxicities are among the commonly used strategies to improve the therapeutic index of treatments in modern oncology. Two general approaches may increase antitumor effectiveness of remotely-triggered thermotherapy: (i) sensitization of tumor cells to remotely-triggered thermotherapy; (ii) interference with cytoprotective molecular responses triggered by remotely-triggered thermotherapy in surviving tumor or stromal cells. The remotely-triggered thermotherapy disclosed herein can be used in combination with surgery, radiation therapy, or chemotherapy as neoadjuvant, adjuvant or repetitive adjuvant treatment.

In combination therapies, especially in synergistic chemotherapy and PTT and/or PDT, either anticancer agents or photoactive agents are lack of tumor selectivity, thus increase potential toxicity in normal tissues. The use of particles to transport photosensitizer and chemotherapy drugs can enhance the drug concentration at the target site, significantly reduces the side effects and improves the effectiveness of thermotherapy and chemotherapy. The well-designed particles possess the functions including targeted drug delivery, sustained chemotherapeutic drug release, and the production of reactive molecular species in response to an exogenouse source.

Targeting cytotoxic chemotherapeutic drugs in oncology is essential because side toxicities limit reaching effective local doses. Functionalization of particle cytotoxic drug delivery system has so far achieved a moderate targeting effect. The nanoscale size of drug preparations favors enhanced permeability and retention (EPR) and reduces renal filtration. In an embodiment, this disclosure provides a synergistic combination therapy for the treatment of cancer comprising: (a) an anticancer agent, and (b) a particle heater having a material interacting with an exogenous source admixed with a carrier, wherein the material absorbs the energy from the exogenous source and converts the energy into heat; and then the heat travels outside the particle to induce localized hyperthermia, wherein the heat causes the particle to alter its structure to release the anticancer agent outside of the particle, wherein the localized hyperthermia and the anticancer agent exhibit synergy in killing cancer cells, and wherein the particle is constructed such that it passes the Extractable Cytotoxicity Test.

In some embodiments, the localized hyperthermia and the anticancer agent exhibit coefficient of drug interaction (CDI)<1.0. In some embodiments, the CDI of the localized hyperthermia and the anticancer agent is about 0.1, about 0.2, about 0.3, about 0.4, about 0.5, about 0.6, about 0.7, about 0.8, about 0.9, or about 1.0.

In some embodiments, the anticancer agent is further encapsulated by the particle heater having the material, and wherein the heat causes the particle heater to alter its structure to release the anticancer agent outside of the particle. In some embodiments, the anticancer agent further comprises the carrier to form a chemotherapy particle free of the material, and wherein the heat causes the chemotherapy particle to alter its structure to release the anticancer agent outside of the particle. In some embodiments, the anticancer agent is in a conventional pharmaceutical dosage.

In some embodiments, the particle heater further passes the Thermal Cytotoxicity Test. In some embodiments, the particle heater further passes the Efficacy Determination Protocol.

In some embodiments, the particle exhibits material process stability that the particle heater preserves greater than 50% of absorbance after being subject to the exogenous source process conditions.

In some embodiments, at least a portion of the exterior surface of the particle has a modification that is polar, non-polar, charged, ionic, basic, acidic, reactive, hydrophobic, or hydrophilic.

In some embodiments, the particle further comprises a shell to enclose the particle to form a core-shell particle. In some embodiments, the shell comprises a crosslinked inorganic polymer selected from the group of mesoporous silica, organo-modified silicate polymer derived from condensation of organotrisilanol or halotrisilanol, and combinations thereof.

In some embodiments, the shell results from the use of an alkyltrimethoxysilane reagent (CnTMS, n is an integer ranging from 1 to 12) in the Stöber synthesis. In some embodiments, the shell results from the use of C1-C7 alkyl trimethoxysilane reagent in the Stöber synthesis. In some embodiments, the shell results from the use of C1-C7 alkenyl trimethoxysilane reagent in the Stöber synthesis. In some embodiments, the shell results from the use of C1-C7 alkynyl trimethoxysilane reagent in the Stöber synthesis. In some embodiments, the C1-C7 alkyl group, the C1-C7 alkenyl group, or the C1-C7 alkynyl group may be linear or branched. In some embodiments, the shell results from the use of C2-C6 linear alkyl trimethoxysilane reagent in the Stöber synthesis. In some embodiments, the shell results from the use of C2-C4 linear alkyl trimethoxysilane reagent in the Stöber synthesis. In some embodiments, the shell results from the use of ethyl (C2) trimethoxysilane reagent in Stöber synthesis. In some embodiments, the shell results from the use of vinyltrimethoxysilane (VTMS) reagent in Stöber synthesis. In some embodiments, the shell results from the condensation reaction of hydroxymethylsilanetriol prepared by the hydrolysis of hydroxymethyltrichlorosilane. In some embodiments, the shell results from the condensation reaction of (3-mercaptopropyl)silanetriol prepared by the hydrolysis of (3-mercaptopropyl)trimethoxysilane. The silicate shell having hydroxymethyl and 3-mercaptopropyl modification on the surface provides reactive functional group for further engineering of the particle with targeting groups and other functional surface modifications.

In some embodiments, the shell comprises a cross-linked inorganic polymer selected from the group of mesoporous silica, organo-modified silicate polymer derived from condensation of organotrisilanol or halotrisilanol, and combinations thereof.

In some embodiments, the shell comprises a plasmonic absorber selected from the group of a thin film of noble metals including gold (Au), silver (Ag), copper (Cu), nanoporous gold thin film, and combinations thereof. In some embodiments, the particle further comprises a coating formed of polydopamine that is capable of photothermal conversion.

In some embodiments, the particle heaters are designed for remotely-triggered thermotherapy combined with chemotherapy, wherein the exogenous source may include an electromagnetic radiation, an electrical field, a microwave, a radio wave, an ultrasound, a magnetic field, and combinations thereof.

In some embodiments, the particle heaters are designed for remotely-triggered thermotherapy combined with chemotherapy, wherein the exogenous source is a magnetic field and the material is iron oxide nanoparticles.

Various particles with different anticancer agents and carriers are prepared using the methods described herein. The particle compositions are summarized in Table 4 below.

TABLE 4
Particle Composition

| | | | Carrier/Anti- | | |
|---|---|---|---|---|---|
| | | | cancer agent | Targeting | |
| | | | weight | Ligand/tumor | |
| Entry | Anti cancer agent | Carrier | ratio/size | type | Material |
| 1 | dabrafenib/trametinib | PLGA 75:25, | 9:1/150 nm | cetuximab/ | ICG, IR 820, IR |
| | | Mn 2000 | | NSCLC | 780, IR 193, |
| | | | | | squaraine dye, |
| | | | | | Epolight ™ 1117, |
| | | | | | Epolight ™ 1175, |
| | | | | | iron oxide |
| | | | | | nanoparticle |
| 2 | trastuzumab and | 80% of PLGA | 4:1/120 nm | Lyp-1/ | ICG, IR 820, IR |
| | paclitaxel | (75:25, Mn | | metastatic | 780, IR 193, |
| | | 8000) to 20% of | | breast cancer | squaraine dye, |
| | | PLGA (75:25)- | | | Epolight ™ 1117, |
| | | PEG, | | | Epolight ™ 1175, |
| | | [Mn 8000 | | | iron oxide |
| | | (PLGA) - Mn | | | nanoparticle |
| | | 2000 (PEG)] | | | |
| 3 | lapatinib and | 75% of PLGA | 7:3/90 nm | Lyp-1/ | ICG, IR 820, IR |
| | trastuzumab | (55:45, Mn | | metastatic | 780, IR 193, |
| | | 10000) to 25% | | breast cancer | squaraine dye, |
| | | of PLGA | | | Epolight ™ 1117, |
| | | (75:25)-PEG, | | | Epolight ™ 1175, |
| | | [Mn 10000 | | | iron oxide |
| | | (PLGA) - Mn | | | nanoparticle |
| | | 2000 (PEG)] | | | |
| 4 | GSK461364/ | 75% of PLGA | 9:1/101 nm | CooP, CLT-1, | ICG, IR 820, IR |
| | gefitinib (1:4.54 | (75:25, Mn | | angiopep-2, | 780, IR 193, |
| | mol/mol) | 8000) to 25% of | | FHK | squaraine dye, |
| | | PLGA (75:25)- | | peptide/brain | Epolight ™ 1117, |
| | | PEG, | | tumor | Epolight ™ 1175, |
| | | [Mn 8000 | | | iron oxide |
| | | (PLGA) - Mn | | | nanoparticle |
| | | 2000 (PEG)] | | | |

### 5(b). Combination Antimicrobial Therapy

In some embodiments, the disclosure provides a particle heater for administering a remotely triggered combination thermo-chemotherapy for the treatment of an infection in a subject. The particle heater disclosed herein and a pharmaceutically acceptable excipient typically in the form of a pill, hard or soft shell capsule, tablet, gel, oral powder, or liquid formulation. In some embodiments, the carrier encapsulates the material and the antimicrobial agent to form a single particle heater. In some embodiments, the carrier encapsulates each of the material and the antimicrobial agent to form two populations of particles independently.

In some embodiments, the invention provides pharmaceutical compositions comprising particle heaters and an antimicrobial agent described herein for remotely-triggered combination treatment of a microbial infection. In some embodiments, the pharmaceutical compositions are formulated to provide a therapeutically effective amount of the material interacting with the exogenous source. In some embodiments, the pharmaceutical compositions contain the particle heaters, and one or more pharmaceutically acceptable excipients, carriers, and adjuvants. In some embodiments, the antimicrobial agent is encapsulated in the particle heater and the heat causes the release of the antimicrobial agent. In some embodiments, the antimicrobial agent is not encapsulated in the particle heater. In some embodiments, the antimicrobial agent is present in a separate pharmaceutical composition from the particle heater.

In some embodiments, the pharmaceutical dosage of the antimicrobial agent is selected from the group of a pill, a hard or soft shell capsule, a tablet, a gel, an oral powder, a buccal tablet, a sublingual tablet, an orally disintegrating tablet, a liquid formulation, a dispersion, an injection preparation, a powder for injection, and a suppository.

In some embodiments, the particle heaters are nanoparticles or microparticles. In some embodiments, the particle heater is further combined with a pharmaceutically acceptable excipient to form a particle heater preparation.

In an embodiment, this disclosure provides a kit for the remotely-triggered combination therapy useful for the treatment of a microbial infection comprising (1) a particle heater preparation having a material interacting with an exogenous source and a carrier, (2) a pharmaceutical dosage containing an antimicrobial agent; wherein the pharmaceutical dosage of an antimicrobial agent containing any of the antimicrobial agents as disclosed herein, and wherein the pharmaceutical dosage of the antimicrobial agent is selected from the group of a capsule, a tablet, a buccal tablet, a sublingual tablet, an orally disintegrating tablet, a liquid formulation, a dispersion, an injection preparation, powder for injection, and suppository. Depending on the infection types and the therapeutic strategy, the particle heater and the pharmaceutical dosage of the antimicrobial agent may be administered concurrently in a unitary dose, concurrently in two separate doses, or sequentially.

In some embodiments, this disclosure provides a dry, removable, sterile multilayered wound dressing, wherein the dressing comprises a matrix holding the particle heaters and the antimicrobial agent as described above, wherein the matrix is selected from a group of a film, hydrogel membrane, non-woven fabric, and woven fabric. In some embodiments, the matrix is made of a biocompatible substance selected from the group of gelatin sponge, calcium alginate, collagen, and oxidized regenerated cellulose. In some embodiments, the particle heater is dispersed within, embedded within, or forms a coating on the matrix.

In some embodiments, the wound dressing is constructed as a band-aid form, where the particle heater formulation containing layer is adhered to an adhesive backing layer. One or more additional layers of wound dressing materials include a layer containing super absorbents to wick the wound exudate.

In some embodiments, the wound dressing is constructed as a patch for use in the treatment of Herpes Labialis, said patch comprises a backing layer, and a layer of a skin-friendly adhesive, where the adhesive comprises particle heaters and the antimicrobial as described above and hydrocolloid particles, and one or more additional layers that contain super absorbents to wick the wound exudate.

### 6. Pharmaceutical Formulations

In some embodiments, the invention provides pharmaceutical compositions comprise a particle heaters described herein for remotely-triggered thermal treatment of a cancer and microbial infections.

In some embodiments, the pharmaceutical compositions are formulated to provide a therapeutically effective amount of the material interacting with the exogenous source. In some embodiments, the material is encapsulated by the carrier to form a remotely-triggered particle heater. In some embodiments, the remotely-triggered particle heaters are nanoparticles or microparticles. In some embodiments, the pharmaceutical compositions contain the remotely-triggered particle heaters, and one or more pharmaceutically acceptable excipients, carriers, and adjuvants. In some embodiments, the remotely-triggered particle heater formulation is selected from the group of a capsule, a tablet, a buccal tablet, a sublingual tablet, an orally disintegrating tablet, a liquid formulation, a dispersion, an injection preparation, powder for injection, and suppository.

In some embodiments, the disclosure provides a pharmaceutical composition for administering a remotely triggered synergistic combination therapy for the treatment of a cancer or microbial infection in a subject. The pharmaceutical compositions comprising remotely-triggered drug delivery particles for the controlled delivery of anticancer agent and antimicrobial agent as disclosed herein and a pharmaceutically acceptable excipient typically in the form of a pill, hard or soft shell capsule, tablet, gel, oral powder, or liquid formulation. In some embodiments, the material and the anticancer agent or antimicrobial agent are encapsulated by the carrier to form a remotely-triggered drug delivery particle. In some embodiments, the material and the anticancer agent or antimicrobial agent are each independently encapsulated by the carrier to form two populations of particles.

In an embodiment, this disclosure provides a composition for use in a remotely-triggered synergistic combination therapy of a cancer or microbial infection comprising (a) a particle heater having a material interacting with an exogenous source and a carrier; and (b) a herein described anticancer agent or antimicrobial agent.

In some embodiments, the particle heater and the anticancer agent or antimicrobial agent pharmaceutical dosage forms a unitary dosage. In some embodiments, the particle heater and the anticancer agent or antimicrobial agent pharmaceutical dosage are two discrete preparations.

In some embodiments, the anticancer agent or antimicrobial agent pharmaceutical dosage is selected from the group of a capsule, a tablet, a buccal tablet, an oral disintegrating tablet, a liquid formulation, a dispersion, an injectable preparation, powder for injection, and suppository.

In some embodiments, the material is chemically conjugated to the particle heater surface via a heat-labile linker. In some embodiments, the heat-labile linker is selected from the group of substituted and unsubstituted carbonates, substituted and unsubstituted carbamates, substituted and unsubstituted esters, substituted and unsubstituted lactams, substituted and unsubstituted lactones, substituted and unsubstituted amides, substituted and unsubstituted imides, substituted and unsubstituted oximes, substituted and unsubstituted sulfonates, substituted and unsubstituted phosphonates, and combinations thereof.

The increase in temperature due to the thermal conversion effects of the material may be as low as 5° C. and as high as 70° C., depending on the chemical structure of the material utilized. A temperature increase of 40° C. induced on the surface of living tissue will generally cause necrosis or thermal damage. Temperature increases of lesser amounts will generally cause discomfort and irritation of the tissue. In order to minimize these problems, heat dissipating agents are introduced into the pharmaceutical composition of the particle heater as adjuvant additive. The heat dissipating agents include liquids or solids.

In some embodiments, the pharmaceutical composition of the particle heaters additionally comprises a heat dissipating agent to reduce the temperature increase due to the localized hyperthermia induced by the exogenous source. In some embodiments, the heat dissipating agent is selected from the group of a volatile liquid, a solid having a melting point of from about 20° C. to about 150° C., and a solid having a sublimation point of from about 20° C. to about 150° C.

In some embodiments, solids that act as a heat sink or those that readily adsorb heat may be utilized. Suitable heat-adsorbing substances include alkaline metal oxide such as aluminum oxide, barium oxide, titanium oxide, manganese oxide and calcium oxide; metal nanoparticles such as copper, lead, nickel, aluminum, and zinc; carbon black and carbides; organic compounds such as urea, paraffin wax and polyvinyl fluoride; and salts such as ammonium nitrate, potassium nitrate, sodium acetate trihydrate, sodium sulfate decahydrate (Glauber's salt), barium hydroxide octahydrate, calcium oxalate dihydrate, magnesium oxalate dihydrate, aluminum hydroxide, ammonium sulfate, zinc sulfate, and ammonium phosphate.

In some embodiments, the heat dissipating agent is selected from the group of potassium nitrate, sodium acetate trihydrate, sodium sulfate decahydrate, barium hydroxide octahydrate, calcium oxalate dihydrate, magnesium oxalate dihydrate, aluminum hydroxide, zinc sulfate, aluminum oxide, barium oxide, titanium oxide, manganese oxide, and calcium oxide; copper nanoparticle, nickel, aluminum and zinc; carbon black and carbides; graphene nanoparticle, graphene oxide nanoparticle, urea, paraffin wax and polyvinyl fluoride; poly(N-isopropylacrylamide) (PNIPAAm) composite incorporating glycidyl methacrylate functionalized graphene oxide (GO-GMA), 2-hydroxy-2-trimethylsilanyl-propionitrile, 1-fluoropentacyclo[6.3.0.02,6.03,10.05,9]undecane, 6,7-diazabicyclo[3.2.1]oct-6-ene, 5,5,6,6-tetramethylbicyclo[2.2.1]heptan-2-ol, complex of dimethyl magnesium and trimethylaluminum, N-benzyl-2,2,3,3,4,4,4-heptafluoro-butyramide, 3-isopropyl-5,8a-dimethyl-decahydronaphthalen-2-ol, 2-hydroxymethyl-1,7,7-trimethyl-bicyclo[2.2.1]heptan-2-ol, 3,5-dichloro-3-methyl-cyclopentane-1,2-dione, (5-methyl-2-oxo-bicyclo[3.3.1.]non-3-en-1-yl)-acetic acid, 4b,6a,11,12-tetrahydro-indeno[2,1-a]fluorene-5,5,6,6-tetracarbonitrile, tetracosafluoro-tetradecahydro-anthracene, 4,5-dichlorobenzene-1,2-dicarbaldehyde, bicyclo[4,3.1]dec-3-en-8-one, 3-tert-butyl-1,2-bis-(3,5-dimethylphenyl)-3-hydroxyguanidine, 1-[2,6-dihydroxy-4-methoxy-3-methylphenyl]butan-1-one, 2,3,6,7-tetrachloronaphthalene, 2,3,6-trimethylnaphthalene, dodecafluoro-cyclohexane, 2,2,6,6-tetramethyl-4-hepten-3-one, 1,1,1-trichloro-2,2,2-trifluoro-ethane, [5-(9H-beta-carbolin-1-yl)-furan-2-yl]methanol, 5-nitro-benzo[1,2,3]thiadiazole, 4,5-dichloro-thiophene-2-carboxylic acid, 2,6-dimethyl-isonicotinonitrile, nonafluoro-2,6-bis-trifluoromethyl-piperidine, (dimethylamino)difluoroborane, dinitrogen pentoxide, chromyl fluoride, and chromium hexacarbonyl; 1-methylcyclohexanol, phenyl ether, nonadecane, 1-tetradecanol, 4-ethylphenol, benzophenone, maleic anhydride, octacosane, dimethyl isophthalate, butylated hydroxytoluene, glycolic acid, vanillin, magnesium nitrate hexahydrate, cyclohexanone oxime, glutaric acid, D-sorbitol, phenanthrene, fluorene, trans-stilbene, neopentyl glycol, pyrogallol, and diglycolic acid; and combinations thereof.

In some embodiments, the temperature reduction because of incorporating the dissipating agent is of about 1° C. to 70° C. In some embodiments, the temperature reduction as a result of incorporating the dissipating agent is selected from: about 1° C., about 5° C., about 10° C., about 15° C., about 20° C., about 25° C., about 30° C., about 35° C., about 40° C., about 45° C., about 50° C., about 55° C., about 60° C., about 65° C., and about 70° C.,

In some embodiments, the amount of the heat-dissipating agent used is in an amount sufficient to prevent necrosis of the living tissue. In some embodiments, the amount of the heat-dissipating agent presented in the pharmaceutical composition of the particle heater is of about 0.1 wt. % to about 50.0 wt. % by the total weight of the composition. In some embodiments, the amount of the heat-dissipating agent presented in the pharmaceutical composition is of about 1 wt. % to about 15.0 wt. % by the total weight of the pharmaceutical composition. In some embodiments, the amount of the heat-dissipating agent presented in the pharmaceutical composition is of about 5.0 wt. % to about 15.0 wt. % by the total weight of the pharmaceutical composition. In some embodiments, the amount of the heat-dissipating agent presented in the pharmaceutical composition is selected from the group of about 1.0 wt. %, about 1.1 wt. %, about 1.2 wt. %, about 1.3 wt. %, about 1.4. wt. %, about 1.5 wt. %, about 1.6 wt. %, about 1.7 wt. %, about 1.8 wt. %, about 1.9 wt. %, about 2.0 wt. %, about 2.1 wt. %, about 2.2 wt. %, about 2.3 wt. %, about 2.4. wt. %, about 2.5 wt. %, about 2.6 wt. %, about 2.7 wt. %, about 2.8 wt. %, about 2.9 wt. %, about 3.0 wt. %, about 3.1 wt. %, about 3.2 wt. %, about 3.3 wt. %, about 3.4. wt. %, about 3.5 wt. %, about 3.6 wt. %, about 3.7 wt. %, about 3.8 wt. %, about 3.9 wt. %, about 4.0 wt. %, about 4.1 wt. %, about 4.2 wt. %, about 4.3 wt. %, about 4.4. wt. %, about 4.5 wt. %, about 4.6 wt. %, about 4.7 wt. %, about 4.8 wt. %, about 4.9 wt. %, about 5.0 wt. %, about 5.1 wt. %, about 5.2 wt. %, about 5.3 wt. %, about 5.4. wt. %, about 5.5 wt. %, about 5.6 wt. %, about 5.7 wt. %, about 5.8 wt. %, about 5.9 wt. %, about 6.0 wt. %, about 6.1 wt. %, about 6.2 wt. %, about 6.3 wt. %, about 6.4. wt. %, about 6.5 wt. %, about 6.6 wt. %, about 6.7 wt. %, about 6.8 wt. %, about 6.9 wt. %, about 7.0 wt. %, about 7.1 wt. %, about 7.2 wt. %, about 7.3 wt. %, about 7.4. wt. %, about 7.5 wt. %, about 7.6 wt. %, about 7.7 wt. %, about 7.8 wt. %, about 7.9 wt. %, about 8.0 wt. %, about 8.1 wt. %, about 8.2 wt. %, about 8.3 wt. %, about 8.4. wt. %, about 8.5 wt. %, about 8.6 wt. %, about 8.7 wt. %, about 8.8 wt. %, about 8.9 wt. %, about 9.0 wt. %, about 9.1 wt. %, about 9.2 wt. %, about 9.3 wt. %, about 9.4. wt. %, about 9.5 wt. %, about 9.6 wt. %, about 9.7 wt. %, about 9.8 wt. %, about 9.9 wt. %, about 10.0 wt. %, about 10.1 wt. %, about 10.2 wt. %, about 10.3 wt. %, about 10.4. wt. %, about 10.5 wt. %, about 10.6 wt. %, about 10.7 wt. %, about 10.8 wt. %, about 10.9 wt. %, about 11.0 wt. %, about 1.1 wt. %, about 11.2 wt. %, about 11.3 wt. %, about 11.4. wt. %, about 11.5 wt. %, about 11.6 wt. %, about 11.7 wt. %, about 11.8 wt. %, about 11.9 wt. %, about 12.0 wt. %, about 12.1 wt. %, about 12.2 wt. %, about 12.3 wt. %, about 12.4. wt. %, about 12.5 wt. %, about 12.6 wt. %, about 12.7 wt. %, about 12.8 wt. %, about 12.9 wt. %, about 13.0 wt. %, about 13.1 wt. %, about 13.2 wt. %, about 13.3 wt. %, about 13.4. wt. %, about 13.5 wt. %, about 13.6 wt. %, about 13.7 wt. %, about 13.8 wt. %, about 13.9 wt. %, about 14.0 wt. %, about 14.1 wt. %, about 14.2 wt. %, about 14.3 wt. %, about 14.4. wt. %, about 14.5 wt. %, about 14.6 wt. %, about 14.7 wt. %, about 14.8 wt. %, about 14.9 wt. %, and about 15.0 wt. %.

In some embodiments, the pharmaceutical formulation may additionally comprises a pharmaceutically acceptable excipient including solvents, dispersion media, diluents, or other liquid vehicles, dispersion or suspension aids, surface active agents, isotonic agents, thickening or emulsifying agents, preservatives, solid binders, lubricants, antimicrobial preservatives, antioxidants and other excipients such as dispersing, suspending, thickening, emulsifying, buffering, wetting, solubilizing, stabilizing, flavoring and sweetening agents. Liquid vehicle may include PBS buffer, saline, sucrose or a suitable polyhydric alcohol or alcohols and which optionally contain ethanol, an elixir or linctus.

Pharmaceutically acceptable excipients used in the manufacture of solid oral dosage include, but are not limited to, inert diluents, dispersing and/or granulating agents, surface active agents and/or emulsifiers, disintegrating agents, binding agents, preservatives, buffering agents, lubricating agents, and/or oils. Such excipients may optionally be included in pharmaceutical formulations. Excipients such as cocoa butter and suppository waxes, coloring agents, coating agents, sweetening, flavoring, and/or perfuming agents can be present in the composition, according to the judgment of the formulator. Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

Liquid dosage forms for oral and parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, oral compositions can include adjuvants such as wetting agents, emulsifying and suspending agents, sweetening, flavoring, and/or perfuming agents. In certain embodiments for parenteral administration, compositions are mixed with solubilizing agents such Cremophor®, alcohols, oils, modified oils, glycols, polysorbates, cyclodextrins, polymers, and/or combinations thereof.

For systemic administration, intravenous injection or infusion may be employed. In some embodiments, the pharmaceutical composition suitable for systemic administration comprises particle heaters disclosed herein and a pharmaceutically acceptable excipient typically in the form of gel, or liquid formulation. In some embodiments pharmaceutically acceptable excipients suitable for liquid formulation include solvents, dispersion media, diluents, or other liquid vehicles, dispersion or suspension aids, surface active agents, isotonic agents, thickening or emulsifying agents, preservatives, antimicrobial preservatives, antioxidants and other excipients such as dispersing, suspending, thickening, emulsifying, buffering, wetting, or stabilizing agents. Liquid vehicle may include PBS buffer, saline, sucrose or a suitable polyhydric alcohol or alcohols and which optionally contain ethanol, an elixir or linctus.

Liquid dosage forms for parenteral administration include, but are not limited to, pharmaceutically acceptable emulsions, microemulsions, solutions, suspensions, syrups, and/or elixirs. In addition to active ingredients, liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. In certain embodiments for parenteral administration, compositions are mixed with Cremophor®, alcohols, oils, modified oils, glycols, polysorbates, cyclodextrins, polymers, and/or combinations thereof.

Injectable preparations, for example, sterile injectable aqueous or oleaginous suspensions may be formulated according to the known art using suitable dispersing agents, wetting agents, and/or suspending agents. Sterile injectable preparations may be sterile injectable solutions, suspensions, and/or emulsions in nontoxic parenterally acceptable diluents and/or solvents, for example, as a solution in 1,3-butanediol. Among the acceptable vehicles and solvents that may be employed are water, Ringer's solution, and isotonic sodium chloride solution. Sterile, fixed oils are conventionally employed as a solvent or suspending medium. For this purpose, any bland fixed oil can be employed including synthetic mono- or diglycerides. Fatty acids such as oleic acid can be used in the preparation of injectable compositions.

Administration of pharmaceutical compositions described herein can be achieved by any method that enables delivery of the active agent to the site of action. These methods include parenteral injection (including intravenous, intra-arterial, subcutaneous, intramuscular, intravascular, intratumoral, intraperitoneal, or infusion), topical (e.g., transdermal application), rectal administration, and via local delivery by catheter or stent or through inhalation.

In some embodiments, the invention provides pharmaceutical compositions comprising a particle heater described herein for treating a cancer.

In some embodiments, the pharmaceutical compositions are formulated to provide a therapeutically effective amount of an active agent thereof. Where desired, the pharmaceutical compositions contain an active agent, and one or more pharmaceutically acceptable excipients and adjuvants. Where desired, other ingredients in addition to an active agent or a pharmaceutically acceptable salt thereof may be mixed into a preparation or both components may be formulated into separate preparations for use in combination separately or at the same time.

In selected embodiments, the concentration of an active agent provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v.

In selected embodiments, the concentration of an active agent provided in the pharmaceutical compositions of the invention is independently greater than 90%, 80%, 70%, 60%, 50%, 40%, 30%, 20%, 19.75%, 19.50%, 19.25% 19%, 18.75%, 18.50%, 18.25%, 18%, 17.75%, 17.50%, 17.25% 17%, 16.75%, 16.50%, 16.25%, 16%, 15.75%, 15.50%, 15.25%, 15%, 14.75%, 14.50%, 14.25%, 14%, 13.75%, 13.50%, 13.25%, 13%, 12.75%, 12.50%, 12.25%, 12%, 11.75%, 11.50%, 11.25%, 11%, 10.75%, 10.50%, 10.25%, 10%, 9.75%, 9.50%, 9.25%, 9%, 8.75%, 8.50%, 8.25%, 8%, 7.75%, 7.50%, 7.25%, 7%, 6.75%, 6.50%, 6.25%, 6%, 5.75%, 5.50%, 5.25%, 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v.

In selected embodiments, the concentration of an active agent provided in the pharmaceutical compositions of the invention is independently in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v.

In selected embodiments, the concentration of an active agent provided in the pharmaceutical compositions of the invention is independently in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v.

In selected embodiments, the amount of an active agent provided in the pharmaceutical compositions of the invention is independently equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g or 0.0001 g.

In selected embodiments, the amount of an active agent provided in the pharmaceutical compositions of the invention is independently equal to or more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g or 10 g.

In an embodiment, this disclosure provides a kit for the remotely-triggered synergistic combination therapy useful for the treatment of a cancer comprises (1) a particle heater preparation having a material interacting with an exogenous source and a carrier, (2) a pharmaceutical dosage containing an active agent; wherein the pharmaceutical dosage of an active agent containing any of the active agent as disclosed herein, and wherein the pharmaceutical dosage of the active agent is selected from the group of a capsule, a tablet, a buccal tablet, an oral disintegrating tablet, a liquid formulation, a dispersion, an injection preparation, powder for injection, and suppository. Depending on the cancer types and the therapeutic strategy, the particle heater and the pharmaceutical dosage of the anticancer agent may be administered simultaneous, or sequentially.

### 7. Remotely-Triggered Synergistic Combination Therapy

7a. Exogenous Source Triggered Thermal Therapies

Photothermal therapy (PTT) is a therapeutic strategy that uses light absorbing materials (material interacting with exogenous source) to cause cell death (e.g. apoptosis or necrosis depending on the laser dosage, type and irradiation duration) by inducing hyperthermia. Compared with traditional chemotherapy, PTT exhibits unique advantages such as higher specificity, minimal invasiveness and higher efficacy.

Under pulsed laser irradiation, cell damages are induced via a series of photothermal and accompanied phenomena, denaturation or breakdown of proteins, cell cavitation, cellular structure rupture, evaporation of cellular liquid and bubble formation by shock waves due to particle thermal expansion, evaporation, or plasma generation of LSPR nanoparticles.

An important physical property of the particle heater for causing cell damages is the increased temperature within a biological system and the scope and spatial span over which the temperature change occurs. In a typical PTT application of particle heaters, the particles are injected into a small cavity inside a tissue and are optically stimulated. When the exogenous light source is applied, the material encapsulated in the particle will interact with the light source, absorb the energy thereof, and convert the energy to heat that travels outside the particle. Tissues typically have the heat conductivity of water and heat from the particle heater is likely to flow isotropically inside the tissue.

In many PTT applications, it is desirable to target cells for localized heating to provide tunable temperature raise. Techniques which effect precise localized heating would allow for producing therapeutic benefits of killing diseased cells while minimizing the collateral damage to nearby cells and tissues. The laser energy used to destroy the cells when the nanoparticles are located on the cytoplasm membrane is 10 times lower than that required when nanoparticles are internalized inside the cytoplasm.

In one embodiment, the disclosure provides a method of generating heat by irradiating particle heaters or particle heater compositions described herein. Irradiating the particle heaters or composition thereof includes directing electromagnetic radiation onto the particle heaters or composition thereof. The electromagnetic radiation may come from any source, including an LED, laser, or lamp. Any source that can provide the appropriate radiation, including wavelength and intensity, is compatible with the disclosed methods. In one embodiment, the source is a narrow-band EMR source, with a particular bandwidth tuned to wavelengths compatible with human tissue. In another embodiment, the source is a broad-band EMR source. In some embodiments, the source is a laser. In some embodiments, the source is a pulsed laser.

In some embodiments, the method further comprises heating an area in the proximity of the particle heaters or composition thereof by transferring heat from the particle heaters or the particle heater composition to the surrounding area. As used herein, the term “in proximity to” is defined as an area containing the particle heaters or composition thereof sufficiently near the particle heaters or composition thereof to receive heat transferred from the particle heaters or composition thereof after irradiation. By this step, heating the particle heaters or composition thereof is used to heat an area around the particle heaters or composition thereof to provide targeted heat. The area can be liquid, solid, gas, or any combinations thereof. The area to be heated by particle heaters or composition thereof can be liquid, solid, gas, or any combinations thereof. In some embodiments, the area is heated to a temperature of 37.5° C. to 120° C.

In some embodiments, this disclosure provides a method of heating an area of tissue having diseased cells in proximity to the particle heaters or composition thereof by the heat from the particle heaters for a sufficient period of time at a temperature ranging from about 38.0° C. to about 52.0° C.

In one embodiment, the method further includes heating a plurality of the particle heaters. While a single particle heater may be effective in a nano- or micron-scale environment, greater area can be heated by irradiating a plurality of the particle heaters.

In an embodiment, this disclosure provides a method for inducing localized hyperthermia at a tissue site in a subject comprising: administering an effective amount of the particle heater comprising a carrier admixed with a material to the tissue site in the subject; exposing the particle to an exogenous source that heats the particle heater for a sufficient period of time to induce localized hyperthermia at a temperature ranging from about 38.0° C. to about 52.0° C. In some embodiments, the subject is a warm-blooded animal. In some embodiments, the subject is a human.

In some embodiments, the method further comprises heating a surrounding area in proximity to the particle heater by transferring heat from the particle heater to the surrounding area. In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C.

In one embodiment, the area is heated to a temperature greater than 42° C. In one embodiment, the area is heated to a temperature of 37.5° C. to 50° C. In one embodiment, the area is heated to a temperature of about 37.5° C., about 38° C., about 38.5° C., about 39° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 47.5° C., about 48° C., about 48.5° C., about 49° C., about 49.5° C., or about 50° C. In some embodiments, the hyperthermia induced the remotely triggered energy-thermal conversion is of a temperature ranging from about 38.0° C. to about 90.0° C.

In an embodiment, this disclosure provides a method of remotely triggered thermal killing of unwanted cells comprising the steps of: (1) administering an therapeutically effective does of heat delivery particles and waiting for a period of time to allow distribution of the particles to the unwanted cells, (2) exposing the tissue site having unwanted cells to an exogenous source for sufficient period of time, wherein the material absorbs the energy from the exogenous source and converts the energy to heat, wherein the heat induces localized hyperthermia at the tissue site, wherein the localized hyperthermia causes the death of the unwanted cells. In some embodiments, for any herein described methods, the “unwanted cells” comprise pathogenic microbial cells. In some embodiments, for any herein described methods, the “unwanted cells” comprise human macrophage cells that are hijacked by pathogenic microbial cells. In some embodiments, for any herein described methods, the “unwanted cells” comprise tumor cells.

In an embodiment, this disclosure provides a method for effecting remotely triggered thermal killing of unwanted cells at a tissue site comprising: (1) administering an therapeutically effective amount of the particle heaters as described herein to the tissue site having the unwanted cells and allowing the cells to associate with the particle heaters, and (2) exposing the particle heaters at the tissue site to an exogenous source for a sufficient period of time, wherein the particle is constructed such that it passes the Extractable Cytotoxicity Test, and the material absorbs the energy from the exogenous source and converts the energy into heat; then the heat travels outside the particle to cause a temperature increase in a tissue area surrounding the particle heaters thereby to induce localized hyperthermia at a temperature ranging from about 38.0° C. to about 52.0° C. that is sufficient to selectively kill the unwanted cells. In some embodiments, for any herein described methods, the “unwanted cells” comprise pathogenic microbial cells. In some embodiments, for any herein described methods, the “unwanted cells” comprise human macrophage cells that are hijacked by pathogenic microbial cells. In some embodiments, for any herein described methods, the “unwanted cells” comprise tumor cells. In some embodiments, the material in the particle exhibits stability such that the particle is considered passing the Efficacy Determination Protocol,

In some embodiments, the exogenous source is selected from the group of an electromagnetic radiation, an electrical field, a microwave, a radio wave, an ultrasound, a magnetic field, and combinations thereof. In some embodiments, the exogenous source comprises microwave.

In some embodiments, the exogenous source comprises an ultrasound. In some embodiments, the material comprises ICG dye.

In some embodiments, the exogenous source is an ultrasound (US) producing machine. In some embodiments, the therapeutic ultrasound is either pulsed or continuous.

The frequency of ultrasound dictates the depth of penetration and affects the efficiency of particle heaters. To reach deeper tissues (up to 5 cm or more), a frequency of 1 MHz should be selected. When the target tissue is within 2.5 cm from the surface of the skin, a frequency of 3 MHz should be selected. It is important to note that 3 MHz will produce heat from particle heaters approximately 3-times faster than 1 MHz, creating a higher efficiency in heating when compared to 1 MHz ultrasound for the same particle heater. For continuous US, frequencies within the range of 1-3 MHz at intensities of 0.5-10 W/cm² for a duration of 1-15 minutes at 100% duty cycle should be useful for in vivo applications. In some embodiments, the US frequencies of 1-2 MHz at intensity ranges from 0.5-5 W/cm² are applied for 1-5 minutes at 100% duty cycle. 3 MHz ultrasound is absorbed more rapidly in the tissues, and therefore is considered most appropriate for superficial lesions, whilst the 1 MHz energy is absorbed less rapidly with deeper progression through the tissues, and can therefore be more effective at greater depth. The boundary between superficial and deep tissues is in some ways arbitrary, but somewhere around the 2 cm depth is often taken as a useful boundary. Hence, if the target tissue is within 2 cm (or just under an inch) of the skin surface, 3 MHz treatments will be effective whilst treatments to deeper tissues will be more effectively achieved with 1 MHz ultrasound. One important factor is that some of the ultrasound energy delivered to the tissue surface will/may be lost before the target tissue (i.e. in the normal or uninjured tissues that lie between the skin surface and the target). In order to account for this, it may be necessary to deliver more US energy at the surface than is required, therefore allowing for some absorption before the target tissue, and allowing sufficient remaining ultrasound to achieve the desired effect. To identify the appropriate dose to set on the machine, one has to determine (a) the estimated depth of the lesion to be treated and (b) the intensity of ultrasound required at that depth to achieve the desired effect. For example, to achieve a 0.5 W/cm² intensity at 1 cm tissue depth, one would select 3 MHz treatment option and set machine to 0.7 W/cm² which will result in 0.5 W/cm² intensity at a 1 cm tissue depth. The rate at which ultrasound is absorbed in the tissues can be approximately determined by the half value depth—this is the tissue depth at which 50% of the ultrasound delivered at the surface has been absorbed. The average half value depth of 3 MHz ultrasound is taken at 2.5 cm and that of 1 MHz ultrasound as 4.0 cm though there are numerous debates that continue with regards the most appropriate half value depth for different frequencies.

In some embodiments, pulsed ultrasound is used. The pulse ratio determines the concentration of the sound energy on a time basis. The pulse ratio determines the proportion of time that the ultrasound machine is “ON” compared with the “OFF” time. A pulse ratio of 1:1 for example means that the machine delivers one ‘unit’ of ultrasound followed by an equal duration during which no energy is delivered. The machine duty cycle is therefore 50%. A machine pulsed at a ratio of 1:4 will deliver one unit of ultrasound followed by 4 units of rest, therefore the machine is on for 20% of the time (some machines use ratios, and some use percentages). The selection of the most appropriate pulse ratio essentially depends on the state of the target tissue(s). The less dense the target tissue state, the more energy sensitive it is, and appears to respond more favorably to energy delivered with a larger pulse ratio (lower duty cycle). As the tissue becomes denser, it appears to respond preferentially to a more ‘concentrated’ energy delivery, thus reducing the pulse ratio (or increasing the duty cycle). It is suggested that pulse ratios of 1:4 would be best suited to the treatment of low density tissues, reducing this as the tissue increases in density, moving through 1:3 and 1:2 to end up with 1:1 or continuous modes As a general rule, the pulse ratio of 1:4 or 1:3 will be for the less dense tissues, 1:2 and 1:1 for the medium density tissues and 1:1 or continuous for the denser tissues. The final compilation of the treatment dose that is most likely to be effective is based on the principle that one needs to deliver about 1-minute worth of ultrasound energy (at an appropriate frequency and intensity) for every treatment head that needs to be covered. The size of the treatment area will influence the treatment time, as will the pulse ratio being used. The larger the treatment area, the longer the treatment will take. The more pulsed the energy output from the machine, the longer it will take to deliver about a 1-minute worth of ultrasound energy. Sound dose will obviously also depend on the particle heater concentration at the target tissue.

In some embodiments, the exogenous source comprises an electromagnetic radiation.

In some embodiments, the electromagnetic radiation source comprises a LED light or a laser light.

In some embodiments, the electromagnetic radiation source comprises a LED light. LEDs are solid state p-n junction devices that emit light when forward biased. An LED is a Light Emitting Diode, a generic term. An IRED is an Infrared Emitting Diode, a term specifically applied to Excelitas IR emitters. Unlike incandescent lamps that emit light over a very broad range of wavelengths, LEDs emit light over such a narrow bandwidth that they appear to be emitting a single “color”.

In some embodiments, the material absorbing optical energy at a wavelength from 750 nm-950 nm (e.g. Infrared Light Emitting Diodes (IRED) by Excelitas). In some embodiments, the material absorbing optical energy at a wavelength from 400 nm to 750 nm (e.g. a LED device). In some embodiments, the material is selected from the group of squaraine dye, IR 193 dye, ICG dye, IR 820 dye (new ICG dye), and combinations thereof.

In some embodiments, it is desirable to keep the temperature in the surrounding area of the heat delivery composition/medium/particle to be sufficiently low to avoid collateral damage to the healthy tissues and also control the temperature rise to be sufficiently high to kill unwanted cells. In some embodiments, for any herein described methods, the “unwanted cells” comprise pathogenic microbial cells. In some embodiments, for any herein described methods, the “unwanted cells” comprise human macrophage cells that are hijacked by pathogenic microbial cells. In some embodiments, for any herein described methods, the “unwanted cells” comprise tumor cells.

In some embodiments, the electromagnetic radiation source is a laser light. In some embodiments, pulsed lasers are utilized in order to provide localized thermal heating. In some embodiments, the laser irradiation is delivered in a pulse duration longer than the thermal relaxation time (TRT) of the particle heaters such that the heat generated by the particle begins to travel outside the particle. In some embodiments, the flow of the heat delivery to the outside of the particles can be achieved by manipulating the fluence of the laser irradiation, particle size and the concentration of the particles. Pulses are at least nanoseconds in duration. In some embodiments, the exogenous source is laser pulse radiation at a determined thermal relaxation time (TRT). In some embodiments, the TRT is selected from the group of picoseconds and nanoseconds. In some embodiments, the TRT is selected from the group of microseconds and milliseconds.

In some embodiments, the laser pulse duration is in a range from milliseconds to nanoseconds, and the laser has an oscillation wavelength at 805 nm, 808 nm, or 1064 nm. In some embodiments, the laser pulse duration is in a range from milliseconds to femtoseconds. In some embodiments, the particle heater absorbs the laser light having a wavelength from 750 nm to 1100 nm. In some embodiments, the material is selected from the group of indocyanine green dye (ICG), new ICG dye (IR820), IR 193 dye, iron oxide, a tetrakis aminium dye, and combinations thereof.

In some embodiments, the exogenous source is a laser. In some embodiments, the device for the laser light delivery comprises a fiber optic conduit coupled to a source of laser energy. This comprises a hollow sheath, which covers the distal end of the fiber optic conduit, defines a pocket, and a fiber optic lens in the pocket, and is modified to receive and direct the laser energy emitted from the fiber optic conduit through the lens onto the occlusion and to form a channel therethrough. The fiber optic conduit can be adapted for the specific application. Optical fibers are hair thin strands of glass or plastic that guide light. The optical fiber has an inner core surrounded by an outer cladding. In order to guide the light, the core refractive index is higher than the cladding index. A fiber grating is formed inside the core of a fiber. This is widely used in the field of fiber-optic communication for wavelength management. The optical grating reflects or transmits a certain portion, wavelength (bandwidth) or intensity, of the light along the optical fibers. A fiber Bragg grating is based on the interference of multiple reflections of a light beam in a fiber segment whose index of refraction varies periodically along the length of the fiber. Variations of the refractive index constitute discontinuities that emulate a Bragg structure. If the spacing of the index periods is equal to one-half of the wavelength of the light, then the waves will interfere constructively (the round trip of each reflected wave is one wavelength) and a large reflection will occur from the periodic array. Optical signals whose wavelengths are not equal to one-half the spacing will travel through the periodic array unaffected. In one embodiment, the optical grating is a Bragg grating or a long period grating. In another embodiment, the optical grating is coated with a composition having a thermal coefficient that is greater than the thermal coefficient of the fiber. In a further embodiment, at least one optical fiber further comprises an optical diffraction means for simultaneously measuring multiple peaks of the reflected light beam. In a further embodiment, the optical grating has a length between 0.2 and 40 mm.

Endoscopes are well-known medical instruments used to visualize the interior of a body cavity or organ. Endoscopes are used in a variety of operative procedures, including laparoscopic surgery where endoscopes are used to visually examine the peritoneal cavity. Typical endoscopes are configured in the form of a probe having a distal end for insertion through a small incision in the body. The probe includes components for delivery of illumination light and collection of an image from inside the body. Optical fibers or optically transmissive composition in a tubular formation typically provides illumination light delivery to a distal end of the probe. Imaging is typically carried out by an objective lens and relay optics that receive and deliver an image to the proximal end of the probe, which may be equipped with an eyepiece or an electronic image capture device such as a CCD (charge coupled device) sensor array. Endoscope probes may be rigid or flexible, with the light delivery and image retrieval components configured accordingly. Flexible bundles of optical fibers are used to produce a flexible probe, while rigid probes may have fused optical fiber assemblies, rigid light pipes and/or imaging rods and lenses. The intended use of the endoscope dictates the length of the probe, the need for flexibility and the necessary image quality.

In some embodiments, method for delivery of therapeutic laser light for particle-based therapy comprises: 1. providing an endoscope with a light delivery optical pathway transmissive of said therapeutic light, said endoscope also including an image retrieval optical pathway and imaging system for generating an image of a target area; 2. providing a light generator that selectively produces said therapeutic light and also generates visible light; 3. inserting said endoscope into a cavity of a living organism to identify and illuminate a target area, said inserting including employing the image to direct said insertion and identify said target area; 4. Exposing the particle heater to the therapeutic light to produce heat to achieve a therapeutic objective at said target area; and 5. Removing said endoscope from said cavity. In some embodiments, the therapeutic light has a wavelength from 750 nm to 1100 nm.

In some embodiments, the therapeutic light delivery endoscope comprises: 1. a broad spectrum light source that generates pulses of light having wavelengths between about 700 nm and about 1100 nm; 2. a control circuit operatively connected to said broad spectrum light source, said control circuit providing adjustable control over the frequency, power and wavelength of said light pulses; 3. a light delivery optical pathway constructed of components selected to transmit light including UV light having a wavelength between 200 nm and 300 nm, said light delivery optical pathway arranged to receive and transmit light generated by said broad spectrum light source to a target area; 4. an image retrieval optical pathway arranged to receive light reflected from said target area; 5. an image generating system which employs light from said image retrieval optical pathway to generate an image of said target area; and 6. an interface allowing a user to adjust the frequency, power and wavelength of said pulses of light, thereby controlling the quantity of the light delivered to the target tissue area.

In some methods for treating a tissue region of a patient, the method will comprise: a) providing an elongated tubular catheter for contacting the tissue region, wherein the elongated tubular catheter comprises a plurality of peripheral optical fibers in a support scaffold structure that is radially expandable and a middle optical fiber, each fiber having at least one optical grating along the axis of the fiber, wherein the middle fiber further comprises a light transmission zone configured for photodynamic (PDT) or photothermal therapy (PTT) or both (PDT+PTT); c) administering a therapeutically effective amount of at least one particle composition, wherein the particle composition is localized to the tissue region. c) monitoring the imaging/contrast agent in the tissue region, wherein a light source having a light beam is coupled into each optic fiber; at least one optical grating reflecting a certain wavelength or intensity of the light beam, and the certain wavelength or intensity of the resulting light is correlated to the contrast agent; d) exposing the particle heaters to the light to produce a phototoxic species or localized heat, wherein the light is transmitted from the middle fiber; and e) treating the tissue region photodynamically or photothermally or both (PDT+PTT). In some embodiments, the step of inserting the elongated tubular catheter is via a percutaneous procedure. In some embodiments, the tissue region is a blood vessel. In alternate embodiments, the tissue region is a tissue of internal organs.

In some methods for remotely triggered combination therapy for treating a tissue region of a patient, the method comprises: a) providing a device with at least one optical fiber, wherein the fiber comprises a light transmission zone intimately contacting the tissue region configured for remotely triggered therapy; b) administering a therapeutically effective amount of at least one particle composition, wherein the particle composition is at least partially localized to the tissue region; c) exposing the localized particle heaters to light to produce a phototoxic species or localized hyperthermia or both, wherein the activating light is transmitted from the at least one optical fiber; and d) treating the tissue region photodynamically or photothermally or both. In some embodiment, the device is selected from a group of a catheter, a cannula, a needle, a basket-type catheter, and an implant.

In some embodiments, the device is a catheter or a cannula.

By way of examples, a particle or particle composition is administered to a patient under a physician's supervision. An optical apparatus as described above is inserted into the blood vessel at about the target tissue. The diagnostic function of the composition is activated to measure the imaging function (e.g. ICG fluorescence) of the particles in the target tissue. Once the region of vulnerable plaque at the target tissue is confirmed, the therapeutic function of the apparatus is activated to provide light energy to activate the needed photodynamic therapy or photothermal therapy or both. The remaining or surrounding tissue outside the irradiation zone would not go through the remotely triggered light therapy due to the absence of the light wavelength. Therefore, the treatment is limited to the target tissue locally when the method of the present invention is used comprising a light transmission zone of an optic fiber apparatus intimately contacting the tissue region configured for therapy.

In some embodiments, the material encapsulated in the particle heaters absorbs the photons of the laser to generate heat. Such heat travels outside the particle heater to the area in proximity to the particle heaters and causes significant observable temperature change thereof.

The advantages of the efficient localized heating achieved by the particle heaters or composition thereof in this disclosure are immediately evident because the temperature change is primary limited to the area surrounding the particle heaters or composition thereof, that is, selective placement of the particle heaters allows heating of targeted regions without significantly affecting the remainder of the tissue. In addition, the photothermal effect enables heat to be generated by the particle heater as opposed to the conventional laser-based photothermal tissue treatments that deliver energy to water and the endogenous natural pigments and IR absorbing agents in the tissue (e.g. melanin). Thus, the process of the energy delivery by the exogenous source to the particle heaters in this disclosure can include selectively applying the exogenous source only to a predefined region of the tissue that is to be treated by the selective placement of the particle heaters.

In some embodiments, the material has strong absorption of photons at wavelengths overlapping with the output of the various commercially available lasers. The selection of laser parameters used to cause a controlled heat generation may include wavelength, average power, instantaneous power, pulse duration and/or total exposure duration. The pulse duration (t_{d}), of the exposure can influence the specificity or confinement of collateral thermal damage, and may be determined from the thermal relaxation time (tᵣ, also known as TRT) of the target material. The transition from specific to non-specific thermal damage can occur when the ratio is as follows: (t_{d}/tᵣ)≥1. For spheres of radius, R, and thermal diffusivity, κ, the thermal relaxation time can be provided by tᵣ=(R²/60.75κ). To transfer of the heat outside the particle selectively, the pulse duration of the laser exposure is greater than the thermal relaxation time of the particle. The power density is selected to be sufficient to induce localized mild hyperthermia (e.g. a temperature increase of at least 5° C. about room temperature) in the surrounding environment of the particles.

In some embodiments, the laser irradiation is delivered in a pulse duration longer than the TRT of the particle heaters such that the heat energy generated in the particle heaters travels outside the particle heaters. In some embodiments, the flow of heat delivered to the outside of the particle heaters can be achieved by manipulating the wavelength of the laser irradiation, pulse duration, particle size and the density of the particle heaters at the targeted tissue site.

To avoid healthy tissue damage, it is important to ensure the energy of laser irradiation is preferentially absorbed by the particles containing the material and not absorbed by the tissue to be treated. When the pulse duration exceeds the TRT of the particle heaters, then the heat energy generated begins to travel outside the particles. In addition, the duration of the pulse can be controlled to ensure that the heat produced by the particles will diffuse out into the surrounding environment.

In some embodiments, laser wavelength has a dual impact attributable to the absorption coefficient of the light energy absorbing material as well as the depth of penetration to the tissue site, which roughly increases as the wavelength increases in the visible and near infrared spectrum. After carefully choosing a proper laser wavelength and pulse duration for a particular light energy absorbing material, delivering the maximum number of photons to the particle heaters or composition thereof having the same light energy absorbing material can be achieved.

In some embodiments, the particle heater offers tunable photon absorption by varying the particle size, particle concentration, and selection of light energy absorbing material with a defined chemical structure to allow facile matching of particle absorption to the output of various commercial lasers. Additionally, the method in this disclosure affords a path to minimize tissue damage by using the least harmful wavelengths of laser light sources.

In some embodiments, laser irradiation is applied until the temperature of the surrounding area is about 40° C. to about 60° C. The exposure time is dependent upon many factors, including but not limited to, the area to be covered, wavelength and intensity of the radiation, type and mass of the composition and particle concentration.

In an embodiment, this disclosure provides a method for causing photothermal cell damage in a subject in need thereof comprising: providing a liquid suspension of therapeutically effective amount of particle heaters comprising a carrier admixed with a material, contacting diseased cells at a tissue site with the particle heaters, irradiating the particle heaters with NIR laser at 1064 nm at an effective energy density for a sufficient period of time to induce localized hyperthermia at the tissue site, wherein the temperature rise is effective to causes cell damages.

In some embodiments, the diseased cells are selected from the group of cancer cells, bacteria cells, protozoan cells, virus, fungal cells, macrophage cells, bone cells, and melanocytes.

In some embodiments, the particle heaters exhibit high photothermal conversion efficiency for killing diseased or unwanted cells under laser irradiation when irradiating the cells that are associated with the particle heaters with the 1064 nm laser at effective power density ranges. In some embodiments, for any herein described methods, the “unwanted cells” comprise pathogenic microbial cells. In some embodiments, for any herein described methods, the “unwanted cells” comprise human macrophage cells that are hijacked by pathogenic microbial cells. In some embodiments, for any herein described methods, the “unwanted cells” comprise tumor cells.

Due to the efficient absorption of the particles, photothermal heating to significant temperatures can be achieved without harming the tissue of a treatment subject. In one embodiment, irradiating the particle heater with a laser irradiation at a wavelength of 650 nm to 1350 nm. In one embodiment, irradiating the particle heater with a laser irradiation at a wavelength of 785 nm to 900 nm. In one embodiment, irradiating the particle heater with a laser irradiation at a wavelength of 650 nm to 1000 nm.

In some embodiments, the laser has a peak oscillation wavelength selected from the group 700 nm, 766 nm, 777 nm, 780 nm, 783 nm, 785 nm, 800 nm, 808 nm, 810 nm, 820 nm, 825 nm, 900 nm, 948 nm, 950 nm, 960 nm, 980 nm, 1000 nm, 1060 nm, 1064 nm, 1070 nm, 1071 nm, 1073 nm, 1098 nm, and 1100 nm. In some embodiments, the laser has an oscillation wavelength at 1071 nm. In some embodiments, the laser has an oscillation wavelength at 1064 nm. In some embodiments, the laser has an oscillation wavelength at 808 nm.

In some embodiments, the pulse duration of the laser is longer than the TRT of the particle heater. In some embodiments, the laser pulse duration is in a range from milliseconds to femtoseconds, and the laser has an oscillation wavelength at 1064 nm. In some embodiments, the laser pulse duration is in a range from milliseconds to femtoseconds, and the laser has an oscillation wavelength at 805 nm.

In some embodiments, the exogenous source comprises light sources such as a laser (ion laser, semiconductor laser, Q-switched laser, free-running laser, or fiber laser). Typically, the energy source is capable of emitting radiation at a wavelength from about 700 nm, 1000 nm, 2000 nm, 5000 nm, about 10,000 nm or more. In some embodiments, the photonic energy is radiation at an intensity from about 0.00005 mW/cm² to about 1000 TW/cm². The optimum intensity is chosen to induce high thermal gradients from heat delivery particles in a range from submicron to about 10 microns in the surrounding tissue but has minimal residual effect on heating tissue in which particles do not reside within a radius of about 100 microns or more from the nanoparticle. In certain embodiments, a differential heat gradient between the target tissue region and other tissue regions (e.g., the skin) is greater than 2-fold, 3-fold, 5-fold, 10-fold, 15-fold, 20-fold, 50-fold, 100-fold, or greater than 100-fold.

Laser sources include a pulsed laser source, which may be a single wavelength polarized (or, alternatively, unpolarized) laser source capable of emitting radiation at a frequency from about 750 nm to about 1400 nm. Alternatively, the optical source is a multiple wavelength laser source capable of emitting radiation at a wavelength from about 1000 nm to about 1200 nm. The pulsed laser source is generally capable of emitting pulsed radiation at a frequency from about 1 Hz to about 1 THz.

In some embodiments, various types of lasers may be suitable for excitation of the particles of this disclosure such as Q-switched (QS) laser such as QS alexandrite lasers (operating at 755 nm), diode lasers (operating at 805 nm) QS Nd:YAG lasers (operating at 1060 nm, 1440 nm, laser that penetrate deeper into the dermis).

Selection of a laser pulse duration is mainly guided by the TRT of the particles to be activated, which itself is related to the size of the activatable particles. Generally, the larger the particle containing the material, the longer is the TRT as larger objects take longer time to cool.

In some embodiments, the laser is operated at 750 nm, 805 nm, 808 nm, 810 nm, 1064 nm with a power density of about 40 mW/cm² to about 450 mW/cm². In some embodiments, the laser is operated at 750 nm, 805 nm, 808 nm, 810 nm, 1064 nm with a power density of about 40 mW/cm² to about 360 mW/cm². In some embodiments, the laser is operated at 750 nm, 805 nm, 808 nm, 810 nm, 1064 nm with a power density of about 100 mW/cm² to about 350 mW/cm². In some embodiments, the 808 nm NIR laser is operated at ultra-low laser power (10 mW) to generate more ROS. Various repetition rates are used from continuous to pulsed, e.g., at less than 1 Hz, or 1-5 Hz.

In some embodiments, the laser pulse duration is longer than the particle TRT. In some embodiments, the laser pulse duration is less than a millisecond or a microsecond in duration. In some embodiments, a source emitting radiation at a wavelength of 755 nm is pulsed at a duration of 0.25-400 milliseconds (ms) per pulse, with a pulse frequency of 1-10 Hz. In some embodiments, a source emitting radiation at a wavelength of 810 nm is pulsed at 5-400 ms with a frequency of 1-10 Hz. In some embodiments, a source emitting radiation at a wavelength of 1064 nm is pulsed at 0.25-400 ms at a frequency of 1-10 Hz. In some embodiments, a source emitting pulsed light at a wavelength of 530-1200 nm is pulsed at 0.5-400 ms at a frequency of 1-10 Hz.

In some embodiments, the particle heaters have TRT ranges from about 250 ns, about 275 ns, about 300 ns, about 325 ns, about 350 ns, about 375 ns, about 400 ns, about 425 ns, about 450 ns, about 475 ns, about 500 ns, about 525 ns, about 550 ns, about 575 ns, about 600 ns, about 625 ns, about 650 ns, about 675 ns, about 700 ns, about 725 ns, about 750 ns, about 775 ns, about 800 ns, about 825 ns, about 900 ns, about 925 ns, about 950 ns, about 975 ns, about 1000 ns, about 1100 ns, about 1200 ns, about 1300 ns, about 1400 ns, about 1500 ns, about 1600 ns, about 1700 ns, about 1800 ns, about 1900 ns, about 2.0 ms, about 3 ms, about 4 ms, about 5 ms, about 6 ms, about 7 ms, about 8 ms, about 9 ms, about 10 ms, about 20 ms, about 30 ms, about 40 ms, about 50 ms, about 60 ms, about 70 ms, about 80 ms, about 90 ms, or about 100 ms.

In some embodiments, short pulses (100 ns to 1000 ms) are used to drive very high transient heat gradients in and around the target tissue structure from embedded particles to localize damage in close proximity to particle location. In other embodiments, longer pulse lengths (1 ms to 10 ms, or 1 ms to 500 ms) are used to drive heat gradients further from the target structure to localize thermal energy to components greater than 100 μm away from the localized particles. In some such embodiments, pulses of varying durations are provided to localize thermal heating regions to be within 0.05, 0.1, 0.5, 1, 2, 5, 10, 20, 30, 50, 75, 100, 200, 300, 500, 1000 microns of the particles.

The skin tolerance threshold values for power densities at 968 nm is about 360 mW/cm². The skin tolerance threshold values of skins at 1064 nm is about 420 mW/cm². In some embodiments, the laser is operated at a power density of about 0.1 W/cm² to about 0.75 W/cm². In some embodiments, the laser is operated at a wavelength of 750 nm, 805 nm, 808 nm, 810 nm, and 1064 nm and an energy density of about 0.1 W/cm², about 0.11 W/cm², about 0.12 W/cm², about 0.13 W/cm², about 0.14 W/cm², about 0.15 W/cm², about 0.16 W/cm², about 0.17 W/cm², about 0.18 W/cm², about 0.19 W/cm², about 0.20 W/cm², about 0.21 W/cm², about 0.22 W/cm², about 0.23 W/cm², about 0.24 W/cm², about 0.25 W/cm², about 0.26 W/cm², about 0.27 W/cm², about 0.28 W/cm², about 0.29 W/cm², about 0.30 W/cm², about 0.31 W/cm², about 0.32 W/cm², about 0.33 W/cm², about 0.34 W/cm², about 0.35 W/cm², about 0.36 W/cm², about 0.37 W/cm², about 0.38 W/cm², about 0.39 W/cm², about 0.40 W/cm², about 0.41 W/cm², about 0.42 W/cm², about 0.43 W/cm², about 0.44 W/cm², about 0.45 W/cm², about 0.46 W/cm², about 0.47 W/cm², about 0.48 W/cm², about 0.49 W/cm², about 0.50 W/cm², about 0.51 W/cm², about 0.52 W/cm², about 0.53 W/cm², about 0.54 W/cm², about 0.55 W/cm², about 0.56 W/cm², about 0.57 W/cm², about 0.58 W/cm², about 0.59 W/cm², about 0.60 W/cm², about 0.61 W/cm², about 0.62 W/cm², about 0.63 W/cm², about 0.64 W/cm², about 0.65 W/cm², about 0.66 W/cm², about 0.67 W/cm², about 0.68 W/cm², about 0.69 W/cm², about 0.70 W/cm², about 0.71 W/cm², about 0.72 W/cm², about 0.73 W/cm², about 0.74 W/cm², about 0.75 W/cm², about 0.8 W/cm², about 0.9 W/cm², about 1.0 W/cm², about 1.1 W/cm², about 1.2 W/cm², about 1.3 W/cm², about 1.4 W/cm², about 1.5 W/cm², about 1.6 W/cm², about 1.7 W/cm², about 1.8 W/cm², about 1.9 W/cm², about 2.0 W/cm², about 2.1 W/cm², about 2.2 W/cm², about 2.3 W/cm², about 2.4 W/cm², about 2.5 W/cm², about 2.6 W/cm², about 2.7 W/cm², about 2.8 W/cm², about 2.9 W/cm², and about 3.0 W/cm², 3.1 W/cm², 3.2 W/cm², 3.3 W/cm², 3.4 W/cm², 3.5 W/cm², 3.6 W/cm², 3.7 W/cm², 3.8 W/cm², 3.9 W/cm², and 4.0 W/cm². In some embodiments, the power density of the laser irradiation ranges from about 0.5 W/cm² to 1.0 W/cm². In some embodiments, the laser is operated at a wavelength of 750 nm, 805 nm, 808 nm, 810 nm, 1064 nm and the power density of the laser irradiation is selected from the group of about 0.1 W/cm², about 0.2 W/cm², about 0.3 W/cm², about 0.4 W/cm², about 0.5 W/cm², about 0.6 W/cm², about 0.7 W/cm², about 0.8 W/cm², about 0.9 W/cm², about 1.0 W/cm², about 1.1 W/cm², about 1.2 W/cm², about 1.3 W/cm², about 1.4 W/cm², and about 1.5 W/cm². In some embodiments, the laser is operated at a wavelength selected from the group of 750 nm, 805 nm, 808 nm, 810 nm, and 1064 nm, and a power density of about 40 mW/cm² to about 450 mW/cm². In some embodiments, the laser is operated at a wavelength selected from the group of 750 nm, 805 nm, 808 nm, 810 nm, and 1064 nm with a power density of about 40 mW/cm² to about 360 mW/cm². In some embodiments, the laser is operated at a wavelength selected from the group of 750 nm, 805 nm, 808 nm, 810 nm, and 1064 nm with a power density of about 100 mW/cm² to about 350 mW/cm².

In some embodiments, the 808 nm NIR laser is operated at ultra-low laser power (10 mW) to induce the generation of ROS. Various repetition rates are used from continuous to pulsed, e.g., at less than 1 Hz, or 1-5 Hz.

In some embodiments, the tissue is irradiated at a fluence of 1-60 J/cm² with laser wavelengths of about, e.g., 750 nm, 810 nm, 1064 nm, or other wavelengths, particularly in the range of infrared light. Various repetition rates are used from continuous to pulsed, e.g., at 1-10 Hz, 10-100 Hz, 100-1000 Hz. While some energy is reflected, it is an advantage of the subject matter described herein is that particle heaters absorb a substantial amount of energy, with a lesser amount absorbed by the surrounding tissue. Particles are delivered to the tissue site at concentration that is sufficient to absorb more energy (e.g., 1.1-100×) than other components of the tissue of a similar volume. This is achieved in some embodiments, by having a concentration of particles at the tissue site with absorbance at the laser peak of 1.1-100× relative to other tissue components of similar volume.

To achieve tunable localized heat delivery, particles are utilized in conjunction with a laser or other excitation source of the appropriate wavelength. The laser light may be applied in pulses with a single pulse or with multiple pulses of light. The intensity of heating and distance over which the photothermal effect will occur are controlled by the intensity and duration of light exposure, and the concentration of the particles.

In some embodiments, the method employs a particle heater formulation applied to the tissue site containing a low concentration of particles and a high intensity laser irradiation such that the local temperature maxima caused by photothermal conversion by the particle heaters are within a nanometer scale distance from the excited particles. In some embodiments, the method employs a composition applied to the tissue site containing a higher concentration of particles and a low intensity laser irradiation such that the local temperature maxima caused by photothermal conversion from the particle heaters are at a millimeter scale distance from the excited particles (also known as collective photo-heating).

In some embodiments, the particle heaters are present in the composition in an amount ranging from about 0.5 wt. % to about 25 wt. % by the total weight of the composition. In some embodiments, the particle heater is present in an amount ranging from about 1.0 wt. % to about 20.0 wt. % by the total of the composition. In some embodiments, the particle heater is present in an amount ranging from about 5.0 wt. % to about 20.0 wt. % by the total of the composition. In some embodiments, the particle heater is present in an amount ranging from about 5.0 wt. % to about 15.0 wt. % by the total of the composition. In some embodiments, the particle heater is present in an amount ranging from about 10.0 wt. % to about 15.0 wt. % by the total of the composition. In some embodiments, the particle heater is present in an amount selected from the group of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. % by the total weight of the composition. In some embodiments, the particle heater is present in an amount selected from the group of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, and about 15.0 wt. % by the total weight of the composition. In some embodiments, the particle heater is present in an amount selected from the group of about 1.0 wt. %, 2.0 wt. %, 3.0 wt. %, 4.0 wt. %, about 5.0 wt. %, about 10.0 wt. %, and about 15.0 wt. %.

In some embodiments, pulsed lasers are utilized in order to provide localized thermal heating. In some embodiments, the laser irradiation is delivered in a pulse duration longer than the thermal relaxation time (TRT) of the particles containing the exogenous source interacting material such that the heat energy generated by the particle begins to travel outside the particle. In some embodiments, the flow of the heat delivery to the outside of the particles can be achieved by manipulating the fluence of the laser irradiation, particle size and the concentration of the particles. Pulses are at least femtoseconds in duration.

Temperatures greater than 50° C. can induce tissue fusion. (“tissue welding”). This is believed to be induced by the denaturation of the proteins and the subsequent entanglement of adjacent protein chains. In some embodiments, the temperature realized at the tissue site by particles is higher than 50° C. In some embodiments, the temperature realized at the tissue site is in a range from about 40° C. to about 50° C. In some embodiments, the peak temperature realized in the tissue from particle heating is at least 38, 39, 40, 41, 42, 43, 44, 45, 46, 47, 48, 49, 50, 51, 52, 53, 54, 55, 56, 57, 58, 59, 60, degrees Celsius (° C.). In some embodiments, that utilize pulsed laser irradiation, high peak temperatures are realized locally within specific regions at the tissue site without raising the bulk tissue temperature.

In some embodiments, the particle heater is used under the guidance of in vivo imaging, including fluorescence imaging. Imaging can provide useful information such as the size and location of the targeted tissue, as well as the relationship of the targeted tissue with its surrounding tissues.

7b. Remotely-Triggered Synergistic Combination Therapy of Cancer and Method of Treating Cancer

Cancers are more susceptible to low hyperthermia effects than normal tissues; therefore both systemic and localized hyperthermia regimes have been successful in treating cancers. Tissue damages is evident within minutes when the temperature of tissue reaches 55-95° C. Many natural light absorbers in tissues (e.g., water, hemoglobin, oxyhemoglobin and melanin) can convert light to heat for causing hyperthermia damage to both tumors and healthy tissues. However, near infrared light (NIR) induces minimal photothermal heating in both tumors and healthy tissues as the absorption of biological tissues is lowest in a NIR region (700 nm to 1400 nm).

Photothermal therapy (PTT) is a therapeutic strategy that uses light absorbing agents (material interacting with exogenous source) to cause cell death (e.g., apoptosis or necrosis depending on the laser dosage, type and irradiation duration) by inducing hyperthermia. Compared with traditional chemotherapy, PTT exhibits unique advantages such as higher specificity, minimal invasiveness and higher efficacy. Under pulsed laser irradiation, cell damages are induced via a series of photothermal and accompanied phenomena, denaturation or breakdown of proteins, cell cavitation, cellular structure rupture, evaporation of cellular liquid and bubble formation by shock waves due to particle thermal expansion, evaporation, or plasma generation of gold nanoparticles (LSPR particles).

PTT for treating cancer employs NIR light induced localized hyperthermia to cause thermal cytotoxic effects on tumor cells (e.g., apoptosis or necrosis depending on the laser dosage, type and irradiation duration). Hyperthermia can lead to tumor cell death via protein denaturation or rupture of the cellular membrane and subsequently result in tumor shrinkage due to removal of cancerous cells by macrophages, which achieve numerous potential benefits over conventional cancer therapies.

Numerous cyanine dyes have employed as photothermal conversion agents due to their strong NIR absorbance and the conversion of the absorbed photonic energy to heat. However, the direct use of free cyanine dyes in PTT is severely limited by its poor aqueous solubility, rapid body clearance, poor cellular uptake, and lacking targeting capacity. Indocyanine green (ICG) is the only NIR dye approved by FDA for clinical imaging and diagnosis, which has been widely studied for photothermal cancer therapy. To address some of the deficiencies associated with the known ICG based PTT procedures, this disclosure provides particle heaters containing a carrier for encapsulating a material interacting with an exogenous source as described above. Upon contacting with the exogenous source, the material absorbs the energy from the exogenous source and converts the energy to heat to induce localized hyperthermia at a temperature range that is sufficient to kill the cancer cells in the tumor tissue. Particle heaters may be nanoparticles having a median particle size less than 250 nm, preferably nanoparticles having a median particle size of about 150 nm. Particles may have hydrophilic polymer surface modifications as described above that allow them to circulate in the body's vascular system for a prolonged period to get high accumulation in the tumor tissue.

Particles may further be surface modified to be localized to the tumor site using multifunctional targeting ligands. For example, particles have epidermal growth factor receptor (EGFR) binding antibody conjugated to its surface; particles have EGFR binding kinase inhibitors conjugated to its surface via glutathione cleavage disulfide bond (—S—S—). This particle design provides the synergistic benefits of enhanced concentration of particle heater at the tumor site via tumor cell membrane receptor targeting. The encapsulation of the material by the carrier minimize the cytotoxicity caused by leakage of the material or toxic degradation component of the material outside the particle after exposure to the exogenous source. The use of EGFR binding antibody or peptides as targeting ligand greatly improves the precision of the delivery of particle heaters to the desired tumor site.

An important physical property of the particle heater for causing tumor cell damage is the increased temperature within a biological system and the scope and spatial span over which the temperature change occurs. In a typical PTT application of particle heaters, the particles are injected into a small cavity inside a tissue and are optically stimulated. When the exogenous light source is applied, the material encapsulated in the particle will interact with the light source, absorb the energy thereof, and convert the energy to heat that travels outside the particle. Tissues typically have the heat conductivity of water and heat from the particle heater is likely to flow isotropically inside the tissue.

In many PTT applications, it is desirable to target cells for localized heating to provide tunable temperature rise. Techniques that effect precise localized heating would allow for producing therapeutic benefits of killing tumor cells while minimizing the collateral damage to nearby cells and tissues.

In one embodiment, the disclosure provides a method of generating heat at a tumor site in a subject by irradiating particle heaters or particle heater compositions described herein. Irradiating the particle heaters or composition thereof includes directing electromagnetic radiation onto the particle heaters or composition thereof. The electromagnetic radiation may come from any source, including an LED, laser, or lamp. Any source that can provide the appropriate radiation, including wavelength and intensity, is compatible with the disclosed methods.

In some embodiments, the method further comprises heating an area in the proximity of the particle heaters or composition thereof by transferring heat from the particle heaters to the surrounding area. As used herein, the term “in proximity to” is defined as an area containing the particle heaters or composition thereof sufficiently near the particle heaters or composition thereof to receive heat diffused out from the particle heaters or composition thereof after irradiation. By this step, heating the particle heaters is used to heat an area around the particle heaters to provide targeted heat. The area can be liquid, solid, gas, or any combinations thereof. In some embodiments, the area is heated to a temperature of 37.5° C. to 52° C.

In some embodiments, this disclosure provides a method of heating an area of tumor tissue having tumor cells in proximity to the particle heaters by the heat from the particle heaters for a sufficient period of time at a temperature ranging from about 38.0° C. to about 52.0° C.

In one embodiment, the method further includes heating a plurality of the particle heaters. While a single particle heater may be effective in a nano- or micron-scale environment, greater area can be heated by irradiating a plurality of the particle heaters.

In an embodiment, this disclosure provides a method for inducing localized hyperthermia at a tumor tissue site in a subject comprising: administering an effective amount of the particle heater as described herein to the tissue site in the subject; exposing the particle heaters to an exogenous source that heats the particle heater for a sufficient period of time to induce localized hyperthermia at a temperature ranging from about 38.0° C. to about 52.0° C. In some embodiments, the subject is a warm-blooded animal. In some embodiments, the subject is a human.

In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C.

In one embodiment, the tumor tissue is heated to a temperature greater than 42° C. In one embodiment, the tumor tissue is heated to a temperature of 37.5° C. to 50° C. In one embodiment, the tumor tissue is heated to a temperature of about 37.5° C., about 38° C., about 38.5° C., about 39° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 47.5° C., about 48° C., about 48.5° C., about 49° C., about 49.5° C., or about 50° C. In some embodiments, the hyperthermia induced is of a temperature ranging from about 38.0° C. to about 90.0° C.

In an embodiment, this disclosure provides a method of remotely-triggered thermal killing of tumor cells comprises the steps of: (1) administering an therapeutically effective does of particle heaters to a subject and waiting for a period of time to allow distribution of the particles to the tumor cells at a tumor tissue site, (2) exposing the tumor tissue having tumor cells to an exogenous source for a sufficient period of time, wherein the material absorbs the energy from the exogenous source and converts the energy to heat, wherein the heat induces localized hyperthermia at the tumor tissue, wherein the localized hyperthermia causes the death of the tumor cells.

In an embodiment, this disclosure provides a method for effecting remotely-triggered thermal killing of tumor cells at a tumor tissue site comprising: (1) administering an therapeutically effective amount of the particle heaters as described herein to the tumor tissue site having the tumor cells and allowing the cells to associate with the particle heaters, and (2) exposing the particle heaters at the tumor tissue site to an exogenous source for a sufficient period of time, wherein the particle heater is constructed such that it passes the Extractable Cytotoxicity Test, and the material absorbs the energy from the exogenous source and converts the energy into heat; then the heat travels outside the particle to cause a temperature increase in a tumor tissue area surrounding the particle heaters thereby to induce localized hyperthermia at a temperature ranging from about 38.0° C. to about 52.0° C. that is sufficient to selectively kill the tumor cells.

In some embodiments, the material in the particle exhibits stability such that the particle is considered passing the Efficacy Determination Protocol. In some embodiments, the material exhibits sufficient material process stability of retaining at least 50% of the absorbance after the exposure to the exogenous source process conditions.

In an embodiment, this disclosure provides a method for causing remotely-triggered synergistic combination therapy for the treatment of cancer in a subject comprising: (1) administering a therapeutically effective amount of the herein described particle heaters; (2) administering a therapeutically effective amount of an anticancer agent to the tumor site in the subject in need thereof and allowing the synergistic combination therapy to associate with cancer cells, and (3) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the anticancer agent exhibit synergy in killing cancer cells, and wherein the particle is constructed such that it passes the Extractable Cytotoxicity Test. In some embodiments, the anticancer agent and the particle heaters may be administered sequentially or concurrently. In some embodiments, the anticancer agent and the particle heaters may be administered sequentially. the anticancer agent and the particle heaters may be administered concurrently.

In some embodiments, the anticancer agent is further encapsulated by the particle heater having the material, and wherein the heat causes the particle heater to alter its structure to release the anticancer agent outside of the particle. In some embodiments, the anticancer agent further comprises the carrier to form a chemotherapy particle free of the material, and wherein the heat causes the chemotherapy particle to alter its structure to release the anticancer agent outside of the particle.

In some embodiments, the exogenous source is selected from the group of an electromagnetic radiation, an electrical field, a microwave, a radio wave, an ultrasound, a magnetic field, and combinations thereof.

In some embodiments, the particle heater and the anticancer agent are administered to the patient simultaneously. In some embodiments, the particle heater and the anticancer agent are administered to the patient sequentially. In some embodiments, the anticancer agent is administered before the administering of the particle heater. In some embodiments, the particle heater is administered before the administering the anticancer agent.

In some embodiments, the method further comprises performing radiation therapy. In some embodiments, the method further comprises performing surgery. Particle heater is used for the imaging guided surgery of the tumor followed by the remotely-triggered destruction of cancer cells along the surgical margins.

In some embodiments, the induced hyperthermia is a mild hyperthermia at a temperature ranging from about 38.0° C. to about 41.0° C. In some embodiments, the induced hyperthermia is a moderate hyperthermia at a temperature ranging from about 41.1° C. to about 45.0° C., wherein the hyperthermia does not cause collateral damage to healthy cells. In some embodiments, the induced hyperthermia is a profound hyperthermia at a temperature ranging from about 45.1° C. to about 52.0° C.

In some embodiments, the particle heaters are designed for photodynamic therapy combined with chemotherapy (combination PDT-chemotherapy), wherein the material is a photosensitizing agent capable of producing reactive oxygen species (ROS) under exposure to laser light. In some embodiments, the particle heaters are designed for photothermal therapy combined with chemotherapy (combination PTT-chemotherapy), wherein the material is an NIR light absorbing agent. In some embodiments, the particle heaters are designed for photothermal therapy combined with photodynamic therapy and chemotherapy (combination PTT-PDT-chemotherapy), wherein the material is a NIR absorbing agent simultaneously having the capacity of producing ROS and heat generation under exposure to laser light. Sound can also be used to produce reactive molecular species and is referred to as Sonodyanmic Therapy (or SDT). For example, ICG can be used for PDT or SDT.

PDT involves the administration of photosensitizer (PS) and then localizing to the tumor using a specific wavelength of light to activate the PS. PDT is a two-stage procedure based on three components including photosensitizer, light and oxygen. In PDT, photosensitizers would generate ROS under appropriate light irradiation. A series of photochemical reactions initiated by PS results in the death of cancer cells. Certain PS (called type I PS) can produce toxic reactive molecular species even in the absence of oxygen and can be used for PDT of hypoxic tumors.

In some embodiments, the material is a bimodal material that exhibits both NIR absorption and an ability to generate ROS under laser irradiation. The bimodal material converts light energy into thermal energy for photothermal therapy and produces reactive oxygen species for photodynamic therapy, which makes it attractive for applications in double or triple combination anticancer therapy involving PTT and/or PDT with chemotherapy. In some embodiments, the bimodal material is selected from the group of indocyanine green (ICG), new ICG dye IR820, IR 780 dye, IR 193 dye, plasmonic absorber, iron nanoparticle, gold nanostructures, and combinations thereof. For example, after the ICG nanoparticles are irradiated with pulsed laser light, excited ICG dye produces ROS in the presence of cellular water, of which ROS is unwanted cells like tumor cells or microbes.

Targeting enzymes participating in ROS scavenging (such as superoxide dismutase, heme oxygenase-1 or nitric oxide synthase) with selective inhibitors has been shown to improve antitumor activity of PDT. In some embodiments, the synergistic combination therapy may include inhibitors of enzymatic antioxidants such as superoxide dismutase (SOD), catalase (CAT), glutathione peroxidase (GPx) and thioredoxin (Trx). These inhibitors include but are not limited by: LCS-1 (4,5-dichloro-2-m-tolylpyridazin-3(2H)-one, salicylic acid, 6-Amino-5-nitroso-3-methyluracil, ATN-224 (bis-choline tetrathiomolybdate); 2-ME (2-methoxyoestradiol); N—N′-diethyldithiocarbamate, 3-amino-1,2,4-triazole, p-Hydroxybenzoic acid, misonidazole, d-penicillamine hydrochloride, 1-penicillamine hydantoin, dl-Buthionine-[S, R]-sulfoximine (BSO), and Au(I) thioglucose etc.

PTT does not require oxygen accessibility to damage targeted tissues. PTT generates localized hyperthermia to destruct tumor cells under near infrared (NIR) light irradiation. As PDT, PTT and chemotherapy each act via different mechanisms, their combination into a single therapeutic system provides a highly efficient approach to treat cancers. Upon NIR laser irradiation, PTT can directly kill cancer cells in the primary tumor sites and lymphatic metastasis via hyperthermia, or even eradicate cancer stem cells and tumor initiating cells. The migration and invasion activities of the cancer cells can be inhibited at mild hyperthermia without inducing unintended killing of healthy cells due to the inhibitory effects of hyperthermia on the expression of a variety of metastasis-related factors including matrix metalloproteinase (MMP-2/9), vascular endothelial growth factor (VEGF), and transforming growth factor-β1 (TGF-β1). It is well understood that the photothermal effects can be significantly suppressed by lowering down the tissue temperature from 37° C. to 4° C., since the photothermal heating effects initiate apoptosis processes only after reaching a threshold value of local intracellular temperature (42-45° C.), lasting for 15 minutes to 60 minutes, or higher than 50° C. lasting for 4 minutes to 6 minutes (See, Hwang et al., Advanced Materials, 2014, p. 1-7).

However, due to the heterogeneous distribution of particle heaters in tumor and the limitation of penetration depth of NIR light in deep tissues, it remains a great challenge to use PTT alone to achieve complete eradication of tumor cells, or the direction eradication of metastatic cancer cells or metastatic nodules in distant organs.

Development of novel target-specific antitumor drugs has enabled examination of a number of concept-based combinations that in various molecular mechanisms sensitize tumor cells to cytotoxic effects of PDT and/or PTT. Proteins are major targets for oxidative reactions, as they constitute nearly 70% of the dry weight of cells. Oxidized proteins can be re-folded by molecular chaperones such as HSPs. Inefficient restoration of their structure leads to accumulation of misfolded proteins and their aggregation that precipitates cell death. Accumulation of damaged or misfolded proteins within endothelial reticulum (ER) triggers a process called ER stress, which can be ameliorated by unfolded protein response (UPR) or can lead to cell death. Therapeutic approaches that interfere with re-folding or removal of oxidized proteins can be used to sensitize tumor cells to PDT and/or PTT. For example, modulation of HSP function with geldanamycin, a HSP90 inhibitor, sensitizes tumor cells to PDT and/or PTT. Bortezomib, a proteasome inhibitor successfully used in the treatment of hematological disorders potentiates cytotoxic effects of PDT and/or PTT by aggravation of ER-stress. Moreover, several apoptosis-modulating factors such as rapamycin, Bcl-2 antagonists, and ursodeoxycholic acid or ceramide analogues have been shown to increase PDT/PTT-mediated cancer cell death.

Antivascular effects of PDT and/or PTT can be further potentiated by COX inhibitors, antiangiogenic or antivascular drugs or monoclonal antibodies targeting factors promoting neovascularization (such as VEGF) significantly improving tumor growth control after PDT and/or PTT. Finally, combining PDT and/or PTT with agents that target signal transduction pathways such as the anti-EGFR agent, cetuximab may also improve the efficacy of PDT and/or PTT.

In some embodiments, the synergistic combination therapy of cancer comprise the particle heaters as disclosed herein and an anticancer agent capable of sensitize tumor cells to remotely-triggered cytotoxic effects of the material (localized hyperthermia and/or ROS); wherein the anticancer agent is selected from the group selected of HSP inhibitor, proteasome inhibitor, rapamycin, Bcl-2 antagonists, ursodeoxycholic acid, ceramide analogues, geldanamycin, bortezomib and combinations thereof.

In some embodiments, the thermotherapy sensitizing agent in the synergistic combination therapy may be administered simultaneously with the application of thermotherapy. In some embodiments, the thermotherapy sensitizing agent and the material exist as two different physical forms, the material is in particle form and the thermotherapy sensitizing agent is in a pharmaceutical dosage. In some embodiments, the thermotherapy sensitizing agent and the material exist as a unitary dosage (e.g. a single tablet). In some embodiments, the thermotherapy sensitizing agent and the material are admixed with the carrier to form two populations of particles independently or forms a single particle. In some embodiments, the thermotherapy sensitizing agent is an anti-cancer agent.

In some embodiments, the synergistic combination therapy comprising sequential administration of the thermotherapy sensitizing agent as a standalone pharmaceutical formulation followed by the deployment of the thermotherapy.

This disclosure provides particle heaters comprising an anticancer agent and a material interacting with an exogenous source admixed with a carrier. Such particles provide synergistic chemotherapy and PTT and/or PDT therapy useful for effective killing cancer cells in the primary tumor or local metastasis, inhibit cell migration and invasion activities, and eradicate the metastatic cancer cells in distant metastatic sites.

The various particle heater and anticancer chemotherapy based synergistic combination therapy designs as described herein provide synergistic multistage tumor-targeting in combined with chemo-photothermal or chemo-photodynamic therapies. Such synergistic combination therapy enhances therapeutic index as well as reduce the exposure of the healthy cells to the toxic effects of any material and the anticancer agent that may have leaked out of the particle into the body. The particle heaters also minimize the entry of body fluids inside the particle at concentrations that can degrade the material and the anticancer agent inside the particle. The particle heaters disclosed herein provides synergistic combination therapy of chemotherapy with PTT and/or PDT that involves cytotoxic mechanisms distinct from the conventional chemo- and radio-therapies.

In an embodiment, this disclosure provides a method of treating a cancer with synergistic combination therapy in a subject comprising the steps of sensitizing the cancer by administering to the subject in need thereof a treatment that will (i) induce apoptosis or autophagy in tumor cells, (ii) induce ferroptosis in tumor cells, (iii) induce necrotic cell death in tumor, (iv) modify the tumor environment, (v) stimulate tumor-infiltrating immune cells, or (vi) a combination of two or more thereof.

In some embodiments, the treatment is a particle heater or an anticancer agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat travels outside the particle to induce localized hyperthermia sufficient to selectively kill cancer cells.

In some embodiments, the anticancer agent is encapsulated in the particle heater and the heat causes the particle to alter its structure to release of the anticancer agent. In some embodiments, the anticancer agent is not encapsulated in the particle heater. In some embodiments, the anticancer agent is present in a separate pharmaceutical composition from the particle heater. In some embodiments, the particle heater is administered before the administration of the anticancer agent. In some embodiments, the particle heater is administered after the administration of the anticancer agent. In some embodiments, the particle heater is administered concurrently with the administration of the anticancer agent.

In some embodiments, the method further comprises the step of activating the particle heater remotely with an exogenous source, wherein the exogenous source is selected from the group of an electromagnetic radiation, an electrical field, a microwave, a radio wave, an ultrasound, a magnetic field, and combinations thereof.

In some embodiments, the particle heater is used to guide the imaging-based surgical debulking of the tumor followed by remotely triggering the particles for the destruction of cancer cells along the surgical margins.

In some embodiments, this disclosure provides for the image-guided surgical resection of tumor tissues followed by specific laser triggered thermal killing of tumor cells in the surgical margins. The location, size and shape of the tumor tissue to be treated is generally diagnosed and characterized by the imaging technique including fluorescence imaging, contrast enhanced computed tomography (CT), and magnetic resonance imaging (MRI). The imaging techniques allow detecting tumor tissues and enabling simultaneous guidance of therapeutic laser irradiation to induce tumor cell death by probing the exogenous delivered imaging agent.

Fluorescence imaging using intraoperative contrast agents is a rapidly growing field for improving visualization in cancer surgery to facilitate resection in order to obtain negative margins. There are multiple strategies for tumor visualization based on antibodies against surface markers or ligands for receptors preferentially expressed in cancer.

In some embodiments, the imaging technique is fluorescence imaging based on ICG dye. In some embodiments, the imaging technique is MRI using iron oxide nanoparticles as contrast agent.

In some embodiments, the activation of the particle heater occurs before the administration of the anticancer agent. In some embodiments, the activation of the particle heater occurs after the administration of the anticancer agent.

In some embodiments, sensitizing the tumor comprises administering to the subject a treatment that will induce apoptosis, autophagy, ferroptosis, or necrotic cell death in tumor cells.

In some embodiments, the tumor sensitizing treatment is selected from the group of thermotherapy, radiation therapy, surgery, chemotherapy, immunotherapy, photodynamic therapy, or a combination thereof. In some embodiments, the tumor sensitizing treatment is thermotherapy. In some embodiments, tumor sensitizing treatment is thermotherapy and chemotherapy. In some embodiments, the tumor sensitizing treatment is photodynamic therapy.

The several functions that are built into the particle heaters as disclosed herein include: (1) to induce localized hyperthermia of a temperature over 50° C. triggered by the exogenous source which would be able to effectively kill tumor cells, (2) to change the tumor microenvironment, such as increase the blood flow, oxygen levels in the tumor, the perfusion and permeability of the tumor vasculature, then improving the accumulation of particle heaters at the tumor site, (3) to trigger chemotherapeutic drug release from the particle heaters by the energy-to-heat conversion effects, (4) to improve the tumor cell membrane penetrability to enhance the cellular uptake of the particle heaters by the localized hyperthermia, (5) particle size control (e.g., nanoparticle having diameter ranging from 10 nm to 250 nm) to ensure high passive tumor accumulation by utilizing the EPR effects of the cancerous tumors, and (6) prolonged blood circulation properties with hydrophilic surface coating on the particle surface.

In an embodiment, this disclosure provides a method for causing remotely-triggered synergistic combination therapy for the treatment of cancer in a subject comprising: (1) administering a therapeutically effective amount of the particle heaters containing the anticancer agent as described herein to the tumor site in the subject in need thereof and allowing the combination therapy to associate with cancer cells, and (2) exposing the particle heaters to an exogenous source for a sufficient period of time, wherein the material absorbs the energy from the exogenous source and converts the energy into heat; and then the heat travels outside the particle to induce localized hyperthermia at a temperature ranging from about 38.0° C. to about 52.0° C., wherein the heat causes the particle heater to alter its structure to release the anticancer agent outside of the particle.

In some embodiments, the material exhibits sufficient material process stability of retaining at least 50% of the absorbance after the exposure to the exogenous source process conditions.

In some embodiments, the exogenous source is selected from the group of an electromagnetic radiation, an electrical field, a microwave, a radio wave, an ultrasound, a magnetic field, and combinations thereof. In some embodiments, the exogenous source comprises microwave.

In some embodiments, the exogenous source comprises an ultrasound. In some embodiments, the material comprises ICG dye.

In some embodiments, the exogenous source is an ultrasonic wave produced by an ultrasound (US) producing machine. In some embodiments, the therapeutic ultrasound is either pulsed or continuous.

In some embodiments, the exogenous source comprises an electromagnetic radiation.

In some embodiments, the electromagnetic radiation source comprises a LED light or a laser light.

In some embodiments, the electromagnetic radiation source comprises a LED light. LEDs are solid state p-n junction devices that emit light when forward biased. An LED is a Light Emitting Diode, a generic term. An IRED is an Infrared Emitting Diode, a term specifically applied to Excelitas IR emitters. Unlike incandescent lamps that emit light over a very broad range of wavelengths, LEDs emit light over such a narrow bandwidth that they appear to be emitting a single “color”.

In some embodiments, the material absorbing optical energy at a wavelength from 750 nm-950 nm (e.g., Infrared Light Emitting Diodes (IRED) by Excelitas). In some embodiments, the material absorbing optical energy at a wavelength from 400 nm to 750 nm (e.g., a LED device). In some embodiments, the material is selected from the group of squraine dye, IR 193 dye, ICG dye, IR 820 dye (new ICG dye), and combinations thereof.

In some embodiments, it is desirable to keep the temperature in the surrounding area of the heat delivery composition/medium/particle to be sufficiently low to avoid collateral damage to the healthy tissues and control the temperature rise to be sufficiently high to accelerate a physical, chemical or biological activity.

In some embodiments, the electromagnetic radiation source is a laser light. In some embodiments, pulsed lasers are utilized in order to provide localized thermal heating. In some embodiments, the laser irradiation is delivered in a pulse duration longer than the thermal relaxation time (TRT) of the particle heaters such that the heat generated by the particle begins to travel outside the particle. In some embodiments, the flow of the heat delivery to the outside of the particles can be achieved by manipulating the fluence of the laser irradiation, particle size and the concentration of the particles. Pulses are at least femtoseconds in duration.

In some embodiments, the laser pulse duration is in a range from milliseconds to microseconds, and the laser has an oscillation wavelength at 805 nm, 808 nm, or 1064 nm. In some embodiments, the laser pulse duration is in a range from milliseconds to femtoseconds. In some embodiments, the particle heater absorbs the laser light having a wavelength from 750 nm to 1100 nm. In some embodiments, the material is selected from the group of indocyanine green dye (ICG), new ICG dye (IR820), IR 193 dye, iron oxide, and a tetrakis aminium dye.

In some methods for remotely-triggered synergistic combination therapy for treating a tissue region of a patient, the method comprises: a) providing a device with at least one optical fiber, wherein the fiber comprises a light transmission zone intimately contacting the tissue region configured for remotely-triggered therapy; b) administering a therapeutically effective amount of at least one particle composition, wherein the particle composition is at least partially localized to the tissue region; c) exposing the localized particle heaters to light to produce a phototoxic species or localized hyperthermia or both, wherein the activating light is transmitted from the at least one optical fiber; and d) treating the tissue region photodynamically or photothermally or both. In some embodiment, the device is selected from a group of a catheter, a cannula, a needle, a basket-type catheter, and an implant.

In some embodiments, the device is a catheter or a cannula.

In some embodiments, the material encapsulated in the particle heaters absorbs the photons of the laser to generate heat. Such heat travels outside the particle heater to the area in proximity to the particle heaters and causes significant observable temperature change thereof.

The advantages of the efficient localized heating achieved by the particle heaters or composition thereof in this disclosure are immediately evident because the temperature change is primary limited to the area surrounding the particle heaters or composition thereof, that is, selective placement of the particle heaters allows heating of targeted regions without significantly affecting the remainder of the tissue. In addition, the remotely-triggered thermal effect enables heat to be generated by the particle heater as opposed to the conventional laser-based photothermal tissue treatments that deliver energy to water and the endogenous natural pigments and dyes in the tissue (e.g., melanin). Thus, the process of the energy delivery by the exogenous source to the particle heaters in this disclosure can include selectively applying the exogenous source only to a predefined region of the tumor tissue that is to be treated by the selective placement of the particle heaters.

In some embodiments, the material has strong absorption of photons at wavelengths overlapping with the output of the various commercially available lasers.

In some embodiments, the method employs a particle heater formulation applied to the tumor site containing a low concentration of particle heaters and a high intensity laser irradiation such that the local temperature maxima caused by photothermal conversion by the particle heaters are within a nanometer scale distance from the excited particles. In some embodiments, the method employs a composition applied to the tumor site containing a higher concentration of particles and a low intensity laser irradiation such that the local temperature maxima caused by photothermal conversion from the particle heaters are at a millimeter scale distance from the excited particles (also known as collective photo-heating).

In some embodiments, the particle heaters are present in the synergistic combination therapy in an amount ranging from about 0.5 wt. % to about 25 wt. % by the total weight of the synergistic combination therapy. In some embodiments, the particle heater is present in an amount ranging from about 1.0 wt. % to about 20.0 wt. % by the total of the synergistic combination therapy. In some embodiments, the particle heater is present in an amount ranging from about 5.0 wt. % to about 20.0 wt. % by the total of the synergistic combination therapy. In some embodiments, the particle heater is present in an amount ranging from about 5.0 wt. % to about 15.0 wt. % by the total of the synergistic combination therapy. In some embodiments, the particle heater is present in an amount ranging from about 10.0 wt. % to about 15.0 wt. % by the total of the synergistic combination therapy. In some embodiments, the particle heater is present in an amount selected from: about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, or about 25.0 wt. % by the total weight of the synergistic combination therapy. In some embodiments, the particle heater is present in an amount selected from: about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, or about 15.0 wt. % by the total weight of the synergistic combination therapy. In some embodiments, the particle heater is present in an amount selected from: about 1.0 wt. %, 2.0 wt. %, 3.0 wt. %, 4.0 wt. %, about 5.0 wt. %, about 10.0 wt. % or about 15.0 wt. %.

In some embodiments, pulsed lasers are utilized in order to provide localized thermal heating. In some embodiments, the laser irradiation is delivered in a pulse duration longer than the thermal relaxation time (TRT) of the particles containing the exogenous source interacting material such that the heat energy generated by the particle begins to travel outside the particle. In some embodiments, the flow of the heat delivery to the outside of the particles can be achieved by manipulating the fluence of the laser irradiation, particle size and the concentration of the particles. Pulses are at least femtosecond in duration.

Temperatures greater than 50° C. can induce tissue fusion. (“tissue welding”). This is believed to be induced by the denaturation of the proteins and the subsequent entanglement of adjacent protein chains. In some embodiments, the temperature realized at the tissue site by particles is higher than 50° C. In some embodiments, the temperature realized at the tissue site is in a range from about 40° C. to about 50° C. In some embodiments, the peak temperature realized in the tissue from particle heating is at least 38, 39, 40, 41, 42, 43, 44, 45, 46, 47, 48, 49, 50, 51, 52, 53, 54, 55, 56, 57, 58, 59, 60, degrees Celsius (° C.). In some embodiments, that utilize pulsed laser irradiation, high peak temperatures are realized locally within specific regions at the tissue site without raising the bulk tissue temperature.

In some embodiments, the particle heater is used under the guidance of in vivo imaging, including fluorescence imaging. Imaging can provide useful information such as the size and location of the targeted tissue, as well as the relationship of the targeted tissue with its surrounding tissues.

7c. Remotely-Triggered Synergistic Combination Antimicrobial Therapy and Method of Treating Infection

In some embodiments, the antimicrobial agent is encapsulated in the particle heater and the heat causes the release of the antimicrobial agent. In some embodiments, the antimicrobial agent is not encapsulated in the particle heater. In some embodiments, the antimicrobial agent is present in a separate pharmaceutical composition from the particle heater. In some embodiments, the particle heater is administered before the administration of the antimicrobial agent. In some embodiments, the particle heater is administered after the administration of the antimicrobial agent. In some embodiments, the particle heater is administered concurrently with the administration of the antimicrobial agent.

In some embodiments, sensitizing the pathogenic microbe comprises administering to the subject a treatment that will induce apoptosis in the pathogenic microbes. In some embodiments, the treatment that will induce apoptosis in pathogenic microbes is selected from the group of thermal therapy, antibiotic, immunotherapy, phototherapy, and combinations thereof. In some embodiments, the treatment that will induce apoptosis in microbes is thermal therapy. In some embodiments, the treatment that will induce apoptosis in cells is thermal therapy and antibiotic.

In some embodiments, the method further comprises the step of exposing the particle heater remotely to an exogenous source, wherein the exogenous source is selected from the group of an electromagnetic radiation, an electrical field, a microwave, a radio wave, an ultrasound, a magnetic field, and combinations thereof.

In some embodiments, the particle heaters are designed for photodynamic therapy combined with chemotherapy (combination PDT-chemotherapy), wherein the material is a photosensitizing agent capable of producing reactive oxygen species (ROS) under exposure to laser light. In some embodiments, the particle heaters are designed for photothermal therapy combined with chemotherapy (combination PTT-chemotherapy), wherein the material is an NIR light absorbing agent. In some embodiments, the particle heaters are designed for photothermal therapy combined with photodynamic therapy and chemotherapy (combination PTT-PDT-chemotherapy), wherein the material is a NIR absorbing agent simultaneously having the capacity of producing ROS and heat generation under exposure to laser light. Sound can also be used to produce reactive molecular species and is referred to as Sonodyanmic Therapy (or SDT). For example, ICG can be used for PDT or SDT.

Reactive oxygen species (ROS) are emerging as important elements in the bacterial response to lethal stress. Bacteria contain protective proteins that can detoxify ROS (SodA, SodB, SodC, AhpCF, KatG, KatE) and counter damage (e.g., SoxRS, OxyRS, and SOS regulons). However, bacteria may also use ROS to self-destruct when stress is severe. Stress-induced, ROS-mediated bacterial self-destruction opened new avenues for antimicrobial enhancement.

There are three naturally occurring species, singlet oxygen ¹O₂, hydrogen peroxide H₂O₂, and hydroxyl radical .OH. Superoxide and hydrogen peroxide arise when molecular oxygen adventitiously oxidizes redox enzymes that normally transfer electrons to other substrates. Hydrogen peroxide, which can also be produced from dismutase of superoxide, serves as a substrate for .OH formation through Fenton chemistry. This oxidative process can kill cells if the hydroxyl radical accumulation is not controlled, since hydroxyl radical breaks nucleic acids, carbonylates proteins, and peroxidates lipids.

Treatment of microbes with a lethal antimicrobial causes primary damage to the microbes (antimicrobial mediated lesion). If the primary damage is severe enough, it can result in microbe death directly. Additionally, the primary damage stimulates a pathway that leads to ROS accumulation. This pathway can be blocked by treating microbes with iron chelators and antioxidants by inhibiting catalase/peroxidases activities. ROS causes secondary damage to nucleic acids, proteins and lipids. Secondary damage stimulates additional ROS production. When secondary damage exceeds a critical threshold, it becomes self-amplifying. Self-amplification of ROS assures microbe death.

Killing microbes by diverse antimicrobials is enhanced by defects in genes that protect against ROS, e.g. deficiency in catalase/peroxidases activities, and is associated with surges in intracellular ROS. The antimicrobial therapeutic efficacy can be compromised by compounds that block hydroxyl radical accumulation, It has been reported that the accumulation of ROS could augment effects of antibiotic-mediated lesions, as well as a deficiency in catalase/peroxidase increased the lethal action of three diverse antimicrobial classes of antibiotics including fluoroquinolones, β-lactams and aminoglycosides.

PDT involves the administration of photosensitizer (PS) and then localizing to the infection site using a specific wavelength of light to activate the PS. PDT is a two-stage procedure based on three components including photosensitizer, light and oxygen. In PDT, photosensitizers would generate ROS under appropriate light irradiation. A series of photochemical reactions initiated by PS results in the death of macrophage and proliferating smooth muscle cells. Some of the IR absorbing agents can simultaneously produce photothermal and photodynamic therapeutic effects for destruction of the pathogenic microbes.

Proteins are major targets for oxidative reactions, as they constitute nearly 70% of the dry weight of cells. Oxidized proteins can be re-folded by molecular chaperones such as HSPs. Inefficient restoration of their structure leads to accumulation of misfolded proteins. Accumulation of damaged or misfolded proteins within endothelial reticulum (ER) triggers a process called ER stress, which can be ameliorated by unfolded protein response (UPR) or can be aggregated. Consequently, the protein misfolding in the ER and ROS act in concert to cause cell death. Thus, ROS generated in the activation of the material by the exogenous source as disclosed herein is unwanted cells like tumor cells or microbes.

Targeting enzymes participating in ROS scavenging (such as superoxide dismutase, heme oxygenase-1, or nitric oxide synthase) with selective inhibitors has been shown to improve targeted cell killing activity of PDT and PTT. The endogenous chromophore such as water, lipid, or cell pigments in the cells can convert light to heat for causing hyperthermia damage to both the microbes and healthy tissues. Tissue damages is evident within minutes when the temperature of tissue reaches 55-95° C.

As PDT, PTT and antimicrobial therapy each act via three different mechanisms, their combination into a single therapeutic system provides a highly efficient approach to treat microbial infections. Upon NIR laser irradiation, PTT can directly kill microbes via hyperthermia. It is well understood that the photothermal heating effects initiate autolysis or apoptosis processes only after reaching a threshold value of local intracellular temperature (42-45° C.), lasting for 15 minutes to 60 minutes, or higher than 50° C. lasting for 4 minutes to 6 minutes (See, Hwang et al., Advanced Materials, 2014, p. 1-7).

Therapeutic approaches that interfere with re-folding or removal of oxidized proteins can be used to sensitize microbes to PDT and/or PTT. For example, modulation of HSP function with geldanamycin, a HSP90 inhibitor, sensitizes microbes to PDT and/or PTT. Bortezomib, a proteasome inhibitor successfully used in the treatment of hematological disorders potentiates cytotoxic effects of PDT and/or PTT by aggravation of ER-stress. Moreover, several apoptosis-modulating factors such as rapamycin, Bcl-2 antagonists, and ursodeoxycholic acid or ceramide analogues have been shown to increase PDT/PTT-mediated cell death.

In some embodiments, the particle heaters are designed for photodynamic therapy combined with antimicrobial agent (combination PDT-chemotherapy), wherein the material is a photosensitizing agent capable of producing reactive oxygen species (ROS) upon exposure to laser light. During the photodynamic process, a variety of ROS is produced including singlet oxygen ¹O₂, H₂O₂ and .OH.

In some embodiments, the particle heaters are designed for photothermal therapy combined with antimicrobial agent (combination PTT-chemotherapy), wherein the material is an NIR light absorbing agent. In some embodiments, the particle heaters are designed for photothermal therapy combined with photodynamic therapy and antimicrobial agent (combination PTT-PDT-chemotherapy), wherein the material is a NIR absorbing agent simultaneously having the capacity of producing ROS and heat generation under exposure to laser light.

Sound can also be used to produce reactive molecular species and is referred to as Sonodyanmic Therapy (or SDT). Sound can be used for combination SDT+sonothermal therapy and release an antimicrobial agent (chemotherapy).

In some embodiments, the material is a bimodal material that exhibits both NIR absorption and an ability to generate ROS under laser irradiation or sound waves. The bimodal material converts light energy into thermal energy for photothermal therapy and produces ROS for photodynamic therapy, which makes it attractive for applications in double or triple combination antimicrobial therapy involving PTT and/or PDT with antimicrobial agent. In some embodiments, the bimodal material is selected from the group of indocyanine green (ICG), new ICG dye IR820, IR 780 dye, IR 193 dye, plasmonic absorber, iron oxide, gold nanostructures, and combinations thereof. For example, after the ICG particles are irradiated with pulsed laser light, excited ICG dye produces ROS in the presence of cellular water.

In some embodiments, the combination therapy of pathogenic microbial infection comprises the particle heaters as disclosed herein and a active agent capable of sensitizing microbes to the remotely-triggered cytotoxic effects of the material (localized hyperthermia and/or ROS); wherein the active agent is selected from the group selected of HSP inhibitor, proteasome inhibitor, rapamycin, Bcl-2 antagonists, ursodeoxycholic acid, ceramide analogues, geldanamycin, bortezomib, superoxide dismutase inhibitor, heme oxygenase-1 inhibitor, or nitric oxide synthase inhibitor, and combinations thereof.

In an embodiment, this disclosure provides a method of treating microbial infection in a subject comprising the steps of sensitizing the microbes by administering to the subject in need thereof a treatment that will (i) induce autolysis or apoptosis in microbes, (ii) aggravation of ER-stress (a process triggered by the accumulation of damaged or misfolded proteins within endothelial reticulum), (iii) sensitizing microbe to PDT and/or PTT, (iv) sensitizing antimicrobial treatment by PDT/PTT, (v) sensitizing antimicrobial treatment by SDT/PDT, or (vi) a combination of two.

In some embodiments, the treatment is a particle heater and/or an antimicrobial agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat is transferred outside the particle to induce localized hyperthermia sufficient to selectively kill microbes.

In some embodiments, the method further comprises a step of detecting the presence or absence of microbes at the infection site to detecting the presence a color change that indicates the presence of microbes by employing the particle heater and the colorimetric agent capped with the material responsive to microbial protease in colorless states, wherein upon contacting the medium at the infection site, the protease secreted by the microbes catalyzed the release of the colorimetric to the colored state. In some embodiments, one or more additional employments of the exogenous source are applied if a color change is detected.

In some embodiments, the antimicrobial agent is encapsulated in the particle heater and the heat causes the release of the active agent. In some embodiments, the antimicrobial agent is not encapsulated in the particle heater. In some embodiments, the antimicrobial agent is present in a separate pharmaceutical composition from the particle heater. In some embodiments, the particle heater is administered before the administration of the antimicrobial agent. In some embodiments, the particle heater is administered after the administration of the antimicrobial agent. In some embodiments, the particle heater is administered concurrently with the administration of the antimicrobial agent.

In some embodiments, the method further comprises the step of exposing the particle heater remotely to an exogenous source, wherein the exogenous source is selected from the group of an electromagnetic radiation, an electrical field, a microwave, a radio wave, an ultrasound, a magnetic field, and combinations thereof.

In some embodiments, sensitizing the microbe comprises administering to the subject a treatment that will induce apoptosis in the microbes. In some embodiments, the treatment that will induce apoptosis in microbes is selected from the group of antimicrobial agent, photosensitizer, a thermotherapy sensitizing agent, or a combination thereof. In some embodiments, the treatment that will induce apoptosis in cells is thermotherapy. In some embodiments, the treatment that will induce apoptosis in cells is thermotherapy and the antimicrobial agent. In some embodiments, the antimicrobial agent is selected from the group of fluoroquinolones, beta-lactams, aminoglycosides, norfloxacin, kanamycin, gentamicin, penicillin, and combinations thereof. In some embodiments, the thermotherapy-sensitizing agent is selected from the group of a HSP90 inhibitor, a proteasome inhibitor, an apoptosis-modulating factor, and combinations thereof. In some embodiments, the thermotherapy-sensitizing agent is selected from the group of geldanamycin (HSP inhibitor), bortezomib (proteasome inhibitor), rapamycin, Bcl-2 antagonists, ursodeoxycholic acid, ceramide analogues, and combinations thereof.

In some embodiments, this disclosure provides a method of treating a microbial infection comprising administration to a subject in need thereof, therapeutically effective amounts of a particle heater in combination with an antimicrobial agent.

In some embodiments, the particle heater comprises (a) an antimicrobial agent, (b) a material interacting with an exogenous source, and (c) a carrier; wherein the particle is constructed such that it passes the Extractable Cytotoxicity Test; wherein the material absorbs the energy from the exogenous source and converts the energy into heat; then the heat travels outside the particle to induce localized hyperthermia sufficient to selectively kill microbes in combination with the antimicrobial agent.

In some embodiments, the antimicrobial agent is encapsulated in the particle heater and the heat causes the release of the antimicrobial agent. In some embodiments, the antimicrobial agent is not encapsulated in the particle heater. In some embodiments, the antimicrobial agent is present in a separate pharmaceutical composition from the particle heater. In some embodiments, the particle heater is administered before the administration of the antimicrobial agent. In some embodiments, the particle heater is administered after the administration of the antimicrobial agent. In some embodiments, the particle heater is administered concurrently with the administration of the antimicrobial agent.

In an embodiment, this disclosure provides a method for treating microbial infection with a synergistic combination therapy in a subject comprising: (1) administering a therapeutically effective amount of the synergistic combination therapy as disclosed herein to the subject in need thereof and allowing the synergistic combination therapy to associate with the microbes at the infection site, and (2) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the antimicrobial agent exhibit synergy in killing microbes, and wherein the particle is constructed such that it passes the Extractable Cytotoxicity Test. In some embodiments, the antimicrobial agent is further encapsulated by the particle heater, and the heat causes the release of the antimicrobial agent outside of the particle.

The synergistic combination therapy of treating microbial infection, wherein the localized hyperthermia and the antimicrobial agent exhibit coefficient of drug interaction (CDI)<1.0.

The synergistic combination therapy of treating microbial infection, wherein the CDI of the localized hyperthermia and the antimicrobial agent is about 0.1, about 0.2, about 0.3, about 0.4, about 0.5, about 0.6, about 0.7, about 0.8, about 0.9, or about 1.0.

In some embodiments, the particle heater and the antimicrobial agent are administered to the patient concurrently. In some embodiments, the particle heater and the antimicrobial agent are administered to the patient sequentially. In some embodiments, the antimicrobial agent is administered before administering of the particle heater. In some embodiments, the particle heater is administered before administering the antimicrobial agent.

In some embodiments, the exogenous source is selected from the group of an electromagnetic radiation, an electrical field, a microwave, a radio wave, an ultrasound, a magnetic field, and combinations thereof.

In some embodiments, the exogenous source may have a cold tip to cool the target tissue area before, during and after application of the exogenous energy. In some embodiments the cold tip may be a temperature from 2-8° C.

In some embodiments, the material exhibits sufficient material process stability of retaining at least 50% of the absorbance after the exposure to the exogenous source process conditions.

In some embodiments, the exogenous source comprises an ultrasound. In some embodiments, the material comprises ICG dye.

In some embodiments, the exogenous source is an ultrasound (US) producing machine. In some embodiments, the therapeutic ultrasound is either pulsed or continuous.

In some embodiments, the exogenous source comprises an electromagnetic radiation. In one embodiment, the disclosure provides a method of generating heat by irradiating particle heaters described herein. Irradiating the particle heaters or composition thereof includes directing electromagnetic radiation onto the particle heaters or composition thereof. The electromagnetic radiation may come from any source, including an LED, laser, or lamp. Any source that can provide the appropriate radiation, including wavelength and intensity, is compatible with the disclosed methods. In one embodiment, the source is a narrow-band EMR source, with a particular bandwidth tuned to wavelengths compatible with human tissue.

In some embodiments, the exogenous source comprises a LED light or a laser light. In some embodiments, the exogenous source comprises a LED light.

In some embodiments, the electromagnetic radiation source comprises a LED light. LEDs are solid state p-n junction devices that emit light when forward biased. An LED is a Light Emitting Diode, a generic term. An IRED is an Infrared Emitting Diode, a term specifically applied to Excelitas IR emitters. Unlike incandescent lamps that emit light over a very broad range of wavelengths, LEDs emit light over such a narrow bandwidth that they appear to be emitting a single “color”.

In some embodiments, the material absorbing optical energy at a wavelength from 400 nm to 750 nm. In some embodiments, the material absorbing optical energy at a wavelength from 630 nm to 670 nm. In some embodiments, the material is a squaraine dye. In some embodiments, the material absorbing optical energy at a wavelength from 750 nm-950 nm (e.g. Infrared Light Emitting Diodes (IRED) by Excelitas). In some embodiments, the material is a squaraine dye.

In some embodiments, the exogenous source is a laser. In some embodiments, the source is a pulsed laser. In some embodiments, the laser pulse duration is in a range from milliseconds to nanoseconds, and the laser has an oscillation wavelength at 805 nm, 808 nm, or 1064 nm. In some embodiments, the laser pulse duration is in a range from milliseconds to femtoseconds. In some embodiments, the particle heater absorbs the laser light having a wavelength from 750 nm to 1100 nm. In some embodiments, the particle heater comprises an IR absorbing agent selected from the group of indocyanine green dye (ICG), new ICG dye (IR820), IR 193 dye, squaraine dye, Epolight™ 1117, Epolight™ 1175, iron oxide, a plasmonic absorber, and combinations thereof.

In some embodiments, the method further comprises heating an area in the proximity of the particle heaters or composition thereof by transferring heat from the particle heaters to the surrounding area. As used herein, the term “in proximity to” is defined as an area containing the particle heaters or composition thereof sufficiently near the particle heaters or composition thereof to receive heat that has diffused out from the particle heaters or composition thereof after irradiation. By this step, heating the particle heaters is used to heat an area around the particle heaters to provide targeted heat. The area can be liquid, solid, gas, or any combinations thereof. In some embodiments, the area is heated to a temperature of 37.5° C. to 120° C.

In some embodiments, this disclosure provides a method of heating an area of infection having microbes in proximity to the particle heaters by the heat from the particle heaters for a sufficient period of time at a temperature ranging from about 38.0° C. to about 52.0° C.

In one embodiment, the method further includes heating a plurality of the particle heaters. While a single particle heater may be effective in a nano- or micron-scale environment, greater area can be heated by irradiating a plurality of the particle heaters.

In an embodiment, this disclosure provides a method for inducing localized hyperthermia at an infection site in a subject comprising: administering an effective amount of the particle heater as described herein to the tissue site in the subject; exposing the particle heaters to an exogenous source that heats the particle heater for a sufficient period of time to induce localized hyperthermia at a temperature ranging from about 38.0° C. to about 52.0° C. In some embodiments, the subject is a warm-blooded animal. In some embodiments, the subject is a human.

In some embodiments, the induced hyperthermia is mild hyperthermia at a temperature ranging from about 38.0° C. to about 41.0° C. In some embodiments, the induced hyperthermia is moderate hyperthermia at a temperature ranging from about 41.1° C. to about 45.0° C., wherein the hyperthermia does not cause collateral damage to healthy cells. In some embodiments, the induced hyperthermia is profound hyperthermia at a temperature ranging from about 45.1° C. to about 52.0° C., wherein the hyperthermia does not cause collateral damage to healthy cells.

In one embodiment, the infection site is heated to a temperature greater than 42° C. In one embodiment, the infection site is heated to a temperature of 37.5° C. to 50° C. In one embodiment, the infection site is heated to a temperature of about 37.5° C., about 38° C., about 38.5° C., about 39° C., about 39.5° C., about 40° C., about 40.5° C., about 41° C., about 41.5° C., about 42° C., about 42.5° C., about 43° C., about 43.5° C., about 44° C., about 44.5° C., about 45° C., about 45.5° C., about 46° C., about 46.5° C., about 47° C., about 47.5° C., about 48° C., about 48.5° C., about 49° C., about 49.5° C., or about 50° C. In some embodiments, the hyperthermia induced is of a temperature ranging from about 38.0° C. to about 90.0° C.

In some embodiments, the induced hyperthermia is profound hyperthermia at a temperature ranging from about 45.1° C. to about 52.0° C., wherein the hyperthermia causes the death of microbial cells, but does not cause collateral damage to healthy host cells.

In some embodiments, the method further comprises a step of detecting the presence or absence of resistant bacteria strains at the infection site to detecting the presence optical response by the spectroscopic probe that indicates the presence of resistant bacteria strain by employing the particle heater having the second material and the spectroscopic probe as described above before employing the exogenous source. In some embodiments, one or more additional employments of the exogenous source are applied if an optical response is detected.

In an embodiment, this disclosure provides with a synergistic combination therapy in a subject comprising: (1) administering a therapeutically effective amount of the synergistic combination therapy as disclosed herein to the subject in need thereof and allowing the synergistic combination therapy to associate with the microbes at the infection site, and (2) exposing the particle heaters to an exogenous source for a sufficient period of time, wherein the material absorbs the energy from the exogenous source and converts the energy into heat; and then the heat travels outside the particle to induce localized hyperthermia at a temperature ranging from about 38.0° C. to about 52.0° C. in a surrounding area of the particle heater, wherein the localized hyperthermia and the antimicrobial agent exhibit synergy in killing microbes.

In some embodiments, the multidrug resistant bacteria is selected from the group of Gram-positive bacteria, Gram-negative bacteria, and combinations thereof cause the infection. In some embodiments, the multidrug resistant bacteria is selected from the group of E. coli, K. pneumonia, M tuberculosis, Streptococcus aureus, P. aeruginosa, Streptococcus epidermidis, Streptococcus haemolyticus, Bacillus anthracis, Clostridium difficile, Streptococcus pyogenes, Streptococcus pneumonia, Enterococcus faecalis, and combinations thereof cause the infection.

In some embodiments, the induced hyperthermia is moderate hyperthermia at a temperature ranging from about 41.1° C. to about 45.0° C. In some embodiments, the material is capable of sensitizing the therapeutic efficacy of the antimicrobial agent such as H₂O₂ and ascorbic acid. In some embodiments, the material is capable of catalyzing the generation of reactive .OH radical from H₂O₂ after the particle heater is exposed to the laser light, whereby the killing of the microbes is at the moderate hyperthermia, but does not cause collateral damage to healthy cells. In some embodiments, the core shell particles having a plasmonic absorbing materials as the shell as described above is capable of catalyzing the generation of reactive .OH radical from H₂O₂ after the particle heater is exposed to the laser light, whereby the killing of the microbes is at the moderate hyperthermia, but does not cause collateral damage to healthy cells. In some embodiments, the material is capable of catalyzing the generation of reactive .OH radical from ascorbic acid after the particle heater is exposed to the laser light, whereby the killing of the microbes is at the moderate hyperthermia, but does not cause collateral damage to healthy cells. In some embodiments, the core shell particles having a plasmonic absorbing materials as the shell as described above is capable of catalyzing the generation of reactive .OH radical from ascorbic after the particle heater is exposed to the laser light, whereby the killing of the microbes is at the moderate hyperthermia, but does not cause collateral damage to healthy cells.

In some embodiments, the particle heaters are co-administered with H₂O₂ as antimicrobial agent having concentration lower than 3%. In some embodiments, the particle heaters are co-administered with 1.0 wt. % H₂O₂ as antimicrobial agent. The household (3%) hydrogen peroxide solution can cause damage to healthy cells. The remotely-triggered activation of the photosensitizer augments the biocidal action of the hydrogen peroxide due to the generation of potent microbe killing agent hydroxyl radical by the photosensitizer.

In some embodiments, this disclosure provides for the image-guided combination treatment of infection for specific laser triggered killing of microbes only at the infection site. The location, size and shape of the infection to be treated is generally diagnosed and characterized by the imaging technique including fluorescence imaging, contrast enhanced computed tomography (CT), and magnetic resonance imaging (MRI). The imaging techniques allow for the detection of the microbes and enabling simultaneous guidance of therapeutic laser irradiation to induce microbe death by imaging the contrast agent.

In some embodiments, the material has bi-modal functions as imaging agent and energy-thermal conversion agent selected from the group of a squarylium dye, indocyanine green (ICG), new ICG (IR 820), squaraine dye, IR 780 dye, IR 193 dye, Epolight™ 1117 dye, zinc iron phosphate pigment, iron oxide nanoparticle, and combinations thereof. In some embodiments, the imaging technique is fluorescence imaging based on ICG dye. In some embodiments, the imaging technique is fluorescence imaging based on new ICG dye. In some embodiments, the imaging technique is fluorescence imaging based on IR 193 dye. In some embodiments, the imaging technique is MM using iron oxide nanoparticles as contrast agent. In some embodiments, the diagnostic imaging technique is computed tomography using iodine in the iodinated polymers used in the carrier.

In some embodiments, the method employs a composition applied to the infection site containing a low concentration of particle heaters and a high intensity laser irradiation such that the local temperature maxima caused by photothermal conversion by the particle heaters are within a nanometer scale distance from the excited particles. In some embodiments, the method employs a composition applied to the infection site containing a higher concentration of particles and a low intensity laser irradiation such that the local temperature maxima caused by photothermal conversion from the particle heaters are at a millimeter scale distance from the excited particles (also known as collective photo-heating).

In some embodiments, the particle heaters are present in the pharmaceutical composition in an amount ranging from about 0.5 wt. % to about 25 wt. % by the total weight of the pharmaceutical composition. In some embodiments, the particle heater is present in an amount ranging from about 1.0 wt. % to about 20.0 wt. % by the total of the pharmaceutical composition. In some embodiments, the particle heater is present in an amount ranging from about 5.0 wt. % to about 20.0 wt. % by the total of the pharmaceutical composition. In some embodiments, the particle heater is present in an amount ranging from about 5.0 wt. % to about 15.0 wt. % by the total of the pharmaceutical composition. In some embodiments, the particle heater is present in an amount ranging from about 10.0 wt. % to about 15.0 wt. % by the total of the pharmaceutical composition. In some embodiments, the particle heater is present in an amount selected from the group of about 0.1 wt. %, about 0.2 wt. %, about 0.3 wt. %, about 0.4 wt. %, about 0.5 wt. %, about 0.6 wt. %, about 0.7 wt. %, about 0.8 wt. %, about 0.9 wt. %, about 1.0 wt. %, about 1.5 wt. %, about 2.0 wt. %, about 2.5 wt. %, about 3.0 wt. %, about 3.5 wt. %, about 4.0 wt. %, about 4.5 wt. %, about 5.0 wt. %, about 5.5 wt. %, about 6.0 wt. %, about 6.5 wt. %, about 7.0 wt. %, about 7.5 wt. %, about 8.0 wt. %, about 8.5 wt. %, about 9.0 wt. %, about 9.5 wt. %, about 10.0 wt. %, about 10.5 wt. %, about 11.0 wt. %, about 11.5 wt. %, about 12.0 wt. %, about 12.5 wt. %, about 13.0 wt. %, about 13.5 wt. %, about 14.0 wt. %, about 14.5 wt. %, about 15.0 wt. %, about 15.5 wt. %, about 16.0 wt. %, about 16.5 wt. %, about 17.0 wt. %, about 17.5 wt. %, about 18.0 wt. %, about 18.5 wt. %, about 19.0 wt. %, about 19.5 wt. %, about 20.0 wt. %, about 20.5 wt. %, about 21.0 wt. %, about 21.5 wt. %, about 22.0 wt. %, about 22.5 wt. %, about 23.0 wt. %, about 23.5 wt. %, about 24.0 wt. %, about 24.5 wt. %, and about 25.0 wt. % by the total weight of the pharmaceutical composition. In some embodiments, the particle heater is present in an amount selected from the group of about 1.0 wt. %, about 2.0 wt. %, about 3.0 wt. %, about 4.0 wt. %, about 5.0 wt. %, about 6.0 wt. %, about 7.0 wt. %, about 8.0 wt. %, about 9.0 wt. %, about 10.0 wt. %, or about 15.0 wt. % by the total weight of the pharmaceutical composition. In some embodiments, the particle heater is present in an amount selected from: about 1.0 wt. %, 2.0 wt. %, 3.0 wt. %, 4.0 wt. %, about 5.0 wt. %, about 10.0 wt. %, and about 15.0 wt. % by the total weight of the pharmaceutical composition.

In some embodiments, pulsed lasers are utilized in order to provide localized thermal heating. In some embodiments, the laser irradiation is delivered in a pulse duration longer than the thermal relaxation time (TRT) of the particles containing the exogenous source interacting material such that the heat energy generated by the particle begins to travel outside the particle. In some embodiments, the flow of the heat delivery to the outside of the particles can be achieved by manipulating the fluence of the laser irradiation, particle size and the concentration of the particles. Pulses are at least femtoseconds in duration.

In some embodiments, the pthogenic microorganism may include, but not limited to bacterial genera staphylococcus, Enterococcus, Escherichia, Streptococcus, Campylobacter, Salmonella, Helicobacter, Bacillus, Clostridium, Corynebacterium, Chlamydia, Coxilla, Ehrlichia, Francisella, Legionella, Pasteurella, Brucella, Proteus, Klebsiella, Enterobacter, Tropheryma, Acinetobacter, Aeromonas, Alcaligenes, Capnocytophaga, Erysipelothrix, Listeria, Yersinia, and the like; and fungi, such as Candida albicans, Microsporum canis, Sporothrix schenckii, Trichophyton rubrunl, Trichophyton mentagrophytes, Malassezia furfur, Pityriasis versicolor, Exophiala werneckii, Trichosporon beigelii, Coccidioides immitis, Blastomyces dermatitidis, Aspergillus fumigatus, Epidermophyton spp., Fusarium spp., Zygomyces spp., Rliizopus spp., and Mucor spp.

In some embodiments, the pathogenic microbes comprise antimicrobial resistant pathogenic microbes. The antimicrobial-resistant strains of pathogenic microorganisms include Staphylococcus aureus, Enterococcus faecium, Enterococcus faecalis, E. coli, Salmonella typhi, Campylobacter jejuni, Klebsielia pneumoniae, Neisseria gonorrhoeae, Candida albicans, and the like. More specifically, such antimicrobial-resistant organisms include methicillin-resistant Staphylococcus aureus (MRSA), vancomycin-resistant enterococci (VRE), ampicillin-resistant E. coli (e.g., E. coli 0157:H7), fluoroquinolone-resistant Salmonella typhi, ceftazidime-resistant Klebsiella pneumoniae, fluoroquinolone-resistant Neisseria gonorrhoeae, methicillin resistant, coagulase-negative staphylococci (“CNS”), penicillin resistant Streptococcus pneumoniae, and combinations thereof.

In some embodiments, the bacteria comprise multidrug resistant bacteria selected from the group of E. coli, K. pneumonia, M. tuberculosis, Streptococcus aureus, P. aeruginosa, Streptococcus epidermidis, Streptococcus haemolyticus, Bacillus anthracia, Clostridium difficile, Streptococcus pyogenes, Streptococcus pneumonia, Enterococcus faecalis, Salmonella typhi, and combinations thereof.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### General Procedures

The compositions of this invention may be made by various methods known in the art. Such methods include those of the following examples, as well as the methods specifically exemplified below.

### Example 1. Particle Fabrication

### Material

Chemical reagents sodium dodecyl sulfate (SDS), aqueous polyvinyl alcohol (PVA), NeoCryl® B-805 polymer (MMA/BMA copolymer, weight average molecular weight=85,000 Da, glass transition temperature Tg=99° C.) was purchased from DSM. Epolight™ 1117 (tetrakis aminium, absorbing at 800 nm-1071 nm, melting point: 185-188° C., soluble in acetone, methylethylketone and cyclohexanone) was purchased from Epolin Inc. Antioxidant Cyanox® 1790 (1,3,5-tris(4-tert-butyl-3-hydroxy-2,6-dimethyl benzyl)-1,3,5-triazine-2,4,6-(1H,3H,5H)-trione, CAS NUMBER 040601-76-1) was purchased from Cytec Industries Inc.

Methods for Preparing Particles from Polymers

(a). Solvent Evaporation. In this method the polymer is dissolved in a volatile organic solvent, such as methylene chloride. The active agent (either soluble or dispersed as fine particles) is added to the polymer solution, and the mixture is suspended in an aqueous solution that contains an emulsifier such as poly(vinyl alcohol). The resulting emulsion is stirred until most of the organic solvent evaporates, leaving solid particles. The resulting particles are washed with water and are dried overnight in a lyophilizer. Particles with different sizes and morphologies can be obtained by this method. This method is useful for PLGA, PLA, or PGA particles.

(b). Hot Melt Microencapsulation. In this method, the polymer is first melted and then mixed with the solid particles of the drug substance. The mixture is suspended in a non-miscible solvent (like silicon oil), and, with continuous stirring, heated to 5° C. above the melting point of the polymer. Once the emulsion is stabilized, it is cooled until the polymer particles solidify. The resulting particles are washed by decantation with petroleum ether to give a free-flowing powder. The external surfaces of the particles prepared with this technique are usually smooth and dense. This procedure is used to prepare particles made of polyesters and polyanhydrides. However, this method is limited to polymers with average molecular weights between 1,000 Da and 50,000 Da.

(c). Solvent Removal. This technique is primarily designed for polyanhydrides. In this method, the active agent is dispersed or dissolved in a solution of the selected polymer in a volatile organic solvent like methylene chloride. This mixture is suspended by stirring in an organic oil (such as silicon oil) to form an emulsion. Unlike solvent evaporation, this method can be used to make particles from polymers with high melting points and different molecular weights. The external morphology of particles produced with this technique is highly dependent on the type of polymer used.

(d). Spray-Drying. In this method, the polymer is dissolved in organic solvent. A known amount of the active agent is suspended (insoluble drugs) or co-dissolved (soluble drugs) in the polymer solution. The solution or the dispersion is then spray-dried.

(e). Phase Inversion. Particles can be formed from polymers using a phase inversion method wherein a polymer is dissolved in a “good” solvent, a substance to be incorporated, such as an active agent, is mixed or dissolved in the polymer solution, and the mixture is poured into a strong non solvent for the polymer, to spontaneously produce, under favorable conditions, polymeric particles, wherein the polymer is either coated with the particles or the particles are dispersed in the polymer. The method can be used to produce particles in a wide range of sizes, including, for example, about 100 nanometers to about 10 microns.

### Example 1 (i) Synthesis and Characterization of Tetrakis Aminium Dye/B805 Particles (Uncoated Particles Synthesized Through Emulsion Method)

Abbreviations: n-BMA: n-butyl methacrylate; MMA: methyl methacrylate

The preparation of the aqueous phase: under the stirring with an IKA Ultra-Turrax® T 25 homogenizer at 8000 RPM, 1.2 g of sodium dodecyl sulfate (SDS) was added into 190 g of 4.9% aqueous polyvinyl alcohol (PVA) solution placed in a round bottom flask. An aqueous solution of SDS containing 4.9% PVA was formed after the dissolution of SDS (the aqueous phase).

The preparation of the organic phase: to 88 g of dichloromethane was added 8.0 g of DSM NeoCryl® B-805 polymer (MMA/BMA copolymer), 1.82 g of Epolight™ IR 1117, and 0.65 g of Cyanox® 1790 in 88 g to allow the formation of a clear solution of B805 polymer and IR absorbing agents (the polymer: IR absorbing agent weight ratio=4.4:1).

The organic phase (polymer and IR absorbing agents dissolved in dichloromethane) was injected directly into the aqueous phase (PVA solution with SDS surfactant) at the tip of the Turrax's roto-stator (i.e. directly into the flow being sheared by the roto-stator). The shear mixing at 8000 RPM was continued for 30 minutes. The resulting mixture was decanted into an open-mouth container and stirred magnetically for 16 hours. A solid suspension of particles containing IR absorbing agent was obtained.

The solid suspension was centrifuged at 5000 RPM for 30 minutes and the particles were collected. The collected particles were washed with distilled water by resuspending the particles into distilled water and centrifuging as before to collect the particles. This washing process was repeated three times to remove residual PVA. The resulting MMA/IA/BMA copolymer particles containing IR absorbing agent were air-dried.

### Example 1 (ii) Synthesis of 25% VTMS Coated Tetrakis Aminium Dye/B805 Particles

In a first vessel, 1.52 g (0.01 mmol) of vinyltrimethoxysilane (CH₂═CHSi(OMe)₃, VTMS, MW=148 Da) was mixed with 4.58 g of dilute aqueous hydrochloric acid at a pH of 3.5 under magnetic stirring (24.9 wt. % solution of CH₂═CHSi(OMe)₃ in diluted HCl). The resulting mixture was stirred for 2 hours to allow complete hydrolysis of VTMS to give vinylsilanetriol (CH₂═CHSi(OH)₃, MW=106 Da).

In a second vessel, under magnetic stirring, 3.0 g of pre-made uncoated IR absorbing agent particles of Example 1 (i) were dispersed in 57 grams of water to provide a 5.0 wt. % IR absorbing agent particle dispersion. The pH value of the resulting IR absorbing agent particle aqueous dispersion was adjusted to 10.0 with the addition of dilute aqueous ammonium hydroxide. To this particle dispersion at pH 10, an aliquot of 3.99 g of the hydrolyzed 25 wt. % VTMS solution was added at a rate of 2 drops per second to the particle suspension. The pH value of the resulting suspension was monitored after the hydrolyzed 25% VTMS solution addition and adjusted with ammonium hydroxide solution to maintain a pH of 10 for 60 minutes. After 60 minutes, the suspension was neutralized with glacial acetic acid to lower the pH from 10 to 4.6-5.7. The weight ratio of VTMS to the uncoated particle was 0.33:1.

The resulting particle suspension was centrifuged for 30 minutes at 5000 RPM to collect the vinylsilicate-coated IR absorbing agent particles. The particles collected after the centrifugation were redispersed in distilled water and subjected to centrifugation to collect the particles. The washing procedure was repeated 3 times to remove any unreacted chemical reagents. The resulting vinylsilicate-coated particles were suspended in distilled water.

Multiple commercially available infrared IR absorbing agents were screened to find a preferred composition to provide localized heat delivery to a tissue site with sufficient temperature rise to accelerate a reaction outside of the particle. The IR absorbing agents screened include Lumogen IR 1050, Epolight™ 1117, Epolight™ 1125, and Epolight™ 1178.

In the emulsion method of encapsulation, a surfactant is necessary to help keep the emulsion stable. While Aerosol® TR-70 (sodium bis(tridecyl) sulfosuccinate) could be used as an emulsifier to prepare polymer particles encapsulating Epolight™ 1117 tetrakis aminium dye, TR-70 only provided limited stabilization effects on the tetrakis aminium dye. Sodium dodecyl sulfate was found to have a better stabilizing effect on the Epolight™ 1117 during the emulsion and evaporation process, shifting retention in the particles from 50% retention, to up to 85-90% retention. Reducing the amount of SDS in the aqueous phase led to lower Epolight™ 1117 retention and larger particle size (Table 5).

TABLE 5
Stabilization effects of the surfactant type and quantity on
tetrakis aminium dye in aqueous phase during emulsification

| | | | | |
|---|---|---|---|---|
| Surfactant in aqueous | 0.6% TR-70 | 0.6% SDS | 0.4% SDS | 0.2% SDS |
| phase | | | | |
| Median Particle size | 1.20 μm | 0.47 μm | 0.68 μm | 1.08 μm |
| % Epolight ™ 1117 | 51.70% | 82.96% | 80.17% | 74.97% |
| Retention | | | | |

The polymer used for this application is preferred to have a glass transition temperature significantly greater than the temperature of the environment for the intended use.

Various commercially available acrylic polymers were screened for preferred particle performance characteristic such as particle size distribution, IR absorbing agent stability and encapsulation efficiency. NeoCryl®B-851, a butyl acrylate/styrene copolymer proved to have a hydroxyl value too high, leading to a more polar particle and poor retention of the embedded tetrakis aminium dyes. NeoCryl® B-818, an ethyl acrylate/ethyl methacrylate copolymer, contained a lower hydroxyl value, but was still swellable in low molecular weight alcohols. NeoCryl® B-805, a methyl methacrylate/butyl methacrylate copolymer, had suitably a low hydroxyl value and a high Tg (99° C.) for human body applications. Use of a pure methyl methacrylate polymer, NeoCryl® B-728, led to greater degradation of the Epolight™ IR 1117.

The loading of IR absorbing agents within the particles is as high as possible without degrading the cohesion of the polymer. The additives that stabilize the IR absorbing agent within the particles have been studied. The antioxidant Cyanox® 1790 was found to have a positive impact on IR absorbing agent stability.

### Example 1(iii). Particle Size Determination

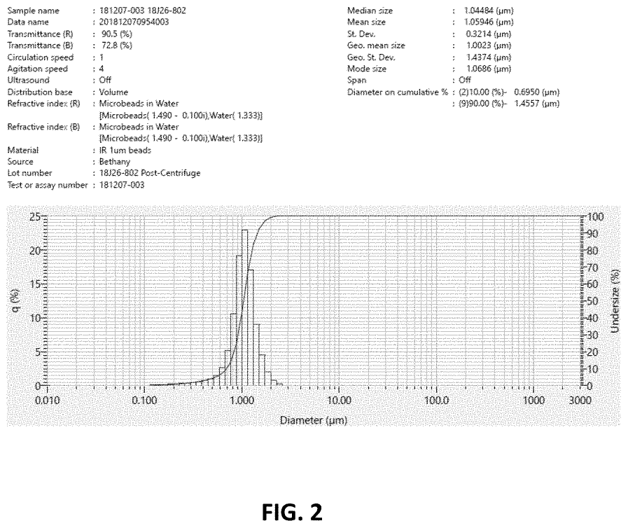

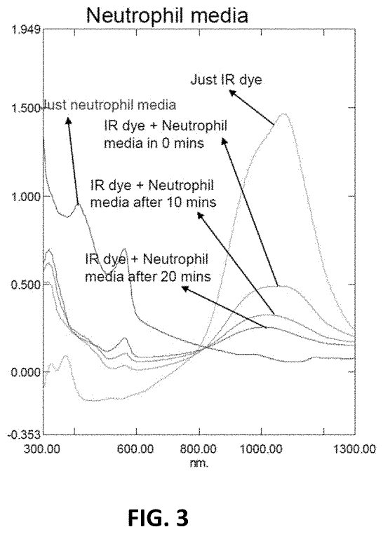

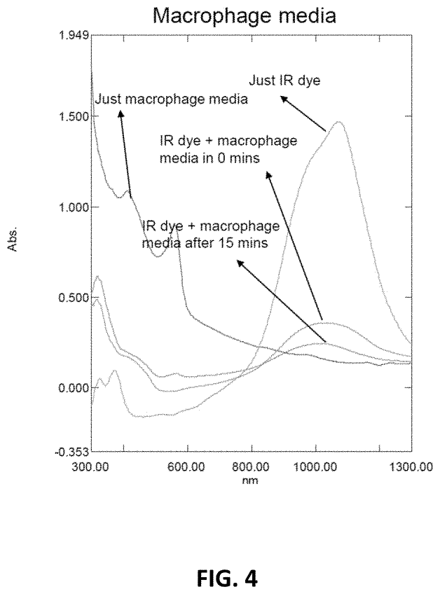

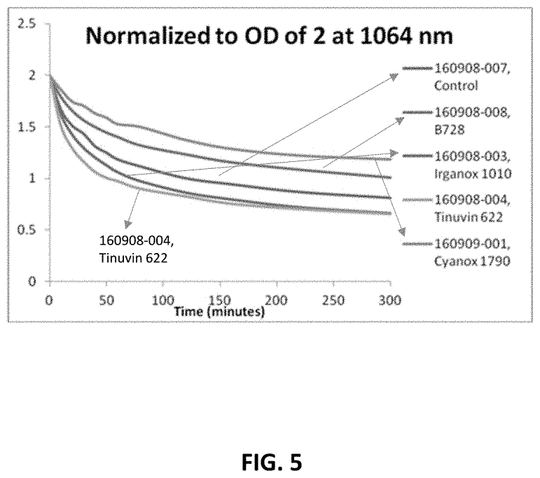

The particle size and size distribution of the NIR dye/MMA/BMA copolymer particles were measured by a Horiba LA-950 Particle Size Analyzer in distilled water with pH 7.4 (FIG. 2). All the particle size measurements were carried out at room temperature (17-23° C.).

Various additional Epolight™ 1117 particles are prepared according to the procedures set forth in the Example 1(i) above. The physicochemical properties of the resulting particles are summarized in Table 6 below.

TABLE 6
Particle Structure

| | | | | polymer/IR | |
|---|---|---|---|---|---|
| | | | particle size | absorbing | |
| | IR absorbing | polymer | range | agent weight | |
| entry | agent | carrier | (micron) | ratio range | additive |
| 1 | Epolight ™ | B805^{a} | 0.47, 0.68, | 4.4:1 | Cyanox ® |
| | 1117 | | 1.08, 1.20 | | 1790^{b} |
| | | | | | SDS^{c} |

^{a}Polymer B805 ®: copolymer of 96% methyl methacrylate and 4% butyl methacrylate.
^{b}Cyanox ®1790: dye stabilizer mixed in the polymer matrix.
^{c}SDS = sodium dodecyl sulfate, surfactant for emulsion solvent evaporation particle fabrication method.

### Example 1(iii) Optical Properties of the Epolight™ IR 1117-B805 Particles

The optical properties of the Epolight™ IR 1117-B805 particles dispersed in an aqueous water are determined by UV-VIS spectroscopy.

TABLE 7
Properties of Epolight ™ IR 1117

| | Molecular | Peak absorption | Extinction coefficient | Non-cytotoxic |
|---|---|---|---|---|
| IR absorbing | Weight | wavelength (nm) | (M⁻¹*cm⁻¹) | concentrations |
| agent | (g/mol) | (in DCM^{a}) | (in DCM) | (μM) |
| Epolight ™ 1117 | 1211 | 1098 | 105,000 | 32 |

^{a}DCM is the abbreviation for dichloromethane.

### Example 1(iv). Biodegradable Particle Fabrication

Poly(lactide-co-glycolide) (PLGA) (MW: 10,000-15,000 Da), Methoxy poly(ethylene glycol)-bpoly(lactide-co-glycolide) (mPEG-PLGA) (MW: 2-15 k.Da) are purchased from PolySciTech® (West Lafayette, Ind., USA). Epolight® 1117 is purchased from Epolin Inc (Newark, N.J., USA) and; ICG is purchased from AFG Biosciences (Northbrook, Ill., USA), IR-193 dye was a gift from Polaroid (Cambridge, Mass.) to Bambu Vault; Paclitaxel is purchased from LC Labs (Woburn, Mass., USA). All cell lines are obtained from ATCC (Manassas, Va.). The 3-(4,5-dimethylthiazol-2-yl)-5-(3-carboxymethoxyphenyl)-2-(4-sulfophenyl)-2H-tetrazolium (MTS) assay kit is purchased from Promega Corporation® (Madison, Wis., USA), Triton-X and other HPLC grade organic solvents are obtained from Fisher Scientific™ (Agawam, Mass., USA).

Multiple commercially available IR absorbing agents are screened to find a preferred composition to provide localized heat delivery to a tissue site with sufficient temperature rise to accelerate a reaction outside of the particle. The IR absorbing agents screened include ICG, IR-193 dye, Lumogen® IR 1050, Epolight® 1117, Epolight® 1125, and Epolight® 1178.

Amphiphilic co-polymers of PLGA and PEG are used to prepare PLGA/PLGA-PEG NPs with a blend of 75:25 of PLGA and PLGA-PEG. NPs are synthesized by adding Epolight™ 1117 or ICG to the polymer solution containing a blend of 75:25 of PLGA and PLGA-PEG along with paclitaxel. Similarly, NPs with only vancomycin, NPs with vancomycin and IR absorbing agent, empty NPs (without the IR absorbing material), NPs with just IR absorbing agent (no paclitaxel) and NPs with only paclitaxel (no IR absorbing agent) are prepared.

IR absorbing agent concentration is measured by NIR spectrophotometry by measuring absorbance and using Beer's law to estimate concentration. Particle size, polydispersity index and zeta potential are confirmed by dynamic light scattering using a Zetasizer (ZS-90 from Malvern Instruments) and scanning/transmission electron microscopy. Encapsulation efficiency is calculated for the IR absorbing agent by estimating the final amount of IR absorbing agent in the purified particles (using concentration measured by UV spectrophotometry) and dividing that by amount that is originally used during the synthesis of the particles.

$$IR absorbing agent loading efficiency ( \% ) = \frac{Amount of IR absorbing agent in mg from spectrophotometry}{Amount of IR absorbing agent used} \times 100 \%$$

Paclitaxel or vancomycin drug concentration is measured by Reversed Phase High Performance Liquid Chromatography (RP-HPLC) using a UV-Vis detector. Standard HPLC methods as described in the literature are used for measuring paclitaxel or vancomycin concentrations. A calibration curve is first obtained for a concentration range of paclitaxel or vancomycin. The accurate knowledge of the encapsulated paclitaxel or vancomycin concentration is essential to define the quantities of particles necessary to achieve the destruction of cancer cells.

$$drug loading efficiency ( \% ) = \frac{Amount of drug in mg from spectrophotometry}{Amount of drug used} \times 100 \%$$

### Example 1(v). Preparation of Curcumin Loaded Particles by Nanoprecipitation

Natural plant dye curcumin is chosen as a model drug. The curcumin loaded nanoparticles are prepared by nanoprecipitation. 100 mg of polymer and 10 mg of curcumin are co-dissolved in 5 mL of dichloromethane and this organic solution is added drop-wise to 15 mL of deionized water under constant magnetic stirring at 50 RPMs. The mixture is allowed to stir overnight, and the resulting particles are purified by ultracentrifugation using a 30 kDa centrifugal filter at 1000 g for 15 min at room temperature followed by three washes with deionized water. The curcumin concentration is measured by NIR spectrophotometry by measuring absorbance and using Beer's law to estimate concentration. Particle size, polydispersity index and zeta potential is confirmed by dynamic light scattering using a Zetasizer (ZS-90 from Malvern Instruments) and transmission electron microscopy. Encapsulation efficiency is calculated for the curcumin by estimating the final amount of curcumin in the purified particles (using concentration measured by UV-VIS-NIR spectrophotometry) and dividing that by the 10 mg of curcumin that was originally used during the synthesis of the particles.

### Example 1 (vi). Preparation of Curcumin and IR Absorbing Agent Loaded Particles by Nanoprecipitation

100 mg of polymer, 20 mg of curcumin, 10 mg of IR absorbing agent is co-dissolved in 5 mL of dichloromethane and this organic solution is added drop-wise to 15 mL of deionized water under constant magnetic stirring at 50 RPMs. The mixture is allowed to stir overnight, and the resulting particles are purified by ultracentrifugation using a 30 kDa centrifugal filter at 1000 g for 15 min at room temperature followed by three washes with deionized water. Curcumin and IR absorbing agent concentrations are measured by UV-VIS-NIR spectrophotometry by measuring absorbance spectrum for curcumin and IR absorbing agent and using Beer's law to estimate concentration. Curcumin concentration is calculated by UV-VIS-NIR spectrophotometry and confirmed by HPLC. Using the Eqn. 2 below, the encapsulation efficiency is calculated for the IR absorbing agent and curcumin respectively by estimating the final amount of IR absorbing agent and curcumin respectively in the purified particles (using concentration measured by NIR (for IR absorbing agent) or UV-VIS (for curcumin) spectrophotometry) and dividing that by the 10 mg of IR absorbing agent (or 20 mg of curcumin) that was originally used during the synthesis of the particles. Particle size, polydispersity index and zeta potential is confirmed by dynamic light scattering using a Zetasizer (ZS-90 from Malvern) and transmission electron microscopy. The particles are freeze-dried using a lyoprotectant for long-term storage as a powder in the −80° C. freezer.

### Example 2: Particle Characterization and Stability Testing

### Example 2a. Particle Size and Distribution for the Particle Heaters

Particle size, polydispersity index and zeta potential are confirmed by dynamic light scattering using a Zetasizer® (ZS-90 from Malvern Instruments All the particle size measurements are carried out at 25° C. All the measurements are performed in triplicate.

### Example 2b In Vitro Stability Study on Drug and IR Absorbing Agent Co-Loaded Particles

In vitro stability of the particles is evaluated by storing the sample at 4° C. and 37° C. The particle size change, the PDI change and the zeta potential change is measured by Zetasizer® Dynamic Light Scattering instrument. Particle formulations containing the material (IR absorbing agent) and the drug (paclitaxel, vancomycin, or curcumin) are stored in a vial covered in foil and stored at 4° C. for a week to study the stability of the particles for their storage shelf life. The particles are also resuspended in 1:1 ratio (by volume) in MEM alpha modification media containing 10% FBS and stored at 37° C. to study their stability under physiological conditions. Samples are periodically removed from these two storage conditions and particle size; polydispersity index and zeta potential are confirmed by dynamic light scattering using a Zetasizer® (ZS-90 from Malvern Instruments) for particles stored under these conditions.

### Example 3: Particle Content Test

UV/VIS/NIR: The absorbance spectrum for the material (IR absorbing agent) is measured using Shimadzu UV-3600 UV-NIR Spectrophotometer. The paclitaxel or vancomycin drug content is confirmed using RP-HPLC with a UV-Vis detector.

### The Percentage of IR Absorbing Agent/Drug Loading Determination

The percentage of IR absorbing agent loaded in to the particles can be determined according to the following procedure: Known quantities of particles in deionized water are added to a solution of 2% Triton-X solution in a 1:1 volume ration. The UV-VIS-NIR absorbance spectrum of the IR absorbing agent and/or the drug is measured using Shimadzu UV-3600 UV-VIS-NIR Spectrophotometer. The concentration of the IR absorbing agent in the particles is determined from application of Beer's law. A similar procedure can be used to measure paclitaxel or vancomycin drug concentration via HPLC as described earlier.

$$[ IR absorbing agent \text{/} Drug ] ( μ M ) = \frac{{Absorbance}_{λ}}{{ɛ}_{λ} \times l} \times {10}^{6}$$

where the path length, 1, is 1 cm.

The quantity of IR absorbing agent/Drug is determined from the product of the concentration, the amount of total solution, and the molecular weight of the IR absorbing agent/Drug. The IR absorbing agent/Drug loading as a percentage of the total particle mass is determined from:

$$IR absorbing agent \text{/} Drug Loading ( \% ) = \frac{Amount of IR absorbing agent \text{/} drug in solution}{Amount of particle used} \times 100 \%$$

### Example 4. Efficacy Determination Protocol

An Efficacy Determination Protocol is used to evaluate the effect of biological chemicals including bodily fluid on the IR absorbing agent (e.g. tetrakis aminium dye) and or the drug (paclitaxel, vancomycin, or curcumin) in the particles described above. Briefly, a known quantity of the particles containing the IR absorbing agent and the drug are incubated with 1 mL of complete cell culture media (for example macrophage or neutrophil cell growth media) containing 10% fetal bovine serum at 37° C. As a negative control, the same quantity of particles containing the IR absorbing agent and the drug is suspended in 1 mL of distilled water and incubated at 37° C. At different time intervals (for example: 1 h, 4 h, 8 h, 24 h) following incubation, for both the test and control, 20 μL of sample is removed and diluted to 6 mL with distilled water. UV-VIS-IR absorbance spectrum of each solution is measured using a UV-VIS spectrophotometer for the IR absorbing agent concentration. Degradation of the IR absorbing agent and the drug by the cell culture medium is determined by comparing the peak absorption in the spectrum of the test sample to the absorption of the control sample at the same spectral peak, and degradation is generally reported as the percentage in the reduction in the peak absorbance. If the IR absorbing agent or the drug does not absorb UV-VIS light, other analytical tools, like NMR, HPLC, LCMS, Circular Dichroism etc., would be used to quantify the concentration of the IR absorbing agent/drug in the test and control. The particles can be designed to ensure that no more than 90% degradation is observed for the IR absorbing agent and the drug at 24 h following incubation with relevant cell culture media.

In some instances, if the degradation of the IR absorbing agent is less than 90% then the particle is considered passing the Efficacy Determination Protocol. In some instances, depending on the potency of the IR absorbing agent, if the degradation of the IR absorbing agent is less than 85%, 80%, 75%, 70%, 65%, 60%, 55%, 50%, 45%, 40%, 35%, 30%, 25%, 20%, 15%, 10%, or 5%, then the particle is considered passing the Efficacy Determination Protocol. In some instances, if the degradation of the drug is less than 90% then the particle is considered passing the Efficacy Determination Protocol. In some instances, depending on the potency of the drug, if the degradation of the drug is less than 85%, 80%, 75%, 70%, 65%, 60%, 55%, 50%, 45%, 40%, 35%, 30%, 25%, 20%, 15%, 10%, or 5%, then the particle is considered passing the Efficacy Determination Protocol.

For example, in one instance, 100 mg of coated or uncoated dry particles were added to in 900 μl fluid, e.g., distilled water or cell media with 10% FBS in a 1.5 ml tube which was then vortexed to mix. Tubes were then incubated at 37° C. for 5 days.

After incubation period, each tube was centrifuged, decanted, and then rinsed by resuspending in 5 ml of distilled water, centrifuging and decanting again. The rinsing process was repeated 3 times. The tubes were then dried of excess water by sitting under vacuum at ambient temperature. The dried pellets were ground in a mortal and pestle.

Dye content was measured of the particle batches before the test was started and again after the incubation period. Measurements were made by taking dry particles and weighing 3-5 mg into 25 ml of dichloromethane, swirling to dissolve and measuring the spectra of the solution. Three measurements were taken for each specimen and each spectrum was divided by the weight of the specimen measured to give absorbance/mg, eliminating differences of concentration of dry particles in dicholormethane. The 3 replicates were averaged and standard deviation calculated to allow estimation of error in the measurement.

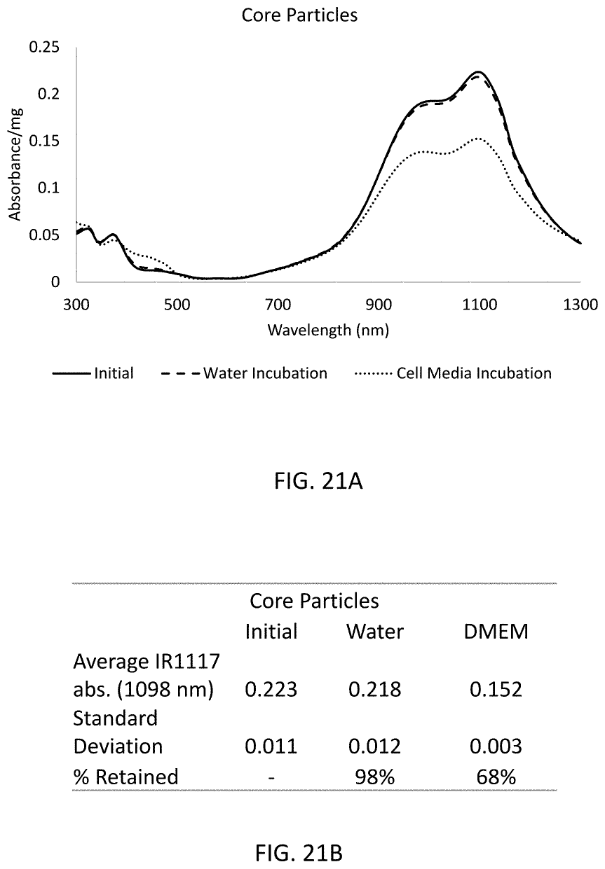

FIGS. 21A and 21B illustrate the results of the efficacy determination protocol (EDP) for core particles with 8% IR dye.

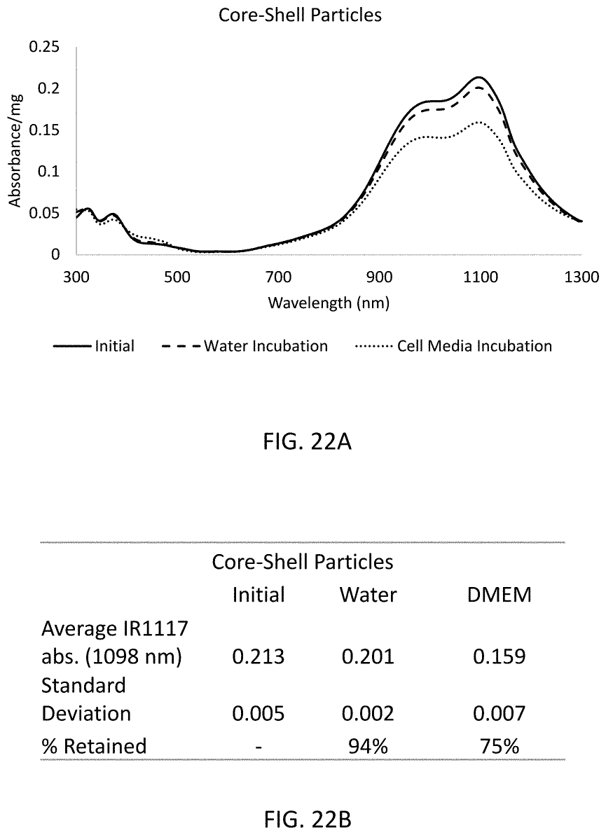

FIGS. 22A and 22B illustrate the results of the EDP for core-shell particles with 8% IR dye.

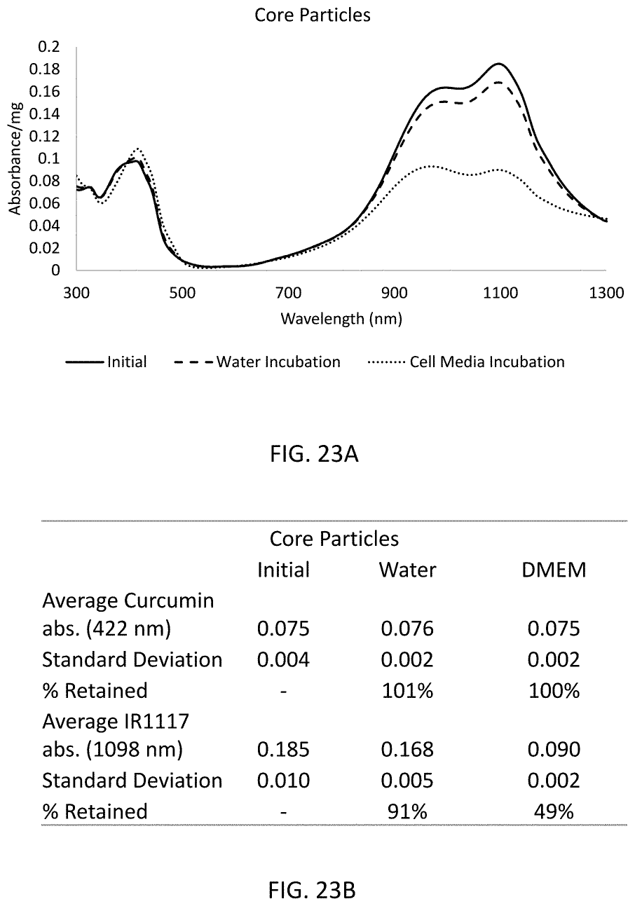

FIGS. 23A and 23B illustrate the results of the EDP for core particles with 2% curcumin and 8% IR dye.

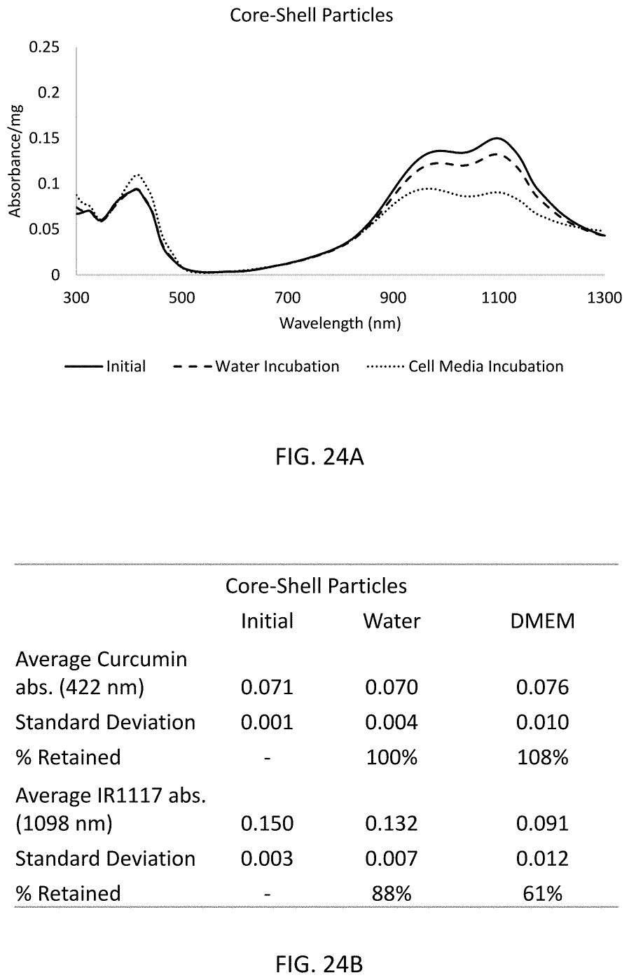

FIGS. 24A and 24B illustrate the results of the EDP for core-shell particles with 2% curcumin and 8% IR dye.

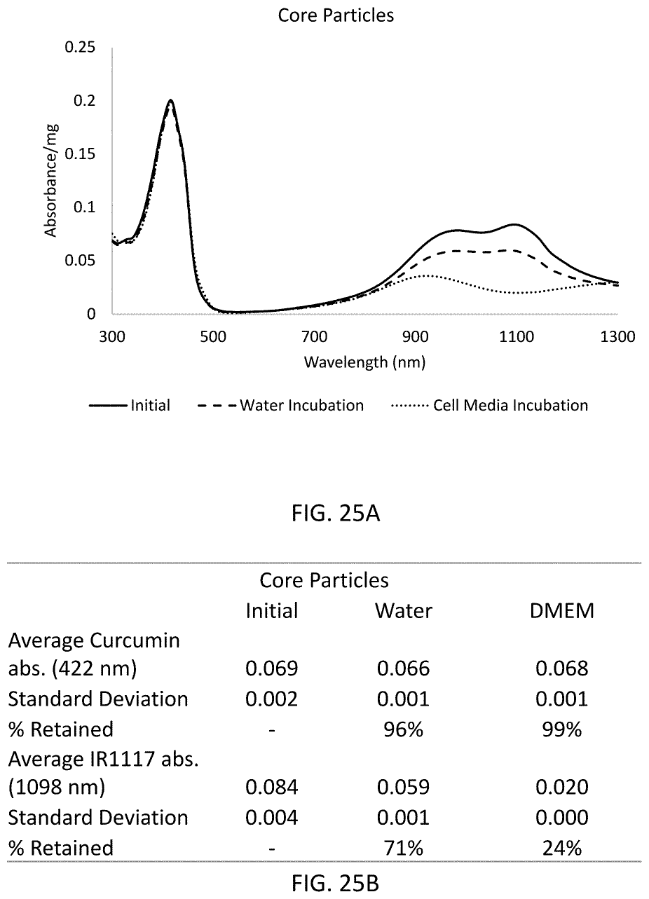

FIGS. 25A and 25B illustrate the results of the EDP for core particles with 5% curcumin and 5% IR dye.

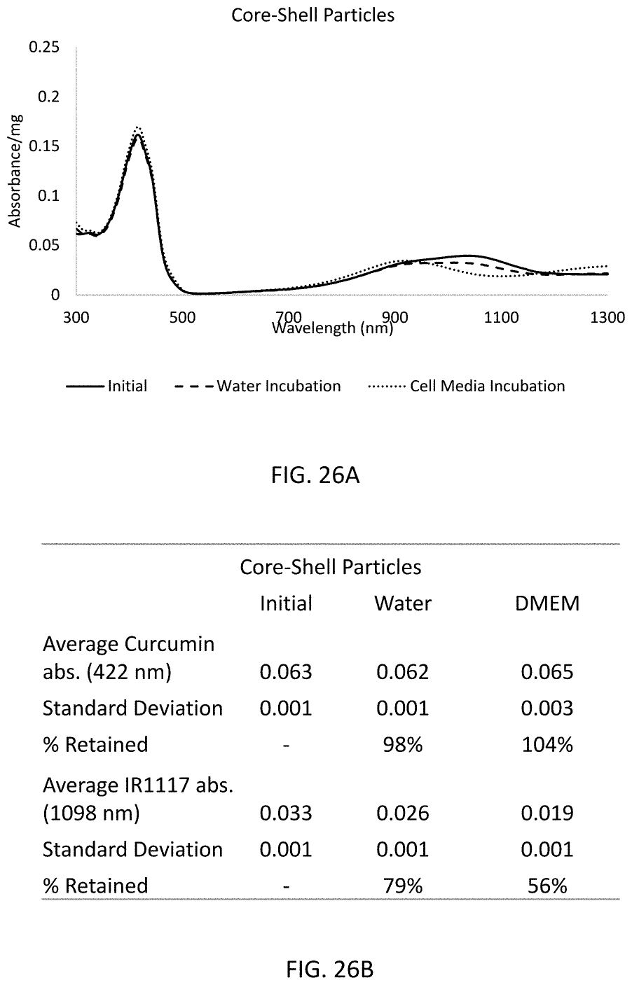

FIGS. 26A and 26B illustrate the results of the EDP for core-shell particles with 5% curcumin and 5% IR dye. Example 5. Extractable Cytotoxicity Test

100 mg of particles are weighed out and then suspended in 1 mL of cell culture media Dulbecco's Modified Eagle's medium (DMEM) containing 10% fetal bovine serum (FBS) and vortexed five times to ensure thorough mixing. This suspension is then incubated at 37° C. in an incubator for 24 hrs. After the incubation period is complete, the suspension is centrifuged at 10,000 g for 10 minutes and the supernatant is collected. The supernatant solution is then filtered through a 0.1 micron syringe filter and is used for cytotoxicity evaluation as the “neat” or 1× sample. This 1× neat extract is serially diluted with media containing 10% FBS for cytotoxicity testing. The following serial dilutions were made using the neat extract and the DMEM supplemented with 10% FBS: 2× (2-fold dilution), 4× (4-fold dilution), 8× (8-fold dilution), 16× (16-fold dilution) and 32× (32-fold dilution), 64× (64-fold dilution) and 1/128× (128-fold dilution).

Inhibitory Concentration for 30% cell killing (IC₃₀) of the extract on AML12 cells (non-malignant hepatocytes obtained from ATCC) is determined by performing an MTS assay, a standard colorimetric method to measure the cell viability following incubation with different dilutions of the 1× extract obtained above. AML12 cells are plated in a 96-well culture plate at a density of 10,000 cells per well and allowed to adhere to the surface overnight. Extract concentrations ranging from 1× to 1/128× are added and incubated for 24 hours at 37° C., in a 5% CO₂ incubator. Controls for the cytotoxicity experiment include “live” and “dead” (cells that are killed due to osmotic pressure by adding D.I. water). “Live” cells have nothing except cell culture media containing 10% FBS added to them and are used to obtain the 100% viability data point. The “dead” control is used to obtain the 0% viability data point for calculating the % viability of cells that are incubated with the different extract concentrations. After 24 hours, to a final volume of 100 μL of media in the cells, 20 μL of PMS activated MTS reagent is added and incubated for 90 minutes. The absorbance is measured at 490 nm using a plate reader (Spectramax M2e, Molecular Devices). Viability of cells is calculated using the absorbance measured at 1× dilution of the extract and the results of absorbance for serial dilutions 1× to 128× of the extract are plotted in MS Excel using linear regression curve fitting algorithm to obtain the IC₃₀. All the samples are tested in triplicate and results are averaged over the three repeats. A particle that results in a 70% cell viability in the cytotoxicity test is considered passing the extractable cytotoxicity test. In some instances, if the neat or dilution concentration of the therapeutic agent and/or the material in the leachate is less than IC₁₀, IC₃₀, IC₄₀, IC₅₀, IC₆₀, IC₇₀, IC₈₀, or IC₉₀, the particle passes the Extractable Cytotoxicity Test.

In another example, Inhibitory Concentration for 30% cell killing (IC₃₀) of the extract on MCF-12A cells (non-malignant mammary breast epithelial cells obtained from ATCC) is determined by performing an MTT assay, a standard colorimetric method to measure the cell viability following incubation with different dilutions of the 1× extract obtained above. Controls for the cytotoxicity experiment include “live” and “dead” (cells that are killed due to osmotic pressure by adding D.I. water). “Live” cells have nothing except cell culture media containing 10% FBS added to them and are used to obtain the 100% viability data point. The “dead” control is used to obtain the 0% viability data point for calculating the % viability of cells that are incubated with the different extract concentrations.

After 24 hours, to a final volume of 50 μL of media in the cells, 50 μL of MTS reagent is added and incubated for 120 minutes. After 120 minutes, 100 μL of MTT solvent is added. The absorbance is measured at 590 nm using a plate reader (Synergy Neo2, BioTek). Viability of cells is calculated using the absorbance measured at 1× dilution of the extract and the results of absorbance for serial dilutions 1× to 16×X of the extract are plotted in MS Excel using linear regression curve fitting algorithm to obtain the IC₃₀. All the samples are tested in triplicate and results are averaged over the three repeats. A particle that results in a 70% cell viability in the cytotoxicity test is considered passing the extractable cytotoxicity test. In some instances, if the neat or dilution concentration of the therapeutic agent and/or the material in the leachate is less than IC₁₀, IC₃₀, IC₄₀, IC₅₀, IC₆₀, IC₇₀, IC₈₀, or IC₉₀, the particle passes the Extractable Cytotoxicity Test.

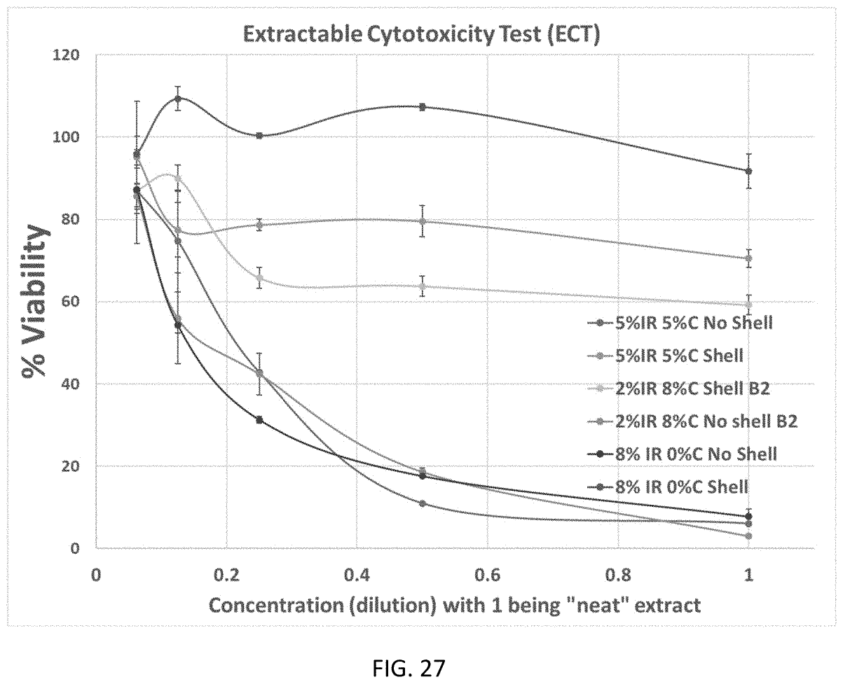

FIG. 27 illustrates ECT results for core and core-shell particles containing either IR dye only or IR dye and curcumin.

Particles that meet the criteria based on the results of the EDP and ECT tests underwent additional testing for thermal cytotoxicity.

### Example 6. Thermal Cytotoxicity Test

The thermal cytotoxicity test uses the 24-well Corning Transwell™ Multiple Well Plate with Permeable Polycarbonate Membrane Inserts. Normal epithelial cells, FHC (ATCC® CRL-1831™) obtained from ATCC, are plated in these 24-well culture plates at a density of 30,000 cells per well and allowed to adhere to the plate surface overnight. Cancer cells (MDA-MB-231 breast cancer cells) are seeded at a density of 30,000 cells and grown on the trans-well inserts of the 24-well Corning plate. The following day, the media in each well is replaced with fresh, cell growth media containing 10% fetal bovine serum. A CellCrown™ insert is used to expose the cancer cells to the particles at different concentrations for testing the thermal cytotoxicity on the cancer cells. These are placed into the trans-well of the Corning plate, such that the insert is submerged in the media but not directly in contact with the cancer cells.

A 1% by weight suspension was prepared for the thermal cytotoxicity tests. 100 mg of dry particles were weighed in 15 ml centrifuge tube and 900 mg of media was added to the tube and vortexed to mix. This was then used for thermal cytotoxicity experiments with 805 nm laser.

A 0.1% by weight suspension was prepared by weighing 10 mg of dry particles in a 15 ml centrifuge tube and adding 990 mg to the tube followed by vortexing to mix the suspension. This was then used for thermal cytotoxicity experiments with 1064 nm laser.

The particles to be irradiated are mixed with cell culture media and added on to the CellCrown™ insert (which includes a transparent PET filter with a pore size of 0.5 μm, allowing heat to easily spread out of the filter into the surrounding media). The CellCrown™ inserts are removed 1 h after incubation of the diseased cells with the particles and media in the trans-well is replaced with fresh complete cell growth media. The incubation period allows for the uptake of the particles into the cells. The cancer cells are then exposed to the exogenous source. This will include irradiation with a laser at three different fluences, each at three different pulse durations to ensure the heat generated is going to kill at least 70% of the cancer cells at different particle concentrations and light doses. The trans-well inserts that have the cancer cells are removed 1-h after irradiation with the exogenous source and placed in a regular 24-well plate for determining the number of cancer cells killed by laser irradiation using an MTS assay, a standard colorimetric method to measure the cell viability 24 h after the irradiation. The non-diseased/normal cells are also incubated for an additional 23 hours at 37° C., in a 5% CO₂ incubator. The viability of the non-diseased, normal cells following the irradiation is also determined by performing an MTS assay to measure the cell viability 24 h after the irradiation. Controls for the thermal cytotoxicity experiment included “live”, “dead” (cells were killed due to osmotic pressure by adding D.I. water) and the particles alone, (i.e. with no laser irradiation) and “light only” for each of the two cell types used. “Live” cells will have nothing except cell culture media containing 10% FBS added to them and are used to obtain the 100% viability data. The “dead” control is used to obtain the 0% data point. “Light only” control includes exposing cells to the equivalent light dose without the composition present in the well. Light doses will be selected to ensure little to no killing of cells is observed using the light only control. At the end of the 24 hours, to a final volume of 200 μL of media in the wells, 40 μL of PMS activated MTS reagent is added and incubated for 90 minutes. The absorbance is measured at 490 nm using a plate reader (Spectramax M2e, Molecular Devices). Viability of both the cell types is calculated using the absorbance measured and the results plotted in MS Excel. The composition and light dose(s) that do not kill any more than 30% of the non-diseased cells but kill at least 70% of the diseased cells are considered passing the thermal cytotoxicity test.

The thermal cytotoxicity test used the 24-well Corning Transwell™ Multiple Well Plate with Permeable Polycarbonate Membrane Inserts. Normal breast epithelial cells, MCF-12A obtained from ATCC, were plated in these 24-well culture plates at a density of 30,000 cells per well and allowed to adhere to the plate surface overnight. Cancer cells (MDA-MB-231 breast cancer cells) were seeded at a density of 30,000 cells and grown on the trans-well inserts of the 24-well Corning plate. The following day, the media in each well wall replaced with fresh, cell growth media containing 10% fetal bovine serum. A CellCrown™ insert was used to expose the cancer cells to the particles at different concentrations for testing the thermal cytotoxicity on the cancer cells. These were placed into the trans-well of the Corning plate, such that the insert is submerged in the media but not directly in contact with the cancer cells.

The particles to be irradiated were mixed with cell culture media and added on to the CellCrown™ insert (which includes a transparent PET filter with a pore size of 0.5 μm, allowing heat to easily spread out of the filter into the surrounding media). The CellCrown™ inserts were removed 1 h after incubation of the diseased cells with the particles and media in the trans-well was replaced with fresh complete cell growth media. The incubation period allows for the uptake of the particles into the cells.

The cancer cells were then exposed to the exogenous source. This included irradiation with a laser at three different fluences, each at three different pulse durations to ensure the heat generated is going to kill at least 70% of the cancer cells at different particle concentrations and light doses. The trans-well inserts that have the cancer cells were removed 1-h after irradiation with the exogenous source and placed in a regular 24-well plate for determining the number of cancer cells killed by laser irradiation using an MTT assay, a standard colorimetric method to measure the cell viability 24 h after the irradiation.

The non-diseased/normal cells were also incubated for an additional 23 hours at 37° C., in a 5% CO₂ incubator. The viability of the non-diseased, normal cells following the irradiation is also determined by performing an MTT assay to measure the cell viability 24 h after the irradiation. Controls for the thermal cytotoxicity experiment included “live”, “dead” (cells were killed due to osmotic pressure by adding D.I. water) and the particles alone, (i.e. with no laser irradiation) and “light only” for each of the two cell types used. “Live” cells have nothing except cell culture media containing 10% FBS added to them and are used to obtain the 100% viability data. The “dead” control is used to obtain the 0% data point. “Light only” control includes exposing cells to the equivalent light dose without the composition present in the well.

Light doses were be selected to ensure little to no killing of cells is observed using the light only control. At the end of the 24 hours, to a volume of 50 μL of media in the wells, 50 of PMS activated MTS reagent is added and incubated for 120 minutes. At the end of 120 minutes, 100 μL of MTT solvent was added and the absorbance was measured at 590 nm using a plate reader (Synergy Neo 2, BioTek). Viability of both the cell types was calculated using the absorbance measured and the results plotted in MS Excel. The composition and light dose(s) that do not kill any more than 30% of the non-diseased cells but kill at least 70% of the diseased cells were considered passing the thermal cytotoxicity test.

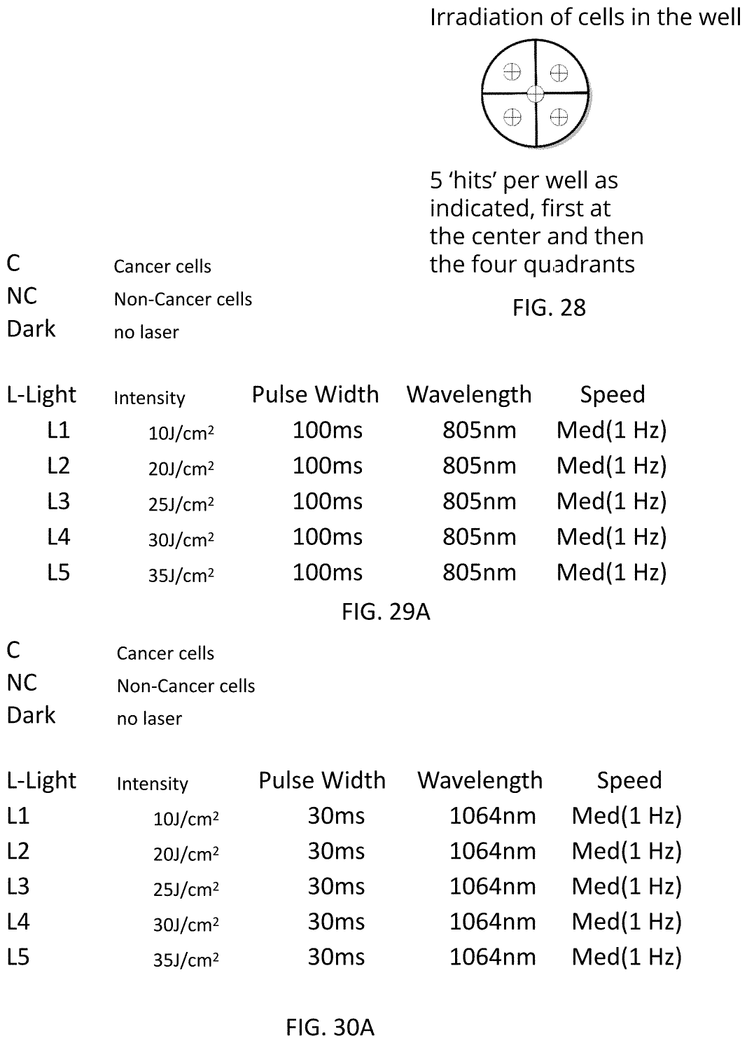

FIG. 28 schematically illustrates the irradiation pattern in which cells in a given well are irradiated.

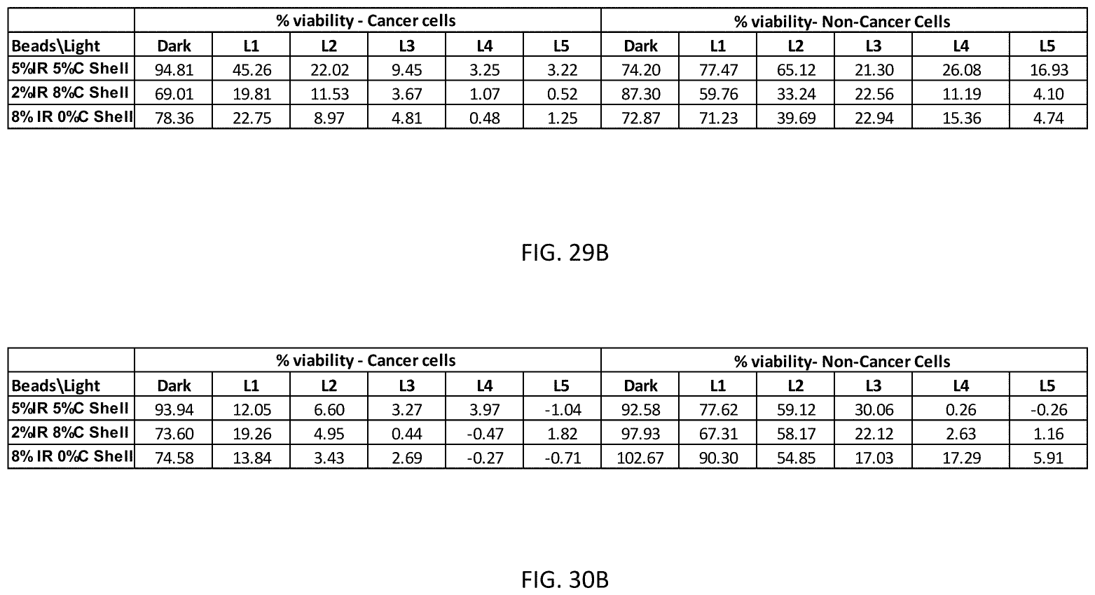

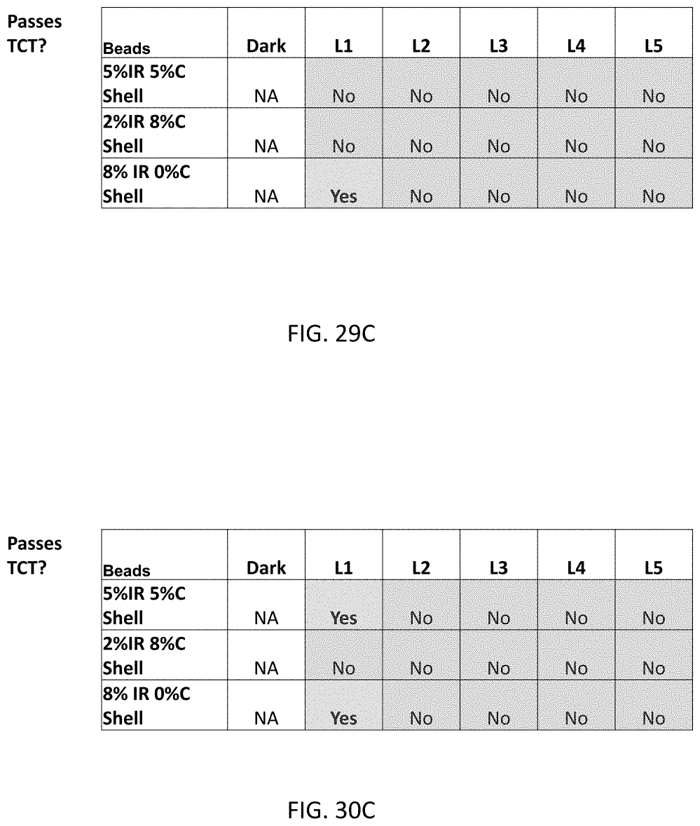

FIGS. 29A-29C show the results of thermal cytotoxicity cells for irradiation by light of 805 nm wavelength.

FIGS. 30A-30C show the results of thermal cytotoxicity cells for irradiation by light of 1064 nm wavelength.

The light dose used was 10 J/cm² at a 1 Hz pulse frequency with a 30 ms pulse. Wavelength used was 1064 nm. Particles with 5% IR, 5% Curcumin shell and 8% IR and 0% Curcumin shell were selected for synergistic killing of cancer cells.

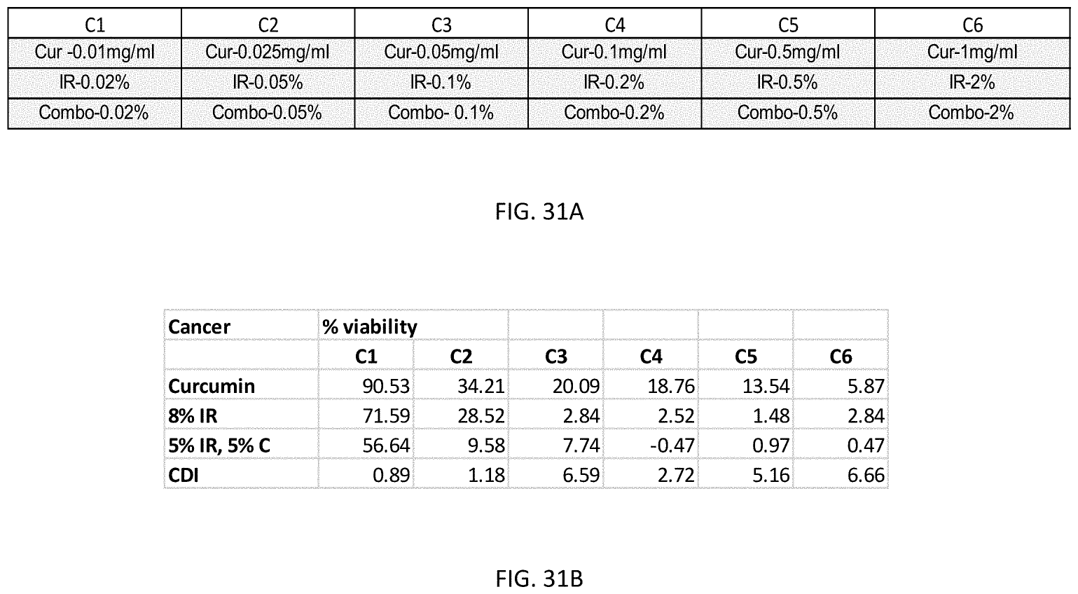

FIGS. 31A and 31B show the results for synergestic combination treatment in which particles with only free curcumin and IR dye were respectively compared to particles containing curcumin and IR dye.

### Example 7. Material Process Stability Test

Particle heaters are dispersed in a 2% solution of gelatin in warm water. The suspension is vortexed and transferred to 50 mm plastic culture dishes and allowed to gel, producing a greenish gel. The optical density is measured by reflectance spectroscopy to provide a baseline absorbance.

Areas on the culture dishes are irradiated over a range of pulse widths and fluences that span the conditions expected for use. Generally, pulse widths range from about 100 μs to about 1 second, with fluences that range from about 0.1 J/cm² to about 60 J/cm². The absorbance is measured for each exposure condition and compared to the baseline absorbance. The preservation greater than 50% absorbance of the material after subject to such process conditions is considered to pass the Material Process Stability Test.

### Example 8. Controlled Heat Generation from Laser-Excited Particle Heaters in Gelatin

The test is to determine threshold conditions for controlled heat generation that produces a thermal increase to 50° C. Heat was generated by exposing a gelatin gel suspension of IR absorbing agent particles as in Example 1(ii) above with a red thermochromic pigment with 50° C. thermal threshold for color loss to laser irradiations with various operating parameters. The gelatin is a degradation product from collagen. The collagen is the primary extracellular matrix protein. The gelatin medium in this example mimics the tumor tissue.

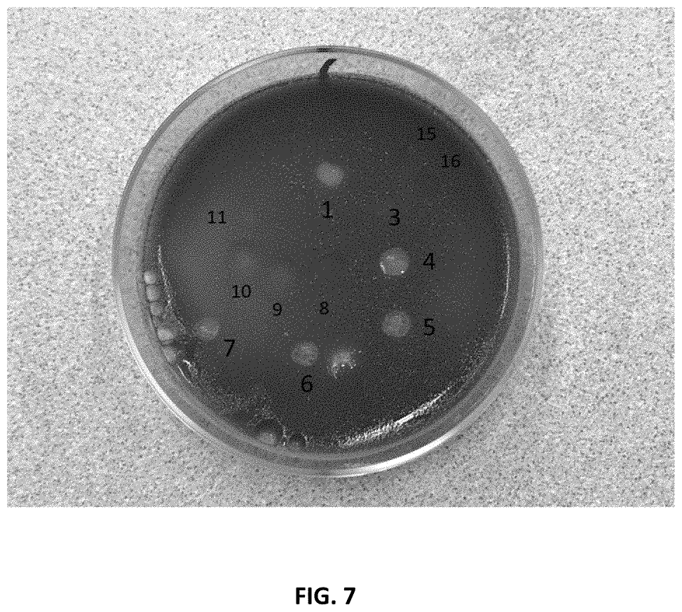

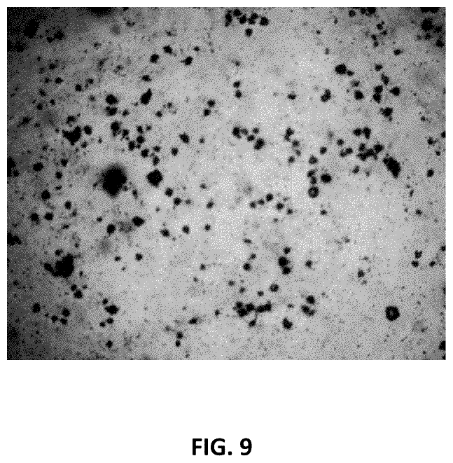

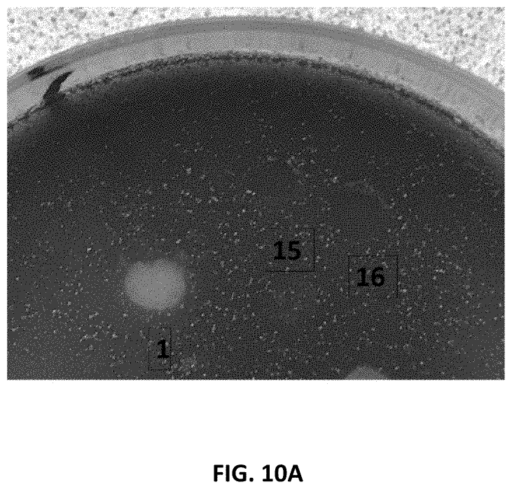

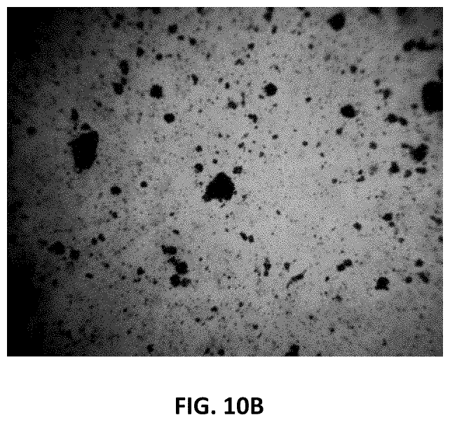

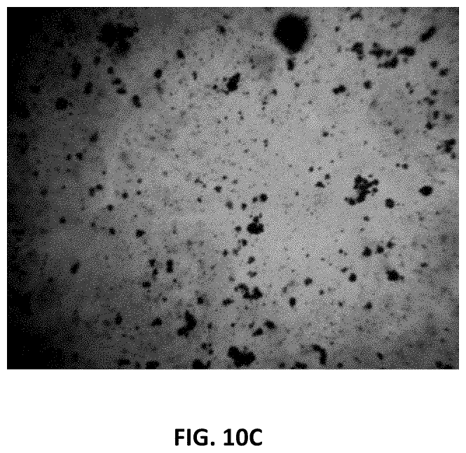

The results of the tests as summarized in the table below demonstrated the capability of the IR absorbing agent particles to absorb energy from laser irradiation and converts the photonic energy to heat. Under the laser operating parameters as set forth below, the heat traveled outside the particle and induced localized hyperthermia in area surrounding the IR absorbing agent particle heaters (see FIGS. 7-10, Table 8).

Thermochromic MC Pigment 50° C. Red (a red thermochromic dye with a threshold temperature for color loss at 50° C., TM PD 50 3111, Lot #MC1204191) was purchased from Sandream Enterprises. Unflavored, commercial, food grade Knox® gelatin was used as received.

A 2.0 wt. % stock solution of gelatin in water was prepared by wetting one gram gelatin with 12 g of cold water, then adding 37 g of water at 75° C., and stirring until dissolved. A 30.0 wt. % stock suspension of particle heaters in water was prepared by suspending of 3.0 g of the particles from Epolight™ IR 1117 particles in 7.0 mL of water.

To 65.0 mg of the particle heater suspension in a 4 dram glass vial was added 25 mg of red thermochromic pigment to form a mixture. To this mixture was added 2.0 g of the 2% gelatin solution, and the glass vial was vortexed for 5 minutes and set aside for use.

The vortexed suspension was transferred by pipette to a 50 mm plastic culture dish, spread evenly, and allowed to cool to form a gel. The particle heaters were spread uniformly within the gelatin gel matrix and gave a greenish color. The particles of the red thermochromic pigment were distributed unevenly within the gelatin matrix (see FIG. 7).

A control sample of red thermochromic pigment, but lacking the particle heaters, was also prepared using the procedure described above by suspending 25 mg of dye in 2 g of 2% gelatin solution, vortexing, spreading evenly in a 50 mm plastic culture dish and allowing to gel.

After the gel had set, it was irradiated with a laser under a variety of different operating parameters. Several regions of the gel (spots 1-3) were first irradiated at 1064 nm in spots of 5 mm diameter with a Lutronic solid state laser, with exposures of 3.51 J/cm² using a 10 ns pulse (Q-switched mode) (Spot 1) and of 2.01 J/cm² (Spot 2) and 3.51 J/cm² (Spot 3) using a 350 μs pulse (Spectra mode). A second set of regions (spots 8-16) were irradiated at 980 nm in spots of about 3 mm diameter with a 10 Watt, electrically switched, CW semiconductor laser with pulse widths ranging from 10-250 ms and delivered energies ranging from 0.5-5 J. The color change effects caused by the laser exposures were photographically recorded using an iPhone camera or microscope camera. The visual results of color changes are shown in FIGS. 7-10. These experiments are summarized in Table 8.

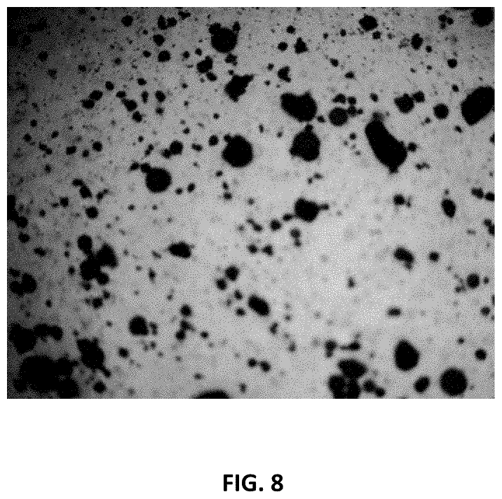

TABLE 8
Results of laser exposure of particle heaters and thermochromic pigment in gelatin

| | | Pulse | | Fluence, | | |
|---|---|---|---|---|---|---|
| Spot | Laser | width | | J/cm² | Result | Image |
| 1 | Lutronic (1064 nm) | 10 | ns | 3.51 | White spot, red pigment decolorized, IR | |
| | | | | | dye color gone | |
| 2 | Lutronic (1064 nm) | 350 | μs | 2.01 | Minimal disturbance of gelatin | |
| 3 | Lutronic (1064 nm) | 350 | μs | 3.51 | Slight depression in gelatin, IR dye not | |
| | | | | | changed. Red pigment melted and color | |
| | | | | | gone. | |
| 8 | Semiconductor | 200 | ms | 28.3 | A spot was formed in the gelatin. IR dye | |
| | laser (980 nm) | | | | was not changed, but red pigment | |
| | | | | | appeared to be melted and color gone. | |
| 9 | Semiconductor | 2 × 250 | ms | 70.7 | Same as spot 8 but bigger spot | FIG. 7 |
| | laser (980 nm) | | | | | |
| 10 | Semiconductor | 250 | ms | 35.4 | Same as spot 8 but slightly bigger spot | |
| | laser (980 nm) | | | | | |
| 11 | Semiconductor | 100 | ms | 14.1 | Approximately 3 mm spot, surface | |
| | laser (980 nm) | | | | particles of red pigment mostly gone | |
| 12 | Semiconductor | 50 | ms | 7.1 | Same effect on gelatin, smaller spot, | |
| | laser (980 nm) | | | | surface particles of red pigment evident | |
| 13 | Semiconductor | 10 | ms | 0.7 | Minimal disturbance of gelatin observed | |
| | laser (980 nm) | | | | | |
| 14 | Semiconductor | 30 | ms | 2.1 | Slight “melting” of gelatin | |
| | laser (980 nm) | | | | | |
| 15 | Semiconductor | 7 × 30 | ms | 14.9 | Similar to spots 11 and 16. Slightly | FIG. 8B |
| | laser (980 nm) | | | | smaller spot than 16 but red pigment | |
| | | | | | melted and color gone | |
| 16 | Semiconductor | 200 | ms | 14.1 | Similar to spot 15 but larger spot. Red | FIG. 8C |
| | laser (980 nm) | | | | pigment melted, color gone. | |

The results in Table 8 show that 1064 nm Q-switched laser irradiation of 3.51 J/cm² led to significant loss of IR absorbing agent and decolorization of red thermochromic pigment. Irradiation with a similar fluence but longer pulse width (Spectra mode) does not show IR absorbing agent degradation but does show melting and decolorization of the red thermochromic pigment. Reducing the fluence to 2.01 J/cm² led to no decolorization and little evidence of heat generation as evidenced by distortion of the gelatin.

Irradiation using the semiconductor laser at 980 nm required greater fluence to produce an equivalent decolorization of the thermochromic pigment. For example, a dose of 14 J/cm² was required to demonstrate complete loss of red color; lower fluence led to no or minimal observable effect. In all cases with this laser, no loss of IR absorbing agent was observed. The retention of the IR absorbing agent was evidenced by the ability to provide enough energy to decolorize the red pigment using several sequential with lower energy pulsed to achieve the same result as irradiation with a single pulse of equivalent total fluence.

The control sample, with red thermochromic pigment only, showed no change when exposed to the semiconductor laser using the settings described in Table 8.

### Example 9. In Vitro Photothermal Performance of Particle Heaters on Killing Cancer Cells

100 mg of particle heaters are weighed out and then suspended in 1 mL of cell culture media Dulbecco's Modified Eagle's medium (DMEM) containing 10% (fetal bovine serum) FBS and vortexed five times to ensure thorough mixing. This suspension is then incubated at 37° C. in an incubator for 24 hrs. After the incubation period is complete, the suspension is centrifuged at 10,000 g for 10 minutes and filtrate is collected. The filtrate is resuspended in 10 mL of cell culture media to give a stock dispersion of particle heaters having a concentration of 10 mg/mL for use in photothermal therapy study.

A Hela cell suspension is prepared by culturing the Hela cells at 37° C. and a 5% CO₂ in Dulbecco's Modified Eagle's medium (DMEM) supplemented with 10% (fetal bovine serum) FBS, 1% penicillin/streptomycin. Hela cells are plated in a 96-well culture plate at a density of 10,000 cells per well and pre-incubated at 37° C. and a 5% CO₂ for 24 hours to allow adhering to the plate well surface. Then the as prepared particle heater dispersion in DMEM medium (10 mg/mL, 1.0 μL) is added into each well of the plate (n=4). The 96-well plate is incubated for another 6 h in the incubator. Subsequently, Hela cells treated or not treated with particle heaters are exposed to an 808 nm laser with 0.28 W/cm² for 4 minutes. After laser irradiation, the Hela cells are washed with fresh culture medic twice to remove the excess particle heaters. After that, the Hela cells are incubated for another 4 hours.

Next the standard MTS assay is conducted to obtain cell viability. The percentage of viable cancer cells is determined by performing an MTS assay, a standard colorimetric method to measure the cell viability following the laser irradiation.

Photothermal ablation of the cancer cells using 1064 nm laser is conducted in the same way. After laser irradiation, the cells are also stained by calcein acetoxymethyl ester (Calcein AM) and propidium iodide (PI) to directly observe the live and dead cells under a fluorescence microscope. For details, the cells are washed with phosphate buffer solution (PBS) twice after laser illumination, and then a PBS solution containing Calcein AM and PI is added to the wells to stain HeLa cells for 10 min under dark conditions. After dye staining, the HeLa cells are washed with PBS twice again and fresh PBS solution is added. Fluorescence images of live and dead HeLa cells are obtained by fluorescence microscopy.

### Example 10. Photothermally Assisted Therapeutic Drug Release

The therapeutic drug release behavior of the particles with and without IR absorbing agent, is investigated to determine the suitability of these polymer-IR absorbing agent composites for photothermally triggered drug delivery. The particles are immersed in a buffer bath and exposed to laser irradiation at 1064 nm. The drug release amount from the control without laser irradiation is measured in each case and compared to the drug release from the particles with the laser irradiation under the same conditions. The amount of paclitaxel or vancomycin release induced by irradiation of the control particle without IR absorbing agent and the particle with the IR absorbing agent is compared. All experiments are performed as three independent repeats.

Measurement of In Vitro Drug Release from the Combination Nanoparticles

In vitro drug release of both the drug (paclitaxel, vancomycin or curcumin) and IR absorbing agent from the particles is evaluated by the dialysis-bag diffusion method. A well characterized particle sample is added to a regenerated cellulose dialysis bag with a MWCO of 20 kDa (Spectra Max®, Chicago, Ill., USA). This dialysis bag is placed in a beaker containing 800 mL of PBS and maintained at 37° C., under 150 rpm stirring. Samples from the dialysis bag are collected from the beaker periodically. To clearly understand the release profiles of both the drugs, the samples are analyzed using reverse phase high performance liquid chromatography (HPLC).

### Peak Separation Using Reverse Phase HPLC

Samples are added with 1% Triton-x to break open the particles and 50 μL of this mixture is injected into an Agilent 1100 High Performance Liquid Chromatography with C18 reverse phase column as stationary phase and 0.1% TFA in acetonitrile as mobile phase. Samples are injected with a flow rate of 1 mL/min with a total run time of 20 minutes. Peak eluents of the signals detected at 227 nm, 273 nm, 295 nm, and 1070 nm at different time points were collected.

### Peak Confirmation Using Mass Spectrometer.

Shimadzu 8040 Triple Quadrupole Liquid chromatograph mass spectrometer (LC/MS) is used to confirm the molecular weights of the peak eluents and clearly distinguish the peaks arising due to either the drug or IR absorbing agent thereby determining the amount of active agent or material present in the sample at different intervals. Once the samples were confirmed, the drug release profiles were plotted using an algorithm [e.g. DDSolver]

### Example 11. Laser-Mediated Release of Drug with Intermittent Irradiation

A set of particles containing the drug (curcumin, vancomycin, or paclitaxel) and IR absorbing agent are prepared as described in the Examples 1(ii), 1(v) above, however the control and drug loaded particles are instead subjected to intermittent irradiation. In this case, corresponding multiple “bursts” of release of the drug from the particles are obtained upon periodic irradiation. The particles are irradiated during the 0-5 minute interval and the 25-35 minute interval. The drug concentrations released at set intervals are collected for the pulsed release of the drug from the particles obtained at 1064 nm with a pulsed Nd:YAG laser (30 J/cm², 30 msec pulse length, 1 Hz repetition rate). The release of drug from particles in response to sequential irradiation at 805 nm (800 mW) by a continuous diode laser at set intervals are measured. The effectiveness of drug release modulated by the continuous laser and the pulsed laser is compared.

### Example 12. Model for In Vivo Triggering of Drug Release

A set of particles containing the drug (curcumin, vancomycin or paclitaxel) and IR absorbing agent are prepared and treated as described above. Prior to irradiation, a section of rat skin is placed inside the vial containing the particle to simulate an in vivo situation. The rat skin is obtained from a hooded Long-Evans rat immediately after sacrifice and placed in a glycerol bath for 3 hours. The skin section is taken from the bath and the excess glycerol is removed with a paper towel. The section is then clamped into place inside the glass vial containing the particle dispersion. The particles subsequently irradiate through the skin with a pulsed Nd:YAG laser at 1064 nm (30 J/cm², 30 msec pulse length, 1 Hz repetition rate). The concentrations of the released model drug paclitaxel or vancomycin are sampled and measured at 0, 5, 10, 15, 20, 25, 30, 35 and 40 minutes during the irradiation sequence. Any damage to the skin samples during the 40-minutes irradiation period is noted. The experiment is done in triplicate for reproducibility.

### Example 13. PMMA/BMA 805-Epolight™ IR 1117-curcumin Particles

Reagents: Chemical reagents sodium dodecyl sulfate (SDS), polyvinyl alcohol (PVA), Curcumin were purchased from Aldrich; vinyltrimethoxysilane (VTMS) was purchased from Gelest, Inc. Neocryl® B-805 polymer (MMA/BMA copolymer, weight average molecular weight=85,000 Da, glass transition temperature T_{g}=99° C.) was purchased from DSM. Epolight® 1117 (tetrakis aminium, absorbing at 800 nm-1071 nm, melting point: 185-188° C., soluble in acetone, methylethylketone and cyclohexanone) was purchased from Epolin Inc.

Abbreviations: n-BMA: n-butyl methacrylate; MMA: methyl-methacrylate

### Example 13 (a): Preparation of PMMA/BMA B-805-Epolight™ IR 1117-curcumin Particles

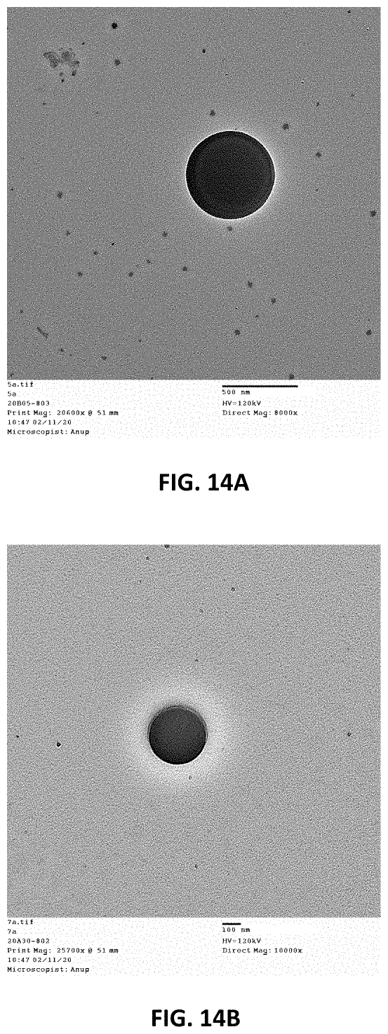

This method results in a primary particle (no shell) both the active agent (curcumin) and the material (IR absorbing agent) are embedded with the PMMA/BMA copolymer matrix (See FIGS. 14A-B).

The preparation of the aqueous phase: 0.86 g of sodium dodecyl sulfate (SDS) was added into 143 g of 8.0% aqueous polyvinyl alcohol (PVA) solution placed in a round bottom flask. An aqueous solution of SDS containing 8.0% PVA was formed after the dissolution of SDS (the aqueous phase). The aqueous phase was stirred with an IKA t-25 Turrax at 8000 RPM.

The preparation of the organic phase: to 66 g of dichloromethane was added 6.0 g of DSM Neocryl® B-805 polymer (MMA/BMA copolymer), 0.13 g curcumin, and 0.53 g of Epolight® IR 1117 to allow the formation of a clear solution of Neocryl® B805 polymer and encapsulants.

The organic phase (polymer and encapsulants dissolved in dichloromethane) was injected directly into the aqueous phase (PVA solution with SDS surfactant) at the tip of the Turrax's rotostator.

The shear mixing at 8000 RPM was continued for 30 minutes. The resulting mixture was decanted into an open-mouth container and stirred for 16 hours. A suspension of solid particles in aqueous fluid was produced.

The suspension of particles was centrifuged at 5000 RPM for 30 minutes and the particles were collected. The collected particles were washed with distilled water by resuspending the particles into distilled water and centrifuging to collect the particles. This particle washing process was repeated three times to remove the residual PVA. The resulting encapsulant/MMA/BMA copolymer particles were suspended in distilled water.

### Example 13 (b): Preparation of VTMS Encased PMMA/BMA B-805-Epolight™ IR 1117-Curcumin Particles Having a 25% VTMS Shell

In this example, a sol-gel vinyl modified silicone polymer shell was made from a VTMS HCl solution containing VTMS at 25 wt. % of the total weight of the VTMS HCl solution. The weight amount of VTMS in the solution comprised 25 wt. % of the total weight of the VTMS reagent and uncoated particle (weight ratio VTMS/uncoated particle=0.33:1), hereafter referred to as “25% VTMS shell”.

In a first vessel, 1.08 g (0.007 mmol) of vinyltrimethoxysilane (CH2=CHSi(OMe)3, VTMS, MW=148 Da) was mixed with 3.26 g of dilute aqueous hydrochloric acid under magnetic stirring (24.9 wt. % solution of CH2=CHSi(OMe)3 in diluted HCl) The resulting mixture was stirred for 2 hours to allow complete hydrolysis of VTMS to give vinylsilanetriol (CH2=CHSi(OH)3, MW=106 Da).

In a second vessel, under magnetic stirring, 2 g of pre-made uncoated IR absorbing agent particles of Example 1a above was dispersed in 72.9 grams of water to provide a 2.7 wt. % particle dispersion. The pH value of the resulting IR absorbing agent particle aqueous dispersion was adjusted to 10.0 with the addition of dilute aqueous sodium hydroxide. To this particle dispersion at pH 10, the 4.34 g of hydrolyzed 25 wt. % VTMS solution was added at a rate of 2 drops per second to the particle suspension. The pH value of the resulting suspension was monitored after the hydrolyzed 25% VTMS solution addition and adjusted with sodium hydroxide solution to maintain a pH of 10 for 60 minutes. After 60 minutes, the suspension was neutralized with glacial acetic acid to lower the pH from 10 to 5.0. The weight ratio of VTMS to the uncoated particle is 0.33:1.

The resulting particle suspension was centrifuged for 30 minutes at 5000 RPM to collect the sol gel vinylsilicate-coated IR absorbing agent particles. The particles collected after the centrifugation were redispersed in distilled water and subjected to centrifugation to collect the particles. This washing procedure was repeated 3 times to remove any unreacted chemical reagents. The resulting sol gel vinylsilicate-coated particles were suspended in distilled water.

### Example 13 (c): Particle Property Characterization

### (i): Particle Size Distribution Measurement

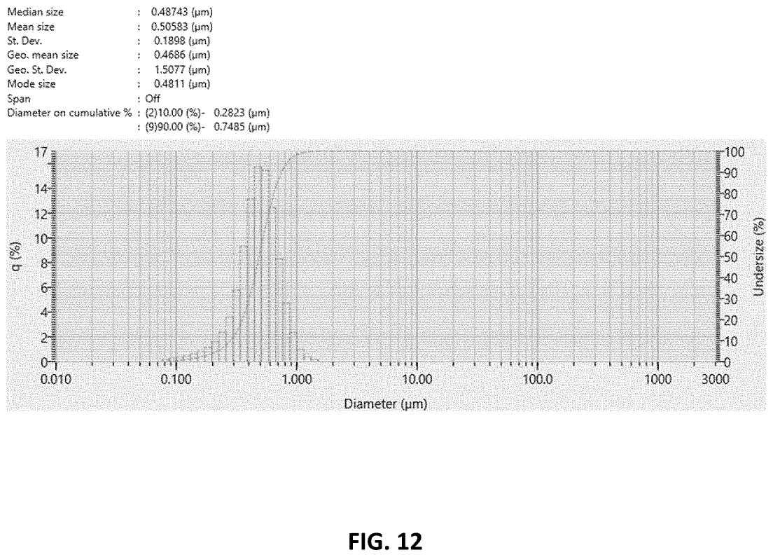

The particle size distribution of the resulting IR absorbing agent/MMA/BMA copolymer particles of Example 13(b) were measured with Horiba LA-950 Particle Size Analyzer in distilled water with pH 7.4 (See FIG. 12). All particle size measurements were carried out at room temperature (about 17-22° C.). The median particle size (D₅₀) for the resulting encapsulant/MMA/BMA copolymer particles is 0.487 μm.

### (ii) The Percentage of Curcumin and Epolight™ IR 1117 Loading Determination

The percentage of curcumin and Epolight™ IR 1117 loaded in to the B-805 PMMA-BMA particles can be determined according to the following procedure: Known quantities of particles in deionized water are added to a solution of 2% Triton-X solution in a 1:1 volume ration. The UV-VIS-NIR absorbance spectrum of the IR absorbing agent and/or the drug is measured using Shimadzu UV-3600 UV-VIS-NIR Spectrophotometer. The concentration of the IR absorbing agent in the particles is determined from application of Beer's law. A similar procedure can be used to measure paclitaxel or vancomycin drug concentration via HPLC as described earlier.

$$[ Epolight IR 1117 \text{/} curcumin ] ( μ M ) = \frac{{Absorbance}_{λ}}{{ɛ}_{λ} \times l} \times {10}^{6}$$

where the path length, 1, is 1 cm.

The quantity of curcumin and Epolight™ IR 1117 is determined from the product of the concentration, the amount of total solution, and the molecular weight of curcumin and Epolight™ IR 1117. The curcumin and Epolight™ IR 1117 loading as a percentage of the total particle mass is determined from:

$$IR absorbing agent \text{/} Drug Loading ( \% ) = \frac{Amount of IR absorbing agent \text{/} drug in solution}{Amount of particle used} \times 100 \%$$

The particles made by Example 13(b) has a starting ingredient composition comprising 90 wt. % of B-805 PMMA/BMA copolymer, 2.0 wt. % curcumin and 8.0 wt. % Epolight™ IR 1117 by the total weight of B-805 polymer, curcumin, and Epolight™ IR 1117 prior to form the particle.

The measured encapsulation of curcumin is 78% and the encapsulation of Epolight™ IR 1117 is 76%.

(iii) The Protective Effect of VTMS Shell

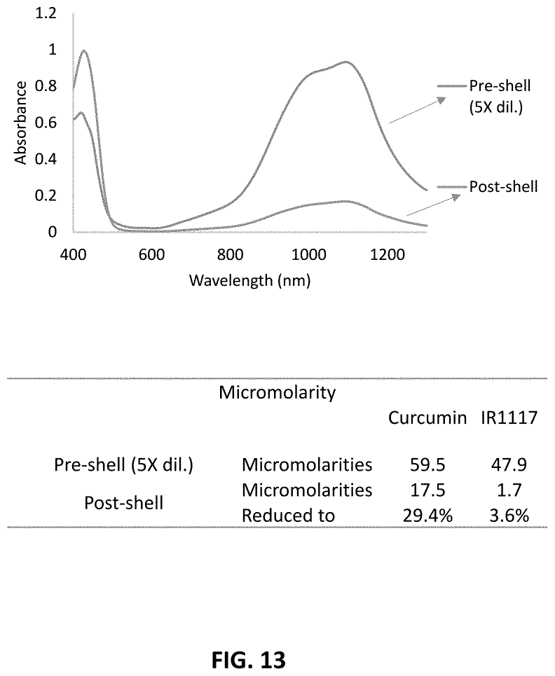

The stability tests were performed on Epolight™ IR 1117 and curcumin in the B-805 particles with and without VTMS shell as prepared in Examples 13(a) and 13(b). Prior to the test, particles were dispersed in water for storage at varying concentrations. Amounts of fluid containing 50 mg of particles—a sample of pre-coating particles and a sample of post-coating particles—were weighed into 15 mL conical centrifuge tubes. The tubes were centrifuged to sediment the particles and supernatant was removed. 3 mL of 1% SDS in water was added to each tube, which were then vortexed to resuspend particles. The tubes containing the particles suspended in SDS solution were placed into a sonicated water bath for 90 minutes. The tubes were then removed and centrifuged to sediment the particles again. The supernatant was retained and filtered through a 0.2 μm syringe filter to remove any remaining particles. Absorption spectra of the supernatants was then measured on a Shimadzu 3600 UV/Vis/NIR spectrophotometer from 400-1300 nm. Molarity of colored components in the solutions and the percentage decrease of absorption between post-coating and pre-coating particles was calculated from the spectra. The results are summarized in FIG. 13.

The leaching testing results demonstrated that the VTMS shell reduced the leaching of the curcumin by 70% and reduced the leaching of Epolight™ IR 1117 by 96% (See FIG. 13).

### (iv): Particle Transmission Electron Microscope (TEM) Image

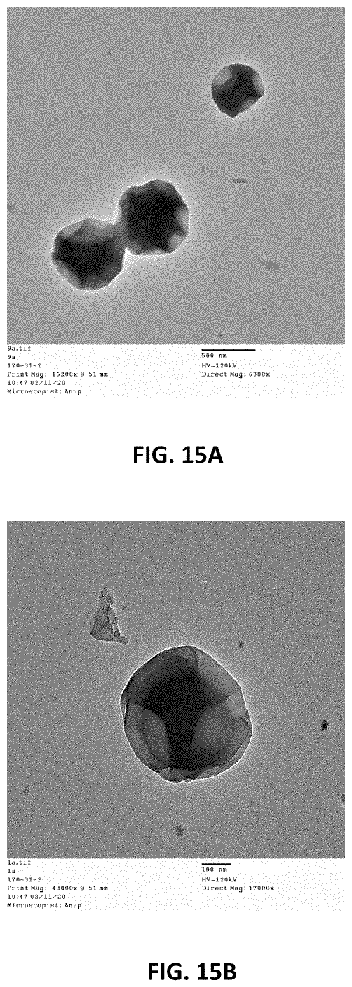

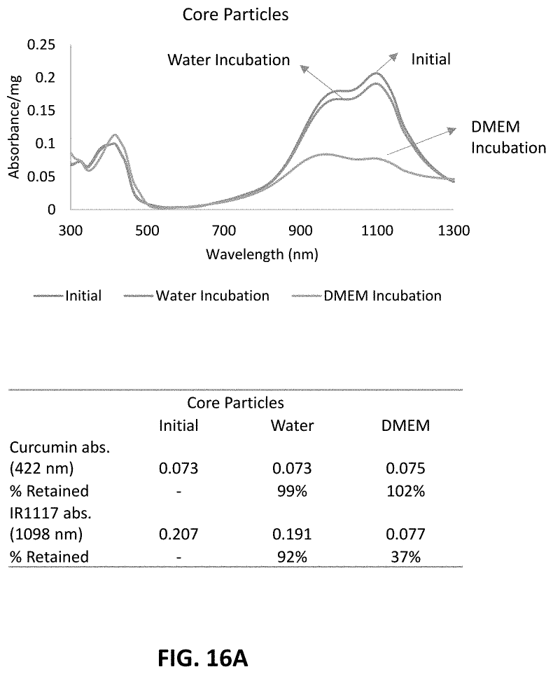

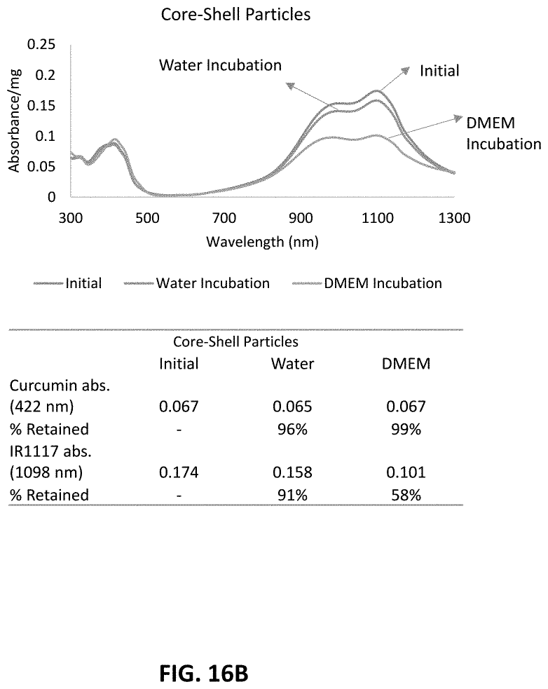

The TEM images for the curcumin and Epolight™ IR 1117 loaded B-805 PMMA-BMA and VTMS shell encased curcumin and Epolight™ IR 1117 loaded B-805 PMMA-BMA are measured using a Philips EM400 TEM instrument (See FIGS. 14A-B, and FIGS. 15A-B).

### Example 13 (d): EDP Test

Procedure: A solution of gelatin was prepared by adding Knox gelatin (1.0 g) to cold water (12.5 g) in a 100 mL glass jar equipped with a magnetic stir bar. The gelatin was stirred 15-30 minutes and then hot water was added (70 C) until the total weight was 50 g. This gelatin was then used in 2 gm aliquots. Drug loaded PMMA beads with silane shells (20-30 mg) were then added to the gelatin solution (2.0 g) and vortexed mixed in a 4 dram vial. The suspension of particles in gelatin was then sonicated for 15-30 minutes before transferring to a 5 cm plastic culture dish. The gelatin suspension was spread evenly and allowed to set. It was then covered and placed in a refrigerator a 6 C until ready for laser exposure.

Laser exposure was accomplished as follows: The cover for the culture dish was removed and a 5 cm clear plastic cover was cut and fit over the gelatin to prevent splatter. The entire surface was then exposed to the Q-Switched laser (Lutronic Spectra™ VRM II Laser with four distinct Q-switched mode wavelengths: 1064 nm, 532 nm, 585 nm, 650 nm, nano second pulse width, and spectra peak energy: 60 MW, 120 MW and 240 MW) using a 5 mm spot size and fluences ranging from 2.0 J/cm2 to 5.0 cm2. Approximately, 200-300 pulses were used to cover the top surface. The cover was then replaced and the sample turned over and if needed, more laser exposure (0-100 pulses) was done from the back side of the sample.

After the sample was exposed as fully as possible to the laser, the beads were reisolated from the gelatin. The gelatin containing the laser exposed particles was then removed from the culture dish by scraping with a spatula and washing with water or phosphate buffered saline (for cytotoxicity testing) and placing the material into a centrifuge tube. Approximately 6 g of additional water or PBS was used in the transfer and to redissolve the gelatin. The centrifuge tube was then sonicated in a warm bath (35-40° C.) until the gelatin redissolved. The beads were then recovered by centrifugation, washed with water (2×8 gm), dried and analyzed for chemical composition and for cytotoxicity.

For chemical composition, approximately 5-10 mg of dried beads were dissolved in 25 mL dichloromethane and analyzed on a Shizmadzu spectrophotometer from 350-1300 nm. The amount of colored dye remaining relative to a control sample (unlasered) was then determined. The amount of IR absorbing agent remaining was also determined relative to the control. The percentage decline in absorbance of the drug was then compared to the percentage decline in the IR absorbing agent. It was observed in all cases that the IR absorbing agent declined more than the drug.

Approximately 5-10.0 mg of dried beads were dissolved in 25 mL dichloromethane and analyzed on a Shizmadzu spectrophotometer from 350-1300 nm. The amount of drug remaining relative to a control sample (unexposed) was then determined. The amount of IR absorbing agent remaining was also determined relative to the control. The percentage decline in absorbance of the drug was then compared to the percentage decline in the IR absorbing agent. It was observed in all cases that the IR absorbing agent declined more than the drug. IR absorbing agent decomposes.

### Example 13 (e): Laser Triggered Drug Release

Procedure: A solution of gelatin was prepared by adding Knox gelatin (1.0 g) to cold water (12.5 g) in a 100 mL glass jar equipped with a magnetic stir bar. The gelatin was stirred 15-30 minutes and then hot water was added (70° C.) until the total weight was 50.0 g. This gelatin was then used in 2.0 gm aliquots. Drug loaded PMMA beads with 25% VTMS shells as prepared in Example 1b above (20-30 mg) were then added to the gelatin solution (2.0 g) and vortexed mixed in a 4 dram vial. The suspension of the tattoo particles in gelatin was then sonicated for 15-30 minutes before transferring to a 5 cm plastic culture dish. The gelatin suspension was spread evenly and allowed to set. It was then covered and placed in a refrigerator a 6° C. until ready for laser exposure.

Laser exposure was accomplished as follows: The cover for the culture dish was removed and a 5 cm clear plastic cover was cut and fit over the gelatin to prevent splatter. The top surface was then completely lasered at 1064 nm with a 5 mm spot using fluences ranging from 2.46 J/cm² to 5.09 J/cm² using Q-switched Lutronic laser.

After lasering the top surface the culture dish was covered with its lid, turned over and lasered from the opposite side to reach any unexposed beads visible only from the bottom side.

The standard fluence for tattoo removal is 3.51 J/cm². 5.09 J/cm² is the maximum fluence on the Lutronic laser using a 5 mm spot.

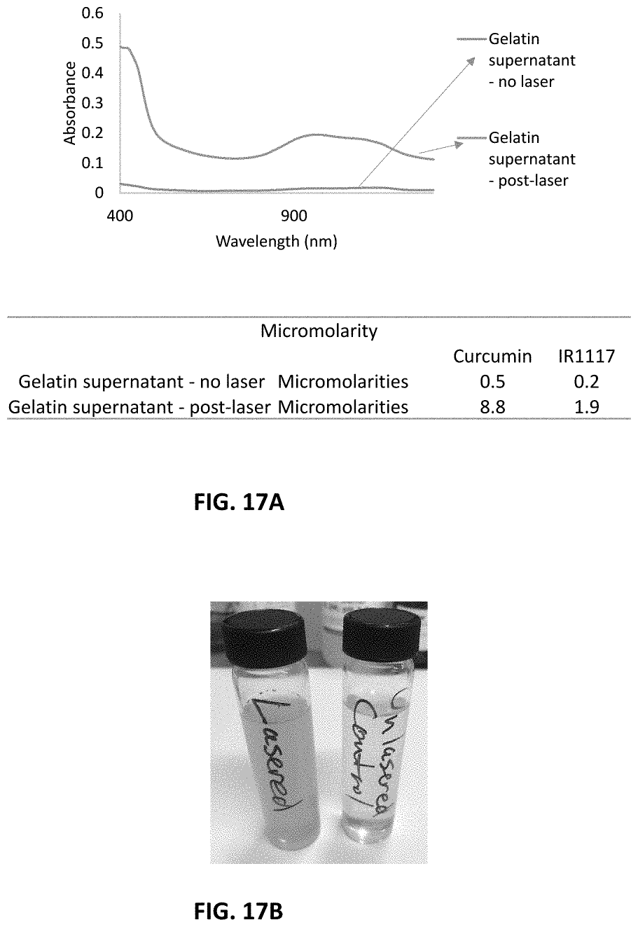

The results are summarized in FIG. 17A. There are about 3 wt. % of curcumin are released from the particles.

The released curcumin caused by the laser exposures to an aqueous solution extract can be visually observed by its yellow color and the yellow colored released curcumin solution was photographically recorded using an iPhone camera or microscope camera (See FIG. 17 B).

This result indicates that a very low level of light dose (3.5 J/cm²) with the Q-switch laser can trigger release of the curcumin which kills cells.

Results demonstrate sustained release from the lasered bead samples which is not the case in the non-lasered samples. Choice of polymer could influence release profile-.

The difference between the two curves is clearly demonstrating a significant difference in the toxicity of the supernatant solution obtained from beads that were exposed to laser.

The difference in color of the two supernatant solutions and the absorption spectrophotometry along with literature on IC₅₀ of curcumin on various cells lines completely align with these findings.

Clearly, lasering of the Curcumin loaded particles triggers some (relatively) immediate (or “burst”) release. Curcumin loaded particles with shell can be triggered to release payload (curcumin) which is toxic to cells. Without laser, Curcumin loaded particles are not cytotoxic. Curcumin loaded particles exposed to laser continued to release payload (curcumin) which is toxic to cells.

### Example 13 (f): Extractable Cytotoxicity Test on PMMA/BMA B-805-Epolight™ IR 1117-Curcumin Particles

Procedure: A solution of gelatin was prepared by adding Knox gelatin (1.0 g) to cold water (12.5 g) in a 100 mL glass jar equipped with a magnetic stir bar. The gelatin was stirred 15-30 minutes and then hot water was added (70° C.) until the total weight was 50.0 g. This gelatin was then used in 2.0 gm aliquots. Active agent loaded PMMA beads with 25% VTMS shells as prepared in Example 1b above (20-30 mg) were then added to the gelatin solution (2.0 g) and vortexed mixed in a 4 dram vial. The suspension of particles in gelatin was then sonicated for 15-30 minutes before transferring to a 5 cm plastic culture dish. The gelatin suspension was spread evenly and allowed to set. It was then covered and placed in a refrigerator a 6° C. until ready for laser exposure. For cytotoxicity experiments as many as 6 dishes containing up to 30 mgs of beads were prepared for laser imaging along with an equal number of control dishes which would not be lasered.

Laser exposure was accomplished as follows: The cover for the culture dish was removed and a 5 cm clear plastic cover was cut and fit over the gelatin to prevent splatter. The entire surface was then exposed to the Q-Switched laser (Lutronic Spectra™ VRM II Laser with four distinct Q-switched mode wavelengths: 1064 nm, 532 nm, 585 nm, 650 nm, nano second pulse width, and spectra peak energy: 60 MW, 120 MW and 240 MW) using a 5 mm spot size and fluences ranging from 2.0 J/cm² to 5.0 cm². Approximately, 200 pulses were used to cover the top surface. The cover was then replaced, and the sample turned over and if needed, more laser exposure (0-100 pulses) was done from the back side of the sample.

After lasering the top surface, the culture dish was covered with its lid, turned over and lasered from the opposite side to reach any unexposed beads visible only from the bottom side.

The standard fluence for tattoo removal is 3.51 J/cm². 5.09 J/cm² is the maximum fluence on the Lutronic laser using a 5 mm spot.

After the sample was exposed as fully as possible to the laser, the VTMS encased curcumin/IR 1117/B-805 particles were reisolated from the gelatin. The gelatin containing the laser exposed VTMS encased curcumin/IR 1117/B-805 particles was then removed from the culture dish by scraping with a spatula and washing with water or phosphate buffered saline (for cytotoxicity testing) and placing the material into a centrifuge tube. Approximately 6 g of additional water or PBS was used in the transfer and to redissolve the gelatin. The centrifuge tube was then sonicated in a warm bath (35-40° C.) until the gelatin redissolved. The beads were then recovered by centrifugation, washed with water (2×8 gm), dried and analyzed for cytotoxicity. The PBS supernatant containing dissolved gelatin and soluble products from the lasering of the beads from the first centrifugation was also analyzed for cytotoxicity.

The supernatant solutions from the laser treated B-805 particles and from the particles without laser treatment as obtained in the Example 13(e) above was then filtered through a 0.1 micron syringe filter and is used for cytotoxicity evaluation as the “neat” or 1× sample. This 1× neat extract is serially diluted with media containing 10% FBS for cytotoxicity testing. The following serial dilutions were made using the neat extract (1× or full strength) and the DMEM supplemented with 10% FBS-0.5× (half-strength), 0.25× (quarter-strength), 0.125×, 0.0625× and 0.03125×

The cytotoxicity of the supernatant solution and the extract was evaluated on NIH-3T3 cells (obtained from ATCC) by performing an MTS assay, a standard colorimetric method to measure the cell viability following incubation with different dilutions of the 1× extract obtained above. NIH-3T3 cells are plated in a 96-well culture plate at a density of 10,000 cells per well and allowed to adhere to the surface overnight. Extract concentrations ranging from 1× to 0.03125× are added and incubated for 24 hours at 37° C., in a 5% CO₂ incubator. Controls for the cytotoxicity experiment include “live” and “dead” (cells that are killed due to osmotic pressure by adding D.I. water). “Live” cells have nothing except cell culture media containing 10% FBS added to them and are used to obtain the 100% viability data point. The “dead” control is used to obtain the 0% viability data point for calculating the % viability of cells that are incubated with the different extract concentrations. After 24 hours, to a final volume of 100 of media in the cells, 20 μL of PMS activated MTS reagent is added and incubated for 90 minutes. The absorbance is measured at 490 nm using a plate reader (Spectramax M2e, Molecular Devices). Viability of cells is calculated using the absorbance measured at 1× dilution of the extract and the results of absorbance for serial dilutions 1× to 128× of the extract are plotted in MS Excel using linear regression curve fitting algorithm to obtain the IC₃₀. All the samples are tested in triplicate and results are averaged over the three repeats. A particle that results in a 70% cell viability in the cytotoxicity test is considered passing the cytotoxicity test.

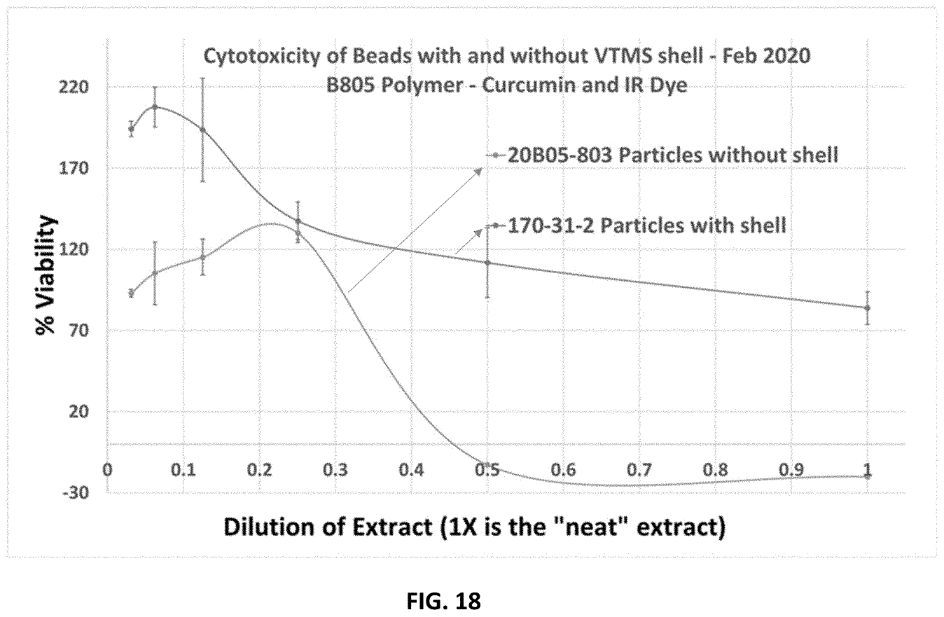

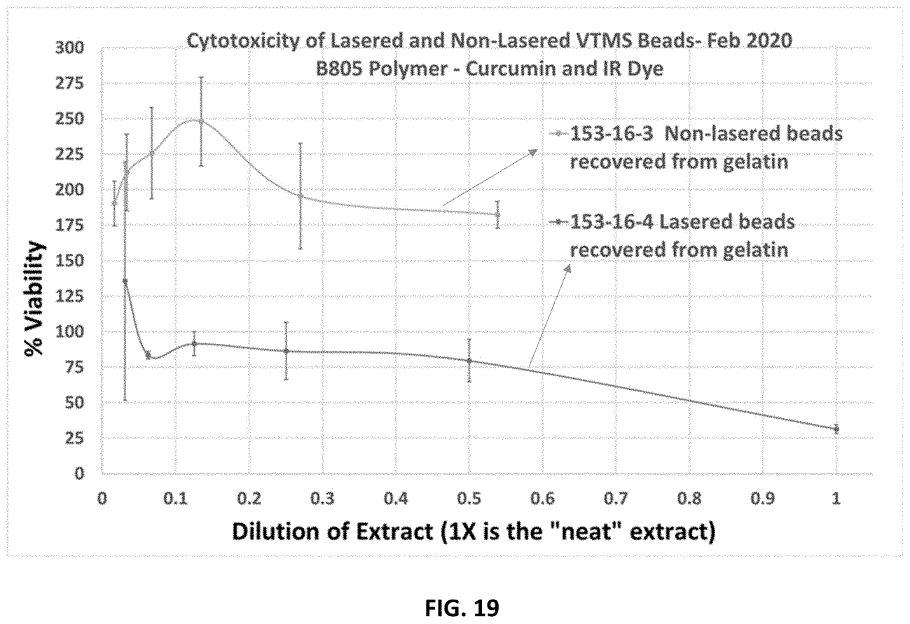

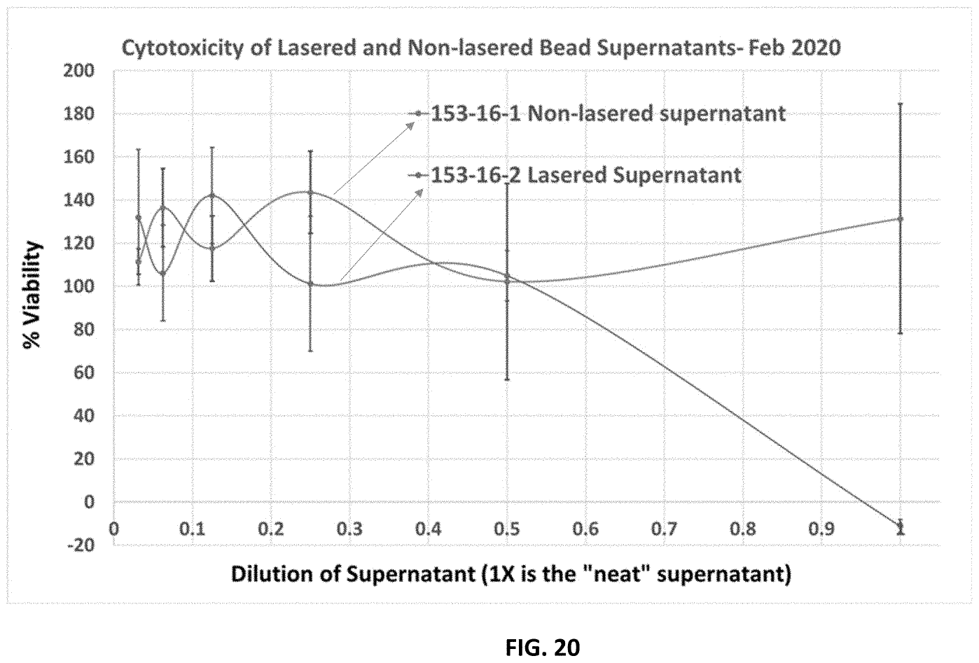

The testing results are summarized in FIGS. 18-20.

The particles without the shell failed the ECT at “neat” or “1×” extract strength and at half strength (“0.5×”) but passed at quarter strength (“0.25×”) and higher dilutions of the extract.

Dilutions in the “particles without shell” samples showing negative viabilities are an artifact since when checked under the microscope they looked just like the “dead” or 0% viability control.

Particles with a 25% VTMS shell passed ECT at 1× extract strength and all subsequent dilutions. These results demonstrated that adding a VTMS shell to encase the curcumin/Neocryl B-805 core particle reduces the cytotoxicity as compared with the curcumin/Neocryl B-805 core particle without shell.

Supernatant from beads suspended in gelatin that were not exposed to laser clearly passed ECT at all dilutions. Results prove that cytotoxicity of particles can be reduced by controlling particle structure (in this case by adding a shell). This result along with the EDP results and the leaching test results confirmed the validity of the Feedback Loop 1A and justify its use to modify particle structure for controlling toxicity prior to interaction with the exogenous source.

Various examples of aspects of the disclosure are described as numbered embodiments (1, 2, 3, etc.) for convenience. These are provided as examples, and do not limit the subject technology. Identifications of the figures and reference numbers are provided herein merely as examples and for illustrative purposes, and the embodiments are not limited by those identifications.

Embodiment 1. A particle for use in treating a cancer comprising: (a) an anticancer agent, (b) a carrier, (c) a material that interacts with an exogenous source, wherein the anticancer agent is encapsulated by the carrier, wherein the anticancer agent and the material in the particle exhibit stability such that the particle is considered passing the Efficacy Determination Protocol; wherein the particle structure is constructed such that it passes the Extractable Cytotoxicity Test; wherein the material absorbs the energy from the exogenous source and converts the energy into heat; and then the anticancer agent is released outside the particle.

Embodiment 2. The particle of embodiment 1, wherein the carrier comprises a polymer having labile bonds susceptible to hydrolysis.

Embodiment 3. The particle of embodiment 2, wherein the hydrolytic degradation of the carrier is accelerated by the heat.

Embodiment 4. The particle of embodiment 1, wherein the unencapsulated anticancer agent has a plasma half-life of less than 30 minutes.

Embodiment 5. The particle of embodiment 2, wherein the anticancer agent is a Class II, Class III or Class IV compound according to the Biopharmaceutics Classification System.

Embodiment 6. The particle of any one of embodiments 1-3, wherein the anticancer agent is selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, dar-inaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, caba-zitaxel, capecitabine, crizotinib, dabrafenib, dasatinib, N1,N11-bis(ethyl)norspermine (BENSM), ibrutinib, idelali sib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipo-tecan, GSK-461364, and combinations thereof.

Embodiment 7. The particle of any one of embodiments 1-3, wherein the anticancer agent is a PI3K inhibitor selected from the group of wortmannin, temsirolimus, everolimus, bupar-lisib (BMK-120), 5-(2,6-dimorpholinopyrimidin-4-yl)-4-(trifluoromethyl)pyridin-2-amine), pictilisib, gedatolisib, apitolisib, pilaralisib, copanli sib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide) and combinations thereof.

Embodiment 8. The particle of any one of embodiments 1-3, wherein the anticancer agent is a proteasome inhibitor selected from the group of bortezomib, ixazomib, marizomib, oprozomib, delanzomib, epoxomicin, disulfiram, lactacystin, beta-hydroxy beta-methylbutyrate, and combinations thereof.

Embodiment 9. The particle of any one of embodiments 1-3, wherein the anticancer agent is an EGFR inhibitor selected from the group of erlotinib, gefitinib, neratinib, osimertinib, vandetanib, dacomitinib, lapatinib, and combinations thereof.

Embodiment 10. The particle of any one of embodiments 1-7, wherein the carrier comprises polymer with heat-labile moieties, or polymer having labile bonds susceptible hydrolysis, or enzymatic degradation.

Embodiment 11. The particle of embodiment 8, wherein the labile bonds are selected from the group of an ester bond, an amide bond, an anhydride bond, an acetal bond, a ketal bond, and combinations thereof.

Embodiment 12. The particle of any one of embodiments 1-9, wherein the carrier is selected from the group of a polyester, a polyanhydride, a polysaccharide, a polyphosphoester, a poly(ortho ester), a poly(amino acid), a protein, polyurea, and combinations thereof.

Embodiment 13. The particle of embodiment 10, wherein the polymer selected from the group of poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (□-lysine), di-block copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

Embodiment 14. The particle of embodiment 10, wherein the polymer comprises a mixture of (i) a first PLGA having number average molecular weight ranging from 2000 Da to 3000 Da, and (ii) a second PLGA having number average molecular weight ranging from 570 Da to 1667 Da.

Embodiment 15. The particle of embodiment 14, wherein the first and second PLGA have a lactide:glycolide molar ratio ranging from 5:95 to 95:5, 10:90 to 90:10, 15:85 to 85:15, 25:75 to 75:25, 40:60 to 60:40, or 45:55 to 55:45.

Embodiment 16. The particle of embodiment 14, wherein the polymer comprises the first PLGA and the second PLGA in a weight ratio of first PLGA to second PLGA ranging from 10:1 to 1:10.

Embodiment 17. The particle of embodiment 10, wherein the polymer comprises a PLGA having a lactide:glycolide molar ratio ranging from 5:95 to 95:5, 10:90 to 90:10, 15:85 to 85:15, 25:75 to 75:25, 40:60 to 60:40, or 45:55 to 55:45 and having number average molecular weight ranging from 570 Da to 3000 Da.

Embodiment 18. The particle of any one of embodiments 9-15, wherein the anticancer agent is present in an amount ranging from about 1 wt. % to about 99 wt. % by the total weight of the particle.

Embodiment 19. The particles of any one of embodiments 9-15, wherein the anticancer agent has a weight ratio to the polymer ranging from about 1:99 to about 99:1, or from about 5:95 to about 95:5.

Embodiment 20. The particle of embodiment 1, wherein the material does not have significant optical absorption in the visible spectrum region.

Embodiment 21. The particle of embodiment 1, wherein the material has significant optical absorption in the near infrared spectrum region.

Embodiment 22. The particle of embodiment 1, wherein the material has optical absorption in the range of 700-1500 nm.

Embodiment 23. The particle of any one of embodiments 1-20, wherein the material is a tris-aminium dye, a di-imonium dye, or a tetrakis aminium dye.

Embodiment 24. The particle of any one of embodiments 1-20, wherein the material is a zinc iron phosphate pigment.

Embodiment 25. The particle of embodiment 1, further comprising a targeting group on the particle surface selected from the group of tumor targeting folate, antibodies, proteins, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, peptidomimetics, glycopeptides, peptoids, aptamer, and combinations thereof.

Embodiment 26. The particle of embodiment 25, wherein the targeting group is selected from the group of EGFR binding peptides, claudin, HYNIC-(Ser)3-J18, FROP-1, and combinations thereof.

Embodiment 27. The particle of embodiment 1, further comprising a shell to enclose the particle.

Embodiment 28. The particle of embodiment 1, further comprising a hydrophilic polymer on the particle surface selected from the group of polyethylene glycols, hyperbranched polyglycerol, hyaluronic acid, and combinations thereof.

Embodiment 29. The particle of embodiment 1, wherein the exogenous source is a microwave.

Embodiment 30. The particle of embodiment 1, wherein the exogenous source is an electrical field.

Embodiment 31. The particle of embodiment 1, wherein the exogenous source is a magnetic field.

Embodiment 32. The particle of embodiment 1, wherein the exogenous source is a sound wave (ultrasonic).

Embodiment 33. A particle for use in treating a cancer comprising: (a) an anticancer agent selected from the group of cisplatin, docetaxel, carboplatin, doxorubicin, etoposide, paclitaxel, and combinations thereof; (b) a carrier comprising polymer selected from the group of poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA), and combinations thereof (c) an IR absorbing agent selected from the group of a tris-aminium dye, a di-imonium dye, a tetrakis aminium dye, a zinc iron phosphate pigment, and combinations thereof, wherein the particle has a median particle size less than 5 μm, wherein the anticancer agent is encapsulated by the carrier, wherein the anticancer agent and the material in the particle exhibit stability such that the particle is considered passing the Efficacy Determination Protocol; wherein the particle structure is constructed such that it passes the Extractable Cytotoxicity Test; wherein the anticancer agent is released outside the particle when the exogenous source is applied.

Embodiment 34. The particle of embodiment 33, wherein the polymer comprises PLGA having a lactide:glycolide molar ratio from 5:95 to 95:5, 10:90 to 90:10, 15:85 to 85:15, 25:75 to 75:25, 40:60 to 60:40, or 45:55 to 55:45 and having a number average molecular weight ranging from 570 Da to 3000 Da.

Embodiment 35. The particle of embodiment 33, wherein the particle further comprises a targeting group selected from EGFR binding peptides, claudin, HYNIC-(Ser)3-J18, FROP-1, and combinations thereof.

Embodiment 36. The particles of any one of embodiments 31-33, wherein the surface of the particle is modified with a hydrophilic polymer selected from the group of polyethylene glycols, hyperbranched polyglycerol, hyaluronic acid, and combinations thereof.

Embodiment 37. The particle of any one of embodiments 1-34, wherein the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma, glioblastoma multiforme, non-small-cell lung cancer, hepatocellular carcinoma, metastatic colon cancer, multiple myeloma, small-cell lung cancer, melanoma, and combinations thereof.

Embodiment 38. A method for treating a cancer in a patient in need thereof comprising: (1) administering to the patient the particle of embodiment 1, and (2) activating the particle with the exogenous source, wherein the material absorbs the energy from the exogenous source and converts the energy into heat; and wherein the heat causes degradation of the carrier, and then the anticancer agent is released outside the particle.

Embodiment 39. The method of embodiment 38, wherein the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, melanoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma, glioblastoma multiforme, non-small-cell lung cancer, hepatocellular carcinoma, metastatic colon cancer, multiple myeloma, small-cell lung cancer, melanoma, and combinations thereof.

Embodiment 40. A particle heater for use in a remotely-triggered thermal therapy comprising: (a) a material interacting with an exogenous source, (b) a carrier, wherein the material interacting with an exogenous source is encapsulated by the carrier to form a particle, wherein the material in the particle exhibit stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells.

Embodiment 41. The particle heater of embodiment 40, wherein the particle heater further passes the Thermal Cytotoxicity Test.

Embodiment 42. The particle heater of embodiment 40, wherein the particle maintains or alters its integrity after its exposure to the exogenous source.

Embodiment 43. The particle heater of any one of embodiments 40-42, wherein the particles are nanoparticles or microparticles.

Embodiment 44. The particle heater of any one of embodiments 40-43, wherein the particle further comprises a shell to enclose the particle to form a core-shell particle.

Embodiment 45. The particle heater of embodiment 44, wherein the shell comprises a cross-linked inorganic polymer selected from the group of mesoporous silica, organo-modified silicate polymer derived from condensation of organotrisilanol or halotrisilanol, and combinations thereof.

Embodiment 46. The particle heater of embodiment 44, wherein the shell comprises an agent selected from the group of Au, Ag, Cu, iron oxide, and combinations thereof.

Embodiment 47. The particle heater of embodiment 40, wherein the carrier comprises a bio-compatible and/or a biodegradable substance.

Embodiment 48. The particle heater of embodiment 47, wherein the carrier comprises a bio-degradable polymer having labile bonds are selected from the group of an ester bond, an amide bond, an anhydride bond, an acetal bond, a ketal bond, and combinations thereof.

Embodiment 49. The particle heater of any one of embodiments 40-48, wherein the carrier is selected from the group of a polyester, a polyanhydride, a polysaccharide, a polyphosphoester, a poly(ortho ester), a poly(amino acid), a protein, polyurea, and combinations thereof.

Embodiment 50. The particle heater of embodiment 10, wherein the carrier is selected from the group of poly(lactic acid) (PLA), poly(glycolic acid) (PGA), poly-lactic acid-co-glycolic acid (PLGA), poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolactone, poly(hydroxy valerate), PCL, γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(butylene terephthalate), poly(β-hydroxyalkanoate)s, poly(hydroxybutyrate), poly(hydroxybuthyrate-co-hydroxyvalerate), poly (□-lysine), diblock copolymer of poly(sebacic acid) and polyethylene glycol (PSA-PEG), trimethylene carbonate, poly(β-hydroxybutyrate), poly(g-ethyl-L-glutamate), poly(iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

Embodiment 51. The particle heater of embodiment 40, wherein the carrier is selected from the group of lipid, polymer-lipid conjugate, carbohydrate-lipid conjugate, peptide-lipid conjugate, protein-lipid conjugate, and combinations thereof.

Embodiment 52. The particle heater of embodiment 51, wherein the carrier comprises a lipid selected from the group of dipalmitoylphosphatidylcholine (DPPC), 1-palmitoyl-2-hydroxy-sn-glycero-3-phosphocholine (MPPC), 1-myristoyl-2-stearoyl-sn-glycero-3-phosphocholine (MSPC); 1,2-dimyristoyl-sn-glycero-3-phosphocholine (DMPC), 1,2-dimyristoyl-sn-glycero-3-phosphorylglycerol (DMPG); 1,2-distearoyl-sn-glycero-3-phosphoethanolamine (DSPE); 1,2-dioleoyl-sn-glycero-3-phosphocholine (DOPC); 1,2-dioleoyl-sn-glycero-3-phosphoethanolamine (DOPE); 1,2-dipalmitoyl-sn-glycero-3-phospho-(1′-rac-glycerol) (DPPG); 1,2-distearoyl-sn-glycero-3-phosphocholine (DSPC), distearoylphosphoethanolamine conjugated with polyethylene glycol (DSPE-PEG); phosphatidylserine (PS), phosphatidylethanolamine (PE), phosphatidylglycerol (PG), phosphatidylcholine (PC), and combinations thereof.

Embodiment 53. The particle heater of embodiment 51, wherein the carrier comprises the lipid selected from the group of DPPC, MPPC, PEG, DMPC, DMPG, DSPE, DOPC, DOPE, DPPG, DSPC, DSPE-PEG, MSPC, cholesterol, PS, PC, PE, PG, 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

Embodiment 54. The particle heater of embodiment 40, wherein the material does not have significant optical absorption in the visible spectrum region.

Embodiment 55. The particle heater of embodiment 40, wherein the material has significant optical absorption in the near infrared spectrum region.

Embodiment 56. The particle heater of embodiment 40, wherein the material has optical ab-sorption in the range of 750-1100 nm or 400-750 nm.

Embodiment 57. The particle heater of embodiment 40, wherein the material is a tris-aminium dye, a di-imonium dye, a cyanine dye, a squaraine dye, a squarylium dye, iron oxide, gold or a tetrakis aminium dye.

Embodiment 58. The particle heater of embodiment 40, wherein the material is a zinc iron phosphate pigment.

Embodiment 59. The particle heater of any one of embodiments 40-58, further comprising a tumor cell targeting group on the particle surface selected from the group of folate, antibodies, proteins, EGFR binding antibodies, EGFR binding peptides, integrin-binding peptides, Neuropilin-1 (NRP-1)-binding peptides, interleukin 13 receptor α2 (IL-13Rα2)-binding pep-tides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, peptidomimetics, glycopeptides, peptoids, aptamer, and combinations thereof.

Embodiment 60. The particle heater of embodiment 59, wherein the targeting group is selected from the group of an EGFR antibody, an EGFR binding peptide, and combinations thereof.

Embodiment 61. The particle heater of embodiments 60, wherein the targeting group is an EGFR binding antibody selected from the group of cetuximab, panitumumab, and combinations thereof.

Embodiment 62. The particle heater of embodiments 60, wherein the targeting group is an EGFR binding peptide selected from the group of YHWYGYTPQNVI, YRWYGYTPQNVI, L-AE (L amino acids in the sequence-FALGEA), D-AE (D-amino acids in the sequence-FALGEA), and combinations thereof.

Embodiment 63. The particle heater of any one of embodiments 60-62, wherein the targeting group is covalently conjugated to the surface of the particle heater via a disulfide bond.

Embodiment 64. The particle heater of embodiment 40, further comprising a hydrophilic polymer on the particle heater surface selected from the group of polyethylene glycols, hyperbranched polyglycerol, hyaluronic acid, and combinations thereof.

Embodiment 65. The particle heater of embodiment 40, wherein the exogenous source is selected from the group of a microwave, an electrical field, a magnetic field, sound wave (ultrasonic), and combinations thereof.

Embodiment 66. A particle heater for use in a remotely-triggered-thermal treatment of a cancer comprising: (a) a material that interacts with an exogenous source, wherein the material is an IR absorbing agent selected from the group of a tris-aminium dye, a di-imonium dye, a tetrakis aminium dye, a cyanine dye, a squaraine dye, a zinc iron phosphate pigment, and combinations thereof, (b) a carrier comprising a polymer selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof; wherein the particle heater has a median particle size less than 5 μm, wherein the carrier to form a particle encapsulates the material interacting with an exogenous source,

wherein the material in the particle heater exhibit stability such that the particle heater is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to induce localized hyperthermia sufficient to selectively kill cancer cells.

Embodiment 67. The particle heater of embodiment 66, wherein the particle heater surface further comprises a targeting group selected from the group of an EGFR binding antibody including cetuximab, and panitumumab; an EGFR binding peptide selected from the group of YHWYGYTPQNVI, YRWYGYTPQNVI, L-AE (L amino acids in the sequence-FALGEA), D-AE (D-amino acids in the sequence-FALGEA), and combinations thereof.

Embodiment 68. The particles of any one of the embodiments 66-67, wherein the particle heater surface is further modified with a hydrophilic polymer selected from the group of polyethylene glycols, hyperbranched polyglycerol, hyaluronic acid, and combinations thereof.

Embodiment 69. A method for causing remotely-triggered thermal killing of tumor cells at a tumor site in a subject in need thereof comprising: (1) administering an effective amount of the particle heater according to any one of embodiments 40 and 66-68 to the tumor site in the subject, and (2) exposing the particle to an exogenous source that heats the particle heater for a sufficient period of time, wherein the material in the particle exhibits stability such that the particle is considered passing the Efficacy Determination Protocol; wherein the particle is constructed such that it passes the Extractable Cytotoxicity Test; wherein the material absorbs the energy from the exogenous source and converts the energy into heat; and then the heat travels outside the particle to cause a temperature increase in a tissue area surrounding the particle thereby to induce localized hyperthermia at a temperature ranging from about 38.0° C. to about 52.0° C. that is sufficient to selectively kill cancer cells, and wherein the collateral damage to the non-cancer cells is minimized.

Embodiment 70. The method of embodiments 69, wherein the induced hyperthermia is a mild hyperthermia at a temperature ranging from about 38.0° C. to about 41.0° C.

Embodiment 71. The method of embodiments 69, wherein the induced hyperthermia is a moderate hyperthermia at a temperature ranging from about 41.1° C. to about 45.0° C., wherein the hyperthermia does not cause collateral damage to healthy cells.

Embodiment 72. The method of embodiments 69, wherein the induced hyperthermia is a profound hyperthermia at a temperature ranging from about 45.1° C. to about 52.0° C.

Embodiment 73. The method of any one embodiments 69-72, wherein the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, intraocular melanoma, oral cavity and oropharyngeal cancers, gastric cancer, stomach cancer, cervical cancer, kidney cancer, liver cancer, esophageal cancer, testicular cancer, gynecological cancer, thyroid cancer, Kaposi's sarcoma, viral-induced cancer, glioblastoma multiforme, non-small-cell lung cancer, hepatocellular carcinoma, small-cell lung cancer, melanoma, and combinations thereof.

Embodiment 74. The method of embodiment 73, wherein the cancer is breast cancer, lung cancer or glioblastoma multiforme.

Embodiment 75. A synergistic combination therapy for the treatment of cancer comprising: (a) an anticancer agent, and (b) a particle heater having a material interacting with an exogenous source admixed with a carrier, wherein the material absorbs the energy from the exogenous source and converts the energy into heat; and then the heat travels outside the particle to induce localized hyperthermia, wherein the localized hyperthermia and the anticancer agent exhibit synergy in killing cancer cells, and wherein the particle is constructed such that it passes the Extractable Cytotoxicity Test.

Embodiment 76. The synergistic combination therapy of embodiment 75, wherein the localized hyperthermia and the anticancer agent exhibit coefficient of drug interaction (CDI)<1.0.

Embodiment 77. The synergistic combination therapy of embodiment 76, wherein the CDI of the localized hyperthermia and the anticancer agent is about 0.1, about 0.2, about 0.3, about 0.4, about 0.5, about 0.6, about 0.7, about 0.8, about 0.9, or about 1.0.

Embodiment 78. The synergistic combination therapy of embodiment 75, wherein the anti-cancer agent is further encapsulated by the particle heater having the material, and wherein the heat causes the release of the anticancer agent outside of the particle.

Embodiment 79. The synergistic combination therapy of embodiment 75, wherein the anti-cancer agent is in a conventional pharmaceutical dosage.

Embodiment 80. The synergistic combination therapy of embodiment 75, wherein the particle heater further passes the Thermal Cytotoxicity Test.

Embodiment 81. The synergistic combination therapy of embodiment 75, wherein the particle heater further passes the Efficacy Determination Protocol.

Embodiment 82. The synergistic combination therapy of any one of the embodiments 75-81, wherein the exogenous source is selected from the group of an electromagnetic radiation, an electrical field, a microwave, a radio wave, an ultrasound, a magnetic field, and combinations thereof.

Embodiment 83. The synergistic combination therapy of embodiment 75, wherein the particle maintains its integrity and/or alters its structure after its exposure to the exogenous source.

Embodiment 84. The synergistic combination therapy of any one of embodiments 75-83, wherein the particles are nanoparticles or microparticles.

Embodiment 85. The synergistic combination therapy of embodiment 84, wherein the nano-particle has a median particle size ranging from about 1 nm to about 250 nm.

Embodiment 86. The synergistic combination therapy of embodiment 85, wherein the nano-particle has a median particle size ranging from about 10 nm to about 50 nm.

Embodiment 87. The synergistic combination therapy of any one of embodiments 75-86, wherein the particle further comprises a shell to enclose the particle to form a core-shell particle.

Embodiment 88. The synergistic combination therapy of embodiment 87, wherein the shell comprises a cross-linked inorganic polymer selected from the group of mesoporous silica, organo-modified silicate polymer derived from condensation of organotrisilanol or halotrisilanol, and combinations thereof.

Embodiment 89. The synergistic combination therapy of any one of embodiments 87-88, wherein the shell comprises a plasmonic absorber selected from the group of a thin film of noble metals including gold (Au), silver (Ag), copper (Cu), nanoporous gold thin film, and combinations thereof.

Embodiment 90. The synergistic combination therapy of embodiment 75, wherein the particle further comprises a coating formed of polydopamine that is capable of converting exogenous energy to heat.

Embodiment 91. The synergistic combination therapy of embodiment 75, wherein the unencapsulated anticancer agent has a plasma half-life of less than 30 minutes.

Embodiment 92. The synergistic combination therapy of embodiment 75, wherein the anti-cancer agent is a Class II, Class III or Class IV compound according to the Biopharmaceutics Classification System.

Embodiment 93. The synergistic combination therapy of any one of embodiments 75-92, wherein the anticancer agent is selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelali sib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

Embodiment 94. The synergistic combination therapy of any one of embodiments 75-93, wherein the anticancer agent is a PI3K inhibitor selected from the group of wortmannin, temsirolimus, everolimus, buparlisib (BMK-120), 5-(2,6-dimorpholinopyrimidin-4-yl)-4-(trifluoromethyl)pyridin-2-amine), pictilisib, gedatolisib, apitolisib, pilaralisib, copanli sib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide), and combinations thereof.

Embodiment 95. The synergistic combination therapy of any one of embodiments 75-93, wherein the anticancer agent is a proteasome inhibitor selected from the group of bortezomib, ixazomib, marizomib, oprozomib, delanzomib, epoxomicin, disulfiram, lactacystin, beta-hydroxy beta-methylbutyrate, and combinations thereof.

Embodiment 96. The synergistic combination therapy of any one of embodiments 75-93, wherein the anticancer agent is an EGFR inhibitor selected from the group of erlotinib, gefitinib, neratinib, osimertinib, vandetanib, dacomitinib, lapatinib, and combinations thereof.

Embodiment 97. The synergistic combination therapy of any one of the embodiments 75-96, wherein the material has significant absorption of photonic energy in the visible spectrum region having a wavelength range from 400 nm to 750 nm.

Embodiment 98. The synergistic combination therapy of any one of the embodiments 75-96, wherein the material has significant absorption of photonic energy in the near infrared spectrum region having a wavelength range from 750 nm to 1100 nm.

Embodiment 99. The synergistic combination therapy of embodiment 98, wherein the material is selected from the group of a tetrakis aminium dye, a cyanine dye, a squarylium dye, indocyanine green (ICG), new ICG (IR 820), squaraine dye, IR 780 dye, IR 193 dye, Epolight™ 1117 dye, iron oxide, zinc iron phosphate pigment, and combinations thereof.

Embodiment 100. The synergistic combination therapy of embodiment 75, wherein the carrier comprises a biocompatible and/or a biodegradable substance.

Embodiment 101. The synergistic combination therapy of any one of the embodiments 100, wherein the biocompatible substance and/or biodegradable substance is selected from the group of a lipid, an inorganic polymer, an organic polymer, and combinations thereof.

Embodiment 102. The synergistic combination therapy of embodiment 75, wherein the carrier comprises a polymer having labile bonds susceptible to hydrolysis.

Embodiment 103. The synergistic combination therapy of any one of embodiments 100-102, wherein the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polylysine; and combinations thereof.

Embodiment 104. The synergistic combination therapy of any one of embodiments 100-102, wherein the carrier comprises a cross-linked biocompatible and biodegradable polymer.

Embodiment 105. The synergistic combination therapy of embodiment 104, wherein the cross-linked biocompatible polymer comprises a cross-linked polysaccharide.

Embodiment 106. The synergistic combination therapy of embodiment 105, wherein the polysaccharide is selected from chitosan, hyaluronic acid, alginate, alginic acid, starch, carrageenan, and combinations thereof.

Embodiment 107. The synergistic combination therapy of embodiment 101, wherein the carrier comprises an inorganic polymer.

Embodiment 108. The synergistic combination therapy of embodiment 107, wherein the in-organic material is selected from the group of mesoporous silica, organo-modified silicate polymer derived from condensation of organotrisilanol or halotrisilanol, and combinations thereof.

Embodiment 109. The synergistic combination therapy of embodiment 101, wherein the carrier is a lipid.

Embodiment 110. The synergistic combination therapy of embodiment 109, wherein the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

Embodiment 111. The synergistic combination therapy of embodiment 109, wherein the lipid comprises a thermoresponsive lipid/polymer hybrid.

Embodiment 112. The synergistic combination therapy of embodiment 111, wherein the thermoresponsive lipid/polymer hybrid is selected from the group of triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite.

Embodiment 113. The synergistic combination therapy of any one of embodiments 111-112, wherein the particle heater further has a thin film of noble metal on the particle surface, wherein the noble metal is selected from the group of gold, silver, and copper.

Embodiment 114. The synergistic combination therapy of any one of embodiments 111-112, wherein the particle heater further comprises iron oxide.

Embodiment 115. The synergistic combination therapy of any one of embodiments 75-114, wherein the particle heater further comprises a cancer targeting group on the particle surface selected from the group of folate, antibodies, proteins, EGFR binding antibodies, EGFR binding peptides, integrin-binding peptides, Neuropilin-1 (NRP-1)-binding peptides, interleukin 13 receptor α2 (IL-13Rα2)-binding peptides, vascular endothelial growth factor receptor 3 (VEGFR-3)-binding peptides, platelet-derived growth factor receptor β (PDGFRβ)-binding peptides, protein tyrosine phosphatase receptor type J (PTPRJ)-binding peptides, VAV3 binding peptides, p32-protein binding peptide, peptidomimetics, glycopeptides, peptoids, aptamer, and combinations thereof.

Embodiment 116. The synergistic combination therapy of embodiment 115, wherein the targeting group is selected from the group of an EGFR antibody, an EGFR binding peptide, p32-protein binding peptide, and combinations thereof.

Embodiment 117. The synergistic combination therapy of embodiments 116, wherein the cancer targeting group is an EGFR binding antibody selected from the group of cetuximab, and panitumumab.

Embodiment 118. The synergistic combination therapy of embodiments 116, wherein the cancer targeting group is an EGFR binding peptide selected from the group of YHWYGYTPQNVI, YRWYGYTPQNVI, L-AE (L amino acids in the sequence-FALGEA), D-AE (D-amino acids in the sequence-FALGEA), and combinations thereof.

Embodiment 119. The synergistic combination therapy of any one of embodiments 116-118, wherein the cancer targeting group is covalently conjugated to the surface of the particle heater via a disulfide bond, or via NHS-EDC chemistry.

Embodiment 120. The synergistic combination therapy of embodiment 75, further comprising a hydrophilic polymer on the particle heater surface selected from the group of polyethylene glycols, hyperbranched polyglycerol, hyaluronic acid, and combinations thereof.

Embodiment 121. The synergistic combination therapy of embodiment 75, wherein the anti-cancer agent is selected from the group of gefitinib, gefitinib, erlotinib, lapatinib, neratinib, osimertinib, vandetanib, dacomitinib, abemaciclib, trastuzumab, cetuximab, panitumumab, and combinations thereof; wherein the material is an IR absorbing agent selected from the group of a indocyanine green (ICG), new ICG (IR 820), IR 193 dye, Epolight™ 1117, Epolight™ 1175, and combinations thereof, (c) a carrier comprising a polymer selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof; and wherein the particle heater has a median particle size less than 200 nm.

Embodiment 122. A composition for use in a remotely-triggered synergistic combination therapy for treatment of a cancer comprising (a) a particle heater having a material interacting with an exogenous source and a carrier; and (b) a pharmaceutical dosage having an anticancer agent.

Embodiment 123. The composition of embodiment 122, wherein the particle heater and the pharmaceutical dosage forms a unitary dosage.

Embodiment 124. The composition of embodiment 122, wherein the particle heater and the pharmaceutical dosage are two discrete preparations.

Embodiment 125. The composition of any one of embodiments 122-124, wherein the pharmaceutical dosage is selected from the group of a capsule, a tablet, a buccal tablet, an oral disintegrating tablet, a liquid formulation, a dispersion, an injection preparation, powder for injection, and suppository.

Embodiment 126. The composition of any one of embodiments 122-125, wherein the particle heaters are nanoparticles or microparticles.

Embodiment 127. The composition of embodiment 122, wherein the particle heater is further combined with a pharmaceutically acceptable excipient to form a particle heater preparation.

Embodiment 128. The composition of embodiment 127, wherein the particle heater preparation is selected from the group of a capsule, a tablet, a buccal tablet, an oral disintegrating tablet, a liquid formulation, a dispersion, an injection preparation, powder for injection, and suppository.

Embodiment 129. A method for causing remotely-triggered synergistic combination therapy for the treatment of cancer in a subject comprising: (1) administering a therapeutically effective amount of the particle heaters according to any one of embodiments 1 and 48-54 to the tumor site in the subject in need thereof and allowing the synergistic combination therapy to associate with cancer cells, and (2) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the anticancer agent exhibit synergy in killing cancer cells, and wherein the particle is constructed such that it passes the Extractable Cytotoxicity Test.

Embodiment 130. The method of embodiment 129, wherein the anticancer agent is further encapsulated by the particle heater having the material, and wherein the heat causes the re-lease of the anticancer agent outside of the particle.

Embodiment 131. The method of embodiment 129, wherein the particle heater and the anti-cancer agent are administered to the patient simultaneously.

Embodiment 132. The method of embodiment 129, wherein the particle heater and the anti-cancer agent are administered to the patient sequentially.

Embodiment 133. The method of embodiment 129, wherein the anticancer agent is administered before the administering of the particle heater.

Embodiment 134. The method of embodiment 129, wherein the particle heater is administered before the administering the anticancer agent.

Embodiment 135. The method of embodiment 129, further comprise performs radiation therapy or surgery.

Embodiment 136. The method of embodiments 129, wherein the induced hyperthermia is a mild hyperthermia at a temperature ranging from about 38.0° C. to about 41.0° C.

Embodiment 137. The method of embodiments 129, wherein the induced hyperthermia is a moderate hyperthermia at a temperature ranging from about 41.1° C. to about 45.0° C., wherein the hyperthermia does not cause collateral damage to healthy cells.

Embodiment 138. The method of embodiments 129, wherein the induced hyperthermia is a profound hyperthermia at a temperature ranging from about 45.1° C. to about 52.0° C.

Embodiment 139. The method of any one embodiments 129-138, wherein the cancer is selected from the group of bladder cancer, head and neck cancer, pancreatic ductal adenocarcinoma (PDA), pancreatic cancer, colon carcinoma, mammary carcinoma, breast cancer, fibrosarcoma, mesothelioma, renal cell carcinoma, lung carcinoma, thymoma, prostate cancer, colorectal cancer, ovarian cancer, brain cancer, squamous cell cancer, skin cancer, eye cancer, retinoblastoma, intraocular melanoma, oral cavity and oropharyngeal cancers, stomach cancer, cervical cancer, kidney cancer, liver cancer, esophageal cancer, testicular cancer, gynecological cancer, thyroid cancer, Kaposi's sarcoma, glioblastoma multiforme, non-small-cell lung cancer, hepatocellular carcinoma, multiple myeloma, small-cell lung cancer, melanoma, and combinations thereof.

Embodiment 140. The method of embodiment 139, wherein the cancer is breast cancer, lung cancer or glioblastoma multiforme.

Embodiment 141. A method of treating a cancer with synergistic combination therapy in a subject comprising the steps of sensitizing the cancer by administering to the subject in need thereof a treatment that will (i) induce apoptosis or autophagy in tumor cells, (ii) induce ferroptosis in tumor cells, (iii) induce necrotic cell death in tumor, (iv) modify the tumor environment, (v) stimulate tumor-infiltrating immune cells, or (vi) a combination of two or more thereof.

Embodiment 142. The method of embodiment 141, wherein the treatment is a combination therapy comprising a particle heater and an anticancer agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat travels outside the particle to induce localized hyperthermia sufficient to selectively kill cancer cells.

Embodiment 143. The method of embodiment 142, wherein the anticancer agent is encapsulated in the particle heater and the heat causes the particle to alter its structure to release of the anticancer agent.

Embodiment 144. The method of embodiment 142, wherein the anticancer agent is not encapsulated in the particle heater.

Embodiment 145. The method of embodiment 142, wherein the anticancer agent is present in a separate pharmaceutical composition from the particle heater.

Embodiment 146. The method of embodiment 142, wherein the particle heater is administered before the administration of the anticancer agent.

Embodiment 147. The method of embodiment 142, wherein the particle heater is administered after the administration of the anticancer agent.

Embodiment 148. The method of embodiment 142, wherein the particle heater is administered concurrently with the administration of the anticancer agent.

Embodiment 149. The method of embodiment 142, the method further comprises the step of activating the particle heater remotely by an exogenous source, wherein the exogenous source is selected from the group of an electromagnetic radiation, an electrical field, a microwave, a radio wave, an ultrasound, a magnetic field, and combinations thereof.

Embodiment 150. The method of embodiment 142, wherein the method further comprises the step of activating the particle heater remotely with an exogenous source selected from the group of an electromagnetic radiation, an electrical field, a microwave, a radio wave, an ultra-sound, a magnetic field, and combinations thereof.

Embodiment 151. The method of embodiment 142, wherein the particle heater is used to guide the imaging-based surgical debulking of the tumor followed by remotely triggering the particles for the destruction of cancer cells along the surgical margins.

Embodiment 152. The method of embodiment 142, wherein sensitizing the tumor comprises administering to the subject a treatment that will induce apoptosis, autophagy, ferroptosis, or Necrotic cell death in tumor cells.

Embodiment 153. The method of embodiment 152, wherein the treatment is selected from the group of thermotherapy, radiation therapy, surgery, chemotherapy, immunotherapy, photodynamic therapy, or a combination thereof.

Embodiment 154. The method of embodiment 152, wherein the treatment is thermotherapy.

Embodiment 155. The method of embodiment 152, wherein the treatment is thermotherapy and chemotherapy.

Embodiment 156. The method of embodiment 152, wherein the treatment is photodynamic therapy.

Embodiment 157. A particle heater comprising a carrier admixed with a material that inter-acts with an exogenous source; wherein the material absorbs and converts the energy from the exogenous source into heat, and the heat travels outside the particle heater to induce localized hyperthermia at a temperature sufficient to selectively kill unwanted cells, and further wherein the particle heater structure is constructed such that it passes the Extractable Cytotoxicity Test.

Embodiment 158. The particle heater of embodiment 157, wherein particle heater further passes the Efficacy Determination Protocol.

Embodiment 159. The particle heater of any one of embodiments 157-158, wherein particle heater further passes the Thermal Cytotoxicity Test.

Embodiment 160. The particle heater of any one of embodiments 157-159, the material exhibits at least 20% efficiency of conversion of the energy from the exogenous source to heat (energy-to-heat conversion efficiency).

Embodiment 161. The particle heater of any one of embodiments 157-169, the material exhibits at least 20% photothermal conversion efficiency.

Embodiment 162. The particle heater of any one of the embodiments 157-158, wherein the exogenous source is selected from the group of an electromagnetic radiation, an electrical field, a microwave, a radio wave, an ultrasound, a magnetic field, and combinations thereof.

Embodiment 163. The particle heater of any one of the embodiments 157-159, wherein the particle heater has a median particle size ranging from about 1 nm to about 250 nm.

Embodiment 164. The particle heater any one of the embodiments 160 and 161, wherein the particle heater has a median particle size ranging from about 1 nm to about 50 nm.

Embodiment 165. The particle heater of any one of the embodiments 157-162, wherein the particle heater maintains integrity or the particle structure is altered after interacting with the exogenous source.

Embodiment 166. The particle heater of any one of the embodiments 157-165, wherein the particle heater has a core-shell structure.

Embodiment 167. The particle heater of embodiment 166, wherein the core comprises a plasmonic absorber or iron oxide nanoparticles.

Embodiment 168. The particle heater of embodiment 166, wherein the shell comprises a plasmonic absorber or iron oxide.

Embodiment 169. The particle heater of any one of embodiments 167-168, wherein the plasmonic absorber comprises plasmonic nanomaterials of noble metal including gold (Au) nanostructure, silver (Ag) nanoparticle, and copper (Cu) nanoparticle having a plasmonic resonance at a NIR wavelength.

Embodiment 170. The particle heater of embodiment 166 the shell comprises an agent selected from the group of gold nanostructures, silver nanoparticles, iron oxide film, iron oxide nanoparticle, and combinations thereof.

Embodiment 171. The particle heater of any one of embodiments 166-170, wherein the shell comprise an agent selected from the group of inorganic polymers, silicates, organosilicate, organo-modified silicone polymers derived from condensation of organotrisilanol or halotrisilanol, cross-linked organic polymers, and combinations thereof.

Embodiment 172. The particle heater of any one of the embodiments 157-171, wherein the material has significant absorption of photonic energy in the near infrared spectrum region having a wavelength range from 750 nm to 1100 nm.

Embodiment 173. The particle heater of any one of the embodiments 157-171, wherein the material interacting with the exogenous source has significant absorption of photonic energy in the visible range.

Embodiment 174. The particle heater of embodiment 173, wherein the material absorbs light at a wavelength selected from the group of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

Embodiment 175. The particle heater of any one of the embodiments 157-171, wherein the material is selected from the group of a tetrakis aminium dye, a cyanine dye, a squaraine dye, a squarylium dye, iron oxide, a plasmonic absorber, a zinc iron phosphate pigment, and combinations thereof.

Embodiment 176. The particle heater of any one of the embodiments 157-175, wherein the carrier is selected from the group of a lipid, an inorganic agent, an organic polymer, and combinations thereof.

Embodiment 177. The particle heater of embodiment 176, wherein the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine, and combinations thereof.

Embodiment 178. The particle heater of any one of the embodiments 176 and 177, wherein the carrier is selected from the group of PLA, PGA, PLGA, PCL, PLL, PLGA-PEG, a poly-mer blend containing PLGA 75:25 and PLGA-PEG 75:25 with lactide:glycolide monomer ratio of 75:25, and combinations thereof.

Embodiment 179. The particle heater of embodiment 157, wherein the carrier comprises a crosslinked biocompatible or biodegradable polymer.

Embodiment 180. The particle heater of embodiment 179, wherein the crosslinked biocompatible polymer comprises a crosslinked polysaccharide.

Embodiment 181. The particle heater of embodiment 180, wherein the polysaccharide is selected from hyaluronic acid, alginate, alginic acid, starch, carrageenan, and combinations thereof.

Embodiment 182. The particle heater of embodiment 157, wherein the carrier comprises an inorganic polymer.

Embodiment 183. The particle heater of embodiment 182, wherein the inorganic polymer is selected from the group of mesoporous silica, organo-modified silicate polymer derived from condensation of organotrisilanol or halotrisilanol, and combinations thereof.

Embodiment 184. The particle heater of embodiment 176, wherein the carrier is a lipid.

Embodiment 185. The particle heater of embodiment 184, wherein the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

Embodiment 186. The particle heater of embodiment 184, wherein the lipid comprises a thermoresponsive lipid/polymer hybrid.

Embodiment 187. The particle heater of embodiment 186, wherein the thermoresponsive lipid/polymer hybrid is selected from the group of triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

Embodiment 188. The particle heater of embodiment 157, wherein the particle further comprises an active agent.

Embodiment 189. The particle heater of embodiment 188, wherein the active agent is selected from the group of agents capable of generating reactive oxygen species, therapeutic drugs, antimicrobial agent, anti-cancer agent, anti-scarring agent, anti-inflammatory agent, metallo-protease inhibitors, treatment sensitizing the unwanted cells to remotely triggered thermal therapy, and combinations thereof.

Embodiment 190. A method for inducing localized hyperthermia at a tissue site in a subject comprising: administering an effective amount of the particle heater of the embodiment 175 to the tissue site in the subject; exposing the particle to an exogenous source that heats the particle heater for a sufficient period of time to induce localized hyperthermia at a temperature ranging from about 38.0° C. to about 52.0° C.

Embodiment 191. The method of embodiment 190, wherein the exogenous source is selected from an electromagnetic radiation, an electrical field, a microwave, a radio wave, an ultra-sound, a magnetic field, or combinations thereof.

Embodiment 192. The method of any one of the embodiments 190 and 191, wherein the exogenous source comprises a LED light or a laser light.

Embodiment 193. The method of embodiment 192, wherein the laser light is a pulsed laser light.

Embodiment 194. The method of embodiment 192, wherein the exogenous source comprises a LED light.

Embodiment 195. The method of embodiment 193, wherein the laser pulse duration is in a range from milliseconds to femtoseconds, and the laser has an oscillation wavelength at 805 nm, 808 nm, or 1064 nm.

Embodiment 196. The method of embodiment 192, wherein the particle heater absorbs the visible light having a wavelength ranging from 400 nm to 750 nm.

Embodiment 197. The method of embodiment 190, wherein the particle heater absorbs the laser light having a wavelength ranging from 750 nm to 1400 nm.

Embodiment 198. The method of any one of embodiments 190-197, wherein the material is a tetrakis aminium dye.

Embodiment 199. The method of any one of embodiments 190-197, wherein the material is indocyanine green.

Embodiment 200. The method of any one of embodiments 190-197, wherein the material is a squaraine dye.

Embodiment 201. The method of any one of embodiments 190-197, wherein the material is a squarylium dye.

Embodiment 202. The method of any one of embodiments 190-197, wherein the material is iron oxide.

Embodiment 203. The method of any one of embodiments 190-197, wherein the material is a plasmonic absorber.

Embodiment 204. The method of embodiment 203, wherein the material is a plasmonic absorber is selected from the group of gold nanostructures including gold nanorod, gold nanocage, gold nanosphere, gold thin film, silver nanoparticle, and combinations thereof.

Embodiment 205. The method of any one of the embodiments 190-204, the method further comprising heating a surrounding area in proximity to the particle heater by transferring heat from the particle heater to the surrounding area to induce localized hyperthermia.

Embodiment 206. The method of any one of the embodiments 190-204, wherein the induced hyperthermia is mild hyperthermia at a temperature ranging from about 38.0° C. to about 41.0° C.

Embodiment 207. The method of any one of the embodiments 190-204, wherein the induced hyperthermia is moderate hyperthermia at a temperature ranging from about 41.1° C. to about 45.0° C., wherein the hyperthermia does not cause collateral damage to healthy cells.

Embodiment 208. The method of any one of the embodiments 190-204, wherein the induced hyperthermia is profound hyperthermia at a temperature ranging from about 45.1° C. to about 52.0° C.

Embodiment 209. A particle comprising: (a) an antimicrobial agent, (b) a carrier, (c) a material that interacts with an exogenous source, wherein the antimicrobial agent is encapsulated by the carrier, wherein the antimicrobial agent and the material in the particle exhibit stability such that the particle is considered passing the Efficacy Determination Protocol; wherein the particle structure is constructed such that it passes the Extractable Cytotoxicity Test; and wherein the antimicrobial agent is released outside the particle when the exogenous source is applied.

Embodiment 210. The particle of embodiment 209, further comprising a shell enclosing the particle to form a core-shell particle.

Embodiment 211. The particle of embodiment 209, wherein the antimicrobial agent is an in-organic compound or an organic compound.

Embodiment 212. The particle of embodiment 209, wherein the antimicrobial agent is an in-organic compound selected from the group of silver particles, gold particles, gallium particles, zinc oxide particles, copper oxide particles, and combinations thereof.

Embodiment 213. The particle of embodiment 209, wherein the antimicrobial agent is an organic compound selected from the group of an organic acid, a phenolic compound, a phyto-antibiotic, an amino acid, a quaternary ammonium compound, a surfactant, an antibiotic, and combinations thereof.

Embodiment 214. The particle of embodiment 209, wherein the antimicrobial agent is an antibiotic selected from the group of ampicillin, sulbactam, cefotaxime, telithromycin, temafloxacin, trovafloxacin, praziquantel, amikacin, ciprofloxacin, vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

Embodiment 215. The particle of embodiment 209, wherein the antimicrobial agent is chemically conjugated to the carrier via a heat-labile linker.

Embodiment 216. The particle of embodiment 209, wherein the heat-labile linker is selected from the group of substituted and unsubstituted carbonates, substituted and unsubstituted carbamates, substituted and unsubstituted esters, substituted and unsubstituted lactams, substituted and unsubstituted lactones, substituted and unsubstituted amides, substituted and unsubstituted imides, substituted and unsubstituted oximes, substituted and unsubstituted sulfonates, substituted and unsubstituted phosphonates, and combinations thereof.

Embodiment 217. The particle of embodiments 209-216, wherein the carrier comprises a polymer with heat-labile moieties, or a polymer having labile bonds susceptible hydrolysis.

Embodiment 218. The particle of embodiment 217, wherein the labile bonds are selected from the group of an ester bond, an amide bond, an anhydride bond, an acetal bond, a ketal bond, and combinations thereof.

Embodiment 219. The particle of embodiment 217, wherein the polymer is selected from the group of a polyester, a polyanhydride, a polysaccharide, a polyphosphoester, a poly(ortho ester), a poly(amino acid), a protein, and combinations thereof.

Embodiment 220. The particle of embodiment 217, wherein the polymer is selected from the group of a polyester including poly(lactic acid) (PLA), poly(glycolic acid) (PGA), poly(lactide-co-glycolide) (PLGA), poly(lactic acid)-polyethylene glycol-poly(lactic acid) (PLA-PEG-PLA), poly (L-co-D, L lactic acid) 70:30 (PLDLA), poly-L-lactic acid-co-glycolic acid, poly-D,L-lactic acid-co-glycol acid, poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (□-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

Embodiment 221. The particle of embodiment 300, wherein the polyester comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45 and having an average molecular weight ranging from 2000 Da to 10500 Da.

Embodiment 222. The particle of embodiments 219-221, wherein the antimicrobial agent is present in an amount ranging from about 1 wt. % to about 95 wt. % by the total weight of the particle.

Embodiment 223. The particles of embodiments 219-221, wherein the antimicrobial agent has a weight ratio to the polymer ranging from about 1:99 to about 99:1, or from about 5:95 to about 95:5.

Embodiment 224. The particle of embodiments 209-223, wherein the particle further comprises a bacteria targeting group on the particle surface.

Embodiment 225. The particle of embodiment 224, wherein the bacteria targeting group is selected from the group of an antibody targeting a surface antigen of the bacteria, a cationic antimicrobial peptide, cell penetrating peptides including apidaecin, tat, buforin, magainin, and combinations thereof.

Embodiment 226. The particle of embodiments 209-225, wherein the particle is amorphous or partially amorphous or partially crystalline.

Embodiment 227. The particle of embodiment 209, wherein the exogenous source is selected from the group of electromagnetic radiation, microwaves, an electric field, a magnetic field, radiowaves, and an ultrasound.

Embodiment 228. The particle of embodiment 209, wherein the exogenous source is electro-magnetic radiation.

Embodiment 229. The particle of embodiment 209, wherein the material does not have significant optical absorption in a visible spectrum.

Embodiment 230. The particle of embodiment 209, wherein the material has significant optical absorption in the range of 700-1500 nm.

Embodiment 231. The particle of embodiment 230, wherein the material has significant optical absorption in the range of 750-1400 nm.

Embodiment 232. The particle of embodiments 209-231, wherein the material is a tri-aminium dye, a diimonium dye, or a tetrakis aminium dye.

Embodiment 233. The particle of embodiments 209-231, wherein the material is a zinc iron phosphate pigment.

Embodiment 234. The particle of embodiment 209, wherein the exogenous source is laser pulse radiation at a determined thermal relaxation time.

Embodiment 235. The particle of embodiment 234, wherein the thermal relaxation time is selected from the group of picoseconds, nanoseconds, microseconds, and milliseconds.

Embodiment 236. A method for treating a localized antimicrobial infection comprising the steps: (1) administering to a patient infected with bacteria one or more particles comprising an antimicrobial agent, a carrier, and a material interacting with an exogenous source, and (2) activating the particles with the exogenous source, wherein the material absorbs energy from the exogenous source and converts the energy into heat; and wherein the heat causes degradation of the carrier, and then the antimicrobial agent is released outside the particle.

Embodiment 237. The method of embodiment 236, wherein the particle further comprises a shell enclosing the particle to form a core-shell particle.

Embodiment 238. The method of embodiment 236, wherein the antimicrobial agent is an in-organic compound or an organic compound.

Embodiment 239. The method of embodiment 236, wherein the antimicrobial agent is an in-organic compound selected from the group of silver particles, gold particles, gallium particles, zinc oxide particles, copper oxide particles, and combinations thereof.

Embodiment 240. The method of embodiment 236, wherein the antimicrobial agent is an organic compound selected from the group of an organic acid, a phenolic compound, phytoantibiotics, amino acids, quaternary ammonium compounds, a detergent, antibiotics, and combinations thereof.

Embodiment 241. The method of embodiment 236, wherein the antimicrobial agent is an antibiotic selected from the group of ampicillin, sulbactam, cefotaxime, telithromycin, temafloxacin, trovafloxacin, praziquantel, amikacin, ciprofloxacin, or vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

Embodiment 242. The method of embodiment 236, wherein the particle further comprises a bacteria targeting group on the particle surface.

Embodiment 243. The method of embodiment 242, wherein the bacteria targeting group is selected from the group of an antibody targeting the surface antigen of the bacteria, a cationic antimicrobial peptide, cell penetrating peptides including apidaecin, tat, buforin, magainin, and combinations thereof.

Embodiment 244. The method of embodiments 242-243, wherein the carrier comprises a polymer with heat-labile moieties, or a polymer having labile bonds susceptible to hydrolysis.

Embodiment 245. The method of embodiment 244, wherein the labile bonds are selected from the group of an ester bond, an amide bond, an anhydride bond, an acetal bond, a ketal bond, and combinations thereof.

Embodiment 246. The method of embodiment 244, wherein the polymer is selected from the group of a polyester, a polyanhydride, a polyphosphoester, a poly(ortho ester), a poly(amino acid), a polysaccharide, a protein, and combinations thereof.

Embodiment 247. The method of embodiment 244, wherein the polymer is selected from the group of a polyester including poly(lactic acid) (PLA), poly(glycolic acid) (PGA), poly(lactide-co-glycolide) (PLGA), poly(lactic acid)-polyethylene glycol-poly(lactic acid) (PLA-PEG-PLA), poly (L-co-D,L lactic acid) 70:30 (PLDLA), poly-L-lactic acid-co-glycolic acid, poly-D,L-lactic acid-co-glycol acid, poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (□-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

Embodiment 248. The method of embodiment 247, wherein the polyester comprises a PLGA having a lactide:glycolide molar ratio from 5:95 to 95:5, 10:90 to 90:10, 15:85 to 85:15, 25:75 to 75:25, 40:60 to 60:40, or 45:55 to 55:45 and has a number average molecular weight ranging from 2000 Da to 10500 Da.

Embodiment 249. The method of embodiments 246-248, wherein the antimicrobial agent is present in an amount ranging from about 1 wt. % to about 95 wt. % by the total weight of the particle.

Embodiment 250. The method of embodiments 246-248, wherein the antimicrobial agent has a weight ratio to the polymer ranging from about 1:99 to about 99:1, or from about 5:95 to about 95:5.

Embodiment 251. The method of embodiment 236, wherein the exogenous source is a laser light.

Embodiment 252. The method of embodiment 251, wherein the laser light is a pulsed laser light.

Embodiment 253. The method of embodiment 252, wherein the laser has a pulse duration less than the thermal relaxation time of the particle.

Embodiment 254. The method of embodiment 253, wherein the laser pulse duration is selected from the group of picoseconds, nanoseconds, microseconds, and milliseconds, and the laser has an oscillation wavelength at 1064 nm.

Embodiment 255. The method of embodiment 236, wherein the material absorbs light having a wavelength ranging from 700 nm to 1500 nm.

Embodiment 256. The method of embodiment 236, wherein the material is a tris-aminium dye, a di-imonium dye, or a tetrakis aminium dye.

Embodiment 257. The method of embodiment 236, wherein the particle comprises a zinc iron phosphate pigment.

Embodiment 258. The method of embodiment 236, wherein a temperature inside the particle is higher than 50° C. when the exogenous source is applied.

Embodiment 259. The method of embodiment 258, wherein the temperature inside the particle is selected from the group of about 55° C., about 60° C., about 70° C., about 75° C., about 80° C., about 85° C., about 90° C., about 95° C., about 100° C., about 110° C., about 120° C., about 130° C., about 140° C., about 150° C., about 160° C., about 170° C., about 180° C., about 190° C., about 200° C., about 210° C., about 220° C., about 230° C., about 240° C., about 250° C., about 260° C., about 270° C., about 280° C., about 290° C., and about 300° C.

Embodiment 260. The method of embodiment 236, wherein the bacteria are multidrug resistant bacteria.

Embodiment 261. The method of embodiment 260, wherein the multidrug resistant bacteria comprise Gram positive bacteria.

Embodiment 262. The method of embodiment 260, wherein the multidrug resistant bacteria comprise Gram negative bacteria.

Embodiment 263. The method of embodiment 260, wherein the multidrug resistant bacteria comprise both Gram positive and Gram negative bacteria.

Embodiment 264. The method of embodiments 260-263, wherein the multidrug resistant bacteria comprise one or more species selected from the group of E. coli, K. pneumonia, M. tuberculosis, Streptococcus aureus, P. aeruginosa, Streptococcus epidermidis, and Streptococcus haemolyticus.

Embodiment 265. A synergistic combination therapy for treating microbial infection comprising: (a) an antimicrobial agent, and (b) a particle heater having a material interacting with an exogenous source admixed with a carrier, wherein the material absorbs the energy from the exogenous source and converts the energy into heat, and then the heat travels outside the particle to induce localized hyperthermia, wherein the hyperthermia and the antimicrobial agent exhibit synergy in killing microbes, and wherein the particle is constructed such that it passes the Extractable Cytotoxicity Test.

Embodiment 266. The synergistic combination therapy of embodiment 265, wherein the localized hyperthermia and the antimicrobial agent exhibit coefficient of drug interaction (CDI)<1.0.

Embodiment 267. The synergistic combination therapy of embodiment 266, wherein the CDI of the localized hyperthermia and the antimicrobial agent is about 0.1, about 0.2, about 0.3, about 0.4, about 0.5, about 0.6, about 0.7, about 0.8, about 0.9, or about 1.0.

Embodiment 268. The synergistic combination therapy of embodiment 266, wherein the carrier encapsulates the material and the antimicrobial agent to form a single particle heater.

Embodiment 269. The synergistic combination therapy of embodiment 268, wherein the heat causes the release of the antimicrobial agent outside of the particle heater.

Embodiment 270. The synergistic combination therapy of embodiment 264, wherein the antimicrobial agent is a conventional pharmaceutical dosage.

Embodiment 271. The synergistic combination therapy of embodiment 264, wherein the particle heater further passes the Efficacy Determination Protocol.

Embodiment 272. The synergistic combination therapy of embodiment 264, wherein the particle heater further passes the Thermal Cytotoxicity Test.

Embodiment 273. The particle heater of any one of the embodiments 264-272, wherein the exogenous source is selected from the group of an electromagnetic radiation, an electrical field, a microwave, a radio wave, an ultrasound, a magnetic field, and combinations thereof.

Embodiment 274. The synergistic combination therapy of embodiment 265, wherein the particle heater maintains integrity after its exposure to the exogenous source.

Embodiment 275. The synergistic combination therapy of embodiment 268, wherein the particle alters its structure to release the antimicrobial agent after exposure to the exogenous source.

Embodiment 276. The synergistic combination therapy of any one of embodiments 265-274, wherein the particle further comprises a shell to enclose the particle to form a core-shell particle.

Embodiment 277. The synergistic combination therapy of embodiment 275, wherein the shell comprises a crosslinked inorganic polymer selected from the group of mesoporous silica, organo-modified silicate polymer derived from condensation of organotrisilanol or halotrisilanol, and combinations thereof.

Embodiment 278. The synergistic combination therapy of any one of embodiments 275-276, wherein the shell comprises a plasmonic absorber selected from the group of a thin film of noble metals including gold (Au), silver (Ag), copper (Cu), nanoporous gold thin film, and combinations thereof.

Embodiment 279. The synergistic combination therapy of embodiment 265, wherein the particle further comprises a coating formed of polydopamine that is capable of converting exogenous energy into heat.

Embodiment 280. The synergistic combination therapy of embodiment 265, wherein the antimicrobial agent is an inorganic compound or an organic compound.

Embodiment 281. The synergistic combination therapy of embodiment 280, wherein the antimicrobial agent is an inorganic compound selected from the group of silver particles, gold particles, gallium particles, zinc oxide particles, copper oxide particles, and combinations thereof.

Embodiment 282. The synergistic combination therapy of embodiment 280, wherein the antimicrobial agent is an organic compound selected from the group of an organic acid, a phenolic compound, a phyto-antibiotic, an amino acid, a quaternary ammonium compound, a surfactant, an antibiotic, and combinations thereof.

Embodiment 283. The synergistic combination therapy of embodiment 265, wherein the antimicrobial agent is an antibiotic selected from the group of ampicillin, sulbactam, cefotaxime, telithromycin, temafloxacin, trovafloxacin, praziquantel, amikacin, ciprofloxacin, vancomycin, gentamicin, tobramycin, penicillin, streptomycin, amoxicillin, doxycycline, minocycline, tetracycline, eravacycline, cephalexin, ciprofloxacin, clindamycin, lincomycin, clarithromycin, erythromycin, metronidazole, azithromycin, sulfamethoxazole, trimethoprim, levofloxacin, moxifloxacin, cefuroxime, ceftriaxone, cefdinir, sulfasalazine, sulfisoxazole, sulfamethoxazole-trimethoprim, dalbavancin, oritavancin, telavancin, ertapenem, doripenem, meropenem, imipenem, cilastatin, bacitracin, neomycin, polymyxin B, amphotericin, and combinations thereof.

Embodiment 284. The synergistic combination therapy of any one of embodiments 265-280, wherein the antimicrobial agent is chemically conjugated to the particle surface via a heat-labile linker.

Embodiment 285. The synergistic combination therapy of embodiment 281, wherein the heat-labile linker is selected from the group of substituted and unsubstituted carbonates, substituted and unsubstituted carbamates, substituted and unsubstituted esters, substituted and unsubstituted lactams, substituted and unsubstituted lactones, substituted and unsubstituted amides, substituted and unsubstituted imides, substituted and unsubstituted oximes, substituted and unsubstituted sulfonates, substituted and unsubstituted phosphonates, and combinations thereof.

Embodiment 286. The synergistic combination therapy of any one of the embodiments 265-281, wherein the antimicrobial agent is encapsulated within the particle.

Embodiment 287. The synergistic combination therapy of any one of the embodiments 265-286, wherein the material has significant absorption of photonic energy in the near infrared spectral region having a wavelength range from 750 nm to 1100 nm.

Embodiment 288. The synergistic combination therapy of embodiment 287, wherein the material is selected from the group of a tetrakis aminium dye, a cyanine dye, a squarylium dye, indocyanine green (ICG), new ICG (IR 820), squaraine dye, IR 780 dye, IR 193 dye, Epolight™ 1117 dye, iron oxide, iron oxide, zinc iron phosphate pigment, and combinations thereof.

Embodiment 289. The synergistic combination therapy of any one of the embodiments 265-288, wherein the carrier comprises a biocompatible substance selected from the group of a lipid, an inorganic polymer, an organic polymer, and combinations thereof.

Embodiment 290. The synergistic combination therapy of embodiment 289, wherein the carrier comprises an organic polymer.

Embodiment 291. The particle heater of embodiment 289, wherein the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polylysine; and combinations thereof.

Embodiment 292. The synergistic combination therapy of embodiment 265, wherein the carrier comprises a crosslinked biocompatible and biodegradable polymer.

Embodiment 293. The synergistic combination therapy of embodiment 289, wherein the crosslinked biocompatible polymer comprises a crosslinked polysaccharide.

Embodiment 294. The synergistic combination therapy of embodiment 293, wherein the polysaccharide is selected from chitosan, hyaluronic acid, alginate, alginic acid, starch, carrageenan, and combinations thereof.

Embodiment 295. The synergistic combination therapy of embodiment 289, wherein the carrier comprises an inorganic polymer.

Embodiment 296. The synergistic combination therapy of embodiment 295, wherein the inorganic material is selected from the group of mesoporous silica, organo-modified silicate polymer derived from condensation of organotrisilanol or halotrisilanol, and combinations thereof.

Embodiment 297. The synergistic combination therapy of embodiment 289, wherein the particle heater further has a thin film of noble metal on the particle surface, wherein the noble metal is selected from the group of gold, silver, copper, and combinations thereof.

Embodiment 298. The particle of embodiment 265, wherein the particle heater has a layer of iron oxide on the particle surface.

Embodiment 299. The synergistic combination therapy of embodiment 265, wherein the carrier is a lipid.

Embodiment 300. The synergistic combination therapy of embodiment 299, wherein the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-diarachidyl-sn-glycero-3-phosphoethanolamine (20:0 PE), 1-stearoyl-2-linoleoyl-sn-glycero-3-phosphoethanolamine, 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

Embodiment 301. The synergistic combination therapy of embodiment 289, wherein the lipid comprises a thermoresponsive lipid/polymer hybrid.

Embodiment 302. The synergistic combination therapy of embodiment 301, wherein the thermoresponsive lipid/polymer hybrid is selected from the group of a triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

Embodiment 303. The synergistic combination therapy of embodiments 265-302, wherein the particle heater further comprises a microbe-targeting group on the particle surface.

Embodiment 304. The synergistic combination therapy of embodiment 303, wherein the microbe-targeting group is selected from the group of antibody targeting the surface antigen of microbe, antibody targeting microbial Toll Like Receptor (TLR), cationic antimicrobial peptide, cell penetrating peptides including apidaecin, tat, buforin, magainin, and combinations thereof.

Embodiment 305. The synergistic combination therapy of embodiment 265, wherein the antimicrobial agent is selected from the group of antibiotics, antiseptic agents, cationic surfactants, biocides, and combinations thereof, (b) the material is an IR absorbing agent selected from the group of a indocyanine green (ICG), new ICG (IR 820), IR 780 dye, IR 193 dye, squaraine dye, Epolight™ 1117, Epolight™ 1175, iron oxide, and combinations thereof, (c) the carrier is selected from the group of poly(lactic acid) (PLA), poly(glycolic acid) (PGA), PLGA 75:25 (weight ratio of lactic acid:glycolic acid=75:25), PLGA 75:25-polyethylene glycol block copolymer (PLGA 75:25-b-PEG) (weight ratio of lactic acid:glycolic acid=75:25), blend of PLGA 75:25 with PLGA 75:25-b-PEG, and combinations thereof;

Embodiment 306. The synergistic combination therapy of embodiment 305, wherein the particle heater has a median particle size ranging from about 1 nm to 6 □m.

Embodiment 307. The synergistic combination therapy of embodiment 303, wherein the particle heater further comprises a microbe-targeting group selected from the group of antibody targeting the surface antigen of microbe, antibody targeting microbial Toll Like Receptor (TLR), cationic antimicrobial peptide, cell penetrating peptides including apidaecin, TAT ((GRKKRRQRRRPQ), buforin, magainin, RGD peptide, and combinations thereof.

Embodiment 308. A composition for use in remotely-triggered combination antimicrobial therapy comprising (a) a particle heater having a material interacting with an exogenous source and a carrier; and (b) a pharmaceutical dosage of an antimicrobial agent.

Embodiment 309. The composition of embodiment 308, wherein the particle heater and the pharmaceutical dosage forms a unitary dosage.

Embodiment 310. The composition of embodiment 308, wherein the particle heater and the pharmaceutical dosage are two discrete preparations.

Embodiment 311. The composition of any one of embodiments 308-310, wherein the pharmaceutical dosage is selected from the group of a capsule, a tablet, a buccal tablet, a sublingual tablet, an orally disintegrating tablet, a liquid formulation, a dispersion, an injection preparation, powder for injection, and suppository.

Embodiment 312. The composition of any one of embodiments 308-311, wherein the particle heaters are nanoparticles or microparticles.

Embodiment 313. The composition of embodiment 312, wherein the particle heater is further combined with a pharmaceutically acceptable excipient to form a particle heater preparation.

Embodiment 314. The composition of embodiment 313, wherein the particle heater preparation is selected from the group of a capsule, a tablet, a buccal tablet, a sublingual tablet, an orally disintegrating tablet, a liquid formulation, a dispersion, an injection preparation, powder for injection, and suppository.

Embodiment 315. A method for treating microbial infection with a synergistic combination therapy in a subject comprising: (1) administering a therapeutically effective amount of the synergistic combination therapy as disclosed herein to the subject in need thereof and allowing the synergistic combination therapy of embodiment 1 to associate with the microbes at the infection site, and (2) exposing the particle heaters to an exogenous source for a sufficient period of time, wherein the material absorbs the energy from the exogenous source and converts the energy into heat; and then the heat travels outside the particle to induce localized hyperthermia, wherein the localized hyperthermia and the antimicrobial agent exhibit synergy in killing microbes, and wherein the particle is constructed such that it passes the Extractable Cytotoxicity Test.

Embodiment 316. The method of embodiment 315, wherein the antimicrobial agent is further encapsulated by the particle heater, and wherein the heat causes the release of the antimicrobial agent outside of the particle.

Embodiment 317. The method of embodiment 315, wherein the particle heater and the anti-microbial agent are administered to the patient simultaneously.

Embodiment 318. The method embodiment 315, wherein the particle heater and the antimicrobial agent are administered to the patient sequentially.

Embodiment 319. The method of embodiment 315, wherein the antimicrobial agent is administered before administering of the particle heater.

Embodiment 320. The method of embodiment 315, wherein the particle heater is administered before administering the antimicrobial agent.

Embodiment 321. The method of embodiment 315, wherein the exogenous source is selected from an electromagnetic radiation, an electrical field, a microwave, a radio wave, an ultra-sound, a magnetic field, or combinations thereof.

Embodiment 322. The method of any one of the embodiments 315-321, wherein the exogenous source comprises a LED light or a laser light.

Embodiment 323. The method of embodiment 321, wherein the exogenous source comprises a LED light.

Embodiment 324. The method of embodiment 323, wherein the material absorbs optical energy at a wavelength from 400 nm to 750 nm.

Embodiment 325. The method of any one of embodiments 321-323, wherein the first material is a squaraine dye, or a squarylium dye.

Embodiment 326. The method of embodiment 322, wherein the laser light is a pulsed laser light.

Embodiment 327. The method of embodiment 326, wherein the laser pulse duration is in a range from milliseconds to microseconds, and the laser has an oscillation wavelength at 805 nm, 808 nm, or 1064 nm.

Embodiment 328. The method of any one of the embodiments 326-327, wherein the particle heater absorbs the laser light having a wavelength from 750 nm to 1100 nm.

Embodiment 329. The method of any one of the embodiments 326-328, wherein the particle heater comprises indocyanine green (ICG), new ICG (IR 820), IR 780 dye, IR 193 dye, squaraine dye, Epolight™ 1117, Epolight™ 1175, iron oxide, and combinations thereof.

Embodiment 330. The method of any one of the embodiments 325-327, wherein the particle heater comprises a zinc iron phosphate pigment.

Embodiment 331. The method of any one of the embodiments 326-330, wherein the induced hyperthermia is mild hyperthermia at a temperature ranging from about 38.0° C. to about 41.0° C.

Embodiment 332. The method of any one of the embodiments 326-330, wherein the induced hyperthermia is moderate hyperthermia at a temperature ranging from about 41.1° C. to about 45.0° C., wherein the hyperthermia does not cause collateral damage to healthy cells.

Embodiment 333. The method of any one of the embodiments 326-330, wherein the induced hyperthermia is profound hyperthermia at a temperature ranging from about 45.1° C. to about 52.0° C., wherein the hyperthermia does not cause collateral damage to healthy cells.

Embodiment 334. The method of any one of the embodiments 326-330, wherein the infection is caused by the multidrug resistant bacteria selected from the group of Gram-positive bacteria, Gram-negative bacteria, and combinations thereof.

Embodiment 335. The method of any one of the embodiments 326-330, wherein the infection is caused by the multidrug resistant bacteria selected from the group of E. coli, K. pneumonia, M. tuberculosis, Streptococcus aureus, P. aeruginosa, Streptococcus epidermidis, Streptococcus haemolyticus, Bacillus anthracia, Clostridium difficile, Streptococcus pyogenes, Streptococcus pneumonia, Enterococcus faecalis, and combinations thereof.

Embodiment 336. A method of treating a microbial infection in a subject in need thereof comprising the step of sensitizing the microbes by administering to the subject a treatment that will (i) induce apoptosis or autolysis in microbes, (ii) inducing the generation of reactive oxygen species, (iii) stimulate microbe-infiltrating immune cells, or (iv) a combination of two or more thereof.

Embodiment 337. The method of embodiment 336, wherein the treatment is the synergistic combination comprising a particle heater and an antimicrobial agent, wherein the particle comprises (a) a material interacting with an exogenous source, and (b) a carrier; wherein the particle is constructed such that it passes the Extractable Cytotoxicity Test; wherein the material absorbs the energy from the exogenous source and converts the energy into heat; then the heat travels outside the particle to induce localized hyperthermia, wherein the hyperthermia and the antimicrobial agent exhibit synergy in killing microbes.

Embodiment 338. The method of embodiment 337, wherein the antimicrobial agent is encapsulated in the particle heater and the heat causes the release of the antimicrobial agent.

Embodiment 339. The method of embodiment 337, wherein the antimicrobial agent is not encapsulated in the particle heater.

Embodiment 340. The method of embodiment 337, wherein the antimicrobial agent is present in a separate pharmaceutical composition from the particle heater.

Embodiment 341. The method of embodiment 340, wherein the particle heater is administered before the administration of the antimicrobial agent.

Embodiment 342. The method of embodiment 340, wherein the particle heater is administered after the administration of the antimicrobial agent.

Embodiment 343. The method of embodiment 340, wherein the particle heater is administered concurrently with the administration of the antimicrobial agent.

Embodiment 344. The method of embodiment 337, the method further comprising the step of exposing the particle heater remotely to an exogenous source, wherein the exogenous source is selected from the group of an electromagnetic radiation, an electrical field, a microwave, a radio wave, an ultrasound, a magnetic field, and combinations thereof.

Embodiment 345. The method of embodiment 337, wherein sensitizing the infection comprises administering to the subject a treatment that will induce apoptosis or autolysis in the pathogenic microbes.

Embodiment 346. The method of embodiment 345, wherein the treatment that will induce apoptosis or autolysis in the pathogenic microbes is selected from the group of thermal therapy, antibiotic, immunotherapy, phototherapy, and combinations thereof.

Embodiment 347. The method of embodiment 345, wherein the treatment that will induce apoptosis or autolysis in pathogenic microbial cells is thermal therapy.

Embodiment 348. The method of embodiment 345, wherein the treatment that will induce apoptosis or autolysis in pathogenic microbial cells is thermal therapy and the antibiotic.

Embodiment 349. A particle heater comprising a carrier admixed with a material that inter-acts with an exogenous source; wherein the material absorbs and converts the energy from the exogenous source into heat, and the heat travels outside the particle heater to induce localized hyperthermia at a temperature sufficient to selectively kill unwanted cells, and further wherein the particle heater structure is constructed such that it passes the Extractable Cytotoxicity Test.

Embodiment 350. The particle heater of embodiment 349, wherein particle heater further passes the Efficacy Determination Protocol.

Embodiment 351. The particle heater of any one of embodiments 349-350, wherein particle heater further passes the Thermal Cytotoxicity Test.

Embodiment 352. The particle heater of any one of embodiments 349-351, the material exhibits at least 20% efficiency of conversion of the energy from the exogenous source to heat (energy-to-heat conversion efficiency).

Embodiment 353. The particle heater of any one of embodiments 349-351, the material exhibits at least 20% photothermal conversion efficiency.

Embodiment 354. The particle heater of any one of the embodiments 349-350, wherein the exogenous source is selected from the group of an electromagnetic radiation, an electrical field, a microwave, a radio wave, an ultrasound, a magnetic field, and combinations thereof.

Embodiment 355. The particle heater of any one of the embodiments 349-351, wherein the particle heater has a median particle size ranging from about 1 nm to about 250 nm.

Embodiment 356. The particle heater of embodiment 352, wherein the particle heater has a median particle size ranging from about 1 nm to about 50 nm.

Embodiment 357. The particle heater of any one of the embodiments 349-354, wherein the particle heater maintains integrity or the particle structure is altered after interacting with the exogenous source.

Embodiment 358. The particle heater of any one of the embodiments 349-357, wherein the particle heater has a core-shell structure.

Embodiment 359. The particle heater of embodiment 358, wherein the core comprises a plasmonic absorber or iron oxide nanoparticles.

Embodiment 360. The particle heater of embodiment 358, wherein the shell comprises a plasmonic absorber or iron oxide.

Embodiment 361. The particle heater of any one of embodiments 359-360, wherein the plasmonic absorber comprises plasmonic nanomaterials of noble metal including gold (Au) nanostructure, silver (Ag) nanoparticle, and copper (Cu) nanoparticle having a plasmonic resonance at a NIR wavelength.

Embodiment 362. The particle heater of embodiment 358, the shell comprises an agent selected from the group of gold nanostructures, silver nanoparticles, iron oxide film, iron oxide nanoparticle, and combinations thereof.

Embodiment 363. The particle heater of any one of embodiments 358-362, wherein the shell comprise an agent selected from the group of inorganic polymers, silicates, organosilicate, organo-modified silicone polymers derived from condensation of organotrisilanol or halotrisilanol, cross-linked organic polymers, and combinations thereof.

Embodiment 364. The particle heater of any one of the embodiments 349-362, wherein the material has significant absorption of photonic energy in the near infrared spectrum region having a wavelength range from 750 nm to 1100 nm.

Embodiment 365. The particle heater of any one of the embodiments 349-362, wherein the material interacting with the exogenous source has significant absorption of photonic energy in the visible range.

Embodiment 366. The particle heater of embodiment 365, wherein the material absorbs light at a wavelength selected from the group of 400 nm, 410 nm, 420 nm, 430 nm, 440 nm, 450 nm, 460 nm, 470 nm, 480 nm, 490 nm, 500 nm, 510 nm, 520 nm, 530 nm, 540 nm, 550 nm, 560 nm, 570 nm, 580 nm, 590 nm, 600 nm, 610 nm, 620 nm, 630 nm, 640 nm, 650 nm, 660 nm, 670 nm, 680 nm, 690 nm, 700 nm, 710 nm, 720 nm, 730 nm, 740 nm, and 750 nm.

Embodiment 367. The particle heater of any one of the embodiments 349-362, wherein the material is selected from the group of a tetrakis aminium dye, a cyanine dye, a squaraine dye, a squarylium dye, iron oxide, a plasmonic absorber, a zinc iron phosphate pigment, and combinations thereof.

Embodiment 368. The particle heater of any one of the embodiments 349-367, wherein the carrier is selected from the group of a lipid, an inorganic agent, an organic polymer, and combinations thereof.

Embodiment 369. The particle heater of embodiment 368, wherein the carrier is selected from the group of poly (lactic acid) (PLA); poly(glycolic acid) (PGA); poly(lactide-co-glycolide) (PLGA); block copolymer of polyethylene glycol-b-poly lactic acid-co-glycolic acid (PEG-PLGA); polycaprolactone (PCL); poly-L-lysine (PLL); random graft co-polymer with a poly(L-lysine) backbone and poly(ethylene glycol) (PLL-g-PEG); dendritic polymer including polyethyleneimine (PEI) and derivatives thereof, dendritic polyglycerol and derivatives thereof, dendritic polylysine, and combinations thereof.

Embodiment 370. The particle heater of any one of the embodiments 366 and 367, wherein the carrier is selected from the group of PLA, PGA, PLGA, PCL, PLL, PLGA-PEG, a poly-mer blend containing PLGA 75:25 and PLGA-PEG 75:25 with lactide:glycolide monomer ratio of 75:25, and combinations thereof.

Embodiment 371. The particle heater of embodiment 349, wherein the carrier comprises a crosslinked biocompatible or biodegradable polymer.

Embodiment 372. The particle heater of embodiment 371, wherein the crosslinked biocompatible polymer comprises a crosslinked polysaccharide.

Embodiment 373. The particle heater of embodiment 372, wherein the polysaccharide is selected from hyaluronic acid, alginate, alginic acid, starch, carrageenan, and combinations thereof.

Embodiment 374. The particle heater of embodiment 349, wherein the carrier comprises an inorganic polymer.

Embodiment 375. The particle heater of embodiment 374, wherein the inorganic polymer is selected from the group of mesoporous silica, organo-modified silicate polymer derived from condensation of organotrisilanol or halotrisilanol, and combinations thereof.

Embodiment 376. The particle heater of embodiment 368, wherein the carrier is a lipid.

Embodiment 377. The particle heater of embodiment 376, wherein the lipid is selected from the group of 1,2-dipalmitoyl-sn-glycero-3-phospho-(1′-rac-glycerol) (DPPG), 1,2-distearoyl-sn-glycero-3-phosphoglycerol, sodium salt (DSPG), 1,2-dimyristoyl-sn-glycero-3-phospho-L-serine sodium salt (DMPS, 14:0 PS), 1,2-dipalmitoyl-sn-glycero-3-phosphoserine, sodium salt (DPPS, 16:0 PS), 1,2-distearoyl-sn-glycero-3-phospho-L-serine (sodium salt) (DSPS, 18:0 PS), 1,2-dimyristoyl-sn-glycero-3-phosphate, sodium salt (DMPA, 14:0 PA), 1,2-dipalmitoyl-sn-glycero-3-phosphate, sodium salt (DPPA, 16:0 PA), 1,2-distearoyl-sn-glycero-3-phosphate, sodium salt (DSPA, 18:0), 1′,3′-bis[1,2-dipalmitoyl-sn-glycero-3-phospho]-glycerol sodium salt (16:0 cardiolipin), 1,2-dimyristoyl-sn-glycero-3-phosphoethanolamine (DMPE, 12:0 PE), 1,2-dipalmitoyl-sn-glycero-3-phosphoethanolamine (DPPE, 16:0), 1,2-distearoyl-sn-glycero-3-phosphoethanolamine (DSPE, 18:0), 1,2-diarachidyl-sn-glycero-3-phosphoethanolamine (20:0 PE), 1-stearoyl-2-linoleoyl-sn-glycero-3-phosphoethanolamine, 1,2-dipalmitoyl-sn-glycero-3-phosphocholine (DPPC, 16:0 PC), 1,2-diheptadecanoyl-sn-glycero-3-phosphocholine (17:0 PC), 1,2-distearoyl-sn-glycero-3-phosphocholine (DSPC, 18:0 PC), 1,2-dinonadecanoyl-sn-glycero-3-phosphocholine (19:0 PC), 1,2-diarachidoyl-sn-glycero-3-phosphocholine (20:0 PC), 1,2-diheneicosanoyl-sn-glycero-3-phosphocholine (21:0 PC), 1,2-dibehenoyl-sn-glycero-3-phosphocholine (22:0 PC), 1,2-ditricosanoyl-sn-glycero-3-phosphocholine (23:0 PC), 1,2-dilignoceroyl-sn-glycero-3-phosphocholine (24:0 PC), 1-myristoyl-2-stearoyl-sn-glycero-3-phosphocholine (14:0-18:0 PC), 1-stearoyl-2-palmitoyl-sn-glycero-3-phosphocholine (16:0-18:0 PC), and combinations thereof.

Embodiment 378. The particle heater of embodiment 376, wherein the lipid comprises a thermoresponsive lipid/polymer hybrid.

Embodiment 379. The particle heater of embodiment 378, wherein the thermoresponsive lipid/polymer hybrid is selected from the group of triblock copolymer of [poly(2-isopropyl-2-oxazoline)-b-poly(dimethylsiloxane)-b-poly(2-isopropyl-2-oxazoline] and 1,2-dimyristoyl-sn-glycero-3-phosphocholine (DMPC) composite, block copolymers poly(cholesteryl acrylate)-b-poly(N-isopropylacrylamide) (PNIPAAm) and lipid composite, and combinations thereof.

Embodiment 380. The particle heater of embodiment 349, wherein the particle further comprises an active agent.

Embodiment 381. The particle heater of embodiment 377, wherein the active agent is selected from the group of agents capable of generating reactive oxygen species, therapeutic drugs, antimicrobial agent, anti-cancer agent, anti-scarring agent, anti-inflammatory agent, metallo-protease inhibitors, treatment sensitizing the unwanted cells to remotely triggered thermal therapy, and combinations thereof.

Embodiment 382. A method for inducing localized hyperthermia at a tissue site in a subject comprising: administering an effective amount of the particle heater of the embodiment 349 to the tissue site in the subject; exposing the particle to an exogenous source that heats the particle heater for a sufficient period of time to induce localized hyperthermia at a temperature ranging from about 38.0° C. to about 52.0° C.

Embodiment 383. The method of embodiment 382, wherein the exogenous source is selected from an electromagnetic radiation, an electrical field, a microwave, a radio wave, an ultra-sound, a magnetic field, or combinations thereof.

Embodiment 384. The method of any one of the embodiments 382 and 383, wherein the exogenous source comprises a LED light or a laser light.

Embodiment 385. The method of embodiment 384, wherein the laser light is a pulsed laser light.

Embodiment 386. The method of embodiment 384, wherein the exogenous source comprises a LED light.

Embodiment 387. The method of embodiment 385, wherein the laser pulse duration is in a range from milliseconds to nanoseconds, and the laser has an oscillation wavelength at 805 nm, 808 nm, or 1064 nm.

Embodiment 388. The method of embodiment 384, wherein the particle heater absorbs the visible light having a wavelength ranging from 400 nm to 750 nm.

Embodiment 389. The method of embodiment 382, wherein the particle heater absorbs the laser light having a wavelength ranging from 750 nm to 1400 nm.

Embodiment 390. The method of any one of embodiments 382-389, wherein the material is a tetrakis aminium dye.

Embodiment 391. The method of any one of embodiments 382-389, wherein the material is indocyanine green.

Embodiment 392. The method of any one of embodiments 382-389, wherein the material is a squaraine dye.

Embodiment 393. The method of any one of embodiments 382-389, wherein the material is a squarylium dye.

Embodiment 394. The method of any one of embodiments 382-389, wherein the material is iron oxide.

Embodiment 395. The method of any one of embodiments 382-389, wherein the material is a plasmonic absorber.

Embodiment 396. The method of embodiment 395, wherein the material is a plasmonic absorber is selected from the group of gold nanostructures including gold nanorod, gold nanocage, gold nanosphere, gold thin film, silver nanoparticle, and combinations thereof.

Embodiment 397. The method of any one of the embodiments 382-396, the method further comprising heating a surrounding area in proximity to the particle heater by transferring heat from the particle heater to the surrounding area to induce localized hyperthermia.

Embodiment 398. The method of any one of the embodiments 382-396, wherein the induced hyperthermia is mild hyperthermia at a temperature ranging from about 38.0° C. to about 41.0° C.

Embodiment 399. The method of any one of the embodiments 382-396, wherein the induced hyperthermia is moderate hyperthermia at a temperature ranging from about 41.1° C. to about 45.0° C., wherein the hyperthermia does not cause collateral damage to healthy cells.

Embodiment 400. The method of any one of the embodiments 382-396, wherein the induced hyperthermia is profound hyperthermia at a temperature ranging from about 45.1° C. to about 52.0° C.

Although the various embodiments of the invention have been described and illustrated with a certain degree of particularity, it is understood that the present disclosure has been made only by way of example, and that numerous changes in the combination and various arrangement of parts, features and components can be resorted to by those skilled in the art without departing from the scope of the invention, as hereinafter claimed.

We claim:

1. A particle for use in treating a cancer comprising:
(a) an anticancer agent,
(b) a carrier,
(c) a material that interacts with an exogenous source,
wherein the anticancer agent is encapsulated by the carrier,
wherein the anticancer agent and the material in the particle exhibit stability such that the particle is considered passing the Efficacy Determination Protocol; wherein the particle structure is constructed such that it passes the Extractable Cytotoxicity Test; wherein the material absorbs the energy from the exogenous source and converts the energy into heat; and then the anticancer agent is released outside the particle.

2. The particle of claim 1, wherein the carrier comprises a polymer having labile bonds susceptible to hydrolysis.

3. The particle of claim 2, wherein the hydrolytic degradation of the carrier is accelerated by the heat.

4. The particle of claim 1, wherein the unencapsulated anticancer agent has a plasma half-life of less than 30 minutes.

5. The particle of claim 2, wherein the anticancer agent is a Class II, Class III or Class IV compound according to the Biopharmaceutics Classification System.

6. The particle of any one of claims 1-3, wherein the anticancer agent is selected from the group of bis[(4-fluorophenyl)methyl] trisulfide (fluorapacin), 5-ethynylpyrimidine-2,4(1H,3H)-dione (eniluracil), saracatinib (azd0530), cisplatin, docetaxel, carboplatin, doxorubicin, etoposide, paclitaxel (taxol), silmitasertib (cx-4945), lenvatinib, irofulven, oxaliplatin, tesetaxel, intoplicine, apomine, cafusertib hydrochloride, ixazomib, alisertib, itraconazole, tafetinib, briciclib, cytarabine, panulisib, picoplatin, chlorogenic acid, pirotinib (kbp-5209), ganetespib (sta 9090), elesclomol sodium, amblyomin-x, irinotecan, darinaparsin, indibulin, tris-palifosfamide, curcumin, XL-418, everolimus, bortexomib, gefitinib, erlotinib, lapatinib, afuresertib, atamestane, azacitidine, brivanib alaninate, buparlisib, cabazitaxel, capecitabine, crizotinib, dabrafenib, dasatinib, N1,N11-bis(ethyl)norspermine (BENSM), ibrutinib, idelalisib, lenalidomide, pomalidomide, mitoxantrone, momelotinib, motesanib, napabucasin, naquotinib, sorafenib, pazopanib, pemetrexed, pimasertib, caricotamide, refametinib, egorafenib, ridaforolimus, rociletinib, sunitinib, talabostat, talimogene laherparepvec, tecemotide, temozolomide, therasphere, tosedostat, vandetanib, vorinostat, lipotecan, GSK-461364, and combinations thereof.

7. The particle of any one of claims 1-3, wherein the anticancer agent is a PI3K inhibitor selected from the group of wortmannin, temsirolimus, everolimus, buparlisib (BMK-120), 5-(2,6-dimorpholinopyrimidin-4-yl)-4-(trifluoromethyl)pyridin-2-amine), pictilisib, gedatolisib, apitolisib, pilaralisib, copanli sib, alpelisib, taselisib, PX-866 ((1E,4S,4aR,5R,6aS,9aR)-5-(acetyloxy)-1-[(di-2-propen-1-ylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione), LY294002 (2-Morpholin-4-yl-8-phenylchromen-4-one), dactolisib (2-Methyl-2-{4-[3-methyl-2-oxo-8-(quinolin-3-yl)-2,3-dihydro-1H-imidazo[4,5-c]quinolin-1-yl]phenyl}propanenitrile), omipalisib (2,4-difluoro-N-(2-methoxy-5-(4-(pyridazin-4-yl)quinolin-6-yl)pyridin-3-yl)benzenesulfonamide), bimiralisib (5-(4,6-dimorpholin-4-yl-1,3,5-triazin-2-yl)-4-(trifluoromethyl)pyridin-2-amine), serabelisib (5-(4-amino-1-propan-2-ylpyrazolo[3,4-d]pyrimidin-3-yl)-1,3-benzoxazol-2-amine), GSK2636771 (2-methyl-1-(2-methyl-3-(trifluoromethyl)benzyl)-6-morpholino-1H-benzo[d]imidazole-4-carboxylic acid), AZD8186 (8-[(1R)-1-(3,5-difluoroanilino)ethyl]-N,N-dimethyl-2-morpholin-4-yl-4-oxochromene-6-carboxamide), SAR260301 (2-[2-[(2S)-2,3-dihydro-2-methyl-1H-indol-1-yl]-2-oxoethyl]-6-(4-morpholinyl)-4(3H)-pyrimidinone), IPI-549 ((S)-2-amino-N-(1-(8-((1-methyl-1H-pyrazol-4-yl)ethynyl)-1-oxo-2-phenyl-1,2-dihydroisoquinolin-3-yl)ethyl)pyrazolo[1,5-a]pyrimidine-3-carboxamide) and combinations thereof.

8. The particle of any one of claims 1-3, wherein the anticancer agent is a proteasome inhibitor selected from the group of bortezomib, ixazomib, marizomib, oprozomib, delanzomib, epoxomicin, disulfiram, lactacystin, beta-hydroxy beta-methylbutyrate, and combinations thereof.

9. The particle of any one of claims 1-3, wherein the anticancer agent is an EGFR inhibitor selected from the group of erlotinib, gefitinib, neratinib, osimertinib, vandetanib, dacomitinib, lapatinib, and combinations thereof.

10. The particle of any one of claims 1-7, wherein the carrier comprises polymer with heat-labile moieties, or polymer having labile bonds susceptible hydrolysis, or enzymatic degradation.

11. The particle of claim 8, wherein the labile bonds are selected from the group of an ester bond, an amide bond, an anhydride bond, an acetal bond, a ketal bond, and combinations thereof.

12. The particle of any one of claims 1-9, wherein the carrier is selected from the group of a polyester, a polyanhydride, a polysaccharide, a polyphosphoester, a poly(ortho ester), a poly(amino acid), a protein, polyurea, and combinations thereof.

13. The particle of claim 10, wherein the polymer selected from the group of poly(lactic acid) (PLA), poly(glycolic acid) (PGA), PLGA, poly(lactic acid)-polyethylene glycol-poly(lactic acid) (PLA-PEG-PLA), poly (L-co-D,L lactic acid) 70:30 (PLDLA); poly-L-lactic acid-co-glycolic acid, poly-D,L-lactic acid-co-glycol acid; poly-valerolacton, poly-hydroxy butyrate and poly-hydroxy valerate, polycaprolactone (PCL), γ-polyglutamic acid graft with poly (L-phenylalanine) (γ-PGA-g-L-PAE), poly(cyanoacrylate) (PCA), polydioxanone, poly(butylene succinate), poly(trimethylene carbonate), poly(p-dioxanone), poly(buthylene terephthalate), poly(β-hydroxyalkanoate)s, poly(hydroxybutyrate), and poly(hydroxybuthyrate-co-hydroxyvalerate), poly (ε-lysine), diblock copolymer of poly(sebacic acid) and polyethylene glycol (PSA-PEG), trimethylene carbonate, poly(β-hydroxybutyrate), poly(g-ethyl glutamate), poly(DTH iminocarbonate), poly(bisphenol A iminocarbonate), polyphosphazene, collagen, albumin, gluten, chitosan, hyaluronate, hyaluronic acid, cellulose, alginate, starch, gelatin, pectin, and combinations thereof.

14. The particle of claim 10, wherein the polymer comprises a mixture of (i) a first PLGA having number average molecular weight ranging from 2000 Da to 3000 Da, and (ii) a second PLGA having number average molecular weight ranging from 570 Da to 1667 Da.

15. The particle of claim 14, wherein the first and second PLGA have a lactide:glycolide molar ratio ranging from 5:95 to 95:5, 10:90 to 90:10, 15:85 to 85:15, 25:75 to 75:25, 40:60 to 60:40, or 45:55 to 55:45.

16. The particle of claim 14, wherein the polymer comprises the first PLGA and the second PLGA in a weight ratio of first PLGA to second PLGA ranging from 10:1 to 1:10.

17. The particle of claim 10, wherein the polymer comprises a PLGA having a lactide:glycolide molar ratio ranging from 5:95 to 95:5, 10:90 to 90:10, 15:85 to 85:15, 25:75 to 75:25, 40:60 to 60:40, or 45:55 to 55:45 and having number average molecular weight ranging from 570 Da to 3000 Da.

18. The particle of any one of claims 9-15, wherein the anticancer agent is present in an amount ranging from about 1 wt. % to about 99 wt. % by the total weight of the particle.

19. The particles of any one of claims 9-15, wherein the anticancer agent has a weight ratio to the polymer ranging from about 1:99 to about 99:1, or from about 5:95 to about 95:5.

20. The particle of claim 1, wherein the material does not have significant optical absorption in the visible spectrum region.
